SEQUENCE LISTING (551 sequences)

SEQ ID NO: 1 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 2 - protein, 1097 aa, synthetic construct
MKIEEGKGHHHHHHMSNKEKNASETRKAYTTKMIPRSHDRMKLLGNFMDYLMDGTPIFFELWNQFGGGIDRDIISGTANKDKISDDLLLAVNWFKVMPINSKPQGVSPSNLANLFQQYSGSEPDIQAQEYFASNFDTEKHQWKDMRVEYERLLAELQLSRSDMHHDLKLMYKEKCIGLSLSTAHYITSVMFGTGAKNNRQTKHQFYSKVIQLLEESTQINSVEQLASIILKAGDCDSYRKLRIRCSRKGATPSILKIVQDYELGTNHDDEVNVPSLIANLKEKLGRFEYECEWKCMEKIKAFLASKVGPYYLGSYSAMLENALSPIKGMTTKNCKFVLKQIDAKNDIKYENEPFGKIVEGFFDSPYFESDTNVKWVLHPHHIGESNIKTLWEDLNAIHSKYEEDIASLSEDKKEKRIKVYQGDVCQTINTYCEEVGKEAKTPLVQLLRYLYSRKDDIAVDKIIDGITFLSKKHKVEKQKINPVIQKYPSFNFGNNSKLLGKIISPKDKLKHNLKCNRNQVDNYIWIEIKVLNTKTMRWEKHHYALSSTRFLEEVYYPATSENPPDALAARFRTKTNGYEGKPALSAEQIEQIRSAPVGLRKVKKRQMRLEAARQQNLLPRYTWGKDFNINICKRGNNFEVTLATKVKKKKEKNYKVVLGYDANIVRKNTYAAIEAHANGDGVIDYNDLPVKPIESGFVTVESQVRDKSYDQLSYNGVKLLYCKPHVESRRSFLEKYRNGTMKDNRGNNIQIDFMKDFEAIADDETSLYYFNMKYCKLLQSSIRNHSSQAKEYREEIFELLRDGKLSVLKLSSLSNLSFVMFKVAKSLIGTYFGHLLKKPKNSKSDVKAPPITDEDKQKADPEMFALRLALEEKRLNKVKSKKEVIANKIVAKALELRDKYGPVLIKGENISDTTKKGKKSSTNSFLMDWLARGVANKVKEMVMMHQGLEFVEVNPNFTSHQDPFVHKNPENTFRARYSRCTPSELTEKNRKEILSFLSDKPSKRPTNAYYNEGAMAFLATYGLKKNDVLGVSLEKFKQIMANILHQRSEDQLLFPSRGGMFYLATYKLDADATSVNWNGKQFWVCNADLVAAYNVGL

SEQ ID NO: 3 - DNA, 20 bp, synthetic construct
  source 1..20: mol_type = other DNA
agcacccagtccgccctgag

SEQ ID NO: 4 - DNA, 20 bp, synthetic construct
  source 1..20: mol_type = other DNA
ctcagggcggactgggtgct

SEQ ID NO: 5 - RNA, 44 nt, synthetic construct
  source 1..44: mol_type = other RNA
aatttttgtgcccatcgttggcacctcagggcggactgggtgct

SEQ ID NO: 6 - RNA, 39 nt, synthetic construct
  source 1..39: mol_type = other RNA
ttgtgcccatcgttggcacctcagggcggactgggtgct

SEQ ID NO: 7 - RNA, 34 nt, synthetic construct
  source 1..34: mol_type = other RNA
cccatcgttggcacctcagggcggactgggtgct

SEQ ID NO: 8 - RNA, 30 nt, synthetic construct
  source 1..30: mol_type = other RNA
tcgttggcacctcagggcggactgggtgct

SEQ ID NO: 9 - RNA, 20 nt, synthetic construct
  source 1..20: mol_type = other RNA
ctcagggcggactgggtgct

SEQ ID NO: 10 - DNA, 20 bp, synthetic construct
  source 1..20: mol_type = other DNA
acagcccatcgactggtgtt

SEQ ID NO: 11 - DNA, 20 bp, synthetic construct
  source 1..20: mol_type = other DNA
tatttagtgtgataatggcg

SEQ ID NO: 12 - DNA, 20 bp, synthetic construct
  source 1..20: mol_type = other DNA
acccctatcacgattagcat

SEQ ID NO: 13 - RNA, 21 nt, synthetic construct
  source 1..21: mol_type = other RNA
caacaccagtcgatgggctgt

SEQ ID NO: 14 - RNA, 22 nt, synthetic construct
  source 1..22: mol_type = other RNA
aacgccattatcacactaaata

SEQ ID NO: 15 - RNA, 23 nt, synthetic construct
  source 1..23: mol_type = other RNA
ttaatgctaatcgtgataggggt

SEQ ID NO: 16 - DNA, 44 bp, synthetic construct
  source 1..44: mol_type = other DNA
acagcccatcgactggtgttaatttttgtgcccatcgttggcac

SEQ ID NO: 17 - DNA, 44 bp, synthetic construct
  source 1..44: mol_type = other DNA
aatttttgtgcccatcgttggcacacagcccatcgactggtgtt

SEQ ID NO: 18 - DNA, 68 bp, synthetic construct
  source 1..68: mol_type = other DNA
aatttttgtgcccatcgttggcacacagcccatcgactggtgttaatttttgtgcccatcgttggcac

SEQ ID NO: 19 - DNA, 41 bp, synthetic construct
  source 1..41: mol_type = other DNA
acagcccatcgactggtgtttagatgtgaatcatctttaat

SEQ ID NO: 20 - DNA, 41 bp, synthetic construct
  source 1..41: mol_type = other DNA
tatttagtgtgataatggcgtagatgtgaatcatctttaat

SEQ ID NO: 21 - DNA, 41 bp, synthetic construct
  source 1..41: mol_type = other DNA
acccctatcacgattagcattagatgtgaatcatctttaat

SEQ ID NO: 22 - DNA, 44 bp, synthetic construct
  source 1..44: mol_type = other DNA
tatttagtgtgataatggcgaatttttgtgcccatcgttggcac

SEQ ID NO: 23 - DNA, 44 bp, synthetic construct
  source 1..44: mol_type = other DNA
acccctatcacgattagcataatttttgtgcccatcgttggcac

SEQ ID NO: 24-26 - skipped (3 entries)

SEQ ID NO: 27 - DNA, 41 bp, synthetic construct
  source 1..41: mol_type = other DNA
agcacccagtccgccctgagtagatgtgaatcatctttaat

SEQ ID NO: 28 - DNA, 44 bp, synthetic construct
  source 1..44: mol_type = other DNA
agcacccagtccgccctgagaatttttgtgcccatcgttggcac

SEQ ID NO: 29 - RNA, 20 nt, synthetic construct
  source 1..20: mol_type = other RNA
ctcagggcggactgggtgct

SEQ ID NO: 30 - RNA, 41 nt, synthetic construct
  source 1..41: mol_type = other RNA
taatttctactaagtgtagatctcagggcggactgggtgct

SEQ ID NO: 31 - RNA, 44 nt, synthetic construct; identical to SEQ ID NO: 5
  source 1..44: mol_type = other RNA

SEQ ID NO: 32 - DNA, 20 bp, synthetic construct
  source 1..20: mol_type = other DNA
agcacccagtccgccctgag

SEQ ID NO: 33 - RNA, 41 nt, synthetic construct; identical to SEQ ID NO: 30
  source 1..41: mol_type = other RNA
  misc_feature 41: note = biotin tag

SEQ ID NO: 34 - RNA, 44 nt, synthetic construct; identical to SEQ ID NO: 5
  source 1..44: mol_type = other RNA
  misc_feature 44: note = biotin tag

SEQ ID NO: 35 - RNA, 20 nt, synthetic construct
  source 1..20: mol_type = other RNA
  misc_feature 20: note = biotin tag
ctcagggcggactgggtgct

SEQ ID NO: 36 - DNA, 20 bp, synthetic construct
  source 1..20: mol_type = other DNA
  misc_feature 20: note = biotin tag
agcacccagtccgccctgag

SEQ ID NO: 37 - DNA, 41 bp, synthetic construct
  source 1..41: mol_type = other DNA
  misc_feature 41: note = biotin tag
tagatgtgaatcatctttaatagcacccagtccgccctgag

SEQ ID NO: 38 - DNA, 41 bp, synthetic construct; identical to SEQ ID NO: 27
  source 1..41: mol_type = other DNA
  misc_feature 41: note = biotin tag

SEQ ID NO: 39 - DNA, 62 bp, synthetic construct
  source 1..62: mol_type = other DNA
  misc_feature 62: note = biotin tag
tagatgtgaatcatctttaatagcacccagtccgccctgagtagatgtgaatcatctttaat

SEQ ID NO: 40 - DNA, 44 bp, synthetic construct
  source 1..44: mol_type = other DNA
  misc_feature 44: note = biotin tag
aatttttgtgcccatcgttggcacagcacccagtccgccctgag

SEQ ID NO: 41 - DNA, 44 bp, synthetic construct; identical to SEQ ID NO: 28
  source 1..44: mol_type = other DNA
  misc_feature 44: note = biotin tag

SEQ ID NO: 42 - DNA, 68 bp, synthetic construct
  source 1..68: mol_type = other DNA
  misc_feature 68: note = biotin tag
aatttttgtgcccatcgttggcacagcacccagtccgccctgagaatttttgtgcccatcgttggcac

SEQ ID NO: 43 - RNA, 60 nt, synthetic construct
  source 1..60: mol_type = other RNA
  misc_feature 1: note = FAM fluorescent tag
  misc_feature 60: note = Cy5 fluorescent tag
agaccgtgcatcatcctaaacctcgtccgccctgaggaacgctgaagcgccttgtggtac

SEQ ID NO: 44 - DNA, 41 bp, synthetic construct
  source 1..41: mol_type = other DNA
tcagcgttcctcgggcggactagatgtgaatcatctttaat

SEQ ID NO: 45 - DNA, 44 bp, synthetic construct
  source 1..44: mol_type = other DNA
tcagcgttcctcgggcggacaatttttgtgcccatcgttggcac

SEQ ID NO: 46 - DNA, 35 bp, synthetic construct
  source 1..35: mol_type = other DNA
gaggtttaggaggctagctacaacgagatgcacgg

SEQ ID NO: 47 - DNA, 1767 bp, synthetic construct
  source 1..1767: mol_type = other DNA
taatacgactcactatagaaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccacagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaa

SEQ ID NO: 48 - DNA, 1724 bp, synthetic construct
  source 1..1724: mol_type = other DNA
taatacgactcactataggggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgg

SEQ ID NO: 49 - DNA, 1677 bp, synthetic construct
  source 1..1677: mol_type = other DNA
taatacgactcactatagagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggc

SEQ ID NO: 50 - DNA, 44 bp, synthetic construct
  source 1..44: mol_type = other DNA
ctggtctaaccagagagaccaatttttgtgcccatcgttggcac

SEQ ID NO: 51 - DNA, 44 bp, synthetic construct
  source 1..44: mol_type = other DNA
ttgaagcactcaaggcaagcaatttttgtgcccatcgttggcac

SEQ ID NO: 52 - DNA, 44 bp, synthetic construct
  source 1..44: mol_type = other DNA
tggcgtactcaccagtcgccaatttttgtgcccatcgttggcac

SEQ ID NO: 53 - DNA, 44 bp, synthetic construct
  source 1..44: mol_type = other DNA
tcccagtatttgtctacagcaatttttgtgcccatcgttggcac

SEQ ID NO: 54 - DNA, 44 bp, synthetic construct
  source 1..44: mol_type = other DNA
ctgacctgattgctgtgtccaatttttgtgcccatcgttggcac

SEQ ID NO: 55 - DNA, 44 bp, synthetic construct
  source 1..44: mol_type = other DNA
gcttcctcattgatggtctcaatttttgtgcccatcgttggcac

SEQ ID NO: 56 - DNA, 44 bp, synthetic construct
  source 1..44: mol_type = other DNA
gtccagaatgctggtagggcaatttttgtgcccatcgttggcac

SEQ ID NO: 57 - DNA, 44 bp, synthetic construct
  source 1..44: mol_type = other DNA
ggtcctcctactccctgacaaatttttgtgcccatcgttggcac

SEQ ID NO: 58 - DNA, 44 bp, synthetic construct
  source 1..44: mol_type = other DNA
attagcctgtctctcagtacaatttttgtgcccatcgttggcac

SEQ ID NO: 59 - DNA, 44 bp, synthetic construct
  source 1..44: mol_type = other DNA
ttgccaaagagtgacctgagaatttttgtgcccatcgttggcac

SEQ ID NO: 60 - DNA, 44 bp, synthetic construct
  source 1..44: mol_type = other DNA
ttgacaggtgtaggtcctacaatttttgtgcccatcgttggcac

SEQ ID NO: 61 - DNA, 44 bp, synthetic construct
  source 1..44: mol_type = other DNA
tcttctgtcaatggccattgaatttttgtgcccatcgttggcac

SEQ ID NO: 62 - DNA, 44 bp, synthetic construct
  source 1..44: mol_type = other DNA
cccagaagtcttgagttctcaatttttgtgcccatcgttggcac

SEQ ID NO: 63 - DNA, 44 bp, synthetic construct
  source 1..44: mol_type = other DNA
tggaatattgctggtgatccaatttttgtgcccatcgttggcac

SEQ ID NO: 64 - DNA, 44 bp, synthetic construct
  source 1..44: mol_type = other DNA
tggcagcactataggctgtaaatttttgtgcccatcgttggcac

SEQ ID NO: 65 - DNA, 44 bp, synthetic construct
  source 1..44: mol_type = other DNA
tgccagttctagctctgcttaatttttgtgcccatcgttggcac

SEQ ID NO: 66 - DNA, 44 bp, synthetic construct
  source 1..44: mol_type = other DNA
gtttccttttgtatgggcagaatttttgtgcccatcgttggcac

SEQ ID NO: 67 - DNA, 44 bp, synthetic construct
  source 1..44: mol_type = other DNA
ttagtctccctgttagctgcaatttttgtgcccatcgttggcac

SEQ ID NO: 68 - DNA, 44 bp, synthetic construct
  source 1..44: mol_type = other DNA
gtacccatgccagatagaccaatttttgtgcccatcgttggcac

SEQ ID NO: 69 - DNA, 44 bp, synthetic construct
  source 1..44: mol_type = other DNA
acagtctacttgtccatgcaaatttttgtgcccatcgttggcac

SEQ ID NO: 70 - DNA, 44 bp, synthetic construct
  source 1..44: mol_type = other DNA
tgctgccattgtcagtatgtaatttttgtgcccatcgttggcac

SEQ ID NO: 71 - DNA, 44 bp, synthetic construct
  source 1..44: mol_type = other DNA
ttctttcccctgcactgtacaatttttgtgcccatcgttggcac

SEQ ID NO: 72 - DNA, 44 bp, synthetic construct
  source 1..44: mol_type = other DNA
caatcatcacctgccatctgaatttttgtgcccatcgttggcac

SEQ ID NO: 73 - DNA, 44 bp, synthetic construct
  source 1..44: mol_type = other DNA
tgggatgtgtacttctgaacaatttttgtgcccatcgttggcac

SEQ ID NO: 74 - DNA, 44 bp, synthetic construct
  source 1..44: mol_type = other DNA
agtattgtagagatcctaccaatttttgtgcccatcgttggcac

SEQ ID NO: 75 - DNA, 44 bp, synthetic construct
  source 1..44: mol_type = other DNA
cagcttcattcttaagctccaatttttgtgcccatcgttggcac

SEQ ID NO: 76 - DNA, 44 bp, synthetic construct
  source 1..44: mol_type = other DNA
ttcctggatgcttccagggcaatttttgtgcccatcgttggcac

SEQ ID NO: 77 - DNA, 44 bp, synthetic construct
  source 1..44: mol_type = other DNA
atggaccacacaactattgcaatttttgtgcccatcgttggcac

SEQ ID NO: 78 - DNA, 44 bp, synthetic construct
  source 1..44: mol_type = other DNA
ctgtagcactacagatcatcaatttttgtgcccatcgttggcac

SEQ ID NO: 79 - DNA, 44 bp, synthetic construct
  source 1..44: mol_type = other DNA
gctttaggctttgatcccataatttttgtgcccatcgttggcac

SEQ ID NO: 80 - DNA, 44 bp, synthetic construct
  source 1..44: mol_type = other DNA
tgactgaggtgttacaacttaatttttgtgcccatcgttggcac

SEQ ID NO: 81 - DNA, 44 bp, synthetic construct
  source 1..44: mol_type = other DNA
tgagttgatactactggcctaatttttgtgcccatcgttggcac

SEQ ID NO: 82 - DNA, 44 bp, synthetic construct
  source 1..44: mol_type = other DNA
tgttacaatgtgcttgtctcaatttttgtgcccatcgttggcac

SEQ ID NO: 83 - DNA, 44 bp, synthetic construct
  source 1..44: mol_type = other DNA
gtgattgtgtcacttccttcaatttttgtgcccatcgttggcac

SEQ ID NO: 84 - DNA, 44 bp, synthetic construct
  source 1..44: mol_type = other DNA
tatctcctcctccaggtctgaatttttgtgcccatcgttggcac

SEQ ID NO: 85 - DNA, 44 bp, synthetic construct
  source 1..44: mol_type = other DNA
tcagcaaattgttctgctgcaatttttgtgcccatcgttggcac

SEQ ID NO: 86 - DNA, 44 bp, synthetic construct
  source 1..44: mol_type = other DNA
catccaggtcgtgtgattccaatttttgtgcccatcgttggcac

SEQ ID NO: 87 - DNA, 44 bp, synthetic construct
  source 1..44: mol_type = other DNA
atccctgcctaactctattcaatttttgtgcccatcgttggcac

SEQ ID NO: 88 - DNA, 44 bp, synthetic construct
  source 1..44: mol_type = other DNA
agtaagtctctcaagcggtgaatttttgtgcccatcgttggcac

SEQ ID NO: 89 - DNA, 44 bp, synthetic construct
  source 1..44: mol_type = other DNA
tattcttctaggtatgtggcaatttttgtgcccatcgttggcac

SEQ ID NO: 90 - DNA, 44 bp, synthetic construct
  source 1..44: mol_type = other DNA
ctcttgtgcttctagccaggaatttttgtgcccatcgttggcac

SEQ ID NO: 91 - DNA, 44 bp, synthetic construct
  source 1..44: mol_type = other DNA
caactggtactagcttgtagaatttttgtgcccatcgttggcac

SEQ ID NO: 92 - DNA, 44 bp, synthetic construct
  source 1..44: mol_type = other DNA
aagtcccttgtagcaagctcaatttttgtgcccatcgttggcac

SEQ ID NO: 93 - DNA, 44 bp, synthetic construct
  source 1..44: mol_type = other DNA
tatatgcaggatctgagggcaatttttgtgcccatcgttggcac

SEQ ID NO: 94 - DNA, 44 bp, synthetic construct
  source 1..44: mol_type = other DNA
gaagcactcaaggcaagcttaatttttgtgcccatcgttggcac

SEQ ID NO: 95 - DNA, 44 bp, synthetic construct
  source 1..44: mol_type = other DNA
atcccgaatcctgcaaagctaatttttgtgcccatcgttggcac

SEQ ID NO: 96 - DNA, 44 bp, synthetic construct
  source 1..44: mol_type = other DNA
tatatccactggctacatgaaatttttgtgcccatcgttggcac

SEQ ID NO: 97 - DNA, 44 bp, synthetic construct
  source 1..44: mol_type = other DNA
gtactgtccatttatcaggaaatttttgtgcccatcgttggcac

SEQ ID NO: 98 - DNA, 41 bp, synthetic construct
  source 1..41: mol_type = other DNA
ctggtctaaccagagagacctagatgtgaatcatctttaat

SEQ ID NO: 99 - DNA, 41 bp, synthetic construct
  source 1..41: mol_type = other DNA
ttgaagcactcaaggcaagctagatgtgaatcatctttaat

SEQ ID NO: 100 - DNA, 41 bp, synthetic construct
  source 1..41: mol_type = other DNA
tggcgtactcaccagtcgcctagatgtgaatcatctttaat

SEQ ID NO: 101 - DNA, 41 bp, synthetic construct
  source 1..41: mol_type = other DNA
tcccagtatttgtctacagctagatgtgaatcatctttaat

SEQ ID NO: 102 - DNA, 41 bp, synthetic construct
  source 1..41: mol_type = other DNA
ctgacctgattgctgtgtcctagatgtgaatcatctttaat

SEQ ID NO: 103 - DNA, 41 bp, synthetic construct
  source 1..41: mol_type = other DNA
gcttcctcattgatggtctctagatgtgaatcatctttaat

SEQ ID NO: 104 - DNA, 41 bp, synthetic construct
  source 1..41: mol_type = other DNA
gtccagaatgctggtagggctagatgtgaatcatctttaat

SEQ ID NO: 105 - DNA, 41 bp, synthetic construct
  source 1..41: mol_type = other DNA
ggtcctcctactccctgacatagatgtgaatcatctttaat

SEQ ID NO: 106 - DNA, 41 bp, synthetic construct
  source 1..41: mol_type = other DNA
attagcctgtctctcagtactagatgtgaatcatctttaat

SEQ ID NO: 107 - DNA, 41 bp, synthetic construct
  source 1..41: mol_type = other DNA
ttgccaaagagtgacctgagtagatgtgaatcatctttaat

SEQ ID NO: 108 - DNA, 41 bp, synthetic construct
  source 1..41: mol_type = other DNA
ttgacaggtgtaggtcctactagatgtgaatcatctttaat

SEQ ID NO: 109 - DNA, 41 bp, synthetic construct
  source 1..41: mol_type = other DNA
tcttctgtcaatggccattgtagatgtgaatcatctttaat

SEQ ID NO: 110 - DNA, 41 bp, synthetic construct
  source 1..41: mol_type = other DNA
cccagaagtcttgagttctctagatgtgaatcatctttaat

SEQ ID NO: 111 - DNA, 41 bp, synthetic construct
  source 1..41: mol_type = other DNA
tggaatattgctggtgatcctagatgtgaatcatctttaat

SEQ ID NO: 112 - DNA, 41 bp, synthetic construct
  source 1..41: mol_type = other DNA
tggcagcactataggctgtatagatgtgaatcatctttaat

SEQ ID NO: 113 - DNA, 41 bp, synthetic construct
  source 1..41: mol_type = other DNA
tgccagttctagctctgctttagatgtgaatcatctttaat

SEQ ID NO: 114 - DNA, 41 bp, synthetic construct
  source 1..41: mol_type = other DNA
gtttccttttgtatgggcagtagatgtgaatcatctttaat

SEQ ID NO: 115 - DNA, 41 bp, synthetic construct
  source 1..41: mol_type = other DNA
ttagtctccctgttagctgctagatgtgaatcatctttaat

SEQ ID NO: 116 - DNA, 41 bp, synthetic construct
  source 1..41: mol_type = other DNA
gtacccatgccagatagacctagatgtgaatcatctttaat

SEQ ID NO: 117 - DNA, 41 bp, synthetic construct
  source 1..41: mol_type = other DNA
acagtctacttgtccatgcatagatgtgaatcatctttaat

SEQ ID NO: 118 - DNA, 41 bp, synthetic construct
  source 1..41: mol_type = other DNA
tgctgccattgtcagtatgttagatgtgaatcatctttaat

SEQ ID NO: 119 - DNA, 41 bp, synthetic construct
  source 1..41: mol_type = other DNA
ttctttcccctgcactgtactagatgtgaatcatctttaat

SEQ ID NO: 120 - DNA, 41 bp, synthetic construct
  source 1..41: mol_type = other DNA
caatcatcacctgccatctgtagatgtgaatcatctttaat

SEQ ID NO: 121 - DNA, 41 bp, synthetic construct
  source 1..41: mol_type = other DNA
tgggatgtgtacttctgaactagatgtgaatcatctttaat

SEQ ID NO: 122 - DNA, 41 bp, synthetic construct
  source 1..41: mol_type = other DNA
agtattgtagagatcctacctagatgtgaatcatctttaat

SEQ ID NO: 123 - DNA, 41 bp, synthetic construct
  source 1..41: mol_type = other DNA
cagcttcattcttaagctcctagatgtgaatcatctttaat

SEQ ID NO: 124 - DNA, 41 bp, synthetic construct
  source 1..41: mol_type = other DNA
ttcctggatgcttccagggctagatgtgaatcatctttaat

SEQ ID NO: 125 - DNA, 41 bp, synthetic construct
  source 1..41: mol_type = other DNA
atggaccacacaactattgctagatgtgaatcatctttaat

SEQ ID NO: 126 - DNA, 41 bp, synthetic construct
  source 1..41: mol_type = other DNA
ctgtagcactacagatcatctagatgtgaatcatctttaat

SEQ ID NO: 127 - DNA, 41 bp, synthetic construct
  source 1..41: mol_type = other DNA
gctttaggctttgatcccattagatgtgaatcatctttaat

SEQ ID NO: 128 - DNA, 41 bp, synthetic construct
  source 1..41: mol_type = other DNA
tgactgaggtgttacaactttagatgtgaatcatctttaat

SEQ ID NO: 129 - DNA, 41 bp, synthetic construct
  source 1..41: mol_type = other DNA
tgagttgatactactggccttagatgtgaatcatctttaat

SEQ ID NO: 130 - DNA, 41 bp, synthetic construct
  source 1..41: mol_type = other DNA
tgttacaatgtgcttgtctctagatgtgaatcatctttaat

SEQ ID NO: 131 - DNA, 41 bp, synthetic construct
  source 1..41: mol_type = other DNA
gtgattgtgtcacttccttctagatgtgaatcatctttaat

SEQ ID NO: 132 - DNA, 41 bp, synthetic construct
  source 1..41: mol_type = other DNA
tatctcctcctccaggtctgtagatgtgaatcatctttaat

SEQ ID NO: 133 - DNA, 41 bp, synthetic construct
  source 1..41: mol_type = other DNA
tcagcaaattgttctgctgctagatgtgaatcatctttaat

SEQ ID NO: 134 - DNA, 41 bp, synthetic construct
  source 1..41: mol_type = other DNA
catccaggtcgtgtgattcctagatgtgaatcatctttaat

SEQ ID NO: 135 - DNA, 41 bp, synthetic construct
  source 1..41: mol_type = other DNA
atccctgcctaactctattctagatgtgaatcatctttaat

SEQ ID NO: 136 - DNA, 41 bp, synthetic construct
  source 1..41: mol_type = other DNA
agtaagtctctcaagcggtgtagatgtgaatcatctttaat

SEQ ID NO: 137 - DNA, 41 bp, synthetic construct
  source 1..41: mol_type = other DNA
tattcttctaggtatgtggctagatgtgaatcatctttaat

SEQ ID NO: 138 - DNA, 41 bp, synthetic construct
  source 1..41: mol_type = other DNA
ctcttgtgcttctagccaggtagatgtgaatcatctttaat

SEQ ID NO: 139 - DNA, 41 bp, synthetic construct
  source 1..41: mol_type = other DNA
caactggtactagcttgtagtagatgtgaatcatctttaat

SEQ ID NO: 140 - DNA, 41 bp, synthetic construct
  source 1..41: mol_type = other DNA
aagtcccttgtagcaagctctagatgtgaatcatctttaat

SEQ ID NO: 141 - DNA, 41 bp, synthetic construct
  source 1..41: mol_type = other DNA
tatatgcaggatctgagggctagatgtgaatcatctttaat

SEQ ID NO: 142 - DNA, 41 bp, synthetic construct
  source 1..41: mol_type = other DNA
gaagcactcaaggcaagctttagatgtgaatcatctttaat

SEQ ID NO: 143 - DNA, 41 bp, synthetic construct
  source 1..41: mol_type = other DNA
atcccgaatcctgcaaagcttagatgtgaatcatctttaat

SEQ ID NO: 144 - DNA, 41 bp, synthetic construct
  source 1..41: mol_type = other DNA
tatatccactggctacatgatagatgtgaatcatctttaat

SEQ ID NO: 145 - DNA, 41 bp, synthetic construct
  source 1..41: mol_type = other DNA
gtactgtccatttatcaggatagatgtgaatcatctttaat

SEQ ID NO: 146 - DNA, 22 bp, Francisella hispaniensis
  source 1..22: mol_type = genomic DNA
aaattatttaaagttcttagac

SEQ ID NO: 147 - DNA, 23 bp, Eubacterium eligens
  source 1..23: mol_type = genomic DNA
aaattatttaaggttattcaaac

SEQ ID NO: 148 - DNA, 22 bp, unidentified
  source 1..22: mol_type = genomic DNA
aaattgtgtaggtcttattgcg

SEQ ID NO: 149 - DNA, 21 bp, Moraxella bovoculi
  source 1..21: mol_type = genomic DNA
aaatttctactgtttgtagat

SEQ ID NO: 150 - DNA, 20 bp, Succinivibrio sp.
  source 1..20: mol_type = genomic DNA
aaatttctacttatgtagat

SEQ ID NO: 151 - DNA, 21 bp, unidentified
  source 1..21: mol_type = genomic DNA
aatcctataggtcgtttagag

SEQ ID NO: 152 - DNA, 21 bp, Prevotella bryantii
  source 1..21: mol_type = genomic DNA
aattaaataagctttatagcc

SEQ ID NO: 153 - DNA, 21 bp, Helcococcus kunzii
  source 1..21: mol_type = genomic DNA
aattaatagtatctcttaaag

SEQ ID NO: 154 - DNA, 21 bp, unidentified
  source 1..21: mol_type = genomic DNA
aattacaggctttatgtagcc

SEQ ID NO: 155 - DNA, 21 bp, unidentified
  source 1..21: mol_type = genomic DNA
aattataaaggcattatagcc

SEQ ID NO: 156 - DNA, 21 bp, Butyrivibrio hungatei
  source 1..21: mol_type = genomic DNA
aattatctttaagtcttagac

SEQ ID NO: 157 - DNA, 21 bp, Pseudobutyrivibrio xylanivorans
  source 1..21: mol_type = genomic DNA
aattatttaaaggttctaagc

SEQ ID NO: 158 - DNA, 21 bp, Francisella sp.
  source 1..21: mol_type = genomic DNA
aattatttaaagttcttagac

SEQ ID NO: 159 - DNA, 21 bp, unidentified
  source 1..21: mol_type = genomic DNA
aattctgaatgagttttagac

SEQ ID NO: 160 - DNA, 21 bp, Flavobacterium sp.
  source 1..21: mol_type = genomic DNA
aatttagtttgtctttaaaac

SEQ ID NO: 161 - DNA, 21 bp, unidentified
  source 1..21: mol_type = genomic DNA
aattttataaggcctttagac

SEQ ID NO: 162 - DNA, 21 bp, Sneathia sp.
  source 1..21: mol_type = genomic DNA
aattttatttgggttctaaac

SEQ ID NO: 163 - DNA, 20 bp, Alicyclobacillus acidoterrestris
  source 1..20: mol_type = genomic DNA
agcgatctgagaagtggcac

SEQ ID NO: 164 - DNA, 21 bp, unidentified
  source 1..21: mol_type = genomic DNA
gaatctctactctttgtagat

SEQ ID NO: 165 - DNA, 21 bp, Eubacterium rectale
  source 1..21: mol_type = genomic DNA
gaatgtctactggggtagatc

SEQ ID NO: 166 - DNA, 20 bp, Lachnospiraceae sp.
  source 1..20: mol_type = genomic DNA
gaatttctactagtgtagat

SEQ ID NO: 167 - DNA, 20 bp, Ruminococcus sp.
  source 1..20: mol_type = genomic DNA
gaatttctactattgtagat

SEQ ID NO: 168 - DNA, 22 bp, Moraxella bovoculi
  source 1..22: mol_type = genomic DNA
taaatttctactgtttgtagat

SEQ ID NO: 169 - DNA, 21 bp, Lachnospiraceae bacterium
  source 1..21: mol_type = genomic DNA
taatttctactaagtgtagat

SEQ ID NO: 170 - DNA, 20 bp, Prevotella bryantii
  source 1..20: mol_type = genomic DNA
taatttctactattgtagat

SEQ ID NO: 171 - DNA, 20 bp, Acidaminococcus sp.
  source 1..20: mol_type = genomic DNA
taatttctactcttgtagat

SEQ ID NO: 172 - DNA, 20 bp, Francisella tularensis
  source 1..20: mol_type = genomic DNA
taatttctactgttgtagat

SEQ ID NO: 173 - DNA, 19 bp, Lachnospira sp.
  source 1..19: mol_type = genomic DNA
taatttctactttgtagat

SEQ ID NO: 174 - DNA, 21 bp, synthetic construct
  source 1..21: mol_type = other DNA
tcacctacgtaaccgtgaact

SEQ ID NO: 175 - DNA, 20 bp, unidentified
  source 1..20: mol_type = genomic DNA
taatttctacttcggtagat

SEQ ID NO: 176 - DNA, 21 bp, unidentified
  source 1..21: mol_type = genomic DNA
taatttctactgtgtgtagat

SEQ ID NO: 177 - DNA, 22 bp, Methylobacterium nodulans
  source 1..22: mol_type = genomic DNA
aggatctggcgcccactgcgac

SEQ ID NO: 178 - DNA, 20 bp, unidentified
  source 1..20: mol_type = genomic DNA
cacatcgcgggaactgcggc

SEQ ID NO: 179 - DNA, 21 bp, Pelobacter propionicus
  source 1..21: mol_type = genomic DNA
ccccgcgcatgcggggaacac

SEQ ID NO: 180 - DNA, 20 bp, Bacillus sp.
  source 1..20: mol_type = genomic DNA
cctcgggtcctcaatgtaac

SEQ ID NO: 181 - DNA, 22 bp, Alicyclobacillus acidoterrestris
  source 1..22: mol_type = genomic DNA
cgagcgatctgagaagtggcac

SEQ ID NO: 182 - DNA, 21 bp, Brevibacillus agri
  source 1..21: mol_type = genomic DNA
ctttccactaagctttcgaac

SEQ ID NO: 183 - DNA, 21 bp, synthetic construct
  source 1..21: mol_type = other DNA
gaaaagctgagaagttagcac

SEQ ID NO: 184 - DNA, 21 bp, Sulfobacillus thermotolerans
  source 1..21: mol_type = genomic DNA
gaatgcttaggttgttggcac

SEQ ID NO: 185 - DNA, 20 bp, unidentified
  source 1..20: mol_type = genomic DNA
gccgtgttggccgatgcggc

SEQ ID NO: 186 - DNA, 22 bp, unidentified
  source 1..22: mol_type = genomic DNA
ggcctgcgccgacccgaaccgc

SEQ ID NO: 187 - DNA, 22 bp, unidentified
  source 1..22: mol_type = genomic DNA
gggccgcgtcggcctccgcggc

SEQ ID NO: 188 - DNA, 21 bp, unidentified
  source 1..21: mol_type = genomic DNA
tgaaacggcattcgctgcggc

SEQ ID NO: 189 - DNA, 18 bp, Sulfobacillus thermotolerans
  source 1..18: mol_type = genomic DNA
tgcttaggttgttggcac

SEQ ID NO: 190 - DNA, 22 bp, Brevibacillus sp.
  source 1..22: mol_type = genomic DNA
tggaaagcttcgggattagcac

SEQ ID NO: 191 - DNA, 20 bp, Brevibacillus sp.
  source 1..20: mol_type = genomic DNA
tggagtgcgtggattgaaac

SEQ ID NO: 192 - DNA, 25 bp, unidentified
  source 1..25: mol_type = genomic DNA
acccgtaaagcagagcgatgaaggc

SEQ ID NO: 193 - DNA, 24 bp, unidentified
  source 1..24: mol_type = genomic DNA
tatttatcggagatatcttcaaac

SEQ ID NO: 194 - DNA, 22 bp, unidentified
  source 1..22: mol_type = genomic DNA
cgcgcccctgatgaatggacac

SEQ ID NO: 195 - DNA, 21 bp, unidentified
  source 1..21: mol_type = genomic DNA
gctagagggaggtcagagcac

SEQ ID NO: 196 - DNA, 24 bp, unidentified
  source 1..24: mol_type = genomic DNA
aatttttgtgcccatcgttggcac

SEQ ID NO: 197 - DNA, 23 bp, unidentified
  source 1..23: mol_type = genomic DNA
agaaatccgtctttcattgacgg

SEQ ID NO: 198 - DNA, 22 bp, unidentified
  source 1..22: mol_type = genomic DNA
tattttgtatggagtaaacaac

SEQ ID NO: 199 - DNA, 22 bp, unidentified
  source 1..22: mol_type = genomic DNA
taaatttctactgtttgtagat

SEQ ID NO: 200 - DNA, 22 bp, unidentified
  source 1..22: mol_type = genomic DNA
taaatttctactgtttgtagat

SEQ ID NO: 201 - DNA, 22 bp, unidentified
  source 1..22: mol_type = genomic DNA
taaatttctactatttgtagat

SEQ ID NO: 202 - DNA, 20 bp, unidentified
  source 1..20: mol_type = genomic DNA
taatttctactgttgtagat

SEQ ID NO: 203 - DNA, 25 bp, unidentified
  source 1..25: mol_type = genomic DNA
agcaggattcaggttgggtttgagg

SEQ ID NO: 204 - DNA, 14 bp, unidentified
  source 1..14: mol_type = genomic DNA
aggtgggttgaaag

SEQ ID NO: 205 - DNA, 36 bp, unidentified
  source 1..36: mol_type = genomic DNA
ggtggaaagggccggagatttaccggctctgacacc

SEQ ID NO: 206 - DNA, 25 bp, unidentified
  source 1..25: mol_type = genomic DNA
gaaaaacgctcttagggaatgaaag

SEQ ID NO: 207 - DNA, 36 bp, unidentified
  source 1..36: mol_type = genomic DNA
gtgtcatagcccagcttggcgggcgaaggccaagac

SEQ ID NO: 208 - DNA, 38 bp, unidentified
  source 1..38: mol_type = genomic DNA
ggatttagaccaccccaaaaatgaaggggactaaaaca

SEQ ID NO: 209 - DNA, 36 bp, unidentified
  source 1..36: mol_type = genomic DNA
gttgtggaaggtccagttttgaggggctattacaac

SEQ ID NO: 210 - DNA, 30 bp, unidentified
  source 1..30: mol_type = genomic DNA
gactaaaaccaagtaaattggtatttaaac

SEQ ID NO: 211 - DNA, 30 bp, unidentified
  source 1..30: mol_type = genomic DNA
aacccctaccaactggtcggggtttgaaac

SEQ ID NO: 212 - DNA, 36 bp, unidentified
  source 1..36: mol_type = genomic DNA
gttttagagctatgctgttttgaatggtcccaaaac

SEQ ID NO: 213 - DNA, 29 bp, unidentified
  source 1..29: mol_type = genomic DNA
gtttcagttgctgaattatttggtaaact

SEQ ID NO: 214 - DNA, 24 bp, unidentified
  source 1..24: mol_type = genomic DNA
gttgtagctcccattctcatttcg

SEQ ID NO: 215 - DNA, 17 bp, unidentified
  source 1..17: mol_type = genomic DNA
gttacagttaaggctct

SEQ ID NO: 216 - DNA, 22 bp, synthetic construct
  source 1..22: mol_type = other DNA
aaaaaaaaaaaaaaaaaaaaaa

SEQ ID NO: 217 - DNA, 22 bp, synthetic construct
  source 1..22: mol_type = other DNA
tttttttttttttttttttttt

SEQ ID NO: 218 - DNA, 22 bp, synthetic construct
  source 1..22: mol_type = other DNA
cccccccccccccccccccccc

SEQ ID NO: 219 - DNA, 22 bp, synthetic construct
  source 1..22: mol_type = other DNA
atatatatatatatatatatat

SEQ ID NO: 220 - DNA, 22 bp, synthetic construct
  source 1..22: mol_type = other DNA
gcgcgcgcgcgcgcgcgcgcgc

SEQ ID NO: 221 - DNA, 22 bp, synthetic construct
  source 1..22: mol_type = other DNA
acacacacacacacacacacac

SEQ ID NO: 222 - DNA, 22 bp, synthetic construct
  source 1..22: mol_type = other DNA
ctctctctctctctctctctct

SEQ ID NO: 223 - DNA, 20 bp, synthetic construct
  source 1..20: mol_type = other DNA
acagcccatcgactggtgtt

SEQ ID NO: 224 - DNA, 170 bp, synthetic construct
  source 1..170: mol_type = other DNA
agtacatattcaggggccaacctctcaacaatgacgaagaccatgctcactggacagaagcaaaaatgctgctggacaacatcaacacaccagaagggattataccagctctctttgaaccagaaagggagaagtcagccgccatagacggtgaataccgcctgaagggt

SEQ ID NO: 225 - DNA, 174 bp, synthetic construct
  source 1..174: mol_type = other DNA
gacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggc

SEQ ID NO: 226 - DNA, 243 bp, synthetic construct
  source 1..243: mol_type = other DNA
agacacactccagtcaattcctggctaggcaacataatcatgtttgcccccacactgtgggcgaggatgatactgatgacccacttctttagcgtcctcatagccagggatcagcttgaacaggcccttgattgcgagatctacggagcctgctactccatagaaccactggatctacctccaatcattcaaagactccatggcctcagcgcgttttcactccacagttactctccaggtgaa

SEQ ID NO: 227 - DNA, 248 bp, synthetic construct
  source 1..248: mol_type = other DNA
cagctagacacactccagtcaactcctggctaggcaacatcatcatgtatgcgcccaccttatgggcaaggatgattctgatgactcacttcttctccatccttctagctcaggagcaacttgaaaaagccctagattgtcagatctacggggcctgttactccattgagccacttgacctacctcagatcattcagcgactccatggtcttagcgcattttcactccatagttactctccaggtgag

SEQ ID NO: 228 - DNA, 50 bp, synthetic construct
  source 1..50: mol_type = other DNA
gaaattaatacgactcactataggggtacatattcaggggccaacctctc

SEQ ID NO: 229 - DNA, 25 bp, synthetic construct
  source 1..25: mol_type = other DNA
tttctggttcaaagagagctggtat

SEQ ID NO: 230 - DNA, 41 bp, synthetic construct
  source 1..41: mol_type = other DNA
tccagtgagcatggtcttcgtaatttctactaagtgtagat

SEQ ID NO: 231 - DNA, 50 bp, synthetic construct
  source 1..50: mol_type = other DNA
gaaattaatacgactcactatagggccacactggaacaacaaagaagcac

SEQ ID NO: 232 - DNA, 30 bp, synthetic construct
  source 1..30: mol_type = other DNA
acagccttccctttgcaccatccatctcag

SEQ ID NO: 233 - DNA, 41 bp, synthetic construct
  source 1..41: mol_type = other DNA
gaaccacgacagtttgcctttaatttctactaagtgtagat

SEQ ID NO: 234 - DNA, 51 bp, synthetic construct
  source 1..51: mol_type = other DNA
gaaattaatacgactcactatagggcacactccagtcaattcctggctagg

SEQ ID NO: 235 - DNA, 26 bp, synthetic construct
  source 1..26: mol_type = other DNA
cacctggagagtaactgtggagtgaa

SEQ ID NO: 236 - DNA, 41 bp, synthetic construct
  source 1..41: mol_type = other DNA
caagggcctgttcaagctgataatttctactaagtgtagat

SEQ ID NO: 237 - DNA, 41 bp, synthetic construct
  source 1..41: mol_type = other DNA
ctagggctttttcaagttgctaatttctactaagtgtagat

SEQ ID NO: 238 - DNA, 38 bp, synthetic construct
  source 1..38: mol_type = other DNA
taatacgactcactataggagcgtctagccatggcgtt

SEQ ID NO: 239 - DNA, 21 bp, synthetic construct
  source 1..21: mol_type = other DNA
gcaagcaccctatcaggcagt

SEQ ID NO: 240 - DNA, 41 bp, synthetic construct
  source 1..41: mol_type = other DNA
tccaagaaaggacccggtcgtaatttctactaagtgtagat

SEQ ID NO: 241 - DNA, 38 bp, synthetic construct
  source 1..38: mol_type = other DNA
taatacgactcactatagacaccaacggcagttggcac

SEQ ID NO: 242 - DNA, 20 bp, synthetic construct
  source 1..20: mol_type = other DNA
aaggtgttgttgcccacccc

SEQ ID NO: 243 - DNA, 41 bp, synthetic construct
  source 1..41: mol_type = other DNA
gcctgaagagttgaatttgttaatttctactaagtgtagat

SEQ ID NO: 244 - DNA, 41 bp, synthetic construct
  source 1..41: mol_type = other DNA
  modified_base 1..3: mod_base = OTHER; note = phosphorothioate linkage
  modified_base 38..40: mod_base = OTHER; note = phosphorothioate linkage
gatgatggccatgttatccttagatgtgaatcatctttaat

SEQ ID NO: 245 - DNA, 41 bp, synthetic construct; identical to SEQ ID NO: 244
  source 1..41: mol_type = other DNA
  modified_base 1..3: mod_base = OTHER; note = phosphorothioate linkage and locked nucleic acids
  modified_base 17..19: mod_base = OTHER; note = locked nucleic acids
  modified_base 38..40: mod_base = OTHER; note = phosphorothioate linkage

SEQ ID NO: 246 - DNA, 41 bp, synthetic construct
  source 1..41: mol_type = other DNA
  modified_base 1..3: mod_base = OTHER; note = phosphorothioate linkage
  modified_base 38..40: mod_base = OTHER; note = phosphorothioate linkage
ttggagccgtacatgaactgtagatgtgaatcatctttaat

SEQ ID NO: 247 - DNA, 34 bp, synthetic construct
  source 1..34: mol_type = other DNA
tcgtcggcagcgtctaatacgactcactataggg

SEQ ID NO: 248 - DNA, 22 bp, synthetic construct
  source 1..22: mol_type = other DNA
gactacttgaagctgtccttcc

SEQ ID NO: 249 - DNA, 20 bp, synthetic construct
  source 1..20: mol_type = other DNA
cgcagcttcaccttgtagat

SEQ ID NO: 250 - DNA, 23 bp, synthetic construct
  source 1..23: mol_type = other DNA
  misc_feature 1: note = FAM fluorophore
  modified_base 9: mod_base = OTHER; note = internal ZEN quencher
  misc_feature 23: note = Iowa black quencher
ttcaagtgggagcgcgtgatgaa

SEQ ID NO: 251 - DNA, 23 bp, synthetic construct
  source 1..23: mol_type = other DNA
gctccctctttctttgcagcaat

SEQ ID NO: 252 - DNA, 23 bp, synthetic construct
  source 1..23: mol_type = other DNA
taccatgagtccttccacgatac

SEQ ID NO: 253 - DNA, 26 bp, synthetic construct
  source 1..26: mol_type = other DNA
  misc_feature 1: note = Cy5 fluorophore
  misc_feature 9: note = internal TAO quencher
  misc_feature 26: note = Iowa Black RQ quencher
tcctgcaccaccaactgcttagcacc

SEQ ID NO: 254 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
tcaccagaagcgtaccatactcacgaacagtagatgtgaatcatctttaat

SEQ ID NO: 255 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
aaacaccacatgcttgccatccaaccactctagatgtgaatcatctttaat

SEQ ID NO: 256 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
gaagttcaggaacttcctcaatacgatgactagatgtgaatcatctttaat

SEQ ID NO: 257 - DNA, 41 bp, synthetic construct
  source 1..41: mol_type = other DNA
  modified_base 1..3: mod_base = OTHER; note = phosphorothioate linkage
  modified_base 38..40: mod_base = OTHER; note = phosphorothioate linkage
ctggtcttggctgaggtttctagatgtgaatcatctttaat

SEQ ID NO: 258 - DNA, 41 bp, synthetic construct
  source 1..41: mol_type = other DNA
  modified_base 1..3: mod_base = OTHER; note = phosphorothioate linkage
  modified_base 38..40: mod_base = OTHER; note = phosphorothioate linkage
cgatgcggtgggctcggtcctagatgtgaatcatctttaat

SEQ ID NO: 259 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
aagccaggaagaaggccatggaagacatgctagatgtgaatcatctttaat

SEQ ID NO: 260 - DNA, 51 bp, synthetic construct
  source 1..51: mol_type = other DNA
  modified_base 1..3: mod_base = OTHER; note = phoshporothioate linkage
  modified_base 48..50: mod_base = OTHER; note = phosphorothioate linkage
atggggcaacttcaaggccagctccagcagtagatgtgaatcatctttaat

SEQ ID NO: 261 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
ccaggttccacgggatgctctgggcaaagatagatgtgaatcatctttaat

SEQ ID NO: 262 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
ggaagacatgcaggatcttggtgaggtatctagatgtgaatcatctttaat

SEQ ID NO: 263 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
ctctgactgcgagaggtgggtctcctcctttagatgtgaatcatctttaat

SEQ ID NO: 264 - RNA, 44 nt, synthetic construct
  source 1..44: mol_type = other RNA
taatttctactcttgtagatgcagggctccgatgtataataatt

SEQ ID NO: 265 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
agccccgcggccatcacgccacagtttccctagatgtgaatcatctttaat

SEQ ID NO: 266 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
gtatgattggggacattatcagagaaacattagatgtgaatcatctttaat

SEQ ID NO: 267 - DNA, 20 bp, synthetic construct
  source 1..20: mol_type = other DNA
catcactgccacccagaaga

SEQ ID NO: 268 - DNA, 22 bp, synthetic construct
  source 1..22: mol_type = other DNA
cagtagaggcagggatgatgtt

SEQ ID NO: 269 - DNA, 21 bp, synthetic construct
  source 1..21: mol_type = other DNA
  modified_base 1: mod_base = OTHER; note = FAM fluorescent tag
  modified_base 9: mod_base = OTHER; note = ZEN internal quencher
  modified_base 21: mod_base = OTHER; note = Iowa Black fluorescence quencher
ccctccgggaaactgtggcgt

SEQ ID NO: 270 - DNA, 20 bp, synthetic construct
  source 1..20: mol_type = other DNA
tgcccagatgtgcgctatta

SEQ ID NO: 271 - DNA, 20 bp, synthetic construct
  source 1..20: mol_type = other DNA
cttctcaagcctccacctga

SEQ ID NO: 272 - DNA, 23 bp, synthetic construct
  source 1..23: mol_type = other DNA
  modified_base 1: mod_base = OTHER; note = FAM fluorescent tag
  modified_base 9: mod_base = OTHER; note = ZEN internal quencher
  modified_base 23: mod_base = OTHER; note = Iowa Black fluorescence quencher
tcgtcaatgccagtctccctggt

SEQ ID NO: 273 - DNA, 20 bp, synthetic construct
  source 1..20: mol_type = other DNA
tcaaggctgagaacgggaag

SEQ ID NO: 274 - DNA, 20 bp, synthetic construct
  source 1..20: mol_type = other DNA
ggactccacgacgtactcag

SEQ ID NO: 275 - DNA, 20 bp, synthetic construct
  source 1..20: mol_type = other DNA
  modified_base 1: mod_base = OTHER; note = Cy5 fluorescent tag
  modified_base 20: mod_base = OTHER; note = Iowa Black RQ quencher
tccaggagcgagatccctcc

SEQ ID NO: 276 - DNA, 51 bp, synthetic construct; identical to SEQ ID NO: 255
  source 1..51: mol_type = other DNA
  modified_base 1..3: mod_base = OTHER; note = phosphorothioate linkage
  modified_base 48..50: mod_base = OTHER; note = phosphorothioate linkage

SEQ ID NO: 277 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
cagtgccattatggcgtgtgaagtcaccactagatgtgaatcatctttaat

SEQ ID NO: 278 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
gtcttggcagtgcagatgaaaaactgggaatagatgtgaatcatctttaat

SEQ ID NO: 279 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
gtatgctttaggatgaagttctcatcttcatagatgtgaatcatctttaat

SEQ ID NO: 280 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
ctggtggttaagataaaacacaagtcaaactagatgtgaatcatctttaat

SEQ ID NO: 281 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
ttcactttgccaaacaccacatgcttgccatagatgtgaatcatctttaat

SEQ ID NO: 282 - DNA, 51 bp, synthetic construct; identical to SEQ ID NO: 256
  source 1..51: mol_type = other DNA
  modified_base 1..3: mod_base = OTHER; note = phosphorothioate linkage
  modified_base 48..50: mod_base = OTHER; note = phosphorothioate linkage

SEQ ID NO: 283 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
tctttggatctctgggcttttcaagattcttagatgtgaatcatctttaat

SEQ ID NO: 284 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
tatcgtttttgggttgtgttcactctacgatagatgtgaatcatctttaat

SEQ ID NO: 285 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
tttttgatatcattccaggctttaagtttctagatgtgaatcatctttaat

SEQ ID NO: 286 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
accagtgctggtagggctgaggcagccaggtagatgtgaatcatctttaat

SEQ ID NO: 287 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
aaggagcaaaacagcttccttggtcttctttagatgtgaatcatctttaat

SEQ ID NO: 288 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
tacattacagacagtgagccaccatgcccatagatgtgaatcatctttaat

SEQ ID NO: 289 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
tgggactggaaagtaaaaaaaatctaagtatagatgtgaatcatctttaat

SEQ ID NO: 290 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
atgccaggacccgtatgctttaggatgaagtagatgtgaatcatctttaat

SEQ ID NO: 291 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
tgggttcttctttaggactctgcgatggattagatgtgaatcatctttaat

SEQ ID NO: 292 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
tgaggcagccagggcagaacagatggcgtatagatgtgaatcatctttaat

SEQ ID NO: 293 - DNA, 51 bp, synthetic construct
  source 1..51: mol_type = other DNA
  modified_base 1..3: mod_base = OTHER; note = phosphorothioate linkage
  modified_base 48..50: mod_base = OTHER; note = phosphorothioate linkage
cttttcggagtacaccgatatcagtgggcgtagatgtgaatcatctttaat

SEQ ID NO: 294 - DNA, 56 bp, synthetic construct
  source 1..56: mol_type = other DNA
acagcccatcgactggtgtttaaaagtgaatcatctttaatctgtctcttatacac

SEQ ID NO: 295 - DNA, 56 bp, synthetic construct
  source 1..56: mol_type = other DNA
acagcccatcgactggtgtttaaacgtgaatcatctttaatctgtctcttatacac

SEQ ID NO: 296 - DNA, 56 bp, synthetic construct
  source 1..56: mol_type = other DNA
acagcccatcgactggtgtttaaaggtgaatcatctttaatctgtctcttatacac

SEQ ID NO: 297 - DNA, 56 bp, synthetic construct
  source 1..56: mol_type = other DNA
acagcccatcgactggtgtttaaatgtgaatcatctttaatctgtctcttatacac

SEQ ID NO: 298 - DNA, 56 bp, synthetic construct
  source 1..56: mol_type = other DNA
acagcccatcgactggtgtttaacagtgaatcatctttaatctgtctcttatacac

SEQ ID NO: 299 - DNA, 56 bp, synthetic construct
  source 1..56: mol_type = other DNA
acagcccatcgactggtgtttaaccgtgaatcatctttaatctgtctcttatacac

SEQ ID NO: 300 - DNA, 56 bp, synthetic construct
  source 1..56: mol_type = other DNA
acagcccatcgactggtgtttaacggtgaatcatctttaatctgtctcttatacac

SEQ ID NO: 301 - DNA, 56 bp, synthetic construct
  source 1..56: mol_type = other DNA
acagcccatcgactggtgtttaactgtgaatcatctttaatctgtctcttatacac

SEQ ID NO: 302 - DNA, 56 bp, synthetic construct
  source 1..56: mol_type = other DNA
acagcccatcgactggtgtttaagagtgaatcatctttaatctgtctcttatacac

SEQ ID NO: 303 - DNA, 56 bp, synthetic construct
  source 1..56: mol_type = other DNA
acagcccatcgactggtgtttaagcgtgaatcatctttaatctgtctcttatacac

SEQ ID NO: 304 - DNA, 56 bp, synthetic construct
  source 1..56: mol_type = other DNA
acagcccatcgactggtgtttaagggtgaatcatctttaatctgtctcttatacac

SEQ ID NO: 305 - DNA, 56 bp, synthetic construct
  source 1..56: mol_type = other DNA
acagcccatcgactggtgtttaagtgtgaatcatctttaatctgtctcttatacac

SEQ ID NO: 306 - DNA, 56 bp, synthetic construct
  source 1..56: mol_type = other DNA
acagcccatcgactggtgtttaatagtgaatcatctttaatctgtctcttatacac

SEQ ID NO: 307 - DNA, 56 bp, synthetic construct
  source 1..56: mol_type = other DNA
acagcccatcgactggtgtttaatcgtgaatcatctttaatctgtctcttatacac

SEQ ID NO: 308 - DNA, 56 bp, synthetic construct
  source 1..56: mol_type = other DNA
acagcccatcgactggtgtttaatggtgaatcatctttaatctgtctcttatacac

SEQ ID NO: 309 - DNA, 56 bp, synthetic construct
  source 1..56: mol_type = other DNA
acagcccatcgactggtgtttaattgtgaatcatctttaatctgtctcttatacac

SEQ ID NO: 310 - DNA, 56 bp, synthetic construct
  source 1..56: mol_type = other DNA
acagcccatcgactggtgtttacaagtgaatcatctttaatctgtctcttatacac

SEQ ID NO: 311 - DNA, 56 bp, synthetic construct
  source 1..56: mol_type = other DNA
acagcccatcgactggtgtttacacgtgaatcatctttaatctgtctcttatacac

SEQ ID NO: 312 - DNA, 56 bp, synthetic construct
  source 1..56: mol_type = other DNA
acagcccatcgactggtgtttacaggtgaatcatctttaatctgtctcttatacac

SEQ ID NO: 313 - DNA, 56 bp, synthetic construct
  source 1..56: mol_type = other DNA
acagcccatcgactggtgtttacatgtgaatcatctttaatctgtctcttatacac

SEQ ID NO: 314 - DNA, 56 bp, synthetic construct
  source 1..56: mol_type = other DNA
acagcccatcgactggtgtttaccagtgaatcatctttaatctgtctcttatacac

SEQ ID NO: 315 - DNA, 56 bp, synthetic construct
  source 1..56: mol_type = other DNA
acagcccatcgactggtgtttacccgtgaatcatctttaatctgtctcttatacac

SEQ ID NO: 316 - DNA, 56 bp, synthetic construct
  source 1..56: mol_type = other DNA
acagcccatcgactggtgtttaccggtgaatcatctttaatctgtctcttatacac

SEQ ID NO: 317 - DNA, 56 bp, synthetic construct
  source 1..56: mol_type = other DNA
acagcccatcgactggtgtttacctgtgaatcatctttaatctgtctcttatacac

SEQ ID NO: 318 - DNA, 56 bp, synthetic construct
  source 1..56: mol_type = other DNA
acagcccatcgactggtgtttacgagtgaatcatctttaatctgtctcttatacac

SEQ ID NO: 319 - DNA, 56 bp, synthetic construct
  source 1..56: mol_type = other DNA
acagcccatcgactggtgtttacgcgtgaatcatctttaatctgtctcttatacac

SEQ ID NO: 320 - DNA, 56 bp, synthetic construct
  source 1..56: mol_type = other DNA
acagcccatcgactggtgtttacgggtgaatcatctttaatctgtctcttatacac

SEQ ID NO: 321 - DNA, 56 bp, synthetic construct
  source 1..56: mol_type = other DNA
acagcccatcgactggtgtttacgtgtgaatcatctttaatctgtctcttatacac

SEQ ID NO: 322 - DNA, 56 bp, synthetic construct
  source 1..56: mol_type = other DNA
acagcccatcgactggtgtttactagtgaatcatctttaatctgtctcttatacac

SEQ ID NO: 323 - DNA, 56 bp, synthetic construct
  source 1..56: mol_type = other DNA
acagcccatcgactggtgtttactcgtgaatcatctttaatctgtctcttatacac

SEQ ID NO: 324 - DNA, 56 bp, synthetic construct
  source 1..56: mol_type = other DNA
acagcccatcgactggtgtttactggtgaatcatctttaatctgtctcttatacac

SEQ ID NO: 325 - DNA, 56 bp, synthetic construct
  source 1..56: mol_type = other DNA
acagcccatcgactggtgtttacttgtgaatcatctttaatctgtctcttatacac

SEQ ID NO: 326 - DNA, 56 bp, synthetic construct
  source 1..56: mol_type = other DNA
acagcccatcgactggtgtttagaagtgaatcatctttaatctgtctcttatacac

SEQ ID NO: 327 - DNA, 56 bp, synthetic construct
  source 1..56: mol_type = other DNA
acagcccatcgactggtgtttagacgtgaatcatctttaatctgtctcttatacac

SEQ ID NO: 328 - DNA, 56 bp, synthetic construct
  source 1..56: mol_type = other DNA
acagcccatcgactggtgtttagaggtgaatcatctttaatctgtctcttatacac

SEQ ID NO: 329 - DNA, 56 bp, synthetic construct
  source 1..56: mol_type = other DNA
acagcccatcgactggtgtttagatgtgaatcatctttaatctgtctcttatacac

SEQ ID NO: 330 - DNA, 56 bp, synthetic construct
  source 1..56: mol_type = other DNA
acagcccatcgactggtgtttagcagtgaatcatctttaatctgtctcttatacac

SEQ ID NO: 331 - DNA, 56 bp, synthetic construct
  source 1..56: mol_type = other DNA
acagcccatcgactggtgtttagccgtgaatcatctttaatctgtctcttatacac

SEQ ID NO: 332 - DNA, 56 bp, synthetic construct
  source 1..56: mol_type = other DNA
acagcccatcgactggtgtttagcggtgaatcatctttaatctgtctcttatacac

SEQ ID NO: 333 - DNA, 56 bp, synthetic construct
  source 1..56: mol_type = other DNA
acagcccatcgactggtgtttagctgtgaatcatctttaatctgtctcttatacac

SEQ ID NO: 334 - DNA, 56 bp, synthetic construct
  source 1..56: mol_type = other DNA
acagcccatcgactggtgtttaggagtgaatcatctttaatctgtctcttatacac

SEQ ID NO: 335 - DNA, 56 bp, synthetic construct
  source 1..56: mol_type = other DNA
acagcccatcgactggtgtttaggcgtgaatcatctttaatctgtctcttatacac

SEQ ID NO: 336 - DNA, 56 bp, synthetic construct
  source 1..56: mol_type = other DNA
acagcccatcgactggtgtttaggggtgaatcatctttaatctgtctcttatacac

SEQ ID NO: 337 - DNA, 56 bp, synthetic construct
  source 1..56: mol_type = other DNA
acagcccatcgactggtgtttaggtgtgaatcatctttaatctgtctcttatacac

SEQ ID NO: 338 - DNA, 56 bp, synthetic construct
  source 1..56: mol_type = other DNA
acagcccatcgactggtgtttagtagtgaatcatctttaatctgtctcttatacac

SEQ ID NO: 339 - DNA, 56 bp, synthetic construct
  source 1..56: mol_type = other DNA
acagcccatcgactggtgtttagtcgtgaatcatctttaatctgtctcttatacac

SEQ ID NO: 340 - DNA, 56 bp, synthetic construct
  source 1..56: mol_type = other DNA
acagcccatcgactggtgtttagtggtgaatcatctttaatctgtctcttatacac

SEQ ID NO: 341 - DNA, 56 bp, synthetic construct
  source 1..56: mol_type = other DNA
acagcccatcgactggtgtttagttgtgaatcatctttaatctgtctcttatacac

SEQ ID NO: 342 - DNA, 56 bp, synthetic construct
  source 1..56: mol_type = other DNA
acagcccatcgactggtgtttataagtgaatcatctttaatctgtctcttatacac

SEQ ID NO: 343 - DNA, 56 bp, synthetic construct
  source 1..56: mol_type = other DNA
acagcccatcgactggtgtttatacgtgaatcatctttaatctgtctcttatacac

SEQ ID NO: 344 - DNA, 56 bp, synthetic construct
  source 1..56: mol_type = other DNA
acagcccatcgactggtgtttataggtgaatcatctttaatctgtctcttatacac

SEQ ID NO: 345 - DNA, 56 bp, synthetic construct
  source 1..56: mol_type = other DNA
acagcccatcgactggtgtttatatgtgaatcatctttaatctgtctcttatacac

SEQ ID NO: 346 - DNA, 56 bp, synthetic construct
  source 1..56: mol_type = other DNA
acagcccatcgactggtgtttatcagtgaatcatctttaatctgtctcttatacac

SEQ ID NO: 347 - DNA, 56 bp, synthetic construct
  source 1..56: mol_type = other DNA
acagcccatcgactggtgtttatccgtgaatcatctttaatctgtctcttatacac

SEQ ID NO: 348 - DNA, 56 bp, synthetic construct
  source 1..56: mol_type = other DNA
acagcccatcgactggtgtttatcggtgaatcatctttaatctgtctcttatacac

SEQ ID NO: 349 - DNA, 56 bp, synthetic construct
  source 1..56: mol_type = other DNA
acagcccatcgactggtgtttatctgtgaatcatctttaatctgtctcttatacac

SEQ ID NO: 350 - DNA, 56 bp, synthetic construct
  source 1..56: mol_type = other DNA
acagcccatcgactggtgtttatgagtgaatcatctttaatctgtctcttatacac

SEQ ID NO: 351 - DNA, 56 bp, synthetic construct
  source 1..56: mol_type = other DNA
acagcccatcgactggtgtttatgcgtgaatcatctttaatctgtctcttatacac

SEQ ID NO: 352 - DNA, 56 bp, synthetic construct
  source 1..56: mol_type = other DNA
acagcccatcgactggtgtttatgggtgaatcatctttaatctgtctcttatacac

SEQ ID NO: 353 - DNA, 56 bp, synthetic construct
  source 1..56: mol_type = other DNA
acagcccatcgactggtgtttatgtgtgaatcatctttaatctgtctcttatacac

SEQ ID NO: 354 - DNA, 56 bp, synthetic construct
  source 1..56: mol_type = other DNA
acagcccatcgactggtgtttattagtgaatcatctttaatctgtctcttatacac

SEQ ID NO: 355 - DNA, 56 bp, synthetic construct
  source 1..56: mol_type = other DNA
acagcccatcgactggtgtttattcgtgaatcatctttaatctgtctcttatacac

SEQ ID NO: 356 - DNA, 56 bp, synthetic construct
  source 1..56: mol_type = other DNA
acagcccatcgactggtgtttattggtgaatcatctttaatctgtctcttatacac

SEQ ID NO: 357 - DNA, 56 bp, synthetic construct
  source 1..56: mol_type = other DNA
acagcccatcgactggtgtttatttgtgaatcatctttaatctgtctcttatacac

SEQ ID NO: 358 - DNA, 56 bp, synthetic construct
  source 1..56: mol_type = other DNA
acagcccatcgactggtgtttcaaagtgaatcatctttaatctgtctcttatacac

SEQ ID NO: 359 - DNA, 56 bp, synthetic construct
  source 1..56: mol_type = other DNA
acagcccatcgactggtgtttcaacgtgaatcatctttaatctgtctcttatacac

SEQ ID NO: 360 - DNA, 56 bp, synthetic construct
  source 1..56: mol_type = other DNA
acagcccatcgactggtgtttcaaggtgaatcatctttaatctgtctcttatacac

SEQ ID NO: 361 - DNA, 56 bp, synthetic construct
  source 1..56: mol_type = other DNA
acagcccatcgactggtgtttcaatgtgaatcatctttaatctgtctcttatacac

SEQ ID NO: 362 - DNA, 56 bp, synthetic construct
  source 1..56: mol_type = other DNA
acagcccatcgactggtgtttcacagtgaatcatctttaatctgtctcttatacac

SEQ ID NO: 363 - DNA, 56 bp, synthetic construct
  source 1..56: mol_type = other DNA
acagcccatcgactggtgtttcaccgtgaatcatctttaatctgtctcttatacac

SEQ ID NO: 364 - DNA, 56 bp, synthetic construct
  source 1..56: mol_type = other DNA
acagcccatcgactggtgtttcacggtgaatcatctttaatctgtctcttatacac

SEQ ID NO: 365 - DNA, 56 bp, synthetic construct
  source 1..56: mol_type = other DNA
acagcccatcgactggtgtttcactgtgaatcatctttaatctgtctcttatacac

SEQ ID NO: 366 - DNA, 56 bp, synthetic construct
  source 1..56: mol_type = other DNA
acagcccatcgactggtgtttcagagtgaatcatctttaatctgtctcttatacac

SEQ ID NO: 367 - DNA, 56 bp, synthetic construct
  source 1..56: mol_type = other DNA
acagcccatcgactggtgtttcagcgtgaatcatctttaatctgtctcttatacac

SEQ ID NO: 368 - DNA, 56 bp, synthetic construct
  source 1..56: mol_type = other DNA
acagcccatcgactggtgtttcagggtgaatcatctttaatctgtctcttatacac

SEQ ID NO: 369 - DNA, 56 bp, synthetic construct
  source 1..56: mol_type = other DNA
acagcccatcgactggtgtttcagtgtgaatcatctttaatctgtctcttatacac

SEQ ID NO: 370 - DNA, 56 bp, synthetic construct
  source 1..56: mol_type = other DNA
acagcccatcgactggtgtttcatagtgaatcatctttaatctgtctcttatacac

SEQ ID NO: 371 - DNA, 56 bp, synthetic construct
  source 1..56: mol_type = other DNA
acagcccatcgactggtgtttcatcgtgaatcatctttaatctgtctcttatacac

SEQ ID NO: 372 - DNA, 56 bp, synthetic construct
  source 1..56: mol_type = other DNA
acagcccatcgactggtgtttcatggtgaatcatctttaatctgtctcttatacac

SEQ ID NO: 373 - DNA, 56 bp, synthetic construct
  source 1..56: mol_type = other DNA
acagcccatcgactggtgtttcattgtgaatcatctttaatctgtctcttatacac

SEQ ID NO: 374 - DNA, 56 bp, synthetic construct
  source 1..56: mol_type = other DNA
acagcccatcgactggtgtttccaagtgaatcatctttaatctgtctcttatacac

SEQ ID NO: 375 - DNA, 56 bp, synthetic construct
  source 1..56: mol_type = other DNA
acagcccatcgactggtgtttccacgtgaatcatctttaatctgtctcttatacac

SEQ ID NO: 376 - DNA, 56 bp, synthetic construct
  source 1..56: mol_type = other DNA
acagcccatcgactggtgtttccaggtgaatcatctttaatctgtctcttatacac

SEQ ID NO: 377 - DNA, 56 bp, synthetic construct
  source 1..56: mol_type = other DNA
acagcccatcgactggtgtttccatgtgaatcatctttaatctgtctcttatacac

SEQ ID NO: 378 - DNA, 56 bp, synthetic construct
  source 1..56: mol_type = other DNA
acagcccatcgactggtgtttcccagtgaatcatctttaatctgtctcttatacac

SEQ ID NO: 379 - DNA, 56 bp, synthetic construct
  source 1..56: mol_type = other DNA
acagcccatcgactggtgtttccccgtgaatcatctttaatctgtctcttatacac

SEQ ID NO: 380 - DNA, 56 bp, synthetic construct
  source 1..56: mol_type = other DNA
acagcccatcgactggtgtttcccggtgaatcatctttaatctgtctcttatacac

SEQ ID NO: 381 - DNA, 56 bp, synthetic construct
  source 1..56: mol_type = other DNA
acagcccatcgactggtgtttccctgtgaatcatctttaatctgtctcttatacac

SEQ ID NO: 382 - DNA, 56 bp, synthetic construct
  source 1..56: mol_type = other DNA
acagcccatcgactggtgtttccgagtgaatcatctttaatctgtctcttatacac

SEQ ID NO: 383 - DNA, 56 bp, synthetic construct
  source 1..56: mol_type = other DNA
acagcccatcgactggtgtttccgcgtgaatcatctttaatctgtctcttatacac

SEQ ID NO: 384 - DNA, 56 bp, synthetic construct
  source 1..56: mol_type = other DNA
acagcccatcgactggtgtttccgggtgaatcatctttaatctgtctcttatacac

SEQ ID NO: 385 - DNA, 56 bp, synthetic construct
  source 1..56: mol_type = other DNA
acagcccatcgactggtgtttccgtgtgaatcatctttaatctgtctcttatacac

SEQ ID NO: 386 - DNA, 56 bp, synthetic construct
  source 1..56: mol_type = other DNA
acagcccatcgactggtgtttcctagtgaatcatctttaatctgtctcttatacac

SEQ ID NO: 387 - DNA, 56 bp, synthetic construct
  source 1..56: mol_type = other DNA
acagcccatcgactggtgtttcctcgtgaatcatctttaatctgtctcttatacac

SEQ ID NO: 388 - DNA, 56 bp, synthetic construct
  source 1..56: mol_type = other DNA
acagcccatcgactggtgtttcctggtgaatcatctttaatctgtctcttatacac

SEQ ID NO: 389 - DNA, 56 bp, synthetic construct
  source 1..56: mol_type = other DNA
acagcccatcgactggtgtttccttgtgaatcatctttaatctgtctcttatacac

SEQ ID NO: 390 - DNA, 56 bp, synthetic construct
  source 1..56: mol_type = other DNA
acagcccatcgactggtgtttcgaagtgaatcatctttaatctgtctcttatacac

SEQ ID NO: 391 - DNA, 56 bp, synthetic construct
  source 1..56: mol_type = other DNA
acagcccatcgactggtgtttcgacgtgaatcatctttaatctgtctcttatacac

SEQ ID NO: 392 - DNA, 56 bp, synthetic construct
  source 1..56: mol_type = other DNA
acagcccatcgactggtgtttcgaggtgaatcatctttaatctgtctcttatacac

SEQ ID NO: 393 - DNA, 56 bp, synthetic construct
  source 1..56: mol_type = other DNA
acagcccatcgactggtgtttcgatgtgaatcatctttaatctgtctcttatacac

SEQ ID NO: 394 - DNA, 56 bp, synthetic construct
  source 1..56: mol_type = other DNA
acagcccatcgactggtgtttcgcagtgaatcatctttaatctgtctcttatacac

SEQ ID NO: 395 - DNA, 56 bp, synthetic construct
  source 1..56: mol_type = other DNA
acagcccatcgactggtgtttcgccgtgaatcatctttaatctgtctcttatacac

SEQ ID NO: 396 - DNA, 56 bp, synthetic construct
  source 1..56: mol_type = other DNA
acagcccatcgactggtgtttcgcggtgaatcatctttaatctgtctcttatacac

SEQ ID NO: 397 - DNA, 56 bp, synthetic construct
  source 1..56: mol_type = other DNA
acagcccatcgactggtgtttcgctgtgaatcatctttaatctgtctcttatacac

SEQ ID NO: 398 - DNA, 56 bp, synthetic construct
  source 1..56: mol_type = other DNA
acagcccatcgactggtgtttcggagtgaatcatctttaatctgtctcttatacac

SEQ ID NO: 399 - DNA, 56 bp, synthetic construct
  source 1..56: mol_type = other DNA
acagcccatcgactggtgtttcggcgtgaatcatctttaatctgtctcttatacac

SEQ ID NO: 400 - DNA, 56 bp, synthetic construct
  source 1..56: mol_type = other DNA
acagcccatcgactggtgtttcggggtgaatcatctttaatctgtctcttatacac

SEQ ID NO: 401 - DNA, 56 bp, synthetic construct
  source 1..56: mol_type = other DNA
acagcccatcgactggtgtttcggtgtgaatcatctttaatctgtctcttatacac

SEQ ID NO: 402 - DNA, 56 bp, synthetic construct
  source 1..56: mol_type = other DNA
acagcccatcgactggtgtttcgtagtgaatcatctttaatctgtctcttatacac

SEQ ID NO: 403 - DNA, 56 bp, synthetic construct
  source 1..56: mol_type = other DNA
acagcccatcgactggtgtttcgtcgtgaatcatctttaatctgtctcttatacac

SEQ ID NO: 404 - DNA, 56 bp, synthetic construct
  source 1..56: mol_type = other DNA
acagcccatcgactggtgtttcgtggtgaatcatctttaatctgtctcttatacac

SEQ ID NO: 405 - DNA, 56 bp, synthetic construct
  source 1..56: mol_type = other DNA
acagcccatcgactggtgtttcgttgtgaatcatctttaatctgtctcttatacac

SEQ ID NO: 406 - DNA, 56 bp, synthetic construct
  source 1..56: mol_type = other DNA
acagcccatcgactggtgtttctaagtgaatcatctttaatctgtctcttatacac

SEQ ID NO: 407 - DNA, 56 bp, synthetic construct
  source 1..56: mol_type = other DNA
acagcccatcgactggtgtttctacgtgaatcatctttaatctgtctcttatacac

SEQ ID NO: 408 - DNA, 56 bp, synthetic construct
  source 1..56: mol_type = other DNA
acagcccatcgactggtgtttctaggtgaatcatctttaatctgtctcttatacac

SEQ ID NO: 409 - DNA, 56 bp, synthetic construct
  source 1..56: mol_type = other DNA
acagcccatcgactggtgtttctatgtgaatcatctttaatctgtctcttatacac

SEQ ID NO: 410 - DNA, 56 bp, synthetic construct
  source 1..56: mol_type = other DNA
acagcccatcgactggtgtttctcagtgaatcatctttaatctgtctcttatacac

SEQ ID NO: 411 - DNA, 56 bp, synthetic construct
  source 1..56: mol_type = other DNA
acagcccatcgactggtgtttctccgtgaatcatctttaatctgtctcttatacac

SEQ ID NO: 412 - DNA, 56 bp, synthetic construct
  source 1..56: mol_type = other DNA
acagcccatcgactggtgtttctcggtgaatcatctttaatctgtctcttatacac

SEQ ID NO: 413 - DNA, 56 bp, synthetic construct
  source 1..56: mol_type = other DNA
acagcccatcgactggtgtttctctgtgaatcatctttaatctgtctcttatacac

SEQ ID NO: 414 - DNA, 56 bp, synthetic construct
  source 1..56: mol_type = other DNA
acagcccatcgactggtgtttctgagtgaatcatctttaatctgtctcttatacac

SEQ ID NO: 415 - DNA, 56 bp, synthetic construct
  source 1..56: mol_type = other DNA
acagcccatcgactggtgtttctgcgtgaatcatctttaatctgtctcttatacac

SEQ ID NO: 416 - DNA, 56 bp, synthetic construct
  source 1..56: mol_type = other DNA
acagcccatcgactggtgtttctgggtgaatcatctttaatctgtctcttatacac

SEQ ID NO: 417 - DNA, 56 bp, synthetic construct
  source 1..56: mol_type = other DNA
acagcccatcgactggtgtttctgtgtgaatcatctttaatctgtctcttatacac

SEQ ID NO: 418 - DNA, 56 bp, synthetic construct
  source 1..56: mol_type = other DNA
acagcccatcgactggtgtttcttagtgaatcatctttaatctgtctcttatacac

SEQ ID NO: 419 - DNA, 56 bp, synthetic construct
  source 1..56: mol_type = other DNA
acagcccatcgactggtgtttcttcgtgaatcatctttaatctgtctcttatacac

SEQ ID NO: 420 - DNA, 56 bp, synthetic construct
  source 1..56: mol_type = other DNA
acagcccatcgactggtgtttcttggtgaatcatctttaatctgtctcttatacac

SEQ ID NO: 421 - DNA, 56 bp, synthetic construct
  source 1..56: mol_type = other DNA
acagcccatcgactggtgtttctttgtgaatcatctttaatctgtctcttatacac

SEQ ID NO: 422 - DNA, 56 bp, synthetic construct
  source 1..56: mol_type = other DNA
acagcccatcgactggtgtttgaaagtgaatcatctttaatctgtctcttatacac

SEQ ID NO: 423 - DNA, 56 bp, synthetic construct
  source 1..56: mol_type = other DNA
acagcccatcgactggtgtttgaacgtgaatcatctttaatctgtctcttatacac

SEQ ID NO: 424 - DNA, 56 bp, synthetic construct
  source 1..56: mol_type = other DNA
acagcccatcgactggtgtttgaaggtgaatcatctttaatctgtctcttatacac

SEQ ID NO: 425 - DNA, 56 bp, synthetic construct
  source 1..56: mol_type = other DNA
acagcccatcgactggtgtttgaatgtgaatcatctttaatctgtctcttatacac

SEQ ID NO: 426 - DNA, 56 bp, synthetic construct
  source 1..56: mol_type = other DNA
acagcccatcgactggtgtttgacagtgaatcatctttaatctgtctcttatacac

SEQ ID NO: 427 - DNA, 56 bp, synthetic construct
  source 1..56: mol_type = other DNA
acagcccatcgactggtgtttgaccgtgaatcatctttaatctgtctcttatacac

SEQ ID NO: 428 - DNA, 56 bp, synthetic construct
  source 1..56: mol_type = other DNA
acagcccatcgactggtgtttgacggtgaatcatctttaatctgtctcttatacac

SEQ ID NO: 429 - DNA, 56 bp, synthetic construct
  source 1..56: mol_type = other DNA
acagcccatcgactggtgtttgactgtgaatcatctttaatctgtctcttatacac

SEQ ID NO: 430 - DNA, 56 bp, synthetic construct
  source 1..56: mol_type = other DNA
acagcccatcgactggtgtttgagagtgaatcatctttaatctgtctcttatacac

SEQ ID NO: 431 - DNA, 56 bp, synthetic construct
  source 1..56: mol_type = other DNA
acagcccatcgactggtgtttgagcgtgaatcatctttaatctgtctcttatacac

SEQ ID NO: 432 - DNA, 56 bp, synthetic construct
  source 1..56: mol_type = other DNA
acagcccatcgactggtgtttgagggtgaatcatctttaatctgtctcttatacac

SEQ ID NO: 433 - DNA, 56 bp, synthetic construct
  source 1..56: mol_type = other DNA
acagcccatcgactggtgtttgagtgtgaatcatctttaatctgtctcttatacac

SEQ ID NO: 434 - DNA, 56 bp, synthetic construct
  source 1..56: mol_type = other DNA
acagcccatcgactggtgtttgatagtgaatcatctttaatctgtctcttatacac

SEQ ID NO: 435 - DNA, 56 bp, synthetic construct
  source 1..56: mol_type = other DNA
acagcccatcgactggtgtttgatcgtgaatcatctttaatctgtctcttatacac

SEQ ID NO: 436 - DNA, 56 bp, synthetic construct
  source 1..56: mol_type = other DNA
acagcccatcgactggtgtttgatggtgaatcatctttaatctgtctcttatacac

SEQ ID NO: 437 - DNA, 56 bp, synthetic construct
  source 1..56: mol_type = other DNA
acagcccatcgactggtgtttgattgtgaatcatctttaatctgtctcttatacac

SEQ ID NO: 438 - DNA, 56 bp, synthetic construct
  source 1..56: mol_type = other DNA
acagcccatcgactggtgtttgcaagtgaatcatctttaatctgtctcttatacac

SEQ ID NO: 439 - DNA, 56 bp, synthetic construct
  source 1..56: mol_type = other DNA
acagcccatcgactggtgtttgcacgtgaatcatctttaatctgtctcttatacac

SEQ ID NO: 440 - DNA, 56 bp, synthetic construct
  source 1..56: mol_type = other DNA
acagcccatcgactggtgtttgcaggtgaatcatctttaatctgtctcttatacac

SEQ ID NO: 441 - DNA, 56 bp, synthetic construct
  source 1..56: mol_type = other DNA
acagcccatcgactggtgtttgcatgtgaatcatctttaatctgtctcttatacac

SEQ ID NO: 442 - DNA, 56 bp, synthetic construct
  source 1..56: mol_type = other DNA
acagcccatcgactggtgtttgccagtgaatcatctttaatctgtctcttatacac

SEQ ID NO: 443 - DNA, 56 bp, synthetic construct
  source 1..56: mol_type = other DNA
acagcccatcgactggtgtttgcccgtgaatcatctttaatctgtctcttatacac

SEQ ID NO: 444 - DNA, 56 bp, synthetic construct
  source 1..56: mol_type = other DNA
acagcccatcgactggtgtttgccggtgaatcatctttaatctgtctcttatacac

SEQ ID NO: 445 - DNA, 56 bp, synthetic construct
  source 1..56: mol_type = other DNA
acagcccatcgactggtgtttgcctgtgaatcatctttaatctgtctcttatacac

SEQ ID NO: 446 - DNA, 56 bp, synthetic construct
  source 1..56: mol_type = other DNA
acagcccatcgactggtgtttgcgagtgaatcatctttaatctgtctcttatacac

SEQ ID NO: 447 - DNA, 56 bp, synthetic construct
  source 1..56: mol_type = other DNA
acagcccatcgactggtgtttgcgcgtgaatcatctttaatctgtctcttatacac

SEQ ID NO: 448 - DNA, 56 bp, synthetic construct
  source 1..56: mol_type = other DNA
acagcccatcgactggtgtttgcgggtgaatcatctttaatctgtctcttatacac

SEQ ID NO: 449 - DNA, 56 bp, synthetic construct
  source 1..56: mol_type = other DNA
acagcccatcgactggtgtttgcgtgtgaatcatctttaatctgtctcttatacac

SEQ ID NO: 450 - DNA, 56 bp, synthetic construct
  source 1..56: mol_type = other DNA
acagcccatcgactggtgtttgctagtgaatcatctttaatctgtctcttatacac

SEQ ID NO: 451 - DNA, 56 bp, synthetic construct
  source 1..56: mol_type = other DNA
acagcccatcgactggtgtttgctcgtgaatcatctttaatctgtctcttatacac

SEQ ID NO: 452 - DNA, 56 bp, synthetic construct
  source 1..56: mol_type = other DNA
acagcccatcgactggtgtttgctggtgaatcatctttaatctgtctcttatacac

SEQ ID NO: 453 - DNA, 56 bp, synthetic construct
  source 1..56: mol_type = other DNA
acagcccatcgactggtgtttgcttgtgaatcatctttaatctgtctcttatacac

SEQ ID NO: 454 - DNA, 56 bp, synthetic construct
  source 1..56: mol_type = other DNA
acagcccatcgactggtgtttggaagtgaatcatctttaatctgtctcttatacac

SEQ ID NO: 455 - DNA, 56 bp, synthetic construct
  source 1..56: mol_type = other DNA
acagcccatcgactggtgtttggacgtgaatcatctttaatctgtctcttatacac

SEQ ID NO: 456 - DNA, 56 bp, synthetic construct
  source 1..56: mol_type = other DNA
acagcccatcgactggtgtttggaggtgaatcatctttaatctgtctcttatacac

SEQ ID NO: 457 - DNA, 56 bp, synthetic construct
  source 1..56: mol_type = other DNA
acagcccatcgactggtgtttggatgtgaatcatctttaatctgtctcttatacac

SEQ ID NO: 458 - DNA, 56 bp, synthetic construct
  source 1..56: mol_type = other DNA
acagcccatcgactggtgtttggcagtgaatcatctttaatctgtctcttatacac

SEQ ID NO: 459 - DNA, 56 bp, synthetic construct
  source 1..56: mol_type = other DNA
acagcccatcgactggtgtttggccgtgaatcatctttaatctgtctcttatacac

SEQ ID NO: 460 - DNA, 56 bp, synthetic construct
  source 1..56: mol_type = other DNA
acagcccatcgactggtgtttggcggtgaatcatctttaatctgtctcttatacac

SEQ ID NO: 461 - DNA, 56 bp, synthetic construct
  source 1..56: mol_type = other DNA
acagcccatcgactggtgtttggctgtgaatcatctttaatctgtctcttatacac

SEQ ID NO: 462 - DNA, 56 bp, synthetic construct
  source 1..56: mol_type = other DNA
acagcccatcgactggtgtttgggagtgaatcatctttaatctgtctcttatacac

SEQ ID NO: 463 - DNA, 56 bp, synthetic construct
  source 1..56: mol_type = other DNA
acagcccatcgactggtgtttgggcgtgaatcatctttaatctgtctcttatacac

SEQ ID NO: 464 - DNA, 56 bp, synthetic construct
  source 1..56: mol_type = other DNA
acagcccatcgactggtgtttgggggtgaatcatctttaatctgtctcttatacac

SEQ ID NO: 465 - DNA, 56 bp, synthetic construct
  source 1..56: mol_type = other DNA
acagcccatcgactggtgtttgggtgtgaatcatctttaatctgtctcttatacac

SEQ ID NO: 466 - DNA, 56 bp, synthetic construct
  source 1..56: mol_type = other DNA
acagcccatcgactggtgtttggtagtgaatcatctttaatctgtctcttatacac

SEQ ID NO: 467 - DNA, 56 bp, synthetic construct
  source 1..56: mol_type = other DNA
acagcccatcgactggtgtttggtcgtgaatcatctttaatctgtctcttatacac

SEQ ID NO: 468 - DNA, 56 bp, synthetic construct
  source 1..56: mol_type = other DNA
acagcccatcgactggtgtttggtggtgaatcatctttaatctgtctcttatacac

SEQ ID NO: 469 - DNA, 56 bp, synthetic construct
  source 1..56: mol_type = other DNA
acagcccatcgactggtgtttggttgtgaatcatctttaatctgtctcttatacac

SEQ ID NO: 470 - DNA, 56 bp, synthetic construct
  source 1..56: mol_type = other DNA
acagcccatcgactggtgtttgtaagtgaatcatctttaatctgtctcttatacac

SEQ ID NO: 471 - DNA, 56 bp, synthetic construct
  source 1..56: mol_type = other DNA
acagcccatcgactggtgtttgtacgtgaatcatctttaatctgtctcttatacac

SEQ ID NO: 472 - DNA, 56 bp, synthetic construct
  source 1..56: mol_type = other DNA
acagcccatcgactggtgtttgtaggtgaatcatctttaatctgtctcttatacac

SEQ ID NO: 473 - DNA, 56 bp, synthetic construct
  source 1..56: mol_type = other DNA
acagcccatcgactggtgtttgtatgtgaatcatctttaatctgtctcttatacac

SEQ ID NO: 474 - DNA, 56 bp, synthetic construct
  source 1..56: mol_type = other DNA
acagcccatcgactggtgtttgtcagtgaatcatctttaatctgtctcttatacac

SEQ ID NO: 475 - DNA, 56 bp, synthetic construct
  source 1..56: mol_type = other DNA
acagcccatcgactggtgtttgtccgtgaatcatctttaatctgtctcttatacac

SEQ ID NO: 476 - DNA, 56 bp, synthetic construct
  source 1..56: mol_type = other DNA
acagcccatcgactggtgtttgtcggtgaatcatctttaatctgtctcttatacac

SEQ ID NO: 477 - DNA, 56 bp, synthetic construct
  source 1..56: mol_type = other DNA
acagcccatcgactggtgtttgtctgtgaatcatctttaatctgtctcttatacac

SEQ ID NO: 478 - DNA, 56 bp, synthetic construct
  source 1..56: mol_type = other DNA
acagcccatcgactggtgtttgtgagtgaatcatctttaatctgtctcttatacac

SEQ ID NO: 479 - DNA, 56 bp, synthetic construct
  source 1..56: mol_type = other DNA
acagcccatcgactggtgtttgtgcgtgaatcatctttaatctgtctcttatacac

SEQ ID NO: 480 - DNA, 56 bp, synthetic construct
  source 1..56: mol_type = other DNA
acagcccatcgactggtgtttgtgggtgaatcatctttaatctgtctcttatacac

SEQ ID NO: 481 - DNA, 56 bp, synthetic construct
  source 1..56: mol_type = other DNA
acagcccatcgactggtgtttgtgtgtgaatcatctttaatctgtctcttatacac

SEQ ID NO: 482 - DNA, 56 bp, synthetic construct
  source 1..56: mol_type = other DNA
acagcccatcgactggtgtttgttagtgaatcatctttaatctgtctcttatacac

SEQ ID NO: 483 - DNA, 56 bp, synthetic construct
  source 1..56: mol_type = other DNA
acagcccatcgactggtgtttgttcgtgaatcatctttaatctgtctcttatacac

SEQ ID NO: 484 - DNA, 56 bp, synthetic construct
  source 1..56: mol_type = other DNA
acagcccatcgactggtgtttgttggtgaatcatctttaatctgtctcttatacac

SEQ ID NO: 485 - DNA, 56 bp, synthetic construct
  source 1..56: mol_type = other DNA
acagcccatcgactggtgtttgtttgtgaatcatctttaatctgtctcttatacac

SEQ ID NO: 486 - DNA, 56 bp, synthetic construct
  source 1..56: mol_type = other DNA
acagcccatcgactggtgttttaaagtgaatcatctttaatctgtctcttatacac

SEQ ID NO: 487 - DNA, 56 bp, synthetic construct
  source 1..56: mol_type = other DNA
acagcccatcgactggtgttttaacgtgaatcatctttaatctgtctcttatacac

SEQ ID NO: 488 - DNA, 56 bp, synthetic construct
  source 1..56: mol_type = other DNA
acagcccatcgactggtgttttaaggtgaatcatctttaatctgtctcttatacac

SEQ ID NO: 489 - DNA, 56 bp, synthetic construct
  source 1..56: mol_type = other DNA
acagcccatcgactggtgttttaatgtgaatcatctttaatctgtctcttatacac

SEQ ID NO: 490 - DNA, 56 bp, synthetic construct
  source 1..56: mol_type = other DNA
acagcccatcgactggtgttttacagtgaatcatctttaatctgtctcttatacac

SEQ ID NO: 491 - DNA, 56 bp, synthetic construct
  source 1..56: mol_type = other DNA
acagcccatcgactggtgttttaccgtgaatcatctttaatctgtctcttatacac

SEQ ID NO: 492 - DNA, 56 bp, synthetic construct
  source 1..56: mol_type = other DNA
acagcccatcgactggtgttttacggtgaatcatctttaatctgtctcttatacac

SEQ ID NO: 493 - DNA, 56 bp, synthetic construct
  source 1..56: mol_type = other DNA
acagcccatcgactggtgttttactgtgaatcatctttaatctgtctcttatacac

SEQ ID NO: 494 - DNA, 56 bp, synthetic construct
  source 1..56: mol_type = other DNA
acagcccatcgactggtgttttagagtgaatcatctttaatctgtctcttatacac

SEQ ID NO: 495 - DNA, 56 bp, synthetic construct
  source 1..56: mol_type = other DNA
acagcccatcgactggtgttttagcgtgaatcatctttaatctgtctcttatacac

SEQ ID NO: 496 - DNA, 56 bp, synthetic construct
  source 1..56: mol_type = other DNA
acagcccatcgactggtgttttagggtgaatcatctttaatctgtctcttatacac

SEQ ID NO: 497 - DNA, 56 bp, synthetic construct
  source 1..56: mol_type = other DNA
acagcccatcgactggtgttttagtgtgaatcatctttaatctgtctcttatacac

SEQ ID NO: 498 - DNA, 56 bp, synthetic construct
  source 1..56: mol_type = other DNA
acagcccatcgactggtgttttatagtgaatcatctttaatctgtctcttatacac

SEQ ID NO: 499 - DNA, 56 bp, synthetic construct
  source 1..56: mol_type = other DNA
acagcccatcgactggtgttttatcgtgaatcatctttaatctgtctcttatacac

SEQ ID NO: 500 - DNA, 56 bp, synthetic construct
  source 1..56: mol_type = other DNA
acagcccatcgactggtgttttatggtgaatcatctttaatctgtctcttatacac

SEQ ID NO: 501 - DNA, 56 bp, synthetic construct
  source 1..56: mol_type = other DNA
acagcccatcgactggtgttttattgtgaatcatctttaatctgtctcttatacac

SEQ ID NO: 502 - DNA, 56 bp, synthetic construct
  source 1..56: mol_type = other DNA
acagcccatcgactggtgttttcaagtgaatcatctttaatctgtctcttatacac

SEQ ID NO: 503 - DNA, 56 bp, synthetic construct
  source 1..56: mol_type = other DNA
acagcccatcgactggtgttttcacgtgaatcatctttaatctgtctcttatacac

SEQ ID NO: 504 - DNA, 56 bp, synthetic construct
  source 1..56: mol_type = other DNA
acagcccatcgactggtgttttcaggtgaatcatctttaatctgtctcttatacac

SEQ ID NO: 505 - DNA, 56 bp, synthetic construct
  source 1..56: mol_type = other DNA
acagcccatcgactggtgttttcatgtgaatcatctttaatctgtctcttatacac

SEQ ID NO: 506 - DNA, 56 bp, synthetic construct
  source 1..56: mol_type = other DNA
acagcccatcgactggtgttttccagtgaatcatctttaatctgtctcttatacac

SEQ ID NO: 507 - DNA, 56 bp, synthetic construct
  source 1..56: mol_type = other DNA
acagcccatcgactggtgttttcccgtgaatcatctttaatctgtctcttatacac

SEQ ID NO: 508 - DNA, 56 bp, synthetic construct
  source 1..56: mol_type = other DNA
acagcccatcgactggtgttttccggtgaatcatctttaatctgtctcttatacac

SEQ ID NO: 509 - DNA, 56 bp, synthetic construct
  source 1..56: mol_type = other DNA
acagcccatcgactggtgttttcctgtgaatcatctttaatctgtctcttatacac

SEQ ID NO: 510 - DNA, 56 bp, synthetic construct
  source 1..56: mol_type = other DNA
acagcccatcgactggtgttttcgagtgaatcatctttaatctgtctcttatacac

SEQ ID NO: 511 - DNA, 56 bp, synthetic construct
  source 1..56: mol_type = other DNA
acagcccatcgactggtgttttcgcgtgaatcatctttaatctgtctcttatacac

SEQ ID NO: 512 - DNA, 56 bp, synthetic construct
  source 1..56: mol_type = other DNA
acagcccatcgactggtgttttcgggtgaatcatctttaatctgtctcttatacac

SEQ ID NO: 513 - DNA, 56 bp, synthetic construct
  source 1..56: mol_type = other DNA
acagcccatcgactggtgttttcgtgtgaatcatctttaatctgtctcttatacac

SEQ ID NO: 514 - DNA, 56 bp, synthetic construct
  source 1..56: mol_type = other DNA
acagcccatcgactggtgttttctagtgaatcatctttaatctgtctcttatacac

SEQ ID NO: 515 - DNA, 56 bp, synthetic construct
  source 1..56: mol_type = other DNA
acagcccatcgactggtgttttctcgtgaatcatctttaatctgtctcttatacac

SEQ ID NO: 516 - DNA, 56 bp, synthetic construct
  source 1..56: mol_type = other DNA
acagcccatcgactggtgttttctggtgaatcatctttaatctgtctcttatacac

SEQ ID NO: 517 - DNA, 56 bp, synthetic construct
  source 1..56: mol_type = other DNA
acagcccatcgactggtgttttcttgtgaatcatctttaatctgtctcttatacac

SEQ ID NO: 518 - DNA, 56 bp, synthetic construct
  source 1..56: mol_type = other DNA
acagcccatcgactggtgttttgaagtgaatcatctttaatctgtctcttatacac

SEQ ID NO: 519 - DNA, 56 bp, synthetic construct
  source 1..56: mol_type = other DNA
acagcccatcgactggtgttttgacgtgaatcatctttaatctgtctcttatacac

SEQ ID NO: 520 - DNA, 56 bp, synthetic construct
  source 1..56: mol_type = other DNA
acagcccatcgactggtgttttgaggtgaatcatctttaatctgtctcttatacac

SEQ ID NO: 521 - DNA, 56 bp, synthetic construct
  source 1..56: mol_type = other DNA
acagcccatcgactggtgttttgatgtgaatcatctttaatctgtctcttatacac

SEQ ID NO: 522 - DNA, 56 bp, synthetic construct
  source 1..56: mol_type = other DNA
acagcccatcgactggtgttttgcagtgaatcatctttaatctgtctcttatacac

SEQ ID NO: 523 - DNA, 56 bp, synthetic construct
  source 1..56: mol_type = other DNA
acagcccatcgactggtgttttgccgtgaatcatctttaatctgtctcttatacac

SEQ ID NO: 524 - DNA, 56 bp, synthetic construct
  source 1..56: mol_type = other DNA
acagcccatcgactggtgttttgcggtgaatcatctttaatctgtctcttatacac

SEQ ID NO: 525 - DNA, 56 bp, synthetic construct
  source 1..56: mol_type = other DNA
acagcccatcgactggtgttttgctgtgaatcatctttaatctgtctcttatacac

SEQ ID NO: 526 - DNA, 56 bp, synthetic construct
  source 1..56: mol_type = other DNA
acagcccatcgactggtgttttggagtgaatcatctttaatctgtctcttatacac

SEQ ID NO: 527 - DNA, 56 bp, synthetic construct
  source 1..56: mol_type = other DNA
acagcccatcgactggtgttttggcgtgaatcatctttaatctgtctcttatacac

SEQ ID NO: 528 - DNA, 56 bp, synthetic construct
  source 1..56: mol_type = other DNA
acagcccatcgactggtgttttggggtgaatcatctttaatctgtctcttatacac

SEQ ID NO: 529 - DNA, 56 bp, synthetic construct
  source 1..56: mol_type = other DNA
acagcccatcgactggtgttttggtgtgaatcatctttaatctgtctcttatacac

SEQ ID NO: 530 - DNA, 56 bp, synthetic construct
  source 1..56: mol_type = other DNA
acagcccatcgactggtgttttgtagtgaatcatctttaatctgtctcttatacac

SEQ ID NO: 531 - DNA, 56 bp, synthetic construct
  source 1..56: mol_type = other DNA
acagcccatcgactggtgttttgtcgtgaatcatctttaatctgtctcttatacac

SEQ ID NO: 532 - DNA, 56 bp, synthetic construct
  source 1..56: mol_type = other DNA
acagcccatcgactggtgttttgtggtgaatcatctttaatctgtctcttatacac

SEQ ID NO: 533 - DNA, 56 bp, synthetic construct
  source 1..56: mol_type = other DNA
acagcccatcgactggtgttttgttgtgaatcatctttaatctgtctcttatacac

SEQ ID NO: 534 - DNA, 56 bp, synthetic construct
  source 1..56: mol_type = other DNA
acagcccatcgactggtgtttttaagtgaatcatctttaatctgtctcttatacac

SEQ ID NO: 535 - DNA, 56 bp, synthetic construct
  source 1..56: mol_type = other DNA
acagcccatcgactggtgtttttacgtgaatcatctttaatctgtctcttatacac

SEQ ID NO: 536 - DNA, 56 bp, synthetic construct
  source 1..56: mol_type = other DNA
acagcccatcgactggtgtttttaggtgaatcatctttaatctgtctcttatacac

SEQ ID NO: 537 - DNA, 56 bp, synthetic construct
  source 1..56: mol_type = other DNA
acagcccatcgactggtgtttttatgtgaatcatctttaatctgtctcttatacac

SEQ ID NO: 538 - DNA, 56 bp, synthetic construct
  source 1..56: mol_type = other DNA
acagcccatcgactggtgtttttcagtgaatcatctttaatctgtctcttatacac

SEQ ID NO: 539 - DNA, 56 bp, synthetic construct
  source 1..56: mol_type = other DNA
acagcccatcgactggtgtttttccgtgaatcatctttaatctgtctcttatacac

SEQ ID NO: 540 - DNA, 56 bp, synthetic construct
  source 1..56: mol_type = other DNA
acagcccatcgactggtgtttttcggtgaatcatctttaatctgtctcttatacac

SEQ ID NO: 541 - DNA, 56 bp, synthetic construct
  source 1..56: mol_type = other DNA
acagcccatcgactggtgtttttctgtgaatcatctttaatctgtctcttatacac

SEQ ID NO: 542 - DNA, 56 bp, synthetic construct
  source 1..56: mol_type = other DNA
acagcccatcgactggtgtttttgagtgaatcatctttaatctgtctcttatacac

SEQ ID NO: 543 - DNA, 56 bp, synthetic construct
  source 1..56: mol_type = other DNA
acagcccatcgactggtgtttttgcgtgaatcatctttaatctgtctcttatacac

SEQ ID NO: 544 - DNA, 56 bp, synthetic construct
  source 1..56: mol_type = other DNA
acagcccatcgactggtgtttttgggtgaatcatctttaatctgtctcttatacac

SEQ ID NO: 545 - DNA, 56 bp, synthetic construct
  source 1..56: mol_type = other DNA
acagcccatcgactggtgtttttgtgtgaatcatctttaatctgtctcttatacac

SEQ ID NO: 546 - DNA, 56 bp, synthetic construct
  source 1..56: mol_type = other DNA
acagcccatcgactggtgttttttagtgaatcatctttaatctgtctcttatacac

SEQ ID NO: 547 - DNA, 56 bp, synthetic construct
  source 1..56: mol_type = other DNA
acagcccatcgactggtgttttttcgtgaatcatctttaatctgtctcttatacac

SEQ ID NO: 548 - DNA, 56 bp, synthetic construct
  source 1..56: mol_type = other DNA
acagcccatcgactggtgttttttggtgaatcatctttaatctgtctcttatacac

SEQ ID NO: 549 - DNA, 56 bp, synthetic construct
  source 1..56: mol_type = other DNA
acagcccatcgactggtgtttttttgtgaatcatctttaatctgtctcttatacac

SEQ ID NO: 550 - DNA, 43 bp, synthetic construct
  source 1..43: mol_type = other DNA
tcgtcggcagcgtcagatgtgtataagagacagattaaagatg

SEQ ID NO: 551 - DNA, 47 bp, synthetic construct
  source 1..47: mol_type = other DNA
gtctcgtgggctcggagatgtgtataagagacagacagcccatcgac

(19) United States
(12) Patent Application Publication
(10) Pub. No.: US 2026/0242852 A1
(43) Pub. Date: Aug. 20, 2026

(54) PROGRAMMABLE RNA DETECTION USING PSEUDO-GUIDE DNA USING CAS12I AND CAS12A

(71) Applicant: University of Florida Research Foundation, Inc., Gainesville, FL (US)

(72) Inventors: Piyush K. Jain, Gainesville, FL (US); Carlos Orosco, Gainesville, FL (US); Santosh Rananaware, Gainesville, FL (US)

(21) Appl. No.: 19/665,064

(22) Filed: May 1, 2026

Related U.S. Application Data
(63) Continuation-in-part of application No. PCT/US2024/051700, filed on Oct. 17, 2024.
(60) Provisional application No. 63/595,923, filed on Nov. 3, 2023.
(60) Provisional application No. 63/609,712, filed on Dec. 13, 2023.

(51) Int. Cl. C12Q 1/6823; C12N 9/10; C12N 9/22; C12N 15/11; C12Q 1/34; C12Q 1/6825; C12Q 1/6883; C12Q 1/689; C12Q 1/70

(52) U.S. Cl. CPC C12Q 1/6823; C12N 9/1007; C12N 9/226; C12N 15/111; C12Q 1/34; C12Q 1/6825; C12Q 1/6883; C12Q 1/689; C12Q 1/703; C12Q 1/707; C07K 2319/80; C12N 2310/20; C12Y 201/01; G01N 2333/922

(57) ABSTRACT

In one aspect, the disclosure relates to methods for RNA detection using pseudo-guide DNA using Cas12i and Cas12a proteins. The methods can detect synthetic mimics of clinically relevant miRNAs including, but not limited to, miR-21, miR-122, and miR-155 The methods can also detect synthetic mimics of viral RNA such as Human Immunodeficiency Virus (HIV) RNA and other clinically relevant RNAs from real-world patient samples. In one aspect, in the disclosed methods, RNA can be detected using pseudo-DNA guides having a DNA scaffold and spacer and do not require amplification procedures prior to detection. Also disclosed herein are methods for silencing a target nucleic acid in a cell and for site-specific modification of RNA.

### CROSS-REFERENCE TO RELATED APPLICATIONS

This application is a continuation-in-part of PCT/US2024/051700 filed Oct. 17, 2024, which claims priority to, and the benefit of, U.S. provisional application Ser. No. 63/609,712 filed Dec. 13, 2023, and U.S. provisional application Ser. No. 63/595,923 filed Nov. 3, 2023, each of which is hereby incorporated by reference in its entirety.

### STATEMENT REGARDING FEDERALLY SPONSORED RESEARCH OR DEVELOPMENT

This invention was made with government support under Grant No. AI156321, Grant No. GM147788, and Grant No. AI168795 awarded by the National Institutes of Health. The government has certain rights in the invention.

### CROSS REFERENCE TO SEQUENCE LISTING

The genetic components described herein are referred to by sequence identifier numbers (SEQ ID NO). The sequence listing is provided in written computer readable format (CRF) as an xml file named “222113-1070_Sequence_Listing.xml” created on Apr. 28, 2026, and having a size of 554,686 bytes, is incorporated by reference in its entirety.

### BACKGROUND

In the dynamic realm of molecular biology, Clustered Regularly Interspaced Short Palindromic Repeats (CRISPR) has emerged as a transformative force. This adaptive immune system, encoded within prokaryotes, has evolved to defend against foreign nucleic acids, such as bacteriophages and plasmids. Its core mechanism involves capturing and integrating sequences from invading DNA into the host genome, creating a genetic memory known as ‘spacers’ for past infections. To bolster prokaryotic immunity, the CRISPR locus is transcribed and processed, generating mature CRISPR RNAs (crRNA). These crRNAs, each encoding a unique spacer sequence, guide CRISPR-associated (Cas) proteins, specialized RNA-guided endonucleases, in silencing genetic material that matches the crRNA sequence.

Of particular interest within the vast diversity of naturally occurring CRISPR/Cas systems are Class 2 systems, exemplified by the highly programmable single-effector Cas nucleases. The Class 2 Type II CRISPR system, featuring the Cas9 effector protein (CRISPR/Cas9), has made waves as a groundbreaking genome editing tool. Recent discoveries have expanded this diagnostic potential to Class 2 Type V and Type VI CRISPR/Cas systems, which exhibit the intriguing ability to non-specifically cleave DNA or RNA sequences after specific target recognition and cleavage. This capability, termed trans-cleavage, has given rise to functional diagnostic tools for nucleic acid detection, leading to the development of influential platforms like SHERLOCK and DETECTR.

Cas12a is a Type V-A Cas effector nuclease; when complexed with crRNA, Cas12a efficiently targets and cleaves DNA sequences. However, it lacks a similar capacity for RNA activators, necessitating a reverse transcription step for their detection. The diagnostic abilities of related enzyme Cas12i remain unexplored.

Altered miRNA expression is associated with various diseases including cancer, cardiovascular diseases, neurological disorders, and others. Detection of specific miRNA profiles can serve as diagnostic or prognostic biomarkers for these conditions. In particular, certain miRNAs may be overexpressed or downregulated in cancer, making them valuable for early detection and monitoring of disease progression. RNA from HIV and other RNA viruses could also be useful biomarkers for disease detection.

Despite advances in RNA detection research, there is still a scarcity of methods capable of detecting RNA, including clinically-relevant RNA, from real-world patient samples, using guide DNA and not requiring amplification procedures prior to detection. Furthermore, it would also be desirable to develop a new platform for RNA targeting, gene silencing, and transient and long-term gene editing with Cas12 enzymes within cells. These needs and other needs are satisfied by the present disclosure.

### SUMMARY

In accordance with the purpose(s) of the present disclosure, as embodied and broadly described herein, the disclosure, in one aspect, relates to methods for RNA detection using pseudo-guide DNA using Cas12i and Cas12a proteins. The methods can detect synthetic mimics of clinically relevant miRNAs including, but not limited to, miR-21, miR-122, and miR-155 The methods can also detect synthetic mimics of viral RNA such as Human Immunodeficiency Virus (HIV) RNA and other clinically relevant RNAs from real-world patient samples. In one aspect, in the disclosed methods, RNA can be detected using pseudo-DNA guides having a DNA scaffold and spacer and do not require amplification procedures prior to detection. Also disclosed herein are methods for silencing a target nucleic acid in a cell and for site-specific modification of RNA.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1N show engineering ΨDNA guides for RNA detection using Cas12 enzymes. (FIG. 1A) Schematic representation of different crRNA constructs used in this study: full-length crRNA with scaffold and guide, and truncated crRNAs with 5, 10, and 15 nucleotides removed from the 5′ end of the scaffold, as well as a no-scaffold guide RNA. (FIG. 1B) Comparison of fold change in trans-cleavage activity of Cas12i1 using different crRNA constructs in the presence of single-stranded DNA (ssDNA) and double-stranded DNA (dsDNA) targets. Full-length crRNA and minus 5 crRNA effectively induce trans-cleavage in the presence of dsDNA, whereas no-scaffold guide RNA induces activity only with ssDNA. (FIG. 1C) Fold change compared to non-target (NTC) control in trans-cleavage activity of various Cas12 enzymes (LbCas12a, AsCas12a, ErCas12a, MbCas12a, BsCas12a, BrCas12b, Cas12i1, Cas12i2, and Cas12j) using no-scaffold guide RNA with ssDNA targets. Cas12i1 and AsCas12a uniquely exhibit strong trans-cleavage activity. Guide is at a 10-fold higher concentration than ssDNA target. (FIG. 1D) Schematic representation of different DNA guide designs: DNA without handle, with a 5′ handle, with a 3′ handle, and with both 3′ and 5′ handles. The handle in each case is a synthetic DNA mimic of the guide RNA scaffold. FoI change compared to NTC in trans-cleavage activity for AsCas12a and Cas12i1 using various DNA guide designs targeting miR-21. The 3′ handle DNA guide (termed ΨDNA) shows the highest detection activity for both enzymes. DNA guide at a 10-fold higher concentration of RNA target. (FIG. 1E) Schematic representation of the experimental workflow for RNA detection using ΨDNA guides and Cas12 enzymes. Synthetic gene fragments are used for RNA synthesis via IVT, which are then combined with targeting and non-targeting ΨDNA guides. The Cas12 enzyme complexed with ΨDNA guides is employed for CRISPR detection, where the presence of target RNA activates trans-cleavage, leading to collateral cleavage of ssDNA fluorescence-based reporters (FQ) and generation of a detectable fluorescence signal. The relative fluorescence units (RFU) are plotted, showing a clear distinction between the signals from targeting ΨDNA guides (green dots) and non-targeting ΨDNA guides (blue dots). Targeting guides show significant fluorescence above the no-guide control (NGC), indicating successful detection of RNA targets (n=3). (FIG. 1F) Schematic illustration of the interchange of scaffold sequences in different Cas enzymes to test the tolerance of scaffold mismatches and the impact on RNA detection efficiency. (FIG. 1G) Heatmap displaying the tolerance of AsCas12a and Cas12i1 to different direct repeat sequences derived from diverse Cas12 systems. The heatmap illustrates the relative fluorescence intensity, indicating the detection efficiency. Some non-canonical scaffold sequences enhance the detection activity compared to their canonical scaffolds, suggesting potential for further optimization of ΨDNA guides (n=3). (FIG. 1H) Schematic illustration of the systematic method to detect the function of stem-loop in ΨDNA. “NNNN” stands for indices with random nucleotides. PBS represents primer binding sequence. Different ΨDNAs are incubated separately to allow self-trans-cleavage of unbound guides, then all reactions are mixed until all free ΨDNAs are exhausted. Protein is denatured and full ΨDNAs left are prepared for amplicon sequencing. (FIG. 1I) ΨDNAs enrichment after sequencing. Log2 fold changes are calculated between AsCas12a-containing samples and no protein controls, normalized to the lowest-performing guide. Data are plotted by R. (FIG. 1J) Violin plot of ΨDNA variants with different numbers of mutations at the stem-loop. Numbers of mutations are compared with LbΨDNA-stem-loop. Log2 fold changes are calculated between AsCas12a-containing samples and no protein controls. (n=3). (FIG. 1K) Characterization of 8 ΨDNA variants by trans-cleavage assay. Numbers are the rank from the stem-loop library. Fold changes are normalized to NTC (n=3). (FIG. 1L) Fold change compared to NTC in trans-cleavage activity for detection of synthetic mimics of viral RNA targets which include HCV (5′UTR, E2, and NS5B genes), Dengue, and Zika virus. (FIGS. 1M-1N) Fold change in trans-cleavage activity for ΨDNA-AsCas12a mediated patient sample detection for HCV-positive and negative targets. A total of 40 samples were tested (20 for the 5′UTR gene and 20 for the E2 gene). The threshold was defined at a fold change equal to 2 for the 5′UTR gene and 1.5 for the E2 gene. For positive samples, IU/mL shown for viral load quantification. (FIGS. 1B-1D and 1K-1L) Error bars represent mean value±standard deviation (SD, n=3).

FIGS. 2A-2B show Cas12i1 demonstrates trans cleavage activity with spacer cRNA for ssDNA targets only. Cas12i1 is the only Type II Cas enzyme that has this property.

FIGS. 3A-3B show that, while Cas12i1 was the only enzyme showing activity with spacer CRNA, Cas12a enzymes, particularly AsCas12a and ErCas12a, also show activity when using a @DNA. AsCas12a shows greater activity than Cas12i1 when using ΨDNA. miRNA detection is also more selective with AsCas12a.

FIG. 4 shows AsCas12a can be used with many cDNAs tiling a long RNA sequence to detect the target. Pooling many cDNAs as shown enhances the desired activity.

FIG. 5 shows detection of HIV genomic RNA with DNA with AsCas12a. 48 different DNAs were tested against a genomic HIV target. 26 ΨDNAs were complementary to the target sequence; 24 showed trans cleavage and 2 did not. 24 ΨDNAs did not target the HIV sequence and none of the non-targeting ΨDNAs showed activity.

FIGS. 6A-6B show the HIV limit of detection with pooled ΨDNA. Pooling the 5 best ΨDNAs from FIG. 5 showed enhanced trans-cleavage for AsCas12a. The limit of detection with 5 pooled ΨDNAs is 1 pM.

FIGS. 7A-7F show translational repression of mCherry with ΨDNA-AsCas12a complex in HEK293T cells. (FIG. 7A) Schematic representation of co-transfection of AsCas12a-GFP, mCherry, and ΨDNA to induce ribosome stalling in HEK293T cells. ΨDNA1 and ΨDNA1+target the start codon of mCherry mRNA, whereas ΨDNA2 targets the coding region. (FIG. 7B) Microscopy images of cells treated with AsCas12a-GFP, mCherry, and four different ΨDNA conditions (no guide, ΨDNA1, ΨDNA1+, and ΨDNA2). Individual GFP and mCherry channels are shown as well as the overlay. (FIG. 7C) Geometric Mean Fluorescence Intensity (MFI) of mCherry for all four ΨDNA conditions with AsCas12a-GFP and GFP only. mCherry MFI is calculated only from GFP-positive cells. Geometric means are used for all calculations. (FIG. 7D) MFI of GFP on all GFP+ cells. The difference in expression of GFP is non-significant throughout all samples compared to the control. Similar GFP MFI demonstrates equal conditions for all samples. Geometric means are used for all calculations. (FIGS. 7C-7D) Statistical analysis for biologically independent replicates (n=3) was performed using Dunnett's multiple comparisons test against the control samples. (FIG. 7E) mCherry MFI fold change for all ΨDNA constructs with and without AsCas12a. Statistical analysis for biologically independent replicates (n=3) was performed using Sidak's multiple comparisons test to compare the effect of AsCas12a on mCherry expression. (FIG. 7F) Relative quantification of mCherry mRNA using 2-ΔΔCt method. GAPDH was chosen as the endogenous control. Statistical analysis for biologically independent replicates (n=3) was performed using Sidak's multiple comparisons test to compare the effect of AsCas12a on mCherry mRNA level. Each biological replicate had 3 technical replicates. (FIGS. 7C-7F) Error bars represent the mean value±standard error of mean (SEM).

FIGS. 8A-8K show mRNA targeting of endogenous genes using ΨDNA-AsCas12a, off-target effects evaluation and integrated RNA-binding and translation profiling. (FIG. 8A) Schematic representation and relative mRNA expression following co-transfection of AsCas12a-GFP and GFP with targeting and non-targeting ΨDNA to induce RNA knockdown. Relative quantification of PPIA, RPL4, and PCSK9 mRNA in HEK293T cells expressing either GFP (gray, control) or AsCas12a (blue) with non-targeting ΨDNA (NT) or gene-specific ΨDNAs (ΨPPIA, ΨRPL4, or WPCSK9). Significant reduction in target gene expression is observed with ΨDNA-mediated knockdown when co-delivered with AsCas12a, demonstrating the specificity and efficiency of the RNA knockdown using the ΨDNA-guided CRISPR-Cas12a system. (FIG. 8B) RNA Immunoprecipitation (RIP) results of PPIA RNA enrichment in cells expressing HA-tagged AsCas12a compared to HA-GFP control. RNA fold change quantification indicates RNA targeting by the AsCas12a-ΨDNA complex. Statistical analysis for biologically independent replicates (n=3) was performed using an unpaired t-test. (FIGS. 8C-8D) Volcano plots from mRNA-Seq data comparing the off-target effects of AsCas12a-ΨDNA, ΨDNA, and RfxCas13d for PPIA gene. Each set is compared to a dCas13d non-target crRNA control to evaluate gene expression differences among different samples. Plots and statistical analyses were performed in DESeq2 with 95% CI with n=2 biologically independent replicates with 30 million reads each. All colored (blue) points represent genes that show statistical significance in downregulation (left) or upregulation (right) with gene of interest (PPIA) labeled. Exact quantification and comparison are displayed to evaluate the difference in off-target effects among each different condition. (FIGS. 8E-8F) mRNA-Seq volcano plots and off-target quantification for RPL4 gene comparing AsCas12a-ΨDNA, ΨDNA, and RfxCas13d. Plots and statistical analyses were performed in DESeq2 with 95% CI with n=2 biologically independent replicates with 30 million reads each. (FIG. 8G) Schematic representation and relative mRNA expression in HEK293T cells transduced with AsCas12a-GFP. (FIG. 8H) RNA knockdown evaluated with relative quantification of PPIA, RPL4, PCSK9, NRAS, and SMARCA4 mRNA with nontargeting ΨDNA (gray) or gene-specific ΨDNAs (blue). (FIG. 8I) Volcano plot from Ribo-seq data, comparing AsCas12a with ΨDNA-PPIA and the ΨDNA-NT group. Ribosomal occupancies are calculated by RiboDiff, while explicitly accounting for RNA-seq data. TE stands for translation efficiency. Colored dots represent statistical significance with 90% CI with n=2 biologically independent replicates with 100 million reads each. (FIGS. 8J-8K) Reads coverage of CLIP-seq data. Read counts are normalized by total reads number. Green arrows represent ΨDNA position. Bigwig files are visualized by IGV. AsCas12a-ΨPPIA exhibits higher relative peaks at the PPIA and PPIAP22 transcripts compared to Ψ-NT (FIG. 8J) PPIA locus. (FIG. 8K) PPIAP22 locus. (FIGS. 8A-8B and 8H) Error bars represent the mean value±standard error of mean (SEM).

FIGS. 9A-9K show programmable applications of the ΨDNA-AsCas12a system. The ΨDNA-AsCas12a platform enables dual DNA-RNA-targeting, multiplexed translational repression, targeted RNA degradation, and epitranscriptomic editing. (FIG. 9A) Schematic representation of the co-transfection of DNA, crRNA, and protein expression plasmids encoding AsCas12a-GFP or GFP. (FIG. 9B) Relative quantification of PPIA and RPL4 mRNA in HEK293T cells following co-transfection of both ΨDNA and crRNA. GFP (gray, control) or AsCas12a (blue) with non-targeting ΨDNA (NT) or gene-specific ΨDNAs (ΨPPIA or ΨRPL4) are detected. (FIG. 9C) Volcano plots from mRNA-Seq data comparing the off-target effects of AsCas12a-ΨDNA+crRNA and ΨDNA+crRNA for PPIA gene. Plots and statistical analyses were performed in DESeq2 with 95% CI with n=2 biologically independent replicates. (FIG. 9D) Genome editing efficiency on CCR5 locus from NGS data. No treatment control, ΨDNA only, crRNA only, and both ΨDNA and crRNA transfected groups are shown on the plot with 18 h (gray) or 50 h (green) editing efficiency. (FIG. 9E) Schematic representation and relative mRNA expression in HEK293T cells transduced with AsCas12a-GFP are shown. RNA knockdown evaluated by relative quantification of PPIA, RPL4, PCSK9, NRAS, and SMARCA4 mRNA with non-targeting ΨDNA (gray) or gene-specific ΨDNAs (blue). (FIG. 9F) Schematic representation and multiplex knockdown of four endogenous transcripts PPIA, RPL4, NRAS, and PCSK9 using a ΨNT (non-targeting ΨDNA) or various ΨDNA combinations as described: ΨPPIA (PPIA only), Ψ-Mix II (PPIA and RPL4), Ψ-Mix III (PPIA, RPL4 and NRAS) and Ψ-Mix IV (PPIA, RPL4, NRAS and PCSK9), showing differential and specific knockdown efficiency across gene targets in HEK293T cells transduced with AsCas12a. Results highlight the potential for multiplex RNA targeting using the ΨDNA-guided CRISPR-Cas12a platform. (FIG. 9G) Microscope images after proximity ligation assay. Green fluorescent represents GFP co-expressed with Cas proteins. Texas Red signal represents RNaseH1 colocalized with Cas protein inside HEK293T cells. (FIG. 9H) Schematic representation showing RNaseH1 fused to the N-terminus of AsCas12a on the N-terminus of AsCas12a and the fusion protein can be guided by ΨDNA and induce RNA degradation. (FIG. 9I) Relative mRNA expression for co-transfection of RNaseH1-C-GFP, AsCas12a-RNaseH1-C-GFP, AsCas12a-GFP or GFP and targeting and non-targeting ΨDNA to induce RNA knockdown. With relative quantification of RPL4 mRNA in HEK293T cells, RNaseH1 C-terminus fused AsCas12a group shows higher RNA knockdown compared to WT AsCas12a-GFP and RNaseH1-C-GFP. (FIG. 9J) Schematic representation showing the RNA methyltransferase METTL3 is fused on the C-terminus of AsCas12a and the fusion protein can be guided by ΨDNA and induce target RNA methylation (FIG. 9K) Relative RNA methylation is detected by methylated RNA immunoprecipitation qPCR (MeRIP-qPCR) for METTL3-AsCas12a or AsCas12a in HEK293T cells. METTL3 fused AsCas12a group shows significantly increased m6A level. (FIGS. 9B, 9D, 9F, 9I, and 9K) Statistical analysis for biologically independent replicates (n=3) were performed using Sidak's multiple comparison test against the NT samples. Error bars represent the mean±SEM.

FIGS. 10A-10L show characterization of ΨDNA-Cas12 trans-cleavage for RNA detection. (FIG. 10A) Schematic showing the mechanism of trans-cleavage activation by short cDNA guides for miRNA detection. Cas12 enzymes complexed with cDNA guides target miRNA, initiating trans-cleavage and subsequent fluorescence activation through the collateral cleavage of fluorescence-based reporters. Fold change compared to No Target Control (NTC) in the trans-cleavage activity of AsCas12a and Cas12i1 with cDNA guides targeting miR-21, miR-122, and miR-155. Cas12i1 shows robust RNA detection with significant fold change values for all three targets. DNA guide at a 10-fold higher concentration than the RNA target (n=3). (FIG. 10B) ΨDNA-induced trans-cleavage for RNA detection. Non-specific collateral cleavage of nucleic acids tested with ssDNA and RNA FQ reporters. (FIG. 10C) Schematic showing the mechanism of trans-cleavage activation by ΨDNA guides for miRNA detection. Cas12 enzymes complexed with ΨDNA guides target miRNA, initiating trans-cleavage and subsequent fluorescence activation through the collateral cleavage of fluorescence-based reporters. (FIGS. 10D-10E) Heatmap showing the specificity of ΨDNA guides (ΨDNA-21, ΨDNA-122, ΨDNA-155) for miRNA targets (miR-21, miR-122, miR-155) using AsCas12a and Cas12i1 (n=3). (FIG. 10F) Fold change compared to NTC in trans-cleavage activity of various Cas12 enzymes (LbCas12a, AsCas12a, ErCas12a, MbCas12a, BsCas12a, BrCas12b, Cas12i2, and Cas12j) using ΨDNA guides with ssRNA targets. AsCas12a uniquely exhibits strong trans-cleavage activity with ΨDNA guide (n=3). (FIG. 10G) Heatmap for the detection of 16 different miRNA targets with ΨDNA and Cas12i1 and AsCas12a. FC indicates the guide region of ΨDNA is fully complementary to the miRNA; C21 denotes the guide region of ΨDNA targets 1-21 nt of miRNA from 5′ end; C22 denotes the guide region of ΨDNA targets 2-22 nt of miRNA; C23 represents the guide region of ΨDNA target 3-23 nt of miRNA if miRNA is 23 nucleotides or longer (n=3). (FIG. 10H) Effect of different divalent cations on ΨDNA-induced trans-cleavage for RNA detection with AsCas12a and Cas12i1 (n=3). (FIGS. 10I-10J) Trans-cleavage activity of Cas12i1 (blue) with ΨDNAs of different guide lengths. Trans-cleavage activity of AsCas12a (green) with ΨDNAs of different guide lengths. Fluorescence is measured at 30 min. Fold change of fluorescence intensity normalized to No Target Control (NTC). The target RNA is 50 nucleotides in length (n=3). (FIG. 10K) Trans-cleavage activity of AsCas12a with 16-mer guide ΨDNA with mismatches at different positions. Fluorescence is measured at 10 min. (FIG. 10L) Trans-cleavage activity of AsCas12a with 20-mer guide ΨDNA with mismatches at different positions. Fluorescence is measured at 10 min. (FIGS. 10A-10B, 10F, and 10I-10L) Error bars represent mean±SD.

FIGS. 11A-11B show miRNA can be detected by adding a cDNA sequence complementary to it. In these examples, cDNA sizes of 20 and 23 nt long are also compared, while a synthetic mimic of endogenous miRNA is used as a surrogate guide.

FIG. 12 shows that the cDNA containing a 3′-handle can now be referred to as psiDNA (ΨDNA) and serves as a type of pseudo-DNA guide for RNA detection. The reaction is specific for detecting the miRNA that is complementary to the ΨDNA. The dotted lines in the bar graphs represent threshold detection levels.

FIG. 13 shows the Cas12i1 limit of detection using ΨDNA for detecting synthetic miR-21. The limit of detection is between 250 pM and 50 pM.

FIGS. 14A-14G show binding and efficiency of RNA cleavage by ΨDNA-Cas12 complexes. (FIG. 14A) Schematic representation of the assembly process of the AsCas12-ΨDNA complex and its interaction with the target RNA. The ΨDNA guide and Cas12 can bind to the RNA target with the 3′ handle of the ΨDNA occupying a position analogous to the 5′ handle of crRNA. (FIG. 14B) Electrophoretic Mobility Shift Assay (EMSA) showing the complex formation of AsCas12a ΨDNA and target RNA. ΨDNA is labeled with HEX dye (red channel) target RNA is labeled by FAM (green channel). This gel demonstrates the ability of ΨDNA guides to form stable complexes with AsCas12 and target RNA. (FIG. 14C) Electrophoretic Mobility Shift Assay (EMSA) comparing the complex formation of AsCas12a and dAsCas12a with FAM labeled ΨDNA guide targeting an RNA target. This gel demonstrates the ability of ΨDNA guides to form stable complexes with AsCas12/dAsCas12a and target RNA. (FIG. 14D) Agarose gel electrophoresis of RNA cis-cleavage by Cas7-11, AsCas12a, and dAsCas12a. The upper band corresponds to uncleaved RNA, and the lower band corresponds to cleaved RNA. This indicates that AsCas12a does not exhibit RNA cis-cleavage activity. (FIG. 14E) Gel electrophoresis showing the cleavage products of an RNA target labeled with FAM (green channel) on the 5′-end and Cy5 (red channel) on the 3′-end. RNA was treated with DNAzyme and ΨDNA-Cas12 complexes. The presence of cleaved RNA fragments is indicated by the separation of FAM and Cy5 signals. Gel demonstrates ΨDNA-Cas12 cannot cleave RNA targets, with distinct bands indicating successful cleavage. (FIG. 14F) DNA footprinting assay. FAM dye is added at the ends of ΨDNA (3′ for upper panel and 5′ for lower panel). All samples are treated with DNase I and lanes 3-6 are treated with RNase If. The gel demonstrates that AsCas12a protects 5′ end of ΨDNA and partially protects 3′ end of ΨDNA. (FIG. 14G) Electrophoretic Mobility Shift Assay (EMSA) showing competition between nonspecific RNA and target RNA. ΨDNA is labeled with HEX, and the target RNA is labeled with FAM. Left panel does not contain ΨDNA while left panel includes the DNA guide. Only the top portion of the EMSA gel, corresponding to protein-bound complexes, is shown for simplicity. Full gel is shown in FIGS. 21A-21B. Gel indicates that ΨDNA stabilizes AsCas12a binding to the target RNA while in presence of non-specific RNA competitors.

FIGS. 15A-15E show enzyme kinetics of AsCas12a-ΨDNA-RNA complex. (FIGS. 15A-15B) Binding affinity (K_{d}) values for different guide constructs obtained via Bio-Layer Interferometry (BLI). This includes crRNA, no-scaffold guide RNA, cDNA, and cDNA with 5′ and 3′ handles (ΨDNA). Log10 concentration plotted on right panels. (FIG. 15C) Table of binding kinetic parameters derived from BLI. K_{d} is reported with steady-state measurements (left) obtained from response curves in (FIG. 15A), and kinetic measurements (right) accounting for the reported ratios between kₒₙ and k_{off}. (FIGS. 15D-15E) Michaelis-Menten analysis reveals robust trans-cleavage activity with a with ΨDNA-ssRNA complexes. Representative plots of initial velocity versus time for ΨDNA and ssRNA target. Using 1.25 nM effective AsCas12a-ΨDNA-RNA complex and increasing fluorophore-quencher (F-Q) substrate concentrations at 37° C. Calculated k_{cat}, Kₘ, k_{cat}/Kₘ and (α, β, γ) values report the mean±SD (n=3).

FIGS. 16A-16I show targeting of long RNAs with ΨDNA-Cas12 and scaffold optimization for limit-of-detection analysis. (FIG. 16A) Detection of mimics of endogenous mRNAs by AsCas12a and ΨDNA. Fluorescence measurements for mRNA detection with ΨDNA and AsCas12a. Endogenous mRNAs from HEK293T cells were reverse-transcribed, amplified by RT-PCR, and subsequently transcribed in vitro. A total of 18 mRNA targets were tested. Fold change of fluorescence intensity normalized to No Target Control (NTC). (FIG. 16B) Heatmap displaying the tolerance of AsCas12a to different direct repeat sequences derived from diverse Cas12 systems. The heatmap illustrates the relative fluorescence intensity, indicating the detection efficiency. Some non-canonical scaffold sequences enhance the detection activity compared to their canonical scaffolds, suggesting potential for further optimization of ΨDNA guides (n=3). (FIGS. 16C-16E) Sensitivity analysis of RNA detection using different scaffold sequences with AsCas12a enzyme. Ratio of RFU at t=60 min to RFU at t=0 min at different target RNA concentrations for (FIG. 16C) AsCas12a, (FIG. 16D) LbCas12a, and (FIG. 16E) OpCas12b scaffold sequences. Threshold set at mean No Target Control (NTC) plus three times the standard deviation (o). (FIG. 16F) AsCas12a and (FIG. 16G) Cas12i1 trans-cleavage activity with crRNAs of different scaffolds to detect an ssDNA target. The scaffolds tested were selected based on their high trans-cleavage activity when used as 3′ handles in ΨDNA-mediated RNA detection. Sequence alignments of HCV patient samples obtained by NGS of (FIG. 16H) the 5′UTR and (FIG. 16I) E2 regions. (FIGS. 16H-16I) Gray sections represent matched bases to the reference genome and colored sections represent mismatched bases. Alignments were visualized by JBrowse. (FIGS. 16A and 16C-16G) Error bars represent mean±SD (n=3).

FIGS. 17A-17H show characterization of ΨDNA screening across multiple targets and Cas effectors. (FIG. 17A) Schematic representation of ΨPCSK9 screening to mediate mRNA knockdown. Relative mRNA expression levels of PCSK9 transcripts in cells transfected with five different ΨDNAs compared to a non-targeting (NT) guide. (FIGS. 17B-17D) Influence of RNA secondary structure on AsCas12a-ΨDNA-mediated knockdown. RNA knockdown evaluated with relative quantification of different ΨDNAs in PPIA, RPL4 and PCSK9 transcripts. All RNA expression levels are normalized with their non-target ΨDNA. Colored dots are groups transfected with ΨDNAs. SHAPE scores were obtained from the RASP v2.0 database. The plots show no influence of RNA secondary structure on AsCas12a-ΨDNA-mediated knockdown. Data points without visible error bars indicate that the SEM values are too small to be displayed in GraphPad Prism. (FIG. 17B) Knockdown of PPIA transcripts. (FIG. 17C) Knockdown of RPL4 transcripts. (FIG. 17D) Knockdown of PCSK9 transcripts. (FIGS. 17E-17F) Influence of RBP binding on AsCas12a-ΨDNA-mediated knockdown. RNA knockdown evaluated with relative quantification of PPIA and RPL4 mRNA with non-targeting ΨDNA (gray) or gene-specific ΨDNAs (colored). The plots show that RBP binding inhibits AsCas12a-DNA-mediated knockdown of PPIA transcripts but not RPL4. (FIG. 17E) Knockdown of PPIA transcripts. (FIG. 17F) Knockdown of RPL4 transcripts. (FIG. 17G) Relative quantification of PPIA expression. Comparing dAsCas12a (gray) and AsCas12a (blue) mediated knockdown with non-target ΨDNA or ΨPPIA. No significant differences were detected. (FIG. 17H) Comparison among GFP control, AsCas12a with ΨDNA, and RfxCas13d with crRNA. Relative quantification of PPIA and RPL4 expression, all groups are normalized by NT. (FIGS. 17B-17H) Error bars represent the mean±SEM (n=3).

FIGS. 18A-18G show specificity of the AsCas12a-ΨDNA system assessed by RNAseq, Ribo-seq, and CLIP-seq. (FIGS. 18A-18B) Volcano plots from mRNA-Seq data comparing the off-target effects of AsCas12a-ΨDNA for (FIG. 18A) PPIA and (FIG. 18B) RPL4 gene. Each set illustrates the off-target effects of AsCas12a-ΨDNA-mediated RNA knockdown of AsCas12a-ΨDNA RNA knockdown when compared to AsCas12a non-target-ΨDNA or dCas13d non-targeting crRNA control to evaluate gene expression differences among different samples. Plots and statistical analyses were in DESeq2 with 95% CI with n=2 biologically independent replicates with 30 million reads each. All colored points (blue for PPIA and green for RPL4) represent genes that show statistically significant in downregulation (left) or upregulation (right) with gene of interest labeled. Exact quantification and comparison are displayed to evaluate the difference in off-target effects among each different control. (FIGS. 18C-18D) Gene expression in counts per million of target genes (PPIA and RPL4) and other specified genes that have similar sequences to evaluate mismatch specificity for AsCas12a-ΨDNA, ΨDNA-only, and Cas13d-crRNA constructs. This analysis was performed based on the approach described by H. Tong et al. (FIG. 18E) Ribo-Seq quality control of trinucleotide periodicity for ΨNT and ΨPPIA. Above 60% percent of filtered reads have no codon shift. Data obtained with Ribowaltz (n=2). (FIG. 18F) Ribo-Seq quality control of coding sequences (CDS) reads for ΨNT and ΨPPIA. This demonstrates experimental procedure successfully captures active ribosomes on coding regions. Above 90% percent of filtered reads are in coding regions. Data obtained with Ribowaltz (n=2). Right panel portrays the distribution of total RNA without ribosomal enrichment. (FIG. 18G) Reads coverage of CLIP-seq data. Ψ-NT represents nontarget ΨDNA; ΨPPIA represents ΨDNA target PPIA transcripts; Ψ-MIX represents mix of 4 different ΨDNAs (ΨPPIA, ΨRPL4, ΨSMARCA4 and ΨPCSK9). Read counts were normalized to the total number of reads. Red arrow represents ΨDNA position. Bigwig files are visualized by IGV. Read coverage shows that AsCas12a specifically captures target transcripts guided by ΨDNA.

FIGS. 19A-19E show applications of the AsCas12a-ΨDNA system. (FIG. 19A) Endogenous mRNA knockdown induced by AsCas12a and ΨDNA in different cell lines. Relative quantification of PPIA mRNA in HeLa, HepG2, and MCF-7 cells expressing either GFP (gray, control) or AsCas12a (blue) with non-targeting ΨDNA (NT) or gene-specific ΨDNA (ΨPPIA). (FIG. 19B) Relative quantification of RPL4 mRNA. Comparison between full-length RNaseH1 (RNaseH1-FL, gray) and the C-terminal domain of RNaseH1 (RNaseH1-C, green). (FIG. 19C) Microscopy images for cells treated with RNaseHAsCas12a-GFP, mCherry, and two different conditions of ΨDNA (ΨNT and YmCherry). Individual GFP and mCherry channels are shown as well as the overlay. (FIG. 19D) Relative mRNA expression for co-transfection of RNase H-AsCas12a-GFP, AsCas12a-GFP or GFP with mCherry and targeting and non-targeting ΨDNA to induce RNA knockdown. Relative quantification of mCherry mRNA in HEK293T cells shows that RNase H-fused AsCas12a induces higher RNA knockdown compared to wild-type AsCas12a. (FIG. 19E) Multiplex knockdown of four endogenous transcripts, PPIA, RPL4, NRAS, and SMARCA4 using non-targeting ΨDNA (ΨNT) or various ΨDNA combinations as shown. Results highlight the potential for multiplex RNA targeting using the ΨDNA-guided CRISPR-Cas12a platform. (FIGS. 19A-19B and 19D-19E) Error bars represent the mean±SEM (n=3).

FIG. 20 shows predicted ΨDNA secondary structure by UNAFold. The prediction is made by UNAFold on IDT website. Spacer region of this ΨDNA is first 20 nucleotides from 5′ end.

FIGS. 21A-21B show non-specific RNA binding evaluation in the presence of ΨDNA-AsCas12a. Electrophoretic Mobility Shift Assay (EMSA) showing competition between nonspecific RNA and target RNA. ΨDNA is labeled with HEX, and the target RNA is labeled with FAM. ΨDNA stabilizes AsCas12a binding to the target RNA while in presence of non-specific RNA competitors.

FIG. 22 shows evaluation of NLS domain in dual DNA-RNA editing construct. Comparison between AsCas12a with and without NLS domain on genome editing efficiency on CCR5 locus from NGS data. No treatment control, ΨDNA only, crRNA only, and both ΨDNA and crRNA transfected groups are shown on the plot with 18 h (gray) or 50 h (green) editing efficiency.

FIG. 23 shows FACS gating strategy for mCherry gene silencing experiments. Gating to determine the MFI of mCherry and GFP for all samples in FIGS. 10A-10L. First gates exclude cell debris and only select for singlets. Later GFP-positive cells are gated and the MFI of GFP is calculated. Finally, all GFP-positive cells are plotted to find the MFI of mCherry.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Clustered Regularly Interspaced Short Palindromic Repeats (CRISPR) constitute an adaptive immune system found in prokaryotes, where CRISPR-associated (Cas) proteins complex with CRISPR RNAs (crRNAs) to precisely target and cleave exogenous nucleic acids. Since their discovery, CRISPR-Cas systems have been extensively developed for DNA and RNA genome editing and further engineered for applications such as diagnostics. Among the most well-known CRISPR-Cas effectors, type II (Cas9) and type V (Cas12) enzymes induce double-stranded breaks in DNA, while type VI (Cas13) targets and degrades RNA. Additionally, Cas12 and Cas9 exhibit non-specific collateral cleavage of single-stranded DNA (ssDNA) once the target sequence is bound, while Cas13 has similar activity against RNA. This functionality has been harnessed to develop various nucleic acid detection platforms. Overall, these advancements exploit the synergy between Cas effectors and crRNAs, enabling precise targeting of diverse nucleic acids.

As a quintessential part of the CRISPR system, crRNAs have been extensively studied to understand their interactions with Cas proteins. Transcribed from a CRISPR array, crRNAs are composed of direct repeats and spacer sequences. Once transcribed, the direct repeats are pre-processed to form a 5′ or 3′ handle, which constitutes the scaffold region of the crRNA. In many CRISPR-Cas systems, this scaffold adopts a conserved stem-loop architecture that folds into an RNA pseudoknot structure essential for stable Cas binding and catalytic activation. Thus, the scaffold region stabilizes the crRNA-Cas complex and aids in the recognition and binding of the target, while the adjacent spacer sequence is fully complementary to the target DNA or RNA. Given its unique structure and functionality, numerous efforts have been made to engineer crRNAs to enhance CRISPR-Cas systems. These efforts include incorporating modified bases, developing chimeric and split crRNAs, and altering their length. However, crRNA production is constrained by the complexity and expense of RNA synthesis, along with its shorter shelf life compared to DNA.

Naturally, CRISPR-Cas enzymes work in tandem with the crRNA to stabilize the complex and target a specific sequence. While there have been efforts to modify the target-binding guide region of the crRNA partially or completely with DNA bases for both Cas9 as well as Cas12, previous research has suggested that complete substitution of DNA bases within the guide RNA (sgRNA or crRNA) is typically not well-tolerated by either enzyme family. Contrary to this understanding, herein is demonstrated the ability of specific Type V systems (AsCas12a and Cas12i1) to withstand DNA guides for RNA targeting for cellular and in vitro applications, when the guide orientation is reversed. Cas12 enzymes were adapted to adopt synthetic DNA mimics of a guide RNA (termed pseudo-guide DNA or ΨDNA) that assemble with Cas proteins, bind the target RNA, and ultimately turn on trans-cleavage activity. ΨDNAs have successfully been engineered to detect RNA substrates in a programmable manner.

Herein it is first shown that truncated crRNA with only the target-binding region retained the trans-cleavage activity of Type V CRISPR-Cas systems, specifically Cas12i1 and AsCas12a, against ssDNA targets. It is then validated that a complementary DNA sequence matching the target-binding region (cDNA) could activate trans-cleavage in the presence of short RNA targets. To further enhance this activity for RNA targeting, the cDNA was engineered with a 3′ DNA handle to mimic the natural structure of crRNAs, forming what is referred to herein as ΨDNA. The AsCas12a-ΨDNA complex was subsequently used to induce efficient and specific degradation of various RNA targets in cells by triggering endogenous RNA degradation mechanisms such as ribosome stalling and RNaseH1 recruitment. Moreover, this novel AsCas12a construct is shown herein to perform dual RNA and DNA targeting, multiplex gene knockdown, and facilitate targeted RNA manipulation when fused to diverse effector proteins, such as RNaseH for site-specific cleavage and the RNA methyltransferase METTL3 for programmable RNA modification with N6-methyladenosine (m6A).

Collectively, the ΨDNA-Cas12 platform disclosed herein provides a versatile toolkit for both high-sensitivity, high-specificity RNA targeting, either alone or in combination with DNA indel formation, enabling precise and programmable post-transcriptional regulation of RNA within living cells.

Herein it is disclosed that the introduction of pseudo-DNA (ΨDNA) guides triggers selective trans-cleavage when these guides recognize RNA targets. These pseudo-DNA guides have a DNA scaffold and spacer and can be used to guide RNA detection with DNA, all without the need for cumbersome amplification procedures. In one aspect, with this approach, pinpoint precision has been achieved in detecting synthetic mimics of HIV genomic RNA and, crucially, real-world HIV-positive patient samples. In a further aspect, the disclosed process is straightforward: supplying Cas12i or AsCas12a, along with a collection of short single-stranded pseudo-DNA guides, to the sample initiates a trans-cleavage reaction, resulting in accurate and cost-effective RNA detection without amplification. In a further aspect, the disclosed method eliminates the complexities associated with guide RNA synthesis and additional pre-amplification protocols.

It has been fortuitously discovered that the introduction of only the spacer of the guide RNA, detached from its scaffold, triggered selective trans-cleavage. In one aspect, this process occurred upon the recognition of single-stranded DNA targets in cis, while sparing double-stranded DNA. In a further aspect, short endogenous RNAs, with a special focus on microRNAs (miRNAs), can be used as surrogate guides for precise nucleic acid detection.

In the disclosed process, pinpoint accuracy has been achieved in detecting synthetic mimics of clinically relevant miRNAs, including miR-21, miR-155, and miR-122. In some aspects, in the disclosed process, Cas12i and a short single-stranded cDNA were supplied to an miRNA-rich sample, initiating a trans-cleavage reaction that accurately identified these miRNA targets.

In an aspect, in the disclosed approach, short endogenous RNAs such as miRNAs can be used as surrogate guides for nucleic acid detection. In an aspect, the method includes supplying Cas12i1 or a related enzyme and a short, modified ssDNA to an miRNA-rich sample to initiate a trans-cleavage reaction. In a further aspect, the disclosed approach circumvents the need for complex guide RNA synthesis.

### Nucleic Acid Detection System

In one aspect, disclosed herein is a nucleic acid detection system including at least the following components:
a CRISPR system including a Cas12 protein and one or more guide nucleotides; and
an ssDNA including a fluorophore and a quencher;
wherein each guide nucleotide comprises a guide sequence capable of binding a target nucleic acid sequence and forming a complex with the Cas12 protein;
wherein the guide nucleotide includes a fluorophore and a quencher; and
wherein when the guide nucleotide binds the target nucleic acid sequence and forms a complex with the Cas12 protein, and the Cas12 protein performs trans cleavage on the ssDNA, separating the fluorophore from the quencher.

In an aspect, the Cas12 protein can be a Cas12i protein such as, for example, Cas12i1 or Cas12i2, a Cas12a protein such as, for example, AsCas12a of ErCas12a, a Cas12j protein, a Cas12b protein, or any combination thereof.

In a further aspect, in the disclosed nucleic acid detection system, the guide nucleotide is or includes a spacer, wherein the spacer includes the guide sequence. In some aspects, the spacer is from about 20 to about 23 nucleotides long, or is about 20, 21, 22, or 23 nucleotides long. In another aspect, the guide nucleotide further includes a scaffold located at a 3′ end of the guide sequence. In an aspect, the scaffold can be from about 19 to about 25 nucleotides long, or is about 19, 20, 21, 22, 23, 24, or 25 nucleotides long.

In one aspect, the fluorophore can be 5 (6)-carboxyfluorescein (56-FAM), 5′-hexachlorofluorescein (5HEX), or any combination thereof, while the quencher can be 3′-Iowa Black FQ (3IABKFQ). In another aspect, the ssDNA can be 56-FAM-TTATT-3IABKFQ or 5HEX-TTTTTTTT-3IABKFQ.

In any of these aspects, the guide nucleotide can be or include DNA, while the target nucleic acid sequence can be RNA.

### Method for Detecting Nucleic Acid

In another aspect, disclosed herein is method for detecting a target nucleic acid, the method including at least the following steps:
(a) contacting a sample suspected of containing the target nucleic acid with the disclosed nucleic acid detection system; and
(b) measuring a fluorescence signal from the nucleic acid detection system;
wherein when the fluorescence signal is present, the sample contains the target nucleic acid.

In an aspect, the method further includes performing at least one processing step prior to performing step (a), such as, for example, nucleic acid extraction, nucleic acid purification, or both.

In some aspects, the sample comprises a biological sample from a subject, although other sample types are also contemplated and should be considered disclosed, such as, for example, artificial or laboratory constructed samples including natural or artificially-synthesized RNAs and the like. In a further aspect, when the sample is a biological sample, the sample can be blood, saliva, urine, stool, cerebrospinal fluid (CSF), sputum, tissue biopsy, amniotic fluid, bone marrow, plasma, serum, or mucus, or another biological fluid type, or any combination thereof.

In one aspect, the subject can be a human. In another aspect, the disclosed method can be useful in veterinary medicine and the subject can be a common pet, experimental animal, or livestock animal such as, for example, a cat, dog, cattle, sheep, horse, swine, goat, guinea pig, hamster, rat, rabbit, mouse, chicken, turkey, or duck. In an alternative aspect, the subject can be a wild animal.

In one aspect, the sample can be an environmental sample such as, for example, soil, water, plant material, or a combination thereof.

In any of these aspects, the target nucleic acid can be an RNA molecule associated with a disease including, but not limited to, cancer, a cardiovascular disease, a neurological disorder, a bacterial disease, or a viral disease, or can be associated with an agricultural pest or an environmental contaminant. In some aspects, the RNA molecule can be a bacterial RNA molecule or an archaeal RNA molecule from an organism, such as, for example, Akkermansiaceae bacterium, Alicyclobacillus acidoterrestris, Bacillus sp. NSP2.1, Brevibacillus agri, Brevibacillus sp. FJAT54423, Brevibacillus sp. HD3.3A, Butyrivibrio hungatei, Catenovulum sp. CCB-QB4, Eubacterium eligens, Eubacterium rectale, Flavobacterium jumunjinense, Francisella cf. novicida Fx1, Lachnospira eligens, Lachnospiraceae bacterium GAM79, Lachnospiraceae bacterium ND2006, Methanomethylophilus alvus, Methanoplasma termitum, Methylacidimicrobium sp. AP8, Methylobacterium nodulans ORS 2060, Moranbacteria bacterium, Muribaculaceae bacterium, Opitutaceae bacterium TAV5, Pelobacter propionicus DSM 2379, Peregrinibacteria bacterium, Phycisphaeraceae bacterium, Prevotella bryantii, Prevotellamassilia sp., Pseudobutyrivibrio xylanivorans, Ruminococcus sp. JE7A12, Sedimentisphaera cyanobacteriorum, Succinivibrio sp., Sulfobacillus thermotolerans, or any combination thereof. In an aspect, this list is non-exhaustive; the disclosed methods and systems are effective in isolating any target RNA from a bacterial or archaeal source.

In an aspect, the viral disease can be HIV, hepatitis C virus (HCV), dengue virus, zika virus, or any combination thereof. In another aspect, the bacterial disease can be caused by Francisella hispaniensis FSC454, Moraxella bovoculi, Helcococcus kunzii, Sneathia amnii SN35, Alicyclobacillus acidoterrestris, Francisella tularensis, or any combination thereof. In an aspect, this list is non-exhaustive; the disclosed methods and systems are effective in isolating a target RNA from any disease-causing bacterium.

In one aspect, the target nucleic acid can be an miRNA, or can be genomic RNA from an RNA virus. In any of these aspects, the method is capable of detecting from about from about 1 pM to about 250 pM, from about 50 pM to about 250 pM, or from about 50 pM to about 100 pM, or about 50, 100, 150, 200, or 250 pM of the target nucleic acid in the biological sample. Further in this aspect, when the enzyme is Cas12i1 and the target nucleic acid is miRNA, the limit of detection can be from about 50 pM to about 250 pM. In an alternative aspect, when the enzyme is AsCas12a and the target nucleic acid is genomic RNA, the lower detection limit can be about 1 pM.

In any of these aspects, the target sequence does not need to be amplified prior to performing the method.

### Method for Silencing a Nucleic Acid

In another aspect, disclosed herein is method for silencing a target nucleic acid, the method including at least the step of contacting a sample or living organism containing the target nucleic acid with the disclosed nucleic acid detection system. In one aspect, the target nucleic acid can be an mRNA molecule.

In some aspects, the sample can be an isolated cell or tissue sample. In an alternative aspect, the living organism can be a human, cat, dog, cattle, sheep, horse, swine, goat, guinea pig, hamster, rat, rabbit, mouse, chicken, turkey, or duck.

In one aspect, performing the method can induce ribosome stalling, thereby repressing protein translation. In some aspects, the silencing is transient. In one aspect, “transient” as used herein refers to a period of from at least 16 to at least 24 hours, or at least 16 to at least 48 hours, or greater than 48 hours. In one aspect, and without wishing to be bound by theory, “transient” silencing lasting longer than 48 hours may be observed more often when genes with relatively low levels of expression are targeted.

### Method for Site-Specific Modification of a Target RNA

In one aspect, disclosed herein is a method for site-specific modification of a target RNA, the method including at least the step of contacting the RNA with a CRISPR system comprising a Cas12 protein fused to an N6-methyladenosine methyltransferase and one or more guide nucleotides; wherein each guide nucleotide includes a guide sequence capable of binding the target RNA and forming a complex with the Cas12 protein; and wherein when the guide nucleotide binds the target RNA and forms a complex with the Cas12 protein, the N6-methyladenosine methyltransferase methylates an N6 position of at least one adenine in the target RNA. In some aspects, the method can be conducted in a cell and the Cas12 protein fused to the N6-methyladenosine methyltransferase and the one or more guide nucleotides are co-transfected into the cell.

Further in this aspect, the N6-methyladenosine methyltransferase can be METTL3; however, other RNA-modification enzymes are contemplated and should be considered disclosed. In a further aspect, the Cas12 protein can be AsCas12a.

In any of these aspects, the guide nucleotide can be or include DNA and can have the characteristics of the guide nucleotides, including but not limited to spacers and scaffolds, as described previously herein.

Method for mRNA Knockdown in Cancer Cells

In yet another aspect, disclosed herein is method for knockdown of at least one mRNA in a cancer cell, the method including at least the step of contacting the at least one mRNA with a CRISPR system that includes a Cas12 protein and one or more guide nucleotides; wherein each guide nucleotide includes a guide sequence capable of binding the at least one mRNA and forming a complex with the Cas12 protein; and wherein when the guide nucleotide binds the target nucleic acid sequence and forms the complex with the Cas12 protein, ribosomal transcription of the at least one mRNA is stalled. Further in this aspect, Cas12 protein can be AsCas12a; however, other Cas12 proteins are contemplated and should be considered disclosed.

In any of these aspects, the guide nucleotide can be or include DNA and can have the characteristics of the guide nucleotides, including but not limited to spacers and scaffolds, as described previously herein. In another aspect, in the disclosed method, the Cas12 protein and the one or more guide nucleotides can be co-transfected into the cancer cell. In some aspects, co-transfection is accomplished using a lentivirus system; however, other transfection methods are contemplated and should be considered disclosed. In one aspect, the cancer cell can be in a culture dish. In another aspect, the cancer cell can be in a living subject. In any of these aspects, the cancer cell can be selected from a cervical cancer cell, a liver cancer cell, or a breast cancer cell.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a Cas12 enzyme,” “a ΨDNA,” or “an miRNA,” include, but are not limited to, mixtures or combinations of two or more such Cas12 enzymes, ΨDNAs, or miRNAs, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), CRISPR RNA (crRNA), Trans-activating crRNA (tracrRNA), or coding mRNA (messenger RNA).

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein with reference to the relationship between DNA, cDNA, CRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, “variant” can refer to a polynucleotide or polypeptide that differs from a reference polynucleotide or polypeptide, but retains essential and/or characteristic properties (structural and/or functional) of the reference polynucleotide or polypeptide. A typical variant of a polypeptide differs in amino acid sequence from another, reference polypeptide. The differences can be limited so that the sequences of the reference polypeptide and the variant are closely similar overall and, in many regions, identical. A variant and reference polypeptide may differ in nucleic or amino acid sequence by one or more modifications at the sequence level or post-transcriptional or post-translational modifications (e.g., substitutions, additions, deletions, methylation, glycosylations, etc.). A substituted nucleic acid may or may not be an unmodified nucleic acid of adenine, thiamine, guanine, cytosine, uracil, including any chemically, enzymatically or metabolically modified forms of these or other nucleotides. A substituted amino acid residue may or may not be one encoded by the genetic code. A variant of a polypeptide may be naturally occurring such as an allelic variant, or it may be a variant that is not known to occur naturally. “Variant” includes functional and structural variants.

As used herein, “gene” refers to a hereditary unit corresponding to a sequence of DNA that occupies a specific location on a chromosome and that contains the genetic instruction for a characteristic(s) or trait(s) in an organism. As used herein, “synthetic gene” can refer to a recombinant gene comprising one or more coding sequences for a protein of interest, or a synthetically purified protein that is not naturally occurring in its purified state.

As used herein, the terms “guide polynucleotide,” “guide sequence,” or “guide RNA” (gRNA or sgRNA) as can refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). A guide polynucleotide (also referred to herein as a guide sequence and includes single guide sequences (sgRNA)) can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The guide polynucleotide (gRNA or sgRNA) can include a nucleotide sequence that is complementary to a target DNA sequence. This portion of the guide sequence can be referred to as the complementary region of the guide RNA or the CRISPR RNA (crRNA). Another portion of the guide sequence serves as a binding scaffold for the CRISPR-associated (Cas) nuclease. This portion of the guide sequence can be referred to as the tracrRNA. In one aspect, crRNA/tracrRNA can also work with the disclosed approach. Further in this aspect, since crRNA is shorter, it may be easier to incorporate the desired DNA modifications to the crRNAs by ligation or synthesis compared to incorporation into sgRNAs. In a further aspect, and without wishing to be bound by theory, tracrRNAs are generally universal and work with any sequence of crRNAs and so the crRNA/tracrRNA system may be more economical for use. The guide sequence can also include one or more miRNA target sequences coupled to the 3′ end of the guide sequence. The guide sequence can include one or more MS2 RNA aptamers incorporated within the portion of the guide strand that is not the complementary portion. As used herein the term guide sequence can include any specially modified guide sequences, including but not limited to those configured for use in synergistic activation mediator (SAM) implemented CRISPR or suppression.

A guide polynucleotide can be less than about 150, 125, 75, 50, 45, 40, 35, 30, 25, 20, 15, 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

As used herein, “identity,” is a relationship between two or more polypeptide or polynucleotide sequences, as determined by comparing the sequences. In the art, “identity” also refers to the degree of sequence relatedness between polypeptide as determined by the match between strings of such sequences. “Identity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48:1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch (J. Mol. Biol., 1970, 48:443-453) algorithm (e.g., NBLAST, and XBLAST). The default parameters are used to determine the identity for the polypeptides or polynucleotides of the present disclosure.

As used herein, “heterologous” refers to compounds, molecules, nucleotide sequences (including genes), and polypeptide sequences (including peptides and proteins) that are different in both activity (function) and sequence or chemical structure. As used herein, “heterologous” can also refer to a gene or gene product that is from a different organism. For example, a human GTP cyclohydrolase or a synthase can be said to be heterologous when expressed in yeast.

As used herein, “homolog” refers to a polypeptide sequence that shares a threshold level of similarity and/or identity as determined by alignment of matching amino acids. Two or more polypeptides determined to be homologs are said to be homologs. Homology is a qualitative term that describes the relationship between polypeptide sequences that is based upon the quantitative similarity.

As used herein, “paralog” refers to a homolog produced via gene duplication of a gene. In other words, paralogs are homologs that result from divergent evolution from a common ancestral gene.

As used herein, “orthologs” refers to homologs produced by speciation followed by divergence of sequence but not activity in separate species. When speciation follows duplication and one homolog sorts with one species and the other copy sorts with the other species, subsequent divergence of the duplicated sequence is associated with one or the other species. Such species specific homologs are referred to herein as orthologs.

As used herein, “similarity” is a quantitative term that defines the degree of sequence match between two compared polypeptide sequences.

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans).

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “culturing” refers to maintaining cells under conditions in which they can proliferate and avoid senescence as a group of cells. “Culturing” can also include conditions in which the cells also or alternatively differentiate.

As used herein, the term “specific binding” or “preferential binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, K_{d}, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a K_{d} of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

“Locked nucleic acids” (LNAs) are modified monomers derived from RNA. LNAs include a methylene bridge bond between the 2′ oxygen of the ribose to the 4′ carbon of the ribose. LNAs follow standard base pairing rules. In one aspect, when LNAs are added to a probe or other sequence intended to hybridize, they can increase structural stability and melting point. In other aspects, LNAs can also add resistance to degradation by nucleases.

“Phosphorothioate” bonds include one substitution of a sulfur for a non-bridging oxygen atom in the backbone of an oligonucleotide. In an aspect, inclusion of a phosphorothioate bond can increase nuclease resistance. In some aspects, phosphorothioate bonds are typically introduced between the first several bases at the 5′ end of an oligonucleotide, the last several bases at the 3′ end of an oligonucleotide, or both.

“Double-quenched probes” include common 5′ fluorophore and 3′ quencher pairs with an additional, internal quencher. In one aspect, double-quenched probes decrease the number of bases between fluorophore and quencher. In a further aspect, this shortened distance can lead to more thorough quenching and/or a quenching with lower background. In another aspect, using double-quenched probes enables the use of longer probes for designing in AT-rich target regions.

Unless otherwise specified, atmospheres referred to herein are based on atmospheric pressure (i.e. one atmosphere) and temperatures are ambient.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.
Aspect 1. A nucleic acid detection system comprising:
a CRISPR system comprising a Cas12 protein and one or more guide nucleotides; and
an ssDNA comprising a fluorophore and a quencher;
wherein each guide nucleotide comprises a guide sequence capable of binding a target nucleic acid sequence and forming a complex with the Cas12 protein;
wherein the guide nucleotide comprises a fluorophore and a quencher; and
wherein when the guide nucleotide binds the target nucleic acid sequence and forms a complex with the Cas12 protein, the Cas12 protein performs trans cleavage on the ssDNA, separating the fluorophore from the quencher.
Aspect 2. The nucleic acid detection system of aspect 1, wherein the Cas12 protein comprises a Cas12i protein, a Cas12j protein, a Cas12b protein, a Cas12a protein, or any combination thereof.
Aspect 3. The nucleic acid detection system of aspect 2, wherein the Cas12i protein comprises Cas12i1, Cas12i2, or any combination thereof.
Aspect 4. The nucleic acid detection system of aspect 2, wherein the Cas12a protein comprises AsCas12a, ErCas12a, or any combination thereof.
Aspect 5. The nucleic acid detection system of aspect 1, wherein the guide nucleotide comprises a spacer, wherein the spacer comprises the guide sequence.
Aspect 6. The nucleic acid detection system of aspect 1, wherein the spacer is from about 20 to about 23 nucleotides long.
Aspect 7. The nucleic acid detection system of aspect 1, wherein the fluorophore comprises 5 (6)-carboxyfluorescein (56-FAM), 5′-hexachlorofluorescein (5HEX), or any combination thereof.
Aspect 8. The nucleic acid detection system of aspect 1, wherein the quencher comprises 3′-Iowa Black FQ (3IABKFQ).
Aspect 9. The nucleic acid detection system of aspect 7, wherein the ssDNA comprises 56-FAM-TTATT-3IABKFQ or 5HEX-TTTTTTTT-3IABKFQ.
Aspect 10. The nucleic acid detection system of aspect 1, wherein the guide nucleotide further comprises a scaffold.
Aspect 11. The nucleic acid detection system of aspect 10, wherein the scaffold is located at a 3′ end of the guide sequence.
Aspect 12. The nucleic acid detection system of aspect 1, wherein the scaffold is from about 19 to about 25 nucleotides long.
Aspect 13. The nucleic acid detection system of aspect 1, wherein the guide nucleotide comprises DNA.
Aspect 14. The nucleic acid detection system of aspect 1, wherein the target nucleic acid sequence comprises RNA.
Aspect 15. A method for detecting a target nucleic acid, the method comprising:
(a) contacting a sample suspected of containing the target nucleic acid with the nucleic acid detection system of any one of aspects 1-10; and
(b) measuring a fluorescence signal from the nucleic acid detection system;
wherein when the fluorescence signal is present, the sample contains the target nucleic acid.
Aspect 16. The method of aspect 15, wherein the sample comprises a biological sample from a subject.
Aspect 17. The method of aspect 15, further comprising performing at least one processing step on the sample prior to performing step (a).
Aspect 18. The method of aspect 17, wherein the at least one processing step comprises nucleic acid extraction, nucleic acid purification, or any combination thereof.
Aspect 19. The method of aspect 15, wherein the biological sample comprises blood, saliva, urine, stool, cerebrospinal fluid (CSF), sputum, tissue biopsy, amniotic fluid, bone marrow, plasma, serum, or mucus.
Aspect 20. The method of aspect 15, wherein the sample is isolated from a human, cat, dog, cattle, sheep, horse, swine, goat, guinea pig, hamster, rat, rabbit, mouse, chicken, turkey, or duck.
Aspect 21. The method of aspect 15, wherein the sample is isolated from a wild animal.
Aspect 22. The method of aspect 15, wherein the sample comprises an environmental sample.
Aspect 23. The method of aspect 22, wherein the environmental sample comprises soil, water, plant material, or any combination thereof.
Aspect 24. The method of aspect 15, wherein the target nucleic acid is an RNA molecule.
Aspect 25. The method of aspect 24, wherein the RNA molecule comprises a bacterial RNA molecule or an archaeal RNA molecule.
Aspect 26. The method of aspect 25, wherein the bacterial RNA molecule or archaeal RNA molecule is isolated from Akkermansiaceae bacterium, Alicyclobacillus acidoterrestris, Bacillus sp. NSP2.1, Brevibacillus agri, Brevibacillus sp. FJAT54423, Brevibacillus sp. HD3.3A, Butyrivibrio hungatei, Catenovulum sp. CCB-QB4, Eubacterium eligens, Eubacterium rectale, Flavobacterium jumunjinense, Francisella cf. novicida Fx1, Lachnospira eligens, Lachnospiraceae bacterium GAM79, Lachnospiraceae bacterium ND2006, Methanomethylophilus alvus, Methanoplasma termitum, Methylacidimicrobium sp. AP8, Methylobacterium nodulans ORS 2060, Moranbacteria bacterium, Muribaculaceae bacterium, Opitutaceae bacterium TAV5, Pelobacter propionicus DSM 2379, Peregrinibacteria bacterium, Phycisphaeraceae bacterium, Prevotella bryantii, Prevotellamassilia sp., Pseudobutyrivibrio xylanivorans, Ruminococcus sp. JE7A12, Sedimentisphaera cyanobacteriorum, Succinivibrio sp., Sulfobacillus thermotolerans, or any combination thereof.
Aspect 27. The method of aspect 15, wherein the RNA molecule is associated with a disease, an agricultural pest, or an environmental contaminant.
Aspect 28. The method of aspect 27, wherein the disease comprises cancer, a cardiovascular disease, a neurological disorder, a bacterial disease, or a viral disease.
Aspect 29. The method of aspect 28, wherein the viral disease comprises HIV, hepatitis C virus (HCV), dengue virus, zika virus, or any combination thereof.
Aspect 30. The method of aspect 28, wherein the bacterial disease is caused by Francisella hispaniensis FSC454, Moraxella bovoculi, Helcococcus kunzii, Sneathia amnii SN35, Alicyclobacillus acidoterrestris, Francisella tularensis, or any combination thereof.
Aspect 31. The method of aspect 15, wherein the target nucleic acid is an miRNA.
Aspect 32. The method of aspect 15, wherein the method is capable of detecting from about 50 pM to about 250 pM of the target nucleic acid in the biological sample.
Aspect 33. The method of aspect 15, wherein the target sequence does not need to be amplified prior to performing the method.
Aspect 34. A method for silencing a target nucleic acid, the method comprising contacting a sample or living organism containing the target nucleic acid with the nucleic acid detection system of aspect 1.
Aspect 35. The method of aspect 34, wherein the target nucleic acid is an mRNA molecule.
Aspect 36. The method of aspect 34, wherein the sample is an isolated cell or tissue sample.
Aspect 37. The method of aspect 34, wherein the living organism is a human, cat, dog, cattle, sheep, horse, swine, goat, guinea pig, hamster, rat, rabbit, mouse, chicken, turkey, or duck.
Aspect 38. The method of aspect 34, wherein performing the method induces ribosome stalling, thereby repressing protein translation.
Aspect 39. The method of aspect 34, wherein silencing is transient.
Aspect 40. A method for site-specific modification of a target RNA, the method comprising contacting the RNA with a CRISPR system comprising a Cas12 protein fused to an N6-methyladenosine methyltransferase and one or more guide nucleotides;
wherein each guide nucleotide comprises a guide sequence capable of binding the target RNA and forming a complex with the Cas12 protein; and
wherein when the guide nucleotide binds the target RNA and forms a complex with the Cas12 protein, the N6-methyladenosine methyltransferase methylates an N6 position of at least one adenine in the target RNA.
Aspect 41. The method of aspect 40, wherein the method is conducted in a cell, and wherein the Cas12 protein fused to the N6-methyladenosine methyltransferase and the one or more guide nucleotides are co-transfected into the cell.
Aspect 42. The method of aspect 40, wherein the N6-methyladenosine methyltransferase comprises METTL3.
Aspect 43. The method of aspect 40, wherein the Cas12 protein comprises AsCas12a.
Aspect 44. The method of aspect 40, wherein the guide nucleotide comprises a spacer, wherein the spacer comprises the guide sequence.
Aspect 45. The method of aspect 44, wherein the spacer is from about 20 to about 23 nucleotides long.
Aspect 46. The method of aspect 40, wherein the guide nucleotide further comprises a scaffold.
Aspect 47. The method of aspect 46, wherein the scaffold is located at a 3′ end of the guide sequence.
Aspect 48. The method of aspect 46, wherein the scaffold is from about 19 to about 25 nucleotides long.
Aspect 49. The method of aspect 40, wherein the guide nucleotide comprises DNA.
Aspect 50. A method for knockdown of at least one mRNA in a cancer cell, the method comprising contacting the at least one mRNA with a CRISPR system comprising a Cas12 protein and one or more guide nucleotides;
wherein each guide nucleotide comprises a guide sequence capable of binding the at least one mRNA and forming a complex with the Cas12 protein; and
wherein when the guide nucleotide binds the target nucleic acid sequence and forms the complex with the Cas12 protein, ribosomal transcription of the at least one mRNA is stalled.
Aspect 51. The method of aspect 50, wherein the Cas12 protein comprises AsCas12a.
Aspect 52. The method of aspect 50, wherein the guide nucleotide comprises a spacer, wherein the spacer comprises the guide sequence.
Aspect 53. The method of aspect 52, wherein the spacer is from about 20 to about 23 nucleotides long.
Aspect 54. The method of aspect 50, wherein the guide nucleotide further comprises a scaffold.
Aspect 55. The method of aspect 54, wherein the scaffold is located at a 3′ end of the guide sequence.
Aspect 56. The method of aspect 54, wherein the scaffold is from about 19 to about 25 nucleotides long.
Aspect 57. The method of aspect 50, wherein the guide nucleotide comprises DNA.
Aspect 58. The method of aspect 50, wherein in the method, the Cas12 protein and the one or more guide nucleotides are co-transfected into the cancer cell.
Aspect 59. The method of aspect 58, wherein co-transfection is accomplished using a lentivirus system.
Aspect 60. The method of aspect 50, wherein the cancer cell is in a culture dish.
Aspect 61. The method of aspect 50, wherein the cancer cell is in a living subject.
Aspect 62. The method of aspect 50, wherein the cancer cell comprises a cervical cancer cell, a liver cancer cell, or a breast cancer cell.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Results

### Engineering of ΨDNAs for RNA Detection Using Cas12 Enzymes

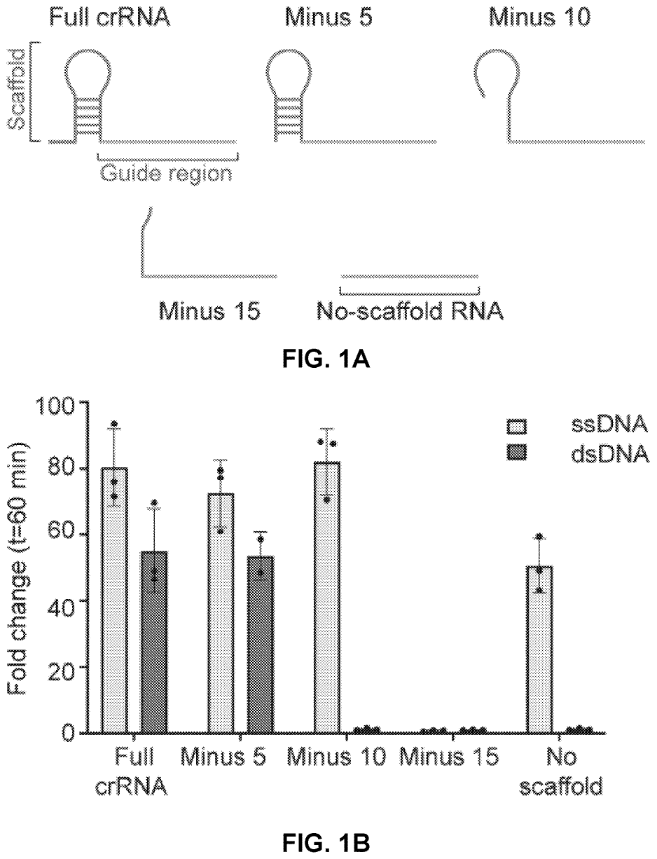

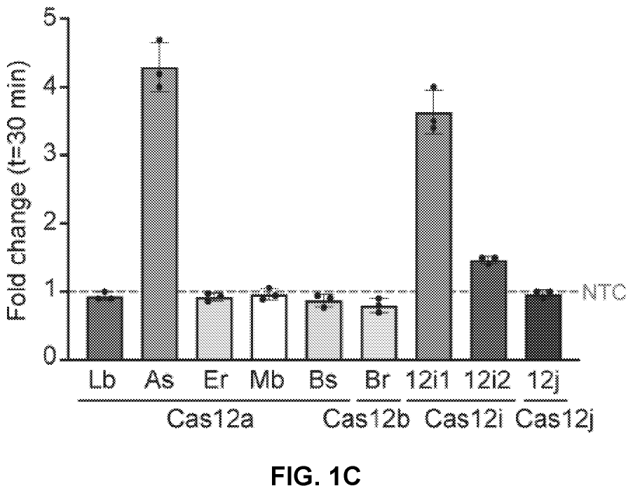

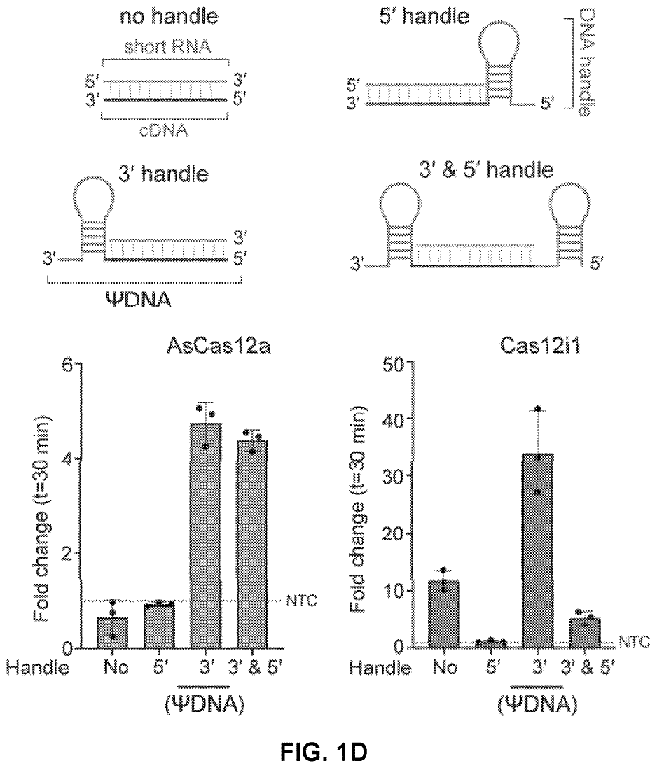

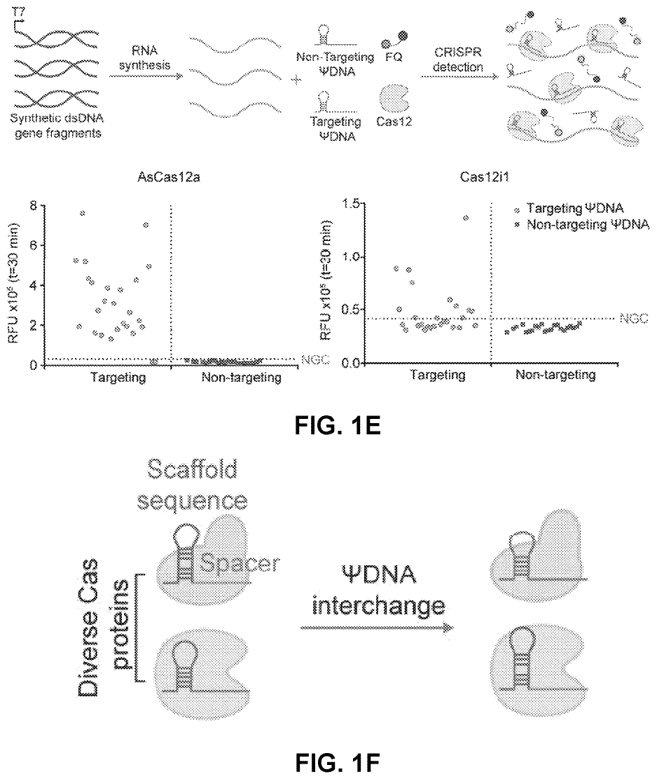

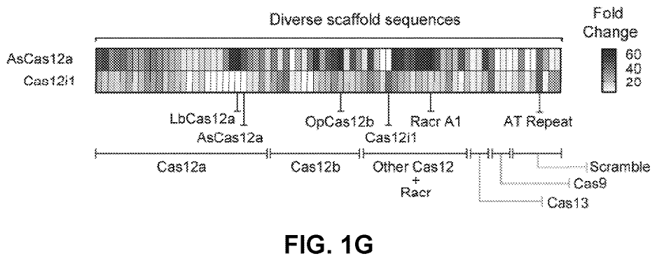

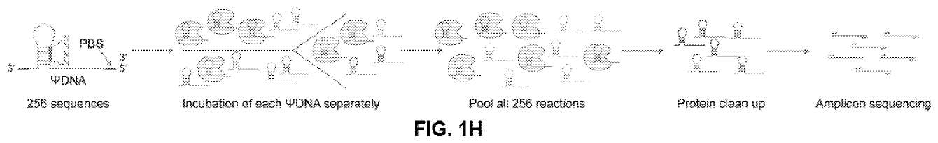

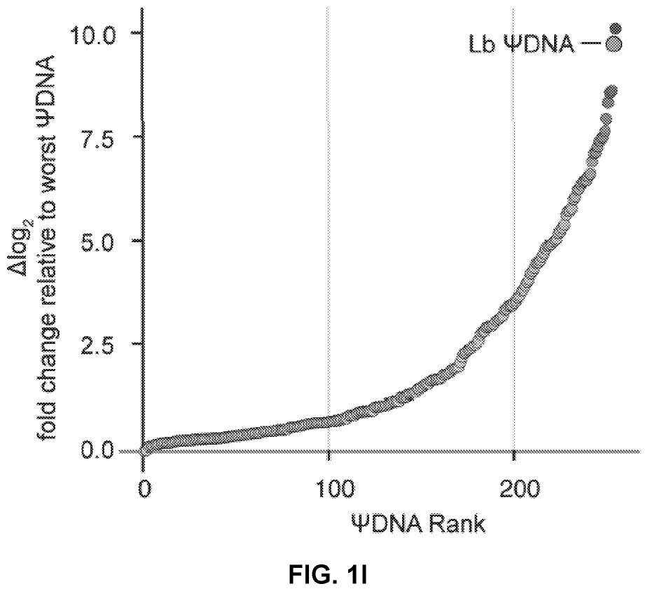

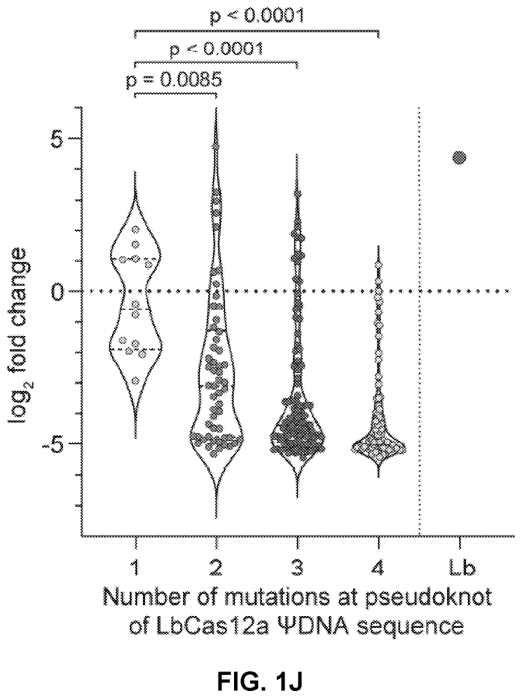

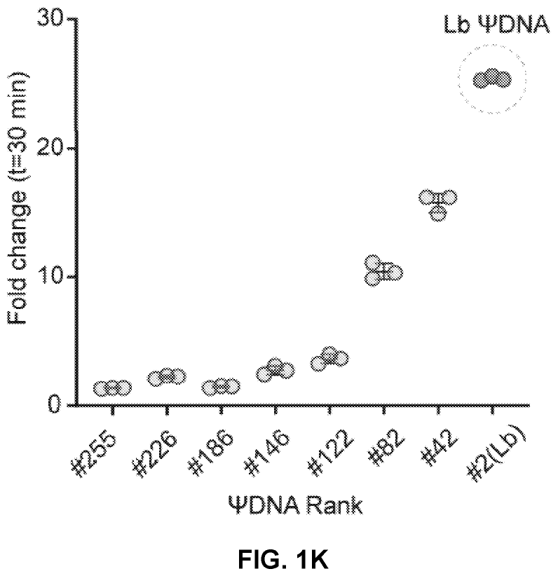

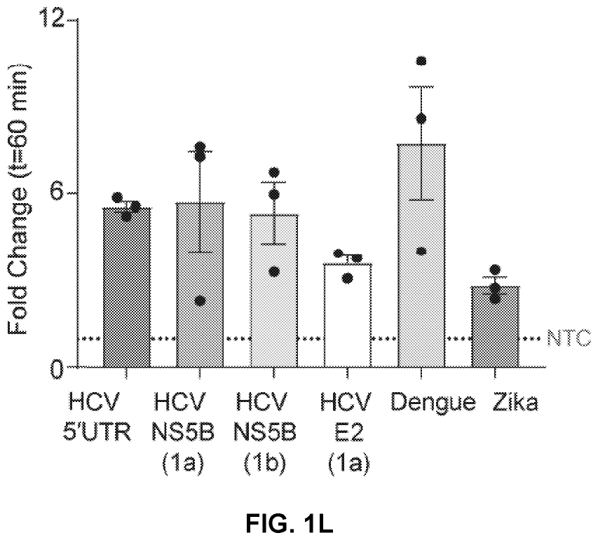

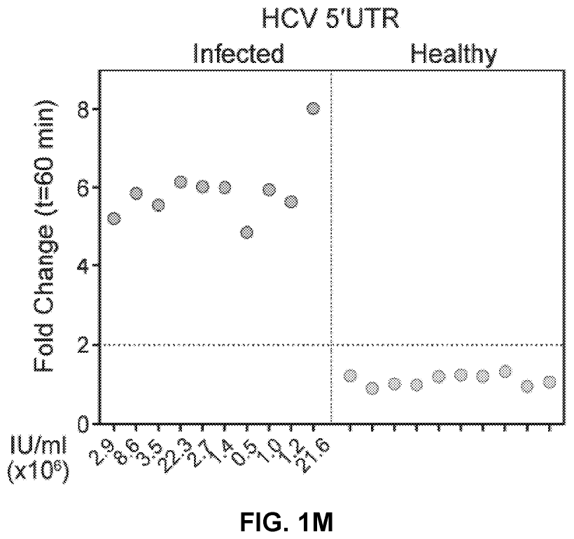

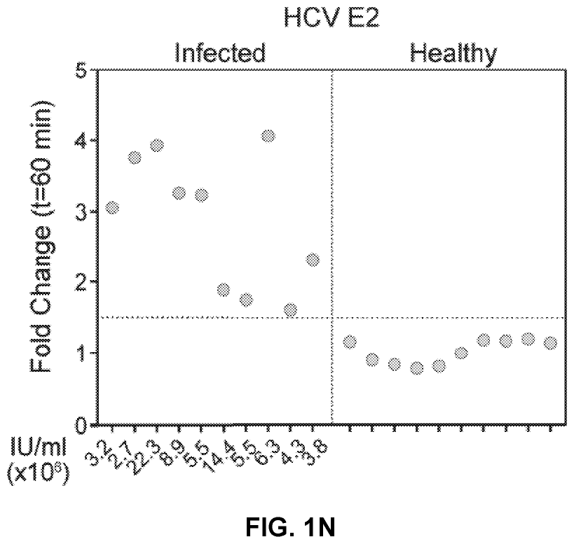

To define how crRNA scaffold length shapes Cas12i1 trans-cleavage, the scaffold was trimmed by 5, 10, or 15 nucleotides and generated a no-scaffold guide RNA (FIG. 1A). Cas12i1 required a near-complete scaffold for potent activity on double-stranded DNA; full-length and minus-five crRNAs were most effective (FIG. 1B). Single-stranded DNA targets were still cleaved with the no-scaffold guide RNA, revealing distinct scaffold demands for the two substrates. Eight Cas12 orthologs with no-scaffold guide RNA were next evaluated on single-stranded DNA (FIG. 1C). Only Cas12i1 and AsCas12a retained strong activity, implying unique conformational flexibility that could permit DNA guides. Sequences for Cas1211 and AsCas12a are shown in Table 1.

TABLE 1
Protein Amino Acid Sequences

| Protein | Amino Acid Sequence |
|---|---|
| AsCas12a | MTQFEGFTNLYQVSKTLRFELIPQGKTLKHIQEQGFIEEDKARNDHYKELKPIIDRIYKTYADQCLQL |
| (SEQ ID | VQLDWENLSAAIDSYRKEKTEETRNALIEEQATYRNAIHDYFIGRTDNLTDAINKRHAEIYKGLFKAE |
| NO. 1) | LFNGKVLKQLGTVTTTEHENALLRSFDKFTTYFSGFYENRKNVFSAEDISTAIPHRIVQDNFPKFKE |
| | NCHIFTRLITAVPSLREHFENVKKAIGIFVSTSIEEVFSFPFYNQLLTQTQIDLYNQLLGGISREAGTEK |
| | IKGLNEVLNLAIQKNDETAHIIASLPHRFIPLFKQILSDRNTLSFILEEFKSDEEVIQSFCKYKTLLRNE |
| | NVLETAEALFNELNSIDLTHIFISHKKLETISSALCDHWDTLRNALYERRISELTGKITKSAKEKVQRS |
| | LKHEDINLQEIISAAGKELSEAFKQKTSEILSHAHAALDQPLPTTLKKQEEKEILKSQLDSLLGLYHLL |
| | DWFAVDESNEVDPEFSARLTGIKLEMEPSLSFYNKARNYATKKPYSVEKFKLNFQMPTLASGWDV |
| | NKEKNNGAILFVKNGLYYLGIMPKQKGRYKALSFEPTEKTSEGFDKMYYDYFPDAAKMIPKCSTQL |
| | KAVTAHFQTHTTPILLSNNFIEPLEITKEIYDLNNPEKEPKKFQTAYAKKTGDQKGYREALCKWIDFT |
| | RDFLSKYTKTTSIDLSSLRPSSQYKDLGEYYAELNPLLYHISFQRIAEKEIMDAVETGKLYLFQIYNKD |
| | FAKGHHGKPNLHTLYWTGLFSPENLAKTSIKLNGQAELFYRPKSRMKRMAHRLGEKMLNKKLKD |
| | QKTPIPDTLYQELYDYVNHRLSHDLSDEARALLPNVITKEVSHEIIKDRRFTSDKFFFHVPITLNYQA |
| | ANSPSKFNQRVNAYLKEHPETPIIGIDRGERNLIYITVIDSTGKILEQRSLNTIQQFDYQKKLDNREKE |
| | RVAARQAWSVVGTIKDLKQGYLSQVIHEIVDLMIHYQAVVVLENLNFGFKSKRTGIAEKAVYQQFEK |
| | MLIDKLNCLVLKDYPAEKVGGVLNPYQLTDQFTSFAKMGTQSGFLFYVPAPYTSKIDPLTGFVDPFV |
| | WKTIKNHESRKHFLEGFDFLHYDVKTGDFILHFKMNRNLSFQRGLPGFMPAWDIVFEKNETQFDA |
| | KGTPFIAGKRIVPVIENHRFTGRYRDLYPANELIALLEEKGIVFRDGSNILPKLLENDDSHAIDTMVALI |
| | RSVLQMRNSNAATGEDYINSPVRDLNGVCFDSRFQNPEWPMDADANGAYHIALKGQLLLNHLKE |
| | SKDLKLQNGISNQDWLAYIQELRN |
| Cas12i1 | MKIEEGKGHHHHHHMSNKEKNASETRKAYTTKMIPRSHDRMKLLGNFMDYLMDGTPIFFELWNQ |
| (SEQ ID | FGGGIDRDIISGTANKDKISDDLLLAVNWFKVMPINSKPQGVSPSNLANLFQQYSGSEPDIQAQEYF |
| NO. 2) | ASNFDTEKHQWKDMRVEYERLLAELQLSRSDMHHDLKLMYKEKCIGLSLSTAHYITSVMFGTGAK |
| | NNRQTKHQFYSKVIQLLEESTQINSVEQLASIILKAGDCDSYRKLRIRCSRKGATPSILKIVQDYELG |
| | TNHDDEVNVPSLIANLKEKLGRFEYECEWKCMEKIKAFLASKVGPYYLGSYSAMLENALSPIKGMT |
| | TKNCKFVLKQIDAKNDIKYENEPFGKIVEGFFDSPYFESDTNVKWVLHPHHIGESNIKTLWEDLNAI |
| | HSKYEEDIASLSEDKKEKRIKVYQGDVCQTINTYCEEVGKEAKTPLVQLLRYLYSRKDDIAVDKIIDG |
| | ITFLSKKHKVEKQKINPVIQKYPSFNFGNNSKLLGKIISPKDKLKHNLKCNRNQVDNYIWIEIKVLNTK |
| | TMRWEKHHYALSSTRFLEEVYYPATSENPPDALAARFRTKTNGYEGKPALSAEQIEQIRSAPVGLR |
| | KVKKRQMRLEAARQQNLLPRYTWGKDFNINICKRGNNFEVTLATKVKKKKEKNYKVVLGYDANIV |
| | RKNTYAAIEAHANGDGVIDYNDLPVKPIESGFVTVESQVRDKSYDQLSYNGVKLLYCKPHVESRRS |
| | FLEKYRNGTMKDNRGNNIQIDFMKDFEAIADDETSLYYFNMKYCKLLQSSIRNHSSQAKEYREEIF |
| | ELLRDGKLSVLKLSSLSNLSFVMFKVAKSLIGTYFGHLLKKPKNSKSDVKAPPITDEDKQKADPEMF |
| | ALRLALEEKRLNKVKSKKEVIANKIVAKALELRDKYGPVLIKGENISDTTKKGKKSSTNSFLMDWLA |
| | RGVANKVKEMVMMHQGLEFVEVNPNFTSHQDPFVHKNPENTFRARYSRCTPSELTEKNRKEILS |
| | FLSDKPSKRPTNAYYNEGAMAFLATYGLKKNDVLGVSLEKFKQIMANILHQRSEDQLLFPSRGGM |
| | FYLATYKLDADATSVNWNGKQFWVCNADLVAAYNVGL VDIQKDFKKK |

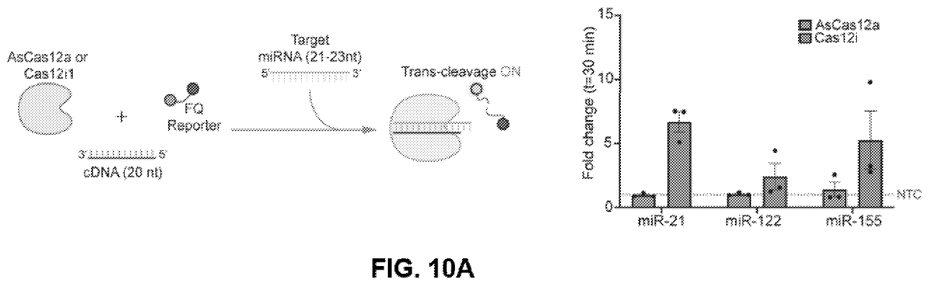

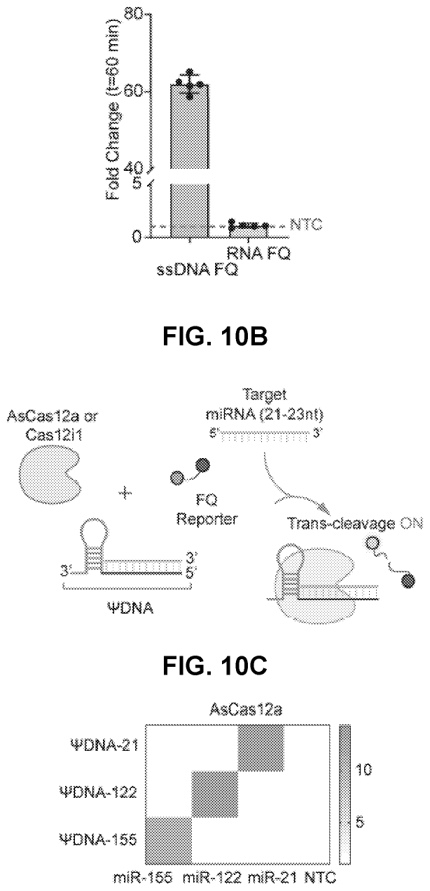

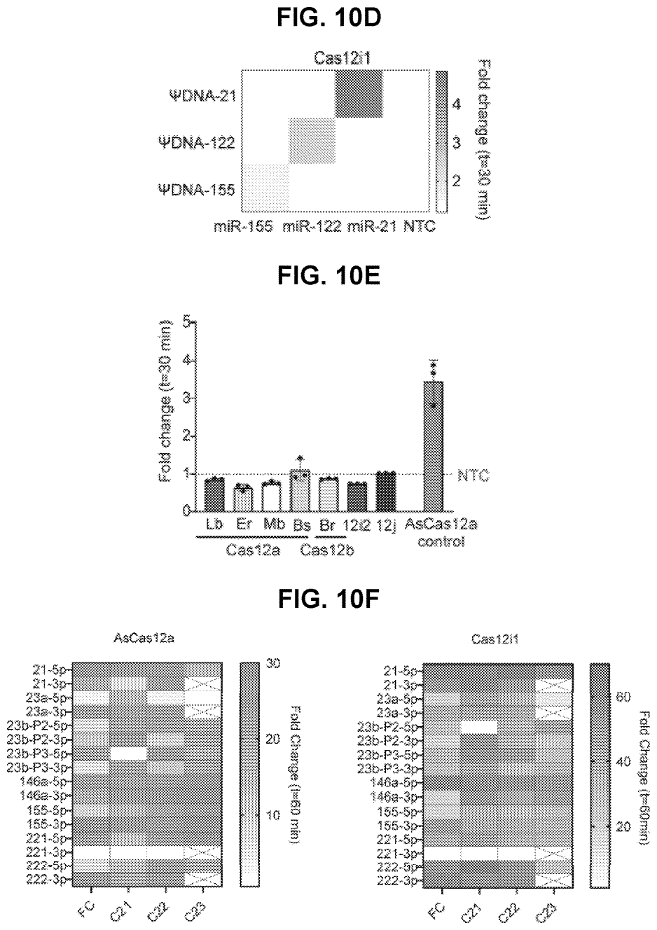

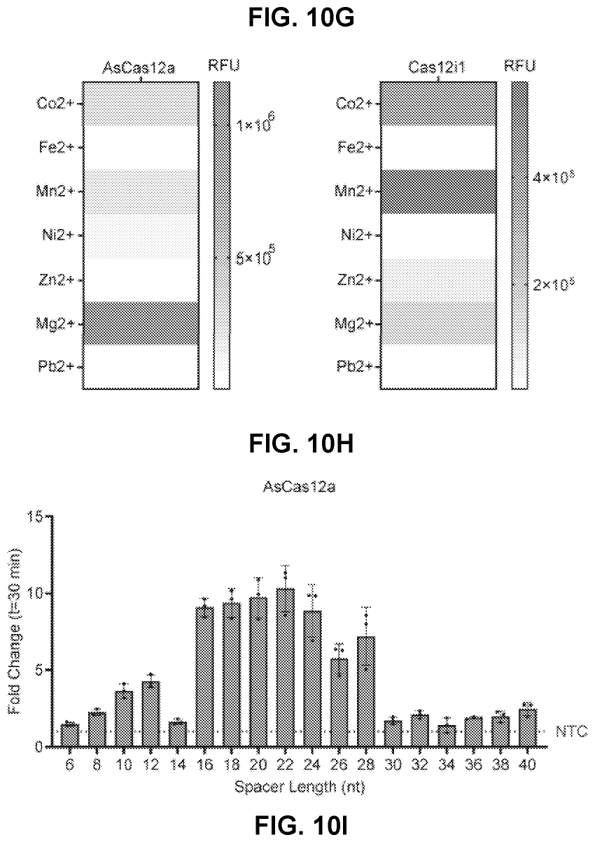

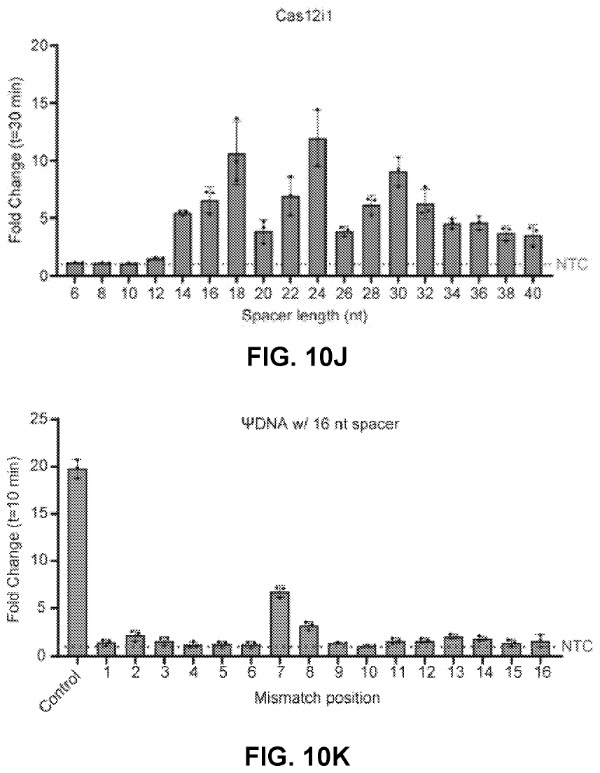

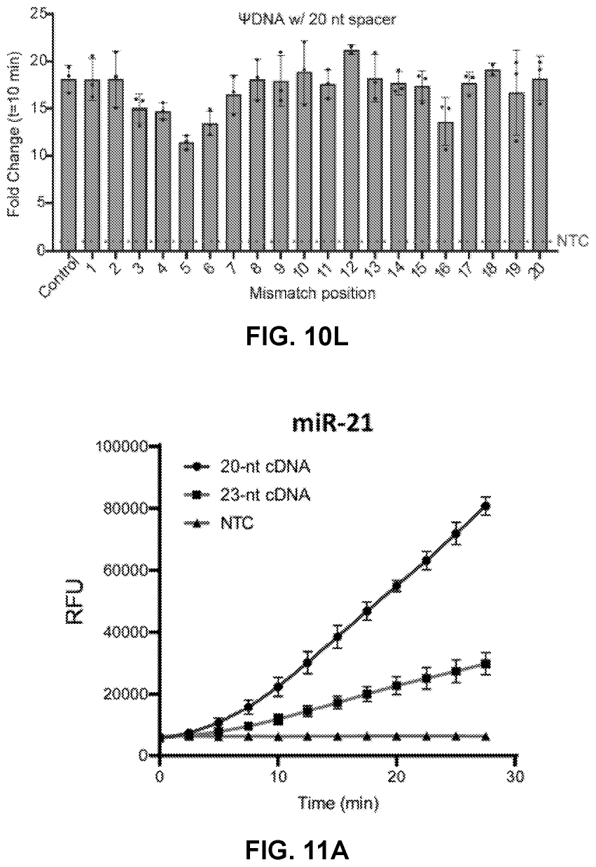

Guided by this observation, the no-scaffold RNA was replaced with 20-nt complementary DNA guides lacking scaffolds and challenged the enzymes with synthetic microRNAs. Only Cas12i1 detected miR-21, miR-122, and miR-155, while AsCas12a showed no activity (FIG. 10A). Characterization of the substrates involved in ΨDNA-mediated trans-cleavage revealed that AsCas12a retains its specificity for ssDNA (FIG. 10B). To optimize Cas12i1 and enable AsCas12a for a DNA-guided system, various structural modifications to the cDNA guide were next engineered (FIG. 1D). Specifically, it was hypothesized that adding DNA mimics of the native crRNA scaffold sequence to the cDNA guide might improve the trans-cleavage activity. Four configurations were tested: cDNA alone (no handle), cDNA with a 5′ handle, cDNA with a 3′ handle, and cDNA with both 5′ and 3′ handles. These results demonstrated that the 3′ handle configuration, which is herein termed pseudo-DNA (ΨDNA), yielded the highest detection activity for both AsCas12a and Cas12i1 enzymes (FIG. 1D). Using this optimized ΨDNA architecture, additional Cas12 orthologs were next evaluated. Consistent with the earlier results, ΨDNA-mediated trans-cleavage activation was observed only for AsCas12a and Cas12i1 (FIGS. 1D and 10F). This structural arrangement likely stabilizes the guide-enzyme complex by positioning the DNA scaffold in a configuration analogous to the native crRNA's 5′ handle. Interestingly, while Cas12i1 showed generally higher activity with the basic cDNA guide, AsCas12a performed notably better with the optimized ΨDNA structure, suggesting different structural requirements between these enzymes.

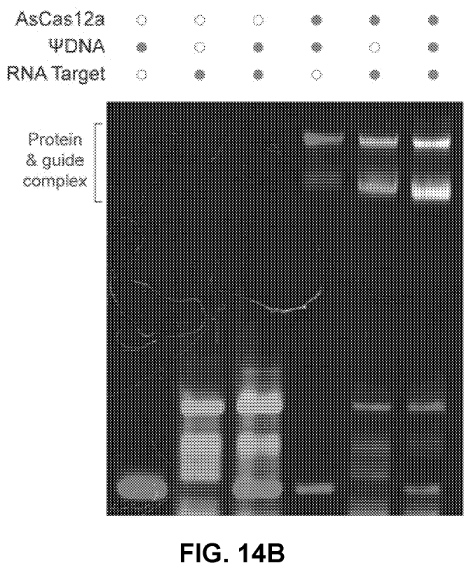

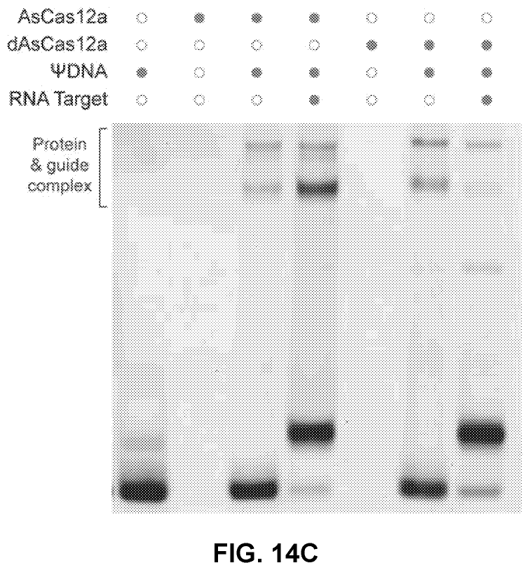

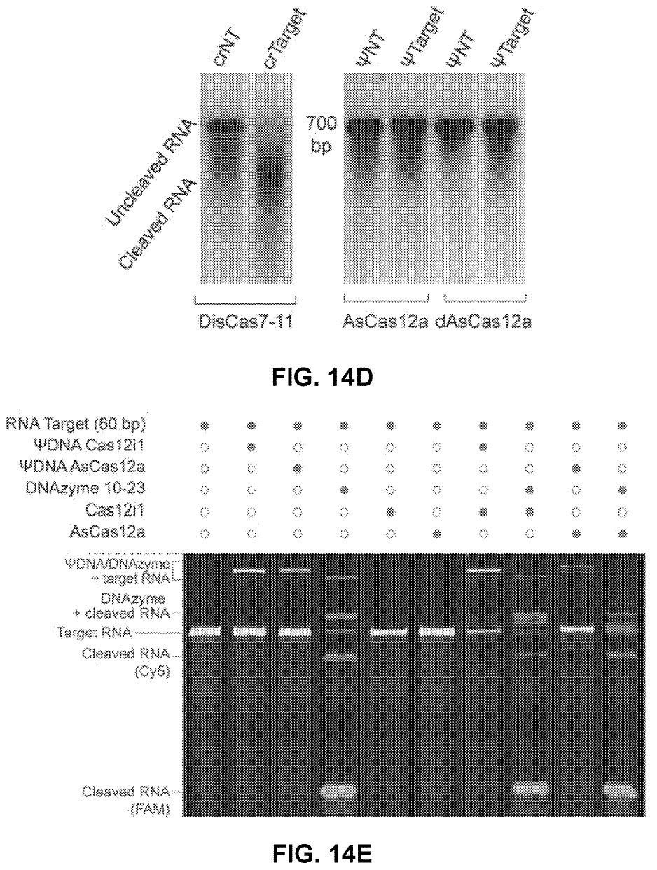

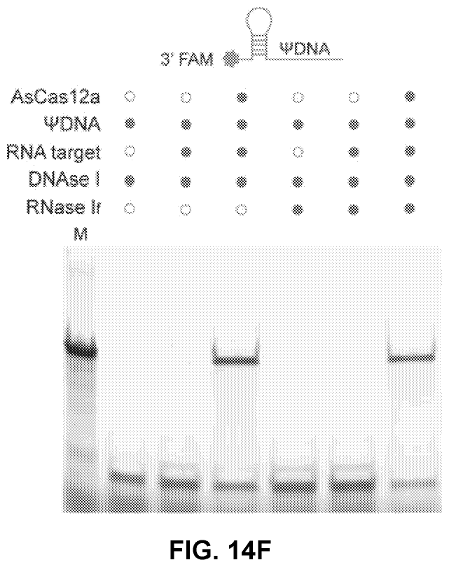

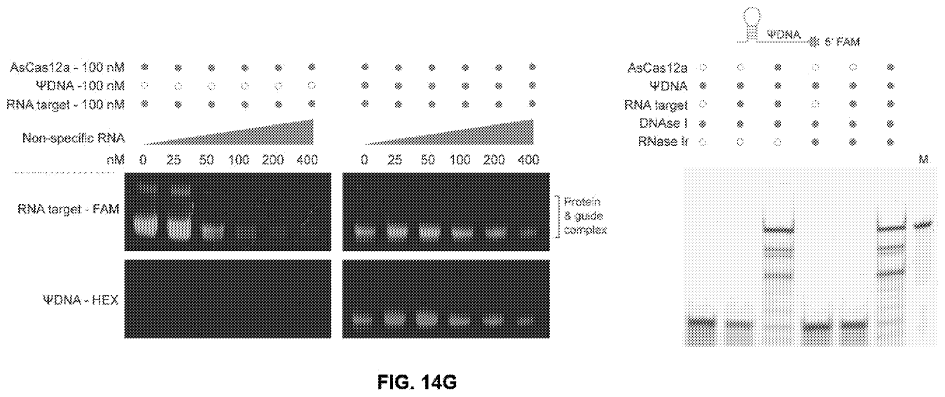

Excellent specificity was observed, as each ΨDNA guide triggered its enzyme only in the presence of the cognate microRNA (FIGS. 10C-10E), and detection was extended to 14 additional microRNAs with similar fidelity (FIG. 10G). Optimal guide length ranged from 16-28 nt, and shorter guides heightened mismatch discrimination (FIGS. 10I-10L). Activity persisted under varied divalent cations, underscoring biochemical robustness (FIG. 10H). Electrophoretic mobility-shift assays confirmed stable assembly of AsCas12a with ΨDNA and target RNA (FIGS. 14A-14C). Although AsCas12a exhibits non-specific RNA binding, it was found that non-specific RNAs compete less effectively with the target RNA in the presence of ΨDNA (FIG. 14G). No cis- or trans-cleavage of RNA was observed, verifying that activation is confined to collateral DNA reporters (FIGS. 14D-14E). In addition, to further assess the mode of interaction among ΨDNA, the target RNA, and AsCas12a, DNase I footprinting assays were performed using fluorescently labeled ΨDNA. Protection from DNase I digestion was observed only in the presence of AsCas12a, particularly at the 5′ end (FIG. 14F).

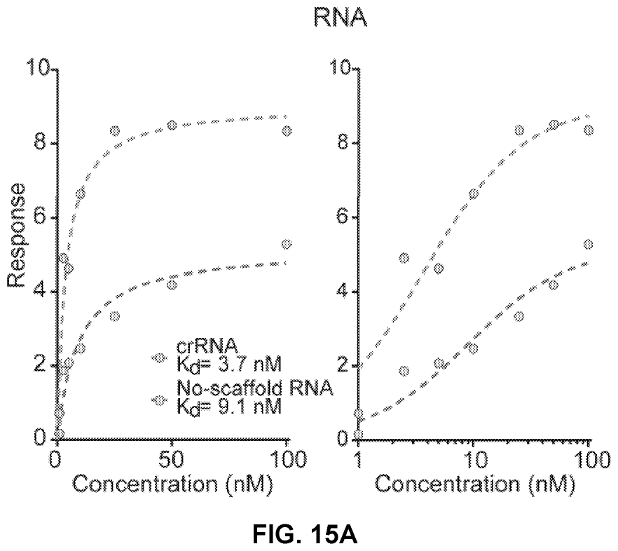

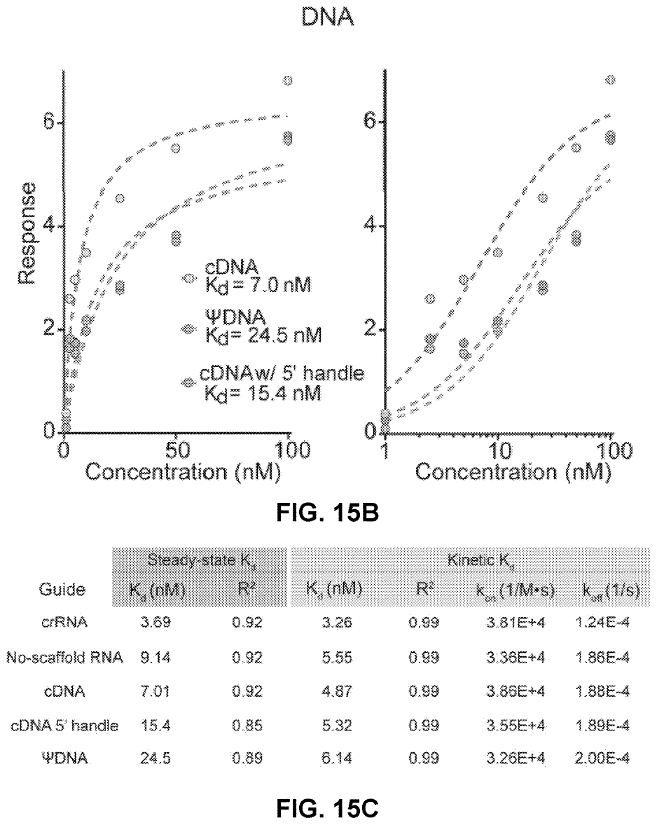

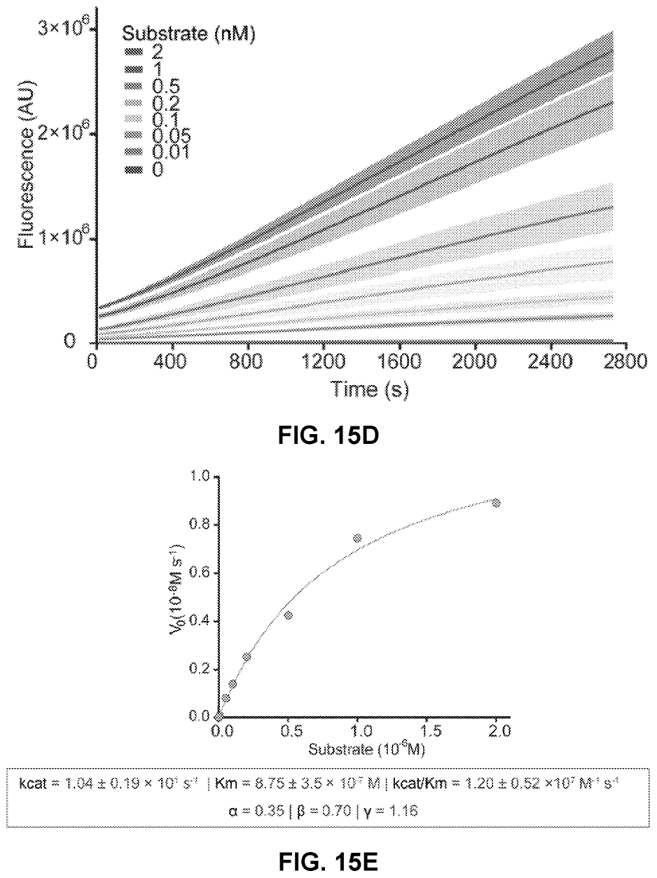

Bio-layer interferometry measured binding affinities of 3.7 nM for crRNA and 24.5 nM for ΨDNA, indicating slightly weaker yet adequate association (FIGS. 15A-15C). To further characterize the enzyme kinetics of the AsCas12a-ΨDNA-RNA ternary complex, initial velocities were measured and Michaelis-Menten values were determined. It was observed that the AsCas12a-ΨDNA system catalyzed trans-ssDNA cleavage at a rate of ~10 turnovers per second, with a catalytic efficiency (k_{cat}/Kₘ) of 1.2×10⁷ s⁻¹ M⁻¹ (FIGS. 15D-15E). These kinetic parameters are consistent with previously reported values for Cas12a.

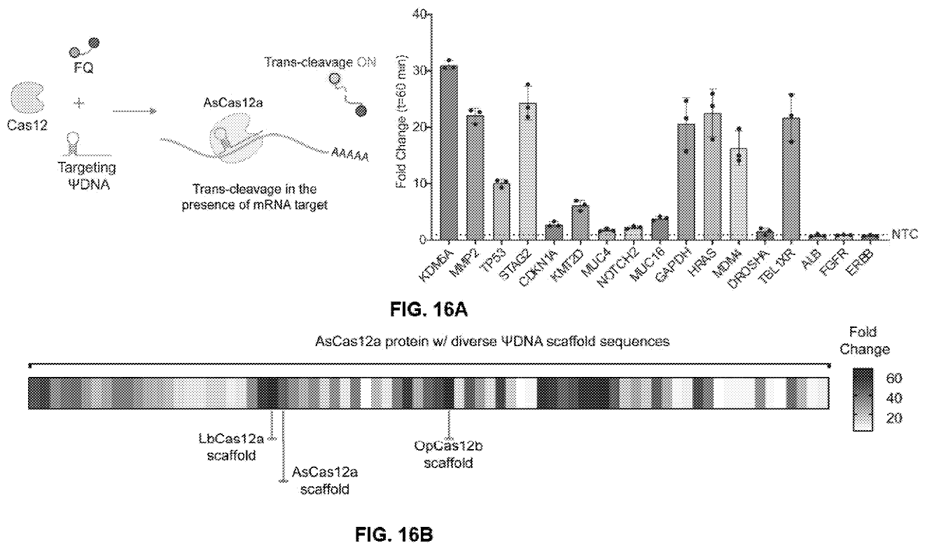

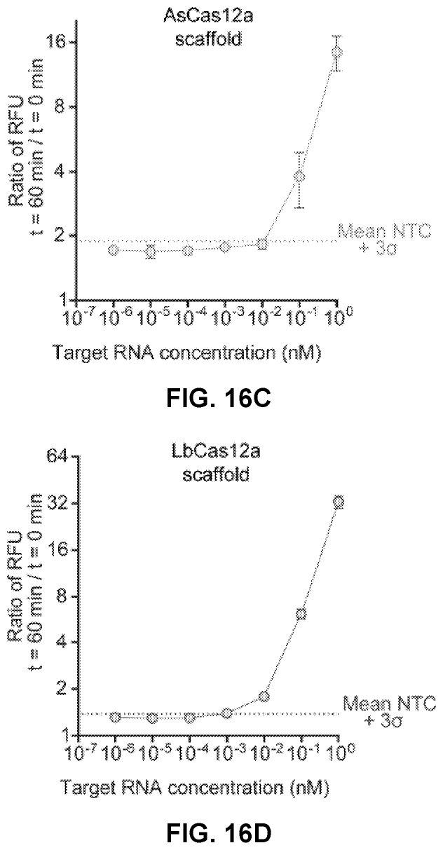

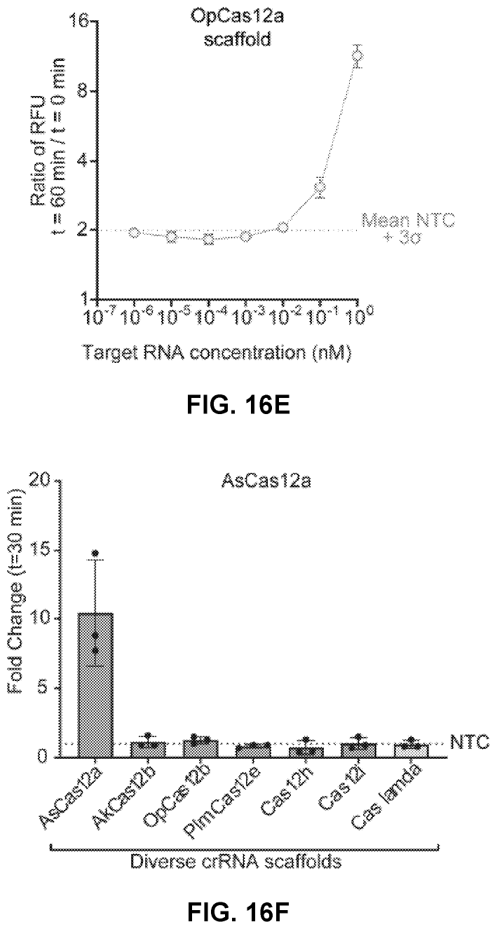

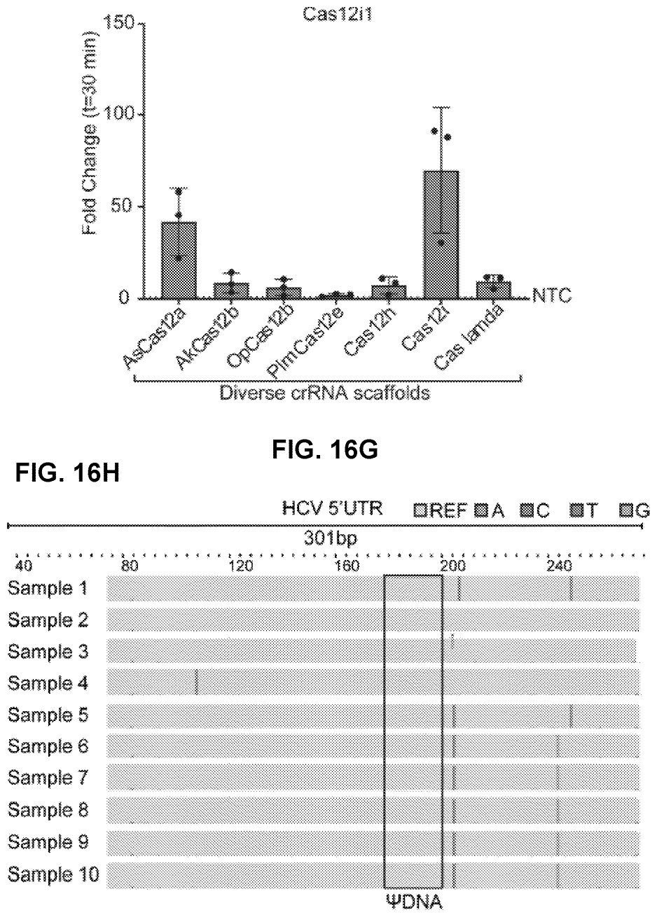

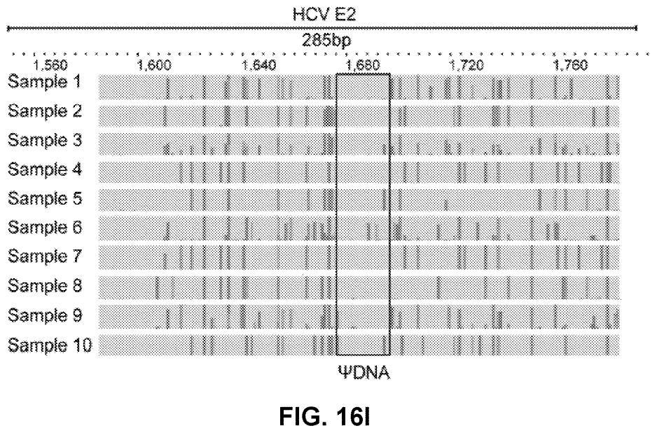

The system was then challenged with long RNA targets (~1.7 kb) using in vitro-transcribed fragments (FIG. 1E). AsCas12a scored on 24/26 ΨDNAs (92.3%), clearly separating controls, while Cas12i1 was active on 9/26 (34.6%). These results indicate that AsCas12a displays increased sequence flexibility. Moreover, validation using mRNA standards with sequences identical to endogenous transcripts demonstrates its potential for endogenous mRNA detection. (FIG. 15A). Seeking further gains, 77 distinct scaffold sequences were screened as 3′ handles (FIGS. 1F-1G). Detection efficiency varied widely. AsCas12a performed best with LbCas12a, OpCas12b, and its own scaffold; Cas12i1 favored its native sequence and simple AT repeats. For AsCas12a, limits of detection with the top three scaffolds reached 1-10 pM, with LbCas12a scaffold surpassing the canonical sequence (FIGS. 16B-16E). Additionally, the ΨDNA-Cas12 system demonstrated greater scaffold flexibility than conventional crRNA-guided systems when targeting DNA (FIGS. 16F-16G), which enhanced flexibility enables guide re-engineering in multiple orientations and design dimensions, thereby providing increased tunability and greater potential for further optimization and broader applications. To systematically evaluate the importance of scaffold structure (stem-loop) in ΨDNA function, a unique high-throughput sequencing-based mutational screening assay was developed. Towards this, a 256-variant ΨDNA library was first constructed by indexing four nucleotides within the stem-loop of the LbCas12a scaffold, the best performing candidates for AsCas12a (FIG. 1H). Each ΨDNA variant was then individually incubated with target RNA and AsCas12a in a trans-cleavage reaction without FQ reporters. Following this initial incubation, all reactions were pooled and further incubated to selectively deplete weakly bound ΨDNAs, and a no Cas control library was used as background. After the next-generation DNA sequencing and ranking the enrichment relative to the least retained variant, it was found that the original LbCas12a scaffold remained among the top-performing sequences (ranked #2 out of 256) (FIG. 1I). Furthermore, depletion levels strongly correlated with the number of mutations within the stem-loop, underscoring its essential role in stabilizing the AsCas12a-ΨDNARNA ternary complex (FIG. 1J). To validate this high-throughput result, eight ΨDNA variants were selected and their activities were confirmed in trans-cleavage assays, which recapitulated the sequencing-based trends (FIG. 1K). Scaffold sequences are provided in Table 2.

TABLE 2
DNA Scaffold Sequences from Diverse CRISPR-Cas Systems for ΨDNA-Mediated RNA Detection

| CRISPR | | |
|---|---|---|
| system | Organism | Predicted Mature Repeat |
| Cas12a | Francisella hispaniensis FSC454 | AAATTATTTAAAGTTCTTAGAC (SEQ ID NO. 146) |
| | Eubacterium eligens | AAATTATTTAAGGTTATTCAAAC (SEQ ID NO. 147) |
| | Candidatus Moranbacteria bacterium | AAATTGTGTAGGTCTTATTGCG (SEQ ID NO. 148) |
| | Moraxella bovoculi | AAATTTCTACTGTTTGTAGAT (SEQ ID NO. 149) |
| | Succinivibrio sp. | AAATTTCTACTTATGTAGAT (SEQ ID NO. 150) |
| | Candidatus Peregrinibacteria bacterium | AATCCTATAGGTCGTTTAGAG (SEQ ID NO. 151) |
| | Prevotella bryantii B14 | AATTAAATAAGCTTTATAGCC (SEQ ID NO. 152) |
| | Helcococcus kunzii | AATTAATAGTATCTCTTAAAG (SEQ ID NO. 153) |
| | Muribaculaceae bacterium | AATTACAGGCTTTATGTAGCC (SEQ ID NO. 154) |
| | Prevotellamassilia sp. | AATTATAAAGGCATTATAGCC (SEQ ID NO. 155) |
| | Butyrivibrio hungatei | AATTATCTTTAAGTCTTAGAC (SEQ ID NO. 156) |
| | Pseudobutyrivibrio xylanivorans | AATTATTTAAAGGTTCTAAGC (SEQ ID NO. 157) |
| | Francisella cf. novicida Fx1 | AATTATTTAAAGTTCTTAGAC (SEQ ID NO. 158) |
| | Candidatus Methanomethylophilus alvus | AATTCTGAATGAGTTTTAGAC (SEQ ID NO. 159) |
| | Flavobacterium jumunjinense | AATTTAGTTTGTCTTTAAAAC (SEQ ID NO. 160) |
| | Sedimentisphaera cyanobacteriorum | AATTTTATAAGGCCTTTAGAC (SEQ ID NO. 161) |
| | L21RPul-D3 | |
| | Sneathia amnii (fusobacteria) SN35 | AATTTTATTTGGGTTCTAAAC (SEQ ID NO. 162) |
| | Alicyclobacillus acidoterrestris | AGCGATCTGAGAAGTGGCAC (SEQ ID NO. 163) |
| | Candidatus Methanoplasma termitum | GAATCTCTACTCTTTGTAGAT (SEQ ID NO. 164) |
| | (euryarchaeotes) MpT1 | |
| | Eubacterium rectale M104/1 (firmicutes) | GAATGTCTACTGGGGTAGATC (SEQ ID NO. 165) |
| | Lachnospiraceae bacterium GAM79 | GAATTTCTACTAGTGTAGAT (SEQ ID NO. 166) |
| | Ruminococcus sp. JE7A12 (firmicutes) | GAATTTCTACTATTGTAGAT (SEQ ID NO. 167) |
| | Moraxella bovoculi | TAAATTTCTACTGTTTGTAGAT (SEQ ID NO. 168) |
| | Lachnospiraceae bacterium ND2006 | TAATTTCTACTAAGTGTAGAT (SEQ ID NO. 169) |
| | Prevotella bryantii | TAATTTCTACTATTGTAGAT (SEQ ID NO. 170) |
| | Acidaminoscoccus Cas12a | TAATTTCTACTCTTGTAGAT (SEQ ID NO. 171) |
| | Francisella tularensis subsp. novicida | TAATTTCTACTGTTGTAGAT (SEQ ID NO. 172) |
| | F6168 (gproteobacteria) | |
| | Lachnospira eligens (firmicutes) | TAATTTCTACTTTGTAGAT (SEQ ID NO. 173) |
| | Catenovulum sp. CCB-QB4 (g-proteobacteria) | TCACCTACGTAACCGTGAACT (SEQ ID NO. 174) |
| | PdCas12a | TAATTTCTACTTCGGTAGAT (SEQ ID NO. 175) |
| | LpCas12a | TAATTTCTACTGTGTGTAGAT (SEQ ID NO. 176) |
| | Methylobacterium nodulans ORS 2060 | AGGATCTGGCGCCCACTGCGAC(SEQ ID NO. 177) |
| | Akkermansiaceae bacterium | CACATCGCGGGAACTGCGGC (SEQ ID NO. 178) |
| | Pelobacter propionicus DSM 2379 | CCCCGCGCATGCGGGGAACAC (SEQ ID NO. 179) |
| | Bacillus sp. NSP2.1 | CCTCGGGTCCTCAATGTAAC (SEQ ID NO. 180) |
| | Alicyclobacillus acidoterrestris | CGAGCGATCTGAGAAGTGGCAC (SEQ ID NO. 181) |
| | Brevibacillus agri (firmicutes) DSM 6348 | CTTTCCACTAAGCTTTCGAAC (SEQ ID NO. 182) |
| | Brevibacillus sp. FJAT54423 | GAAAAGCTGAGAAGTTAGCAC (SEQ ID NO. 183) |
| | Sulfobacillus thermotolerans (firmicutes) | GAATGCTTAGGTTGTTGGCAC (SEQ ID NO. 184) |
| | Phycisphaeraceae bacterium | GCCGTGTTGGCCGATGCGGC (SEQ ID NO. 185) |
| | Methylacidimicrobium sp. AP8 | GGCCTGCGCCGACCCGAACCGC (SEQ ID NO. 186) |
| | Phycisphaeraceae bacterium | GGGCCGCGTCGGCCTCCGCGGC (SEQ ID NO. 187) |
| | Opitutaceae bacterium TAV5 | TGAAACGGCATTCGCTGCGGC (SEQ ID NO. 188) |
| | Sulfobacillus thermotolerans | TGCTTAGGTTGTTGGCAC (SEQ ID NO. 189) |
| | Brevibacillus sp. HD3.3A | TGGAAAGCTTCGGGATTAGCAC (SEQ ID NO. 190) |
| | Brevibacillus sp. FJAT54423 | TGGAGTGCGTGGATTGAAAC (SEQ ID NO. 191) |
| Other | Cas12d | ACCCGTAAAGCAGAGCGATGAAGGC (SEQ ID NO. 192) |
| Cas12 & | PlmCas12e | TATTTATCGGAGATATCTTCAAAC (SEQ ID NO. 193) |
| Racr | Cas12f | CGCGCCCCTGATGAATGGACAC (SEQ ID NO. 194) |
| | Cas12h1 | GCTAGAGGGAGGTCAGAGCAC (SEQ ID NO. 195) |
| | Cas12i1 | AATTTTTGTGCCCATCGTTGGCAC (SEQ ID NO. 196) |
| | Cas12i2 | AGAAATCCGTCTTTCATTGACGG (SEQ ID NO. 197) |
| | Cas12Lambda2 | TATTTTGTATGGAGTAAACAAC (SEQ ID NO. 198) |
| | Racr VA1 | TAAATTTCTACTGTTTGTAGAT (SEQ ID NO. 199) |
| | Racr VA2 | TAAATTTCTACTGTTTGTAGAT (SEQ ID NO. 200) |
| | Racr VA3 | TAAATTTCTACTATTTGTAGAT (SEQ ID NO. 201) |
| | Cas12a2 | TAATTTCTACTGTTGTAGAT (SEQ ID NO. 202) |
| | Cas12c2 | AGCAGGATTCAGGTTGGGTTTGAGG (SEQ ID NO. 203) |
| | Cas12k | AGGTGGGTTGAAAG (SEQ ID NO. 204) |
| | Cas12g | GGTGGAAAGGGCCGGAGATTTACCGGCTCTGACACC |
| | | (SEQ ID NO. 205) |
| | Cas12L | GAAAAACGCTCTTAGGGAATGAAAG (SEQ ID NO. 206) |
| | Cas12m | GTGTCATAGCCCAGCTTGGCGGGCGAAGGCCAAGAC |
| | | (SEQ ID NO. 207) |
| Cas13 | Cas13a | GGATTTAGACCACCCCAAAAATGAAGGGGACTAAAACA |
| | | (SEQ ID NO. 208) |
| | Cas13b | GTTGTGGAAGGTCCAGTTTTGAGGGGCTATTACAAC |
| | | (SEQ ID NO. 209) |
| | Cas13c | GACTAAAACCAAGTAAATTGGTATTTAAAC |
| | | (SEQ ID NO. 210) |
| | Cas13d | AACCCCTACCAACTGGTCGGGGTTTGAAAC |
| | | (SEQ ID NO. 211) |
| Cas9 | Cas9a | GTTTTAGAGCTATGCTGTTTTGAATGGTCCCAAAAC |
| | | (SEQ ID NO. 212) |
| | Cas9b | GTTTCAGTTGCTGAATTATTTGGTAAACT |
| | | (SEQ ID NO. 213) |
| | Cas9c | GTTGTAGCTCCCATTCTCATTTCG (SEQ ID NO. 214) |
| | Cas9d | GTTACAGTTAAGGCTCT (SEQ ID NO. 215) |
| Scramble | Poly A | AAAAAAAAAAAAAAAAAAAAAA (SEQ ID NO. 216) |
| | Poly T | TTTTTTTTTTTTTTTTTTTTTT (SEQ ID NO. 217) |
| | Poly C | CCCCCCCCCCCCCCCCCCCCCC (SEQ ID NO. 218) |
| | AT | ATATATATATATATATATATAT (SEQ ID NO. 219) |
| | GC | GCGCGCGCGCGCGCGCGCGCGC (SEQ ID NO. 220) |
| | AC | ACACACACACACACACACACAC (SEQ ID NO. 221) |
| | CT | CTCTCTCTCTCTCTCTCTCTCT (SEQ ID NO. 222) |
| | Spacer sequence | ACAGCCCATCGACTGGTGTT (SEQ ID NO. 223) |

For clinical translation, a two-step workflow was constructed. Reverse-transcription polymerase chain reaction followed by T7 transcription produced RNA amplicons that the ΨDNA-Cas12 reaction detected at picomolar levels. Synthetic mimics from Hepatitis C Virus (HCV), Dengue, and Zika were successfully identified (FIG. 1L). Finally, 40 Hepatitis C patient samples, 20 positive and 20 negative, were evaluated using ΨDNA guides targeting the 5′ untranslated region and the E2 gene (FIGS. 1M-1N). The 5′ untranslated ΨDNA guide detected every positive sample across genotypes 1a and 1b. The E2 ΨDNA selectively detected genotype 1a. Next-generation sequencing confirmed viral presence and sequence identity (FIGS. 16H-16I). Overall diagnostic accuracy was 100%, illustrating the promise of ΨDNA-guided Cas12 detection in clinical settings. Primers and ΨDNA used for viral RNA and HCV patient sample detection are presented in Table 3.

TABLE 3
Primers and ΨDNA Used for Viral RNA and HCV Patient Sample Detection

| Name | Sequence |
|---|---|
| DENV Geneblock w/T7 (SEQ ID NO. 224) | AGTACATATTCAGGGGCCAACCTCTCAACAATGACGAAGACCATG |
| | CTCACTGGACAGAAGCAAAAATGCTGCTGGACAACATCAACACA |
| | CCAGAAGGGATTATACCAGCTCTCTTTGAACCAGAAAGGGAGAA |
| | GTCAGCCGCCATAGACGGTGAATACCGCCTGAAGGGT |
| ZIKV Geneblock w/T7 (SEQ ID NO. 225) | GACACCGGAACTCCACACTGGAACAACAAAGAAGCACTGGTAGA |
| | GTTCAAGGACGCACATGCCAAAAGGCAAACTGTCGTGGTTCTAG |
| | GGAGTCAAGAAGGAGCAGTTCACACGGCCCTTGCTGGAGCTCT |
| | GGAGGCTGAGATGGATGGTGCAAAGGGAAGGCTGTCCTCTGGC |
| HCV-1a Geneblock w/T7 (SEQ ID NO. 226) | AGACACACTCCAGTCAATTCCTGGCTAGGCAACATAATCATGTTT |
| | GCCCCCACACTGTGGGCGAGGATGATACTGATGACCCACTTCTT |
| | TAGCGTCCTCATAGCCAGGGATCAGCTTGAACAGGCCCTTGATT |
| | GCGAGATCTACGGAGCCTGCTACTCCATAGAACCACTGGATCTAC |
| | CTCCAATCATTCAAAGACTCCATGGCCTCAGCGCGTTTTCACTCC |
| | ACAGTTACTCTCCAGGTGAA |
| HCV-1b Geneblock w/T7 (SEQ ID NO. 227) | CAGCTAGACACACTCCAGTCAACTCCTGGCTAGGCAACATCATCA |
| | TGTATGCGCCCACCTTATGGGCAAGGATGATTCTGATGACTCACT |
| | TCTTCTCCATCCTTCTAGCTCAGGAGCAACTTGAAAAAGCCCTAG |
| | ATTGTCAGATCTACGGGGCCTGTTACTCCATTGAGCCACTTGACC |
| | TACCTCAGATCATTCAGCGACTCCATGGTCTTAGCGCATTTTCACT |
| | CCATAGTTACTCTCCAG GTGAG |
| DENV For Primer with T7 (SEQ ID NO. | GAAATTAATACGACTCACTATAGGGGTACATATTCAGGGGCCAACC |
| 228) | TCTC |
| DENV Rev Primer (SEQ ID NO. 229) | TTTCTGGTTCAAAGAGAGCTGGTAT |
| DENV AsCas12a ΨDNA (SEQ ID NO. 230) | TCC AGT GAG CAT GGT CTT CGT AAT TTC TAC TAA GTG TAG AT |
| ZIKV For Primer with T7 (SEQ ID NO. 231) | GAAATTAATACGACTCACTATAGGGCCACACTGGAACAACAAAGAAGCAC |
| ZIKV Rev Primer (SEQ ID NO. 232) | ACAGCCTTCCCTTTGCACCATCCATCTCAG |
| ZIKV AsCas 12a ΨDNA (SEQ ID NO. 233) | GAA CCA CGA CAG TTT GCC TTT AAT TTC TAC TAA GTG TAG AT |
| HCV NS5B For Primer with T7 (SEQ ID | GAAATTAATACGACTCACTATAGGGCACACTCCAGTCAATTCCTG |
| NO. 234) | GCT AGG |
| HCV NS5B Rev Primer (SEQ ID NO. 235) | CACCTGGAGAGTAACTGTGGAGTGAA |
| HCV NS5B 1a AsCas12a | CAA GGG CCT GTT CAA GCT GAT AAT TTC TAC TAA GTG TAG AT |
| ΨDNA (SEQ ID NO. 236) | |
| HCV NS5B 1b AsCas12a | CTA GGG CTT TTT CAA GTT GCT AAT TTC TAC TAA GTG TAG AT |
| ΨDNA (SEQ ID NO. 237) | |
| HCV 5UTR For Primer with T7 (SEQ ID | TAATACGACTCACTATAGGAGCGTCTAGCCATGGCGTT |
| NO. 238) | |
| HCV 5UTR Rev Primer (SEQ ID NO. 239) | GCAAGCACCCTATCAGGCAGT |
| HCV 5UTR AsCas12a | TCCAAGAAAGGACCCGGTCGTAATTTCTACTAAGTGTAGAT |
| ΨDNA (SEQ ID NO. 240) | |
| HCV E2 T7 For Primer with T7 | TAATACGACTCACTATAGACACCAACGGCAGTTGGCAC |
| (SEQ ID NO. 241) | |
| HCV E2 Rev Primer (SEQ ID NO. 242) | AAGGTGTTGTTGCCCACCCC |
| HCV E2 AsCas12a ΨDNA (SEQ ID NO. 243) | GCCTGAAGAGTTGAATTTGTTAATTTCTACTAAGTGTAGAT |

### Further Development of Guide RNA

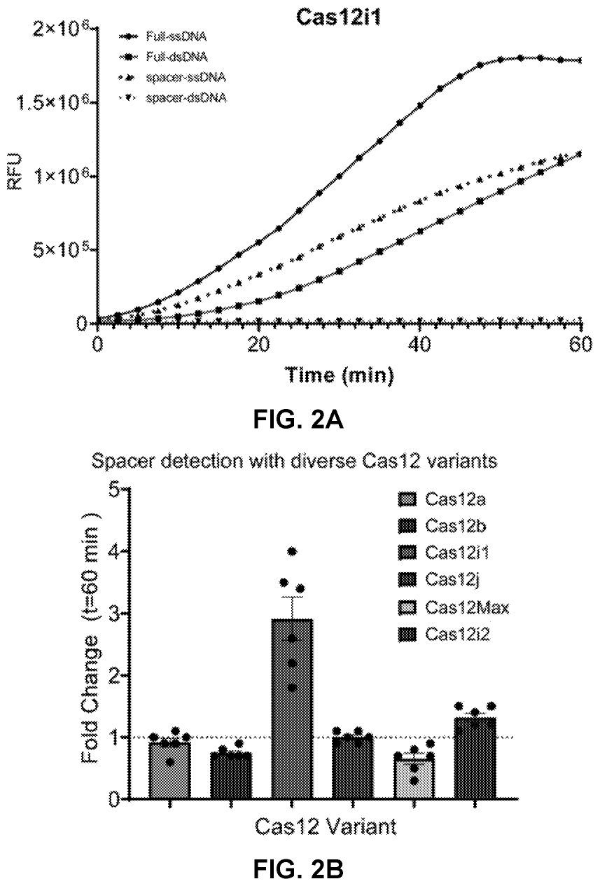

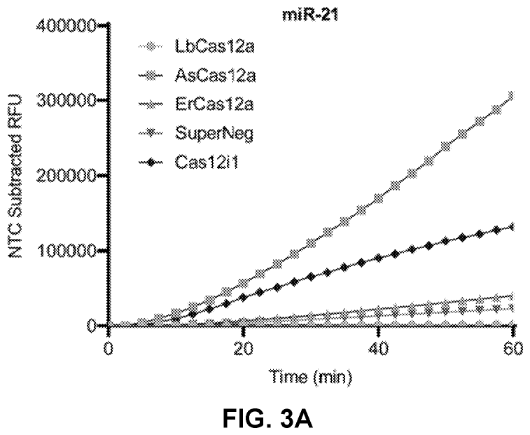

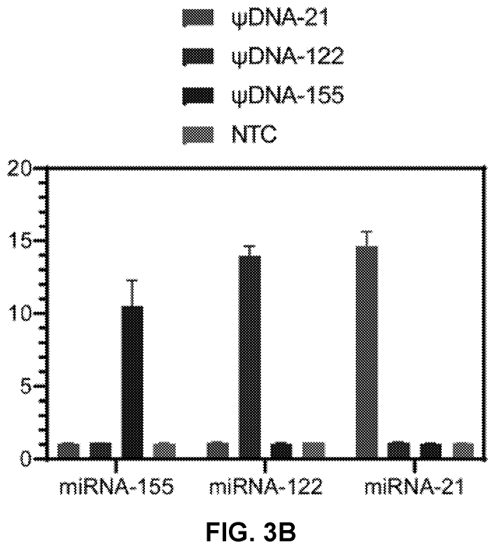

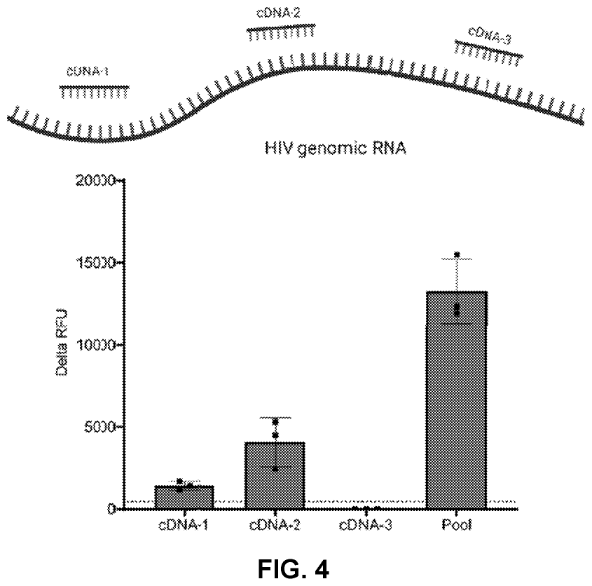

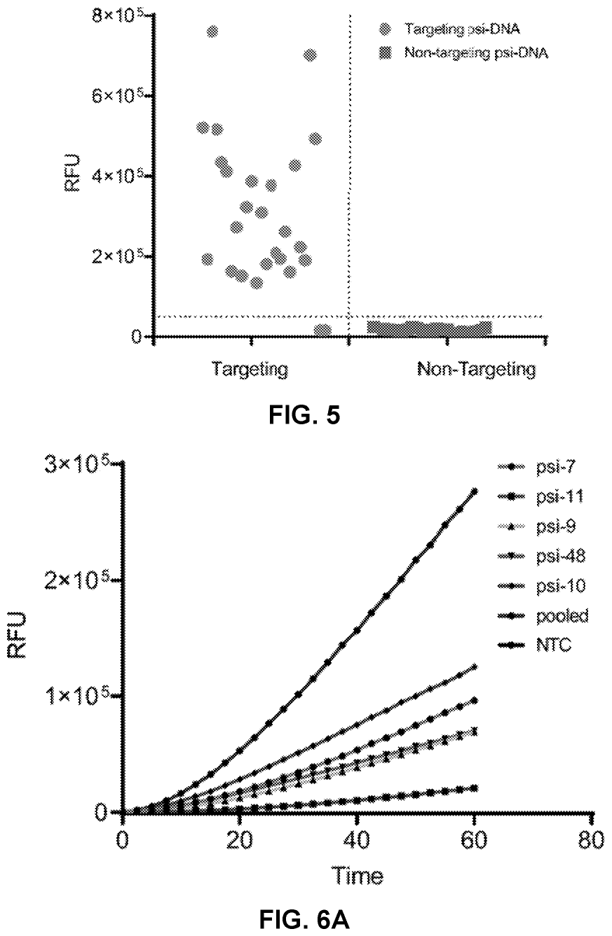

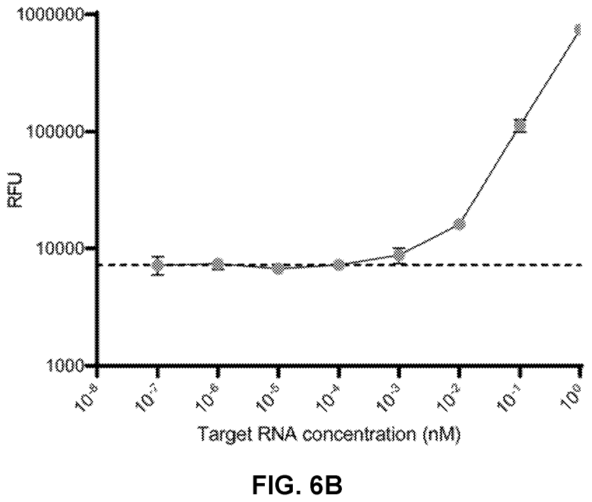

Tolerance of Cas12i1 for different lengths of crRNAs was evaluated. Cas12i1 exhibited trans-cleavage activity for full guide crRNAs and guide crRNAs shortened by 5 bases for both ssDNA and dsDNA. However, Cas12i1 had trans cleavage activity for crRNAs consisting of a spacer element only for ssDNA targets (FIGS. 1A-1N). Among numerous Type II Cas enzymes tested, only Cas12i1 exhibited this property (FIGS. 2A-2B).

Detection of miRNA

It was found that miRNA could be detected by adding a complementary cDNA sequence, with cDNA sequences of 20 nucleotides long performing somewhat better than cDNA sequences of 23 nucleotides long for miR-21 and miR-122. Endogenous miRNA was used as a surrogate guide.

Detection was accomplished by including a fluorophore and a quencher on the cDNA. When trans cleavage was activated, the quencher was separated from the fluorophore, and a fluorescence signal was obtained.

Sequences useful for the detection of miRNAs are found in Table 4.

TABLE 4
DNA and RNA Sequences Used to Detect miRNA with Cas12i1 and AsCas12a

| Name | Sequence |
|---|---|
| GFP Target Sense Strand | AGCACCCAGTCCGCCCTGAG |
| (SEQ ID NO. 3) | |
| GFP Target Antisense Strand | CTCAGGGCGGACTGGGTGCT |
| (SEQ ID NO. 4) | |
| Ca12i1 crGFP (SEQ ID NO. 5) | rArArUrUrUrUrUrGrUrGrCrCrCrArUrCrGrUrUrGrGrCrArC |
| | rCrUrCrArGrGrGrCrGrGrArCrUrGrGrGrUrGrCrU |
| Cas12i crGFP -5 scaf | rUrUrGrUrGrCrCrCrArUrCrGrUrUrGrGrCrArCrCrUrCrArG |
| (SEQ ID NO. 6) | rGrGrCrGrGrArCrUrGrGrGrUrGrCrU |
| Cas12i crGFP -10 scaf | rCrCrCrArUrCrGrUrUrGrGrCrArCrCrUrCrArGrGrGrCrGrG |
| (SEQ ID NO. 7) | rArCrUrGrGrGrUrGrCrU |
| Cas12i crGFP -14 scaf | rUrCrGrUrUrGrGrCrArCrCrUrCrArGrGrGrCrGrGrArCrUrG |
| (SEQ ID NO. 8) | rGrGrUrGrCrU |
| Cas12i GFP Spacer Only | rCrUrCrArGrGrGrCrGrGrArCrUrGrGrGrUrGrCrU |
| (SEQ ID NO. 9) | |
| miR-21 20 nt cDNA | ACAGCCCATCGACTGGTGTT |
| (SEQ ID NO. 10) | |
| miR-122 20 nt cDNA | TATTTAGTGTGATAATGGCG |
| (SEQ ID NO. 11) | |
| miR-155 20 nt cDNA | ACCCCTATCACGATTAGCAT |
| (SEQ ID NO. 12) | |
| miR-21 RNA Target | rCrArArCrArCrCrArGrUrCrGrArUrGrGrGrCrUrGrU |
| (SEQ ID NO. 13) | |
| miR-122 RNA Target | rArArCrGrCrCrArUrUrArUrCrArCrArCrUrArArArUrA |
| (SEQ ID NO. 14) | |
| miR-155 RNA Target | rUrUrArArUrGrCrUrArArUrCrGrUrGrArUrArGrGrGrGrU |
| (SEQ ID NO. 15) | |
| miR-21 20 nt 3′ Handle Cas12i1 | ACAGCCCATCGACTGGTGTTAATTTTTGTGCCCATCGTTGGCAC |
| (ΨDNA) (SEQ ID NO. 16) | |
| miR-21 20 nt 5′ Handle Cas12i1 | AATTTTTGTGCCCATCGTTGGCACACAGCCCATCGACTGGTGTT |
| (SEQ ID NO. 17) | |
| miR-21 20 nt double handle Cas12i1 | AATTTTTGTGCCCATCGTTGGCACACAGCCCATCGACTGGTGTTAATT |
| (SEQ ID NO. 18) | TTTGTGCCCATCGTTGGCAC |
| miR-21 ΨDNA AsCas12a (SEQ ID | ACAGCCCATCGACTGGTGTTTAGATGTGAATCATCTTTAAT |
| NO. 19) | |
| miR-122 ΨDNA AsCas12a (SEQ ID | TATTTAGTGTGATAATGGCGTAGATGTGAATCATCTTTAAT |
| NO. 20) | |
| miR-155 ΨDNA AsCas12a (SEQ ID | ACCCCTATCACGATTAGCATTAGATGTGAATCATCTTTAAT |
| NO. 21) | |
| miR-122 ΨDNA Cas12i1 (SEQ ID NO. | TATTTAGTGTGATAATGGCGAATTTTTGTGCCCATCGTTGGCAC |
| 22) | |
| miR-155 ΨDNA Cas12i1 (SEQ ID NO. | ACCCCTATCACGATTAGCATAATTTTTGTGCCCATCGTTGGCAC |
| 23) | |
| FQ Reporter (ssDNA) (SEQ ID NO. | FAM/TTATT/Iowa Black |
| 24) | |
| FAM Reporter (ssDNA) (SEQ ID NO. | FAM/TTATT |
| 25) | |
| FQ ssRNA (SEQ ID NO. 26) | FAM/rUrUrUrUrU/Iowa Black |

### Further Development of ΨDNA

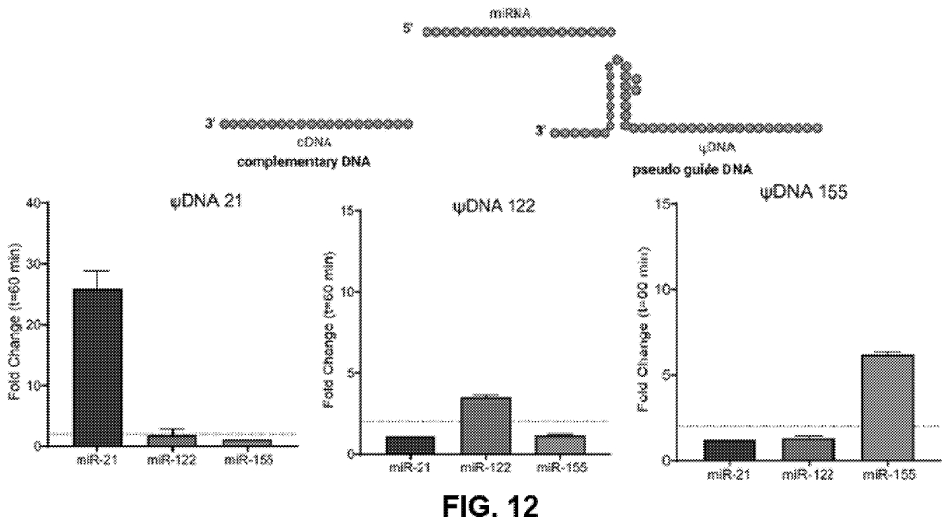

Adding a scaffold-like handle to the target ssDNA sequence enhanced the trans-cleavage reaction; it was found that the handle only works when added to the 3′ end of the DNA sequence. Herein, cDNA with a 3′ handle is referred to as ΨDNA or psiDNA and forms a pseudo-DNA guide for RNA detection. The reaction is specific for detection of an miRNA that is complementary to the ΨDNA (FIG. 12).

### Limit of Detection

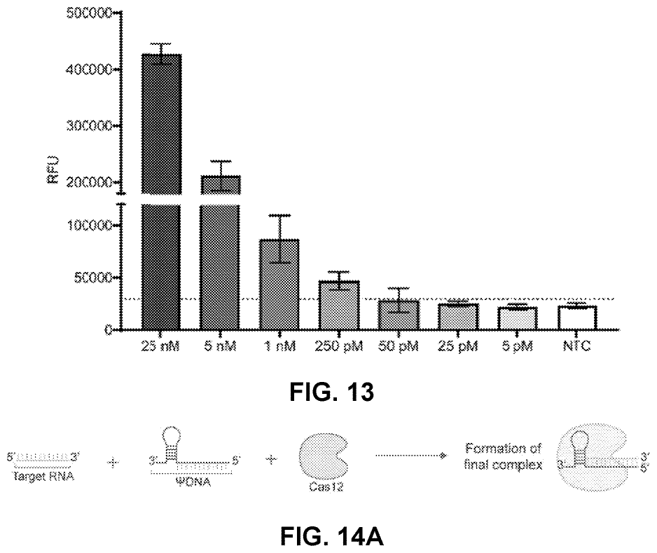

The detection limit using ΨDNA for detecting synthetic miR-21 was examined and found to be between 250 pM and 50 pM (FIG. 13). These concentrations are similar to those for conventional trans-cleavage assays with Cas12 enzymes targeting dsDNA with a full crRNA.

### Additional Cas Enzymes

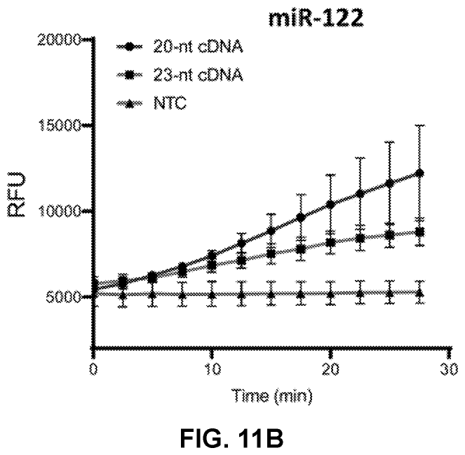

Although Cas12i1 was the only enzyme initially showing activity with spacer cRNA, AsCas12a showed greater activity than Cas12i1 when using ΨDNA. MiRNA detection was also more selective (FIGS. 11A-11B).

### Detection of HIV Genomic RNA

AsCas12a could be used with many cDNAs tiling the long RNA sequence of HIV genomic DNA to detect the target RNA. It was found that pooling many cDNAs enhanced detection activity.

48 different ΨDNAs were tested against a genomic HIV target; 26 were complementary to the target sequence and, of these, 24 showed trans-cleavage. 24 of the ΨDNAs did not target the HIV sequence and none showed trans-cleavage activity (FIG. 12).

The 5 best-performing DNAs were pooled and the limit of detection for HIV genomic RNA was tested and found to be 1 pM (FIG. 13).

Translational Repression in HEK293T Cells with ΨDNA and AsCas12a

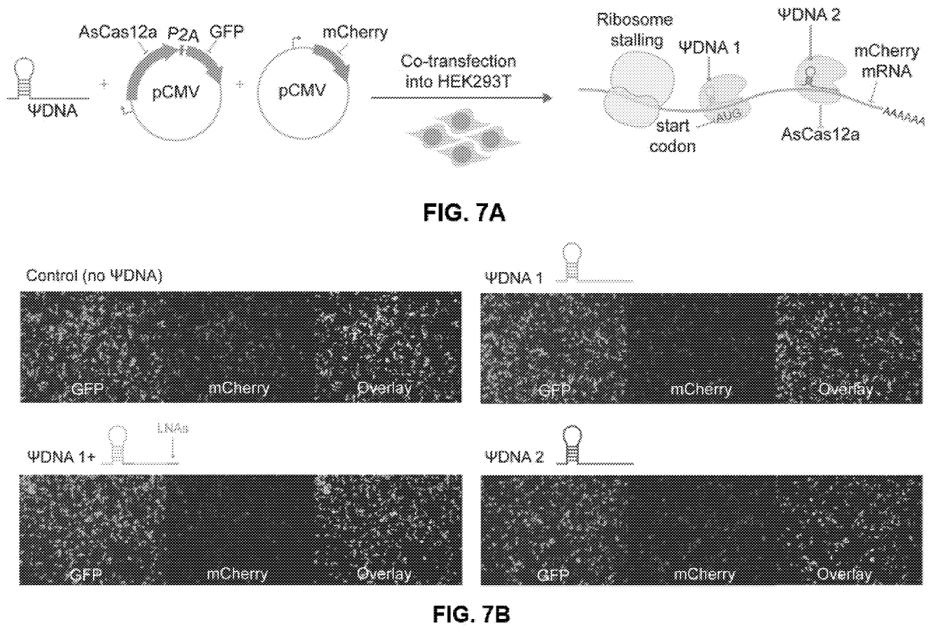

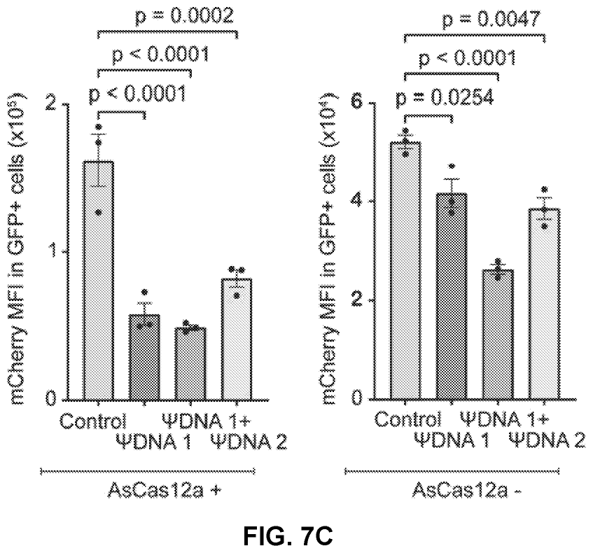

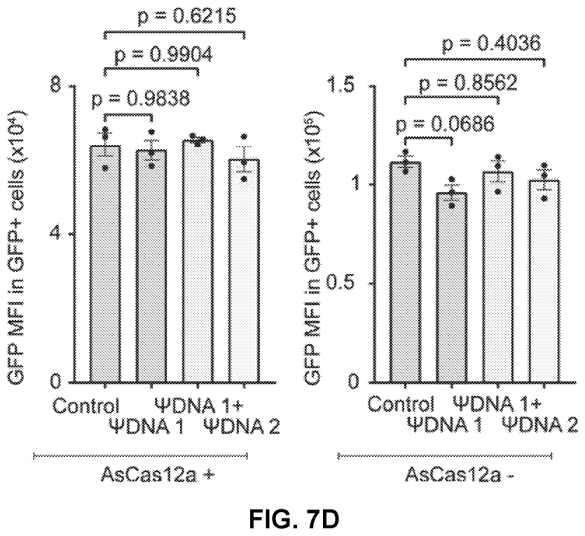

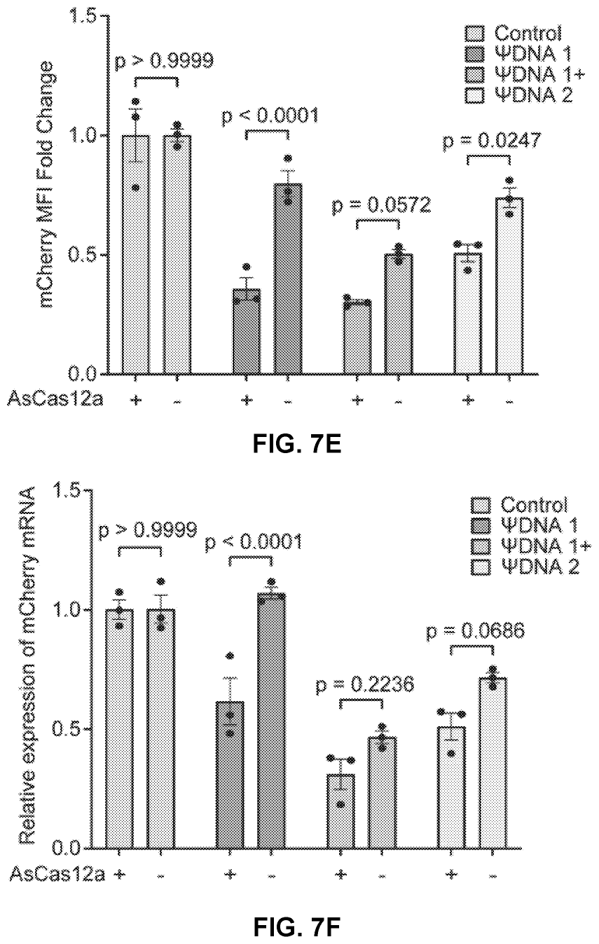

After characterizing the ΨDNA-Cas12 complex in vitro, its functionality in living cells was evaluated, aiming for potential in vivo applications. The ability of the DNA-guided AsCas12a complex to bind RNA within a cellular environment was tested by targeting a specific mRNA sequence to induce ribosome stalling, thereby repressing translation, reducing protein synthesis, and trigger RNA degradation pathways. To achieve this, a reporter system was designed in which HEK293T cells were co-transfected with two plasmids: one encoding AsCas12a tagged with GFP and the other encoding mCherry. Additionally, ΨDNA constructs targeting the mCherry mRNA were introduced either at the start codon (ΨDNA1 and ΨDNA1+) or downstream (ΨDNA2), to observe the effect on mCherry translation repression (FIG. 7A). The ΨDNA constructs were modified with phosphorothioate ends to prevent exonuclease degradation within cells, and ΨDNA1+ included locked nucleic acids (LNAs) flanking the guide region to enhance heteroduplex binding.

To assess the effects of ΨDNA and AsCas12a, the levels of GFP and mCherry were monitored 16 hours post-transfection using fluorescence microscopy (FIG. 7B). In initial observations, the control group, which lacked ΨDNA, exhibited higher overall red fluorescence compared to the ΨDNA-treated samples. Among the treated groups, those with ΨDNA targeting the start codon showed a particularly noticeable reduction in red fluorescence intensity. ΨDNA sequences for mCherry gene silencing and qPCR primers are presented in Table 5:

TABLE 5
ΨDNA Sequences for mCherry Gene Silencing and qPCR Primers

| Name | Sequence |
|---|---|
| ΨDNA 1 (targets mCherry start | G*A*T*GATGGCCATGTTATCCTTAGATG |
| codon) (SEQ ID NO. 244) | TGAATCATCTTT*A*A*T |
| ΨDNA 1+ (targets mCherry start | +G*+A*+T*GATGGCCATGTTAT+C+C+T |
| codon) (SEQ ID NO. 245) | TAGATGTGAATCATCTTT*A*A*T |
| ΨDNA 2 (targets mCherry) | T*T*G*GAGCCGTACATGAACTGTAGATG |
| (SEQ ID NO. 246) | TGAATCATCTTT*A*A*T |
| Scramble DNA (SEQ ID NO. 247) | TCGTCGGCAGCGTCTAATACGACTCACTATAGGG |
| qPCR mCherry For (SEQ ID NO. 248) | GACTACTTGAAGCTGTCCTTCC |
| qPCR mCherry Rev (SEQ ID NO. 249) | CGCAGCTTCACCTTGTAGAT |
| qPCR mCherry FAM Probe (SEQ ID | FAM/TTCAAGTGG/ZEN/GAGCGCGTGATGAA/ |
| NO. 250) | Iowa Black |
| qPCR GAPDH For (SEQ ID NO. 251) | GCTCCCTCTTTCTTTGCAGCAAT |
| qPCR GAPDH Rev (SEQ ID NO. 252) | TACCATGAGTCCTTCCACGATAC |
| qPCR GAPDH Cy5 Probe (SEQ ID NO. | Cy5/TCCTGCACC/TAO/ACCAACTGCTTAGCACC/ |
| 253) | Iowa Black RQ |

*signifies phosphorothioate bonds
+indicates LNAs

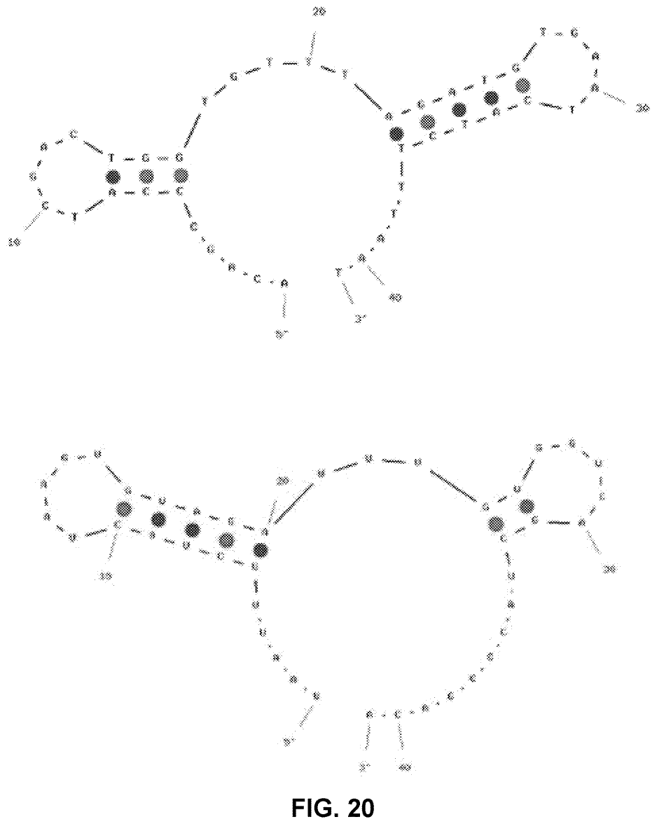

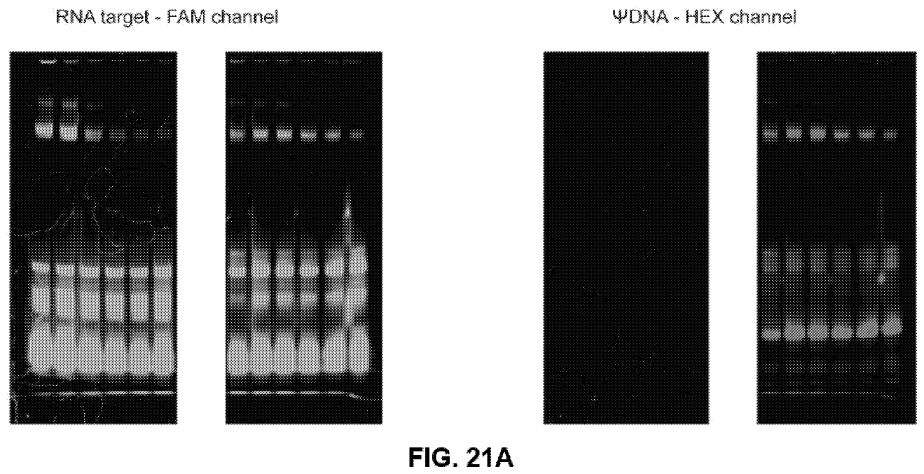

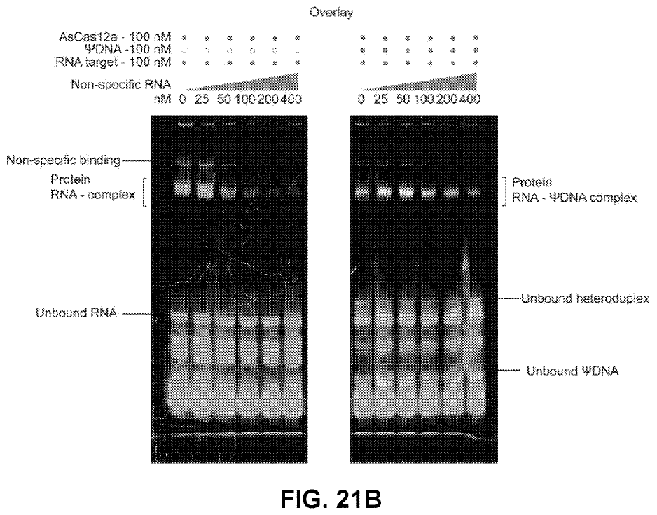

To further quantify the changes in mCherry production, the samples were subjected to flow cytometry. To ensure that any observed effects were not due to the ΨDNA alone triggering an antisense oligonucleotide (ASO) RNA degradation pathway, an additional control group that received GFP only was included, instead of the AsCas12a-GFP construct. Following flow cytometry, the mean fluorescence intensity (MFI) of mCherry in all GFP-positive cells was compared to determine whether the presence of AsCas12a and ΨDNA led to a reduction in overall red fluorescence intensity. The MFI of cells treated with AsCas12a and ΨDNA was significantly lower, confirming the initial observation that targeting the start codon is more effective (FIGS. 7C and 20). While samples treated with ΨDNA alone also showed reduced mCherry levels, the decrease was less pronounced compared to those treated with AsCas12a. To rule out any potential bias due to differences in transfection efficiency of AsCas12a-GFP or GFP alone, it was confirmed that the MFI of GFP across all groups remained consistent (FIG. 7D). Comparing the fold change in mCherry MFI across the different ΨDNA constructs relative to the control clearly demonstrated that mCherry expression level was significantly reduced in AsCas12a group. Although ΨDNA alone reduced mCherry fluorescence, particularly with ΨDNA1+, the presence of AsCas12a further enhanced this effect, leading to a significant decrease in protein expression across all constructs (FIG. 7E). Notably, ΨDNA1 exhibited the highest activity when paired with AsCas12a.

To assess RNA expression within the samples, total RNA was extracted and mRNA expression levels were evaluated (FIG. 7F). In all samples, the presence of AsCas12a led to a noticeable decrease in mCherry mRNA levels. Given that the ΨDNA complex is incapable of RNA cis or trans-cleavage (FIGS. 14D-14E) it is hypothesized that this mRNA degradation is induced by a no-go decay (NGD) mechanism. NGD is triggered when ribosomes stall and fail to elongate, leading to the degradation of mRNA that cannot produce functional proteins. Additionally, it was observed that ΨDNA1+ alone triggered ASO-driven RNA degradation, as indicated by both MFI and mRNA levels, likely due to the strong hybridization induced by the incorporated LNAs. Nonetheless, AsCas12a significantly enhances gene repression, resulting in overall lower protein levels when both the protein and guide are present, confirming the effectiveness of the ΨDNA-Cas12a complex in HEK293T cells.

### Effective Gene Silencing in Cellular Environments

It was demonstrated that AsCas12a can complex with ΨDNA and target mRNA inside cells, leading to reduced protein production through translational repression. Importantly, since this DNA-guided system lacks RNA cis- or trans-cleavage activity, herein it is shown that the ΨDNA-Cas12 complex is effective for transient gene silencing and holds potential for RNA editing within cellular environments as other researchers have achieved with inactive variants of Cas13. This finding expands the functionality of Cas12 while also increasing the versatility and efficiency of CRISPR-based technologies in both research and therapeutic settings. Sequences used for assessing cis-cleavage are provided in Table 6.

TABLE 6
DNA and RNA Sequences for RNA cis-Cleavage

| Name | Sequence |
|---|---|
| Target | FAM/rArGrArCrCrGrUrGrCrA |
| (SEQ ID NO. 43) | rUrCrArUrCrCrUrArArArCrC |
| | rUrCrGrUrCrCrGrCrCrCrUrG |
| | rArGrGrArArCrGrCrUrGrArA |
| | rGrCrGrCrCrUrUrGrUrGrGrU |
| | rArC/Cy5 |
| AsCas12a ΨDNA | TCAGCGTTCCTCGGGCGGAC |
| (SEQ ID NO. 44) | TAGATGTGAATCATCTTTAAT |
| Cas12i1 ΨDNA | TCAGCGTTCCTCGGGCGGACAATT |
| (SEQ ID NO. 45) | TTTGTGCCCATCGTTGGCAC |
| DNAzyme 10-23 | GAGGTTTAGGA GGCTAGCTACAACGA |
| (SEQ ID NO. 46) | GATGCACGG |

The ability of the disclosed tested DNA-guided complex to bind RNA within a cellular environment was tested by targeting a specific mRNA sequence to induce ribosome stalling, thereby repressing translation and reducing protein synthesis.

FIG. 7A shows a schematic representation for co-transfection of AsCas12a-GFP, mCherry, and ΨDNA to induce ribosome stalling inside HEK293T cells. ΨDNA1 and ΨDNA1+ target the start codon of mCherry mRNA and ΨDNA2 targets downstream. A mechanism for the ribosome stalling has not yet been determined. Herein it is demonstrated that when targeting RNA, the DNA-guided AsCas12a construct does not have any RNA cleavage in cis (target RNA) or trans (non-target RNAs) but rather only trans-cleavage of ssDNA. Thus, having no capabilities for cleaving RNA, it is hypothesized that the lower production of mCherry is induced by the binding of the Cas12 to the mRNA, which can reduce translation by the stalling of the ribosome.

FIG. 7B shows microscopy images for cells treated with AsCas12a-GFP, mCherry, and four different conditions of ΨDNA. Individual GFP (center panels) and mCherry (right panels) channels are shown as well as the overlay (left panels).

FIG. 7C shows geometric Mean Fluorescence Intensity (MFI) of mCherry for all four ΨDNA conditions with AsCas12a-GFP and GFP only. mCherry MFI is calculated only from GFP-positive cells. Geometric means are used for all calculations. Statistical analysis for n=3 biologically independent replicates was performed using Dunnet's multiple comparison test against the control samples. Error bars represent the mean value±standard error of mean (SEM). FIG. 7D shows MFI of GFP on all GFP+ cells. The difference in expression of GFP is non-significant throughout all samples compared to the control. Similar GFP MFI demonstrates equal levels of AsCas12a-GFP or GFP for all samples resulting in no protein concentration bias. Geometric mean is used for all calculations. Statistical analysis for n=3 biologically independent replicates was performed using Dunnet's multiple comparison test against the control samples. Error bars represent the mean value±standard error of mean (SEM).

FIG. 7E shows mCherry MFI fold change for all ΨDNA constructs with and without AsCas12a. Statistical analysis for n=3 biologically independent replicates was performed using Sidaks's multiple comparison test to compare the effect of AsCas12a on mCherry production. FIG. 7F shows relative quantification of mCherry mRNA using 2^{-ΔΔCt method. GAPDH was chosen as the endogenous control. Statistical analysis for n=}3 biologically independent replicates was performed using Sidaks's multiple comparison test to compare the effect of AsCas12a on mCherry mRNA degradation. Each biological replicate had n=3 technical replicates.

Efficient Endogenous RNA Knockdown Using ΨDNA-Guided CRISPR-Cas12a with Low Off-Target Effects

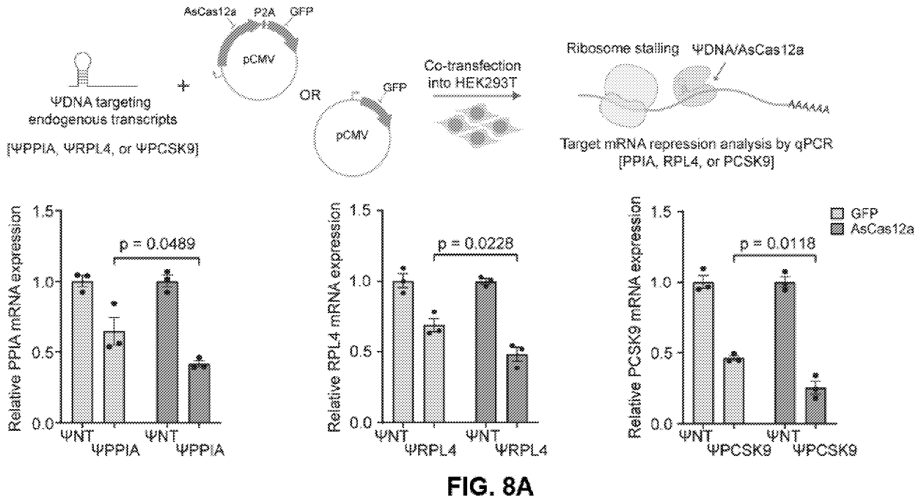

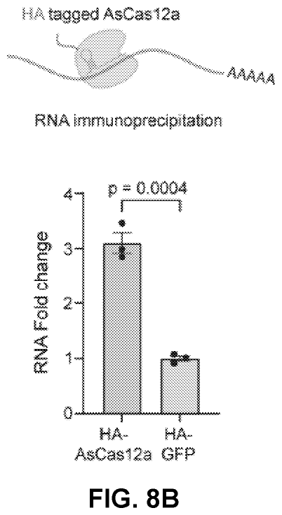

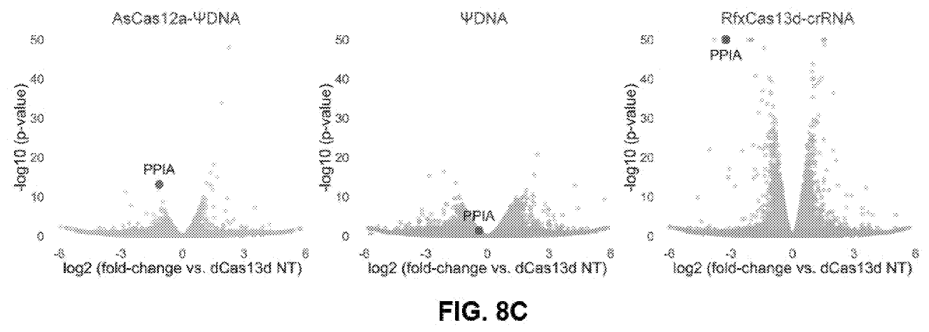

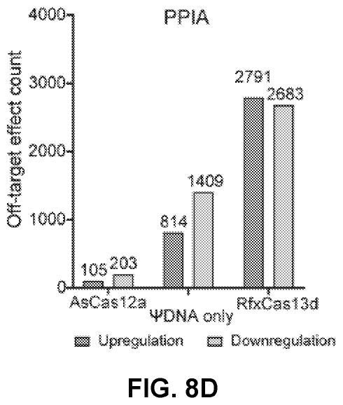

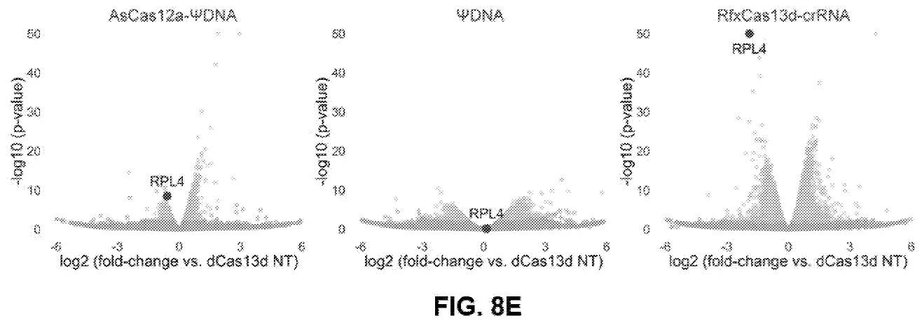

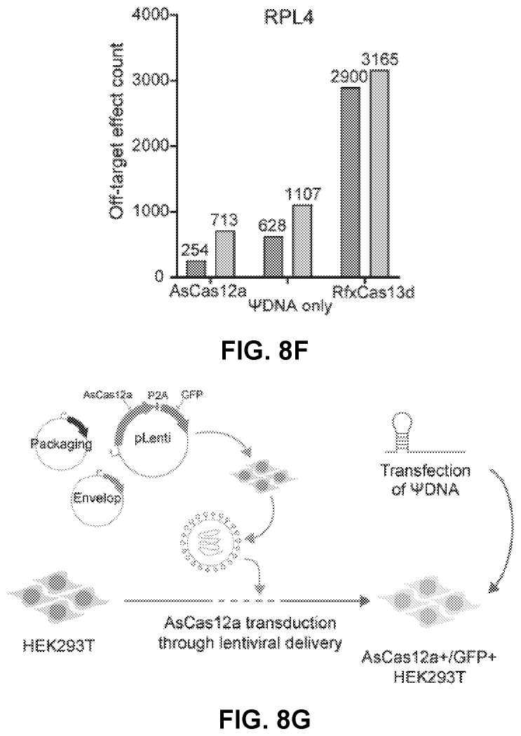

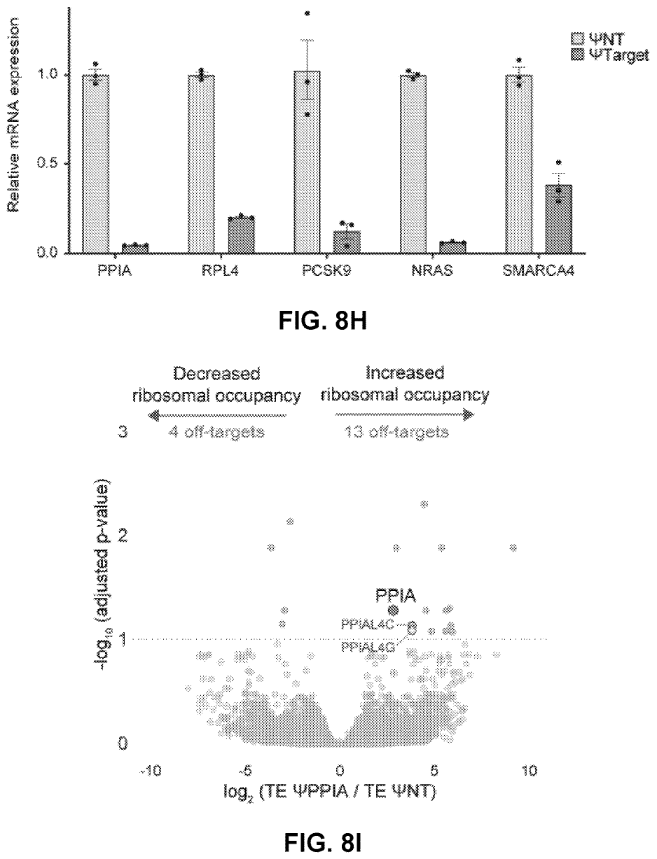

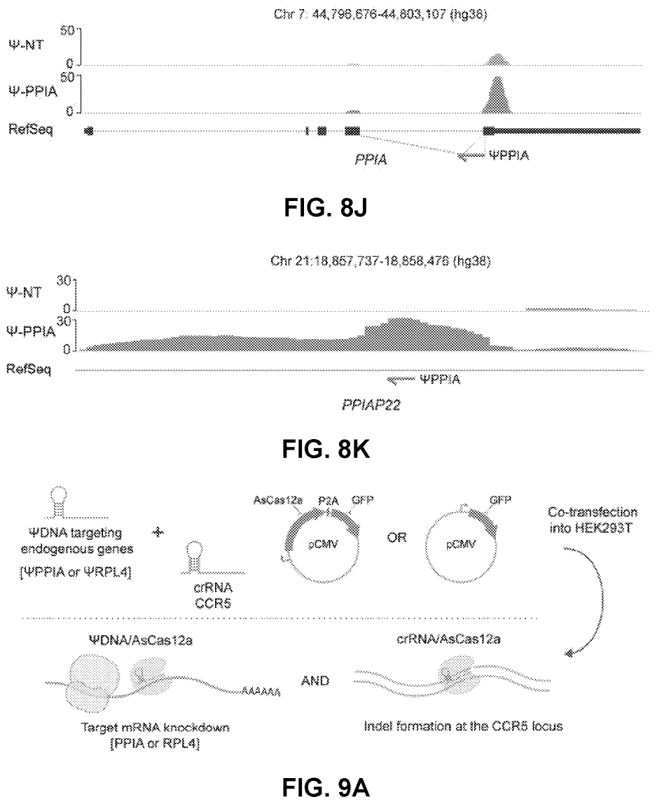

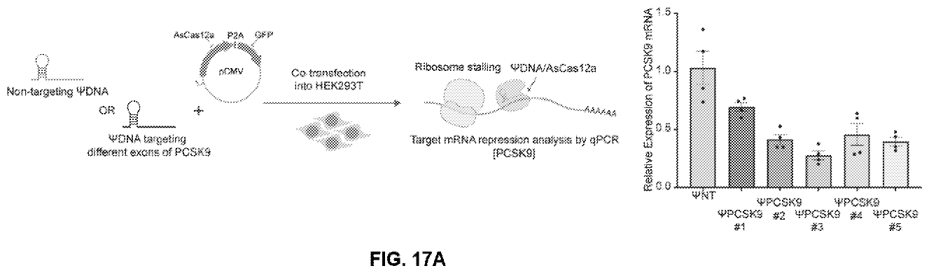

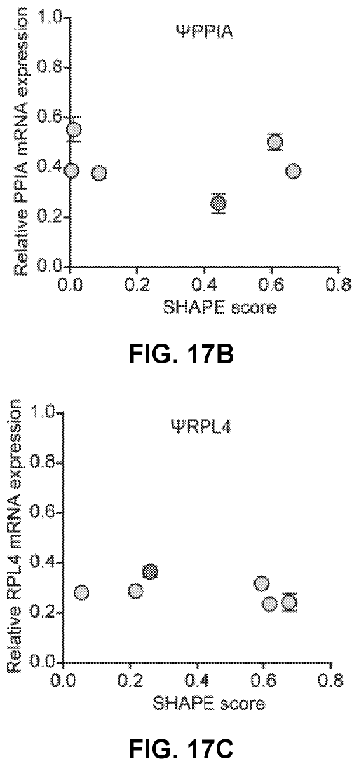

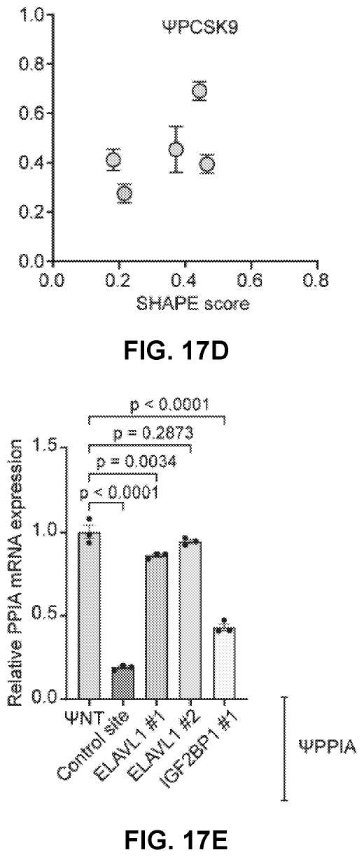

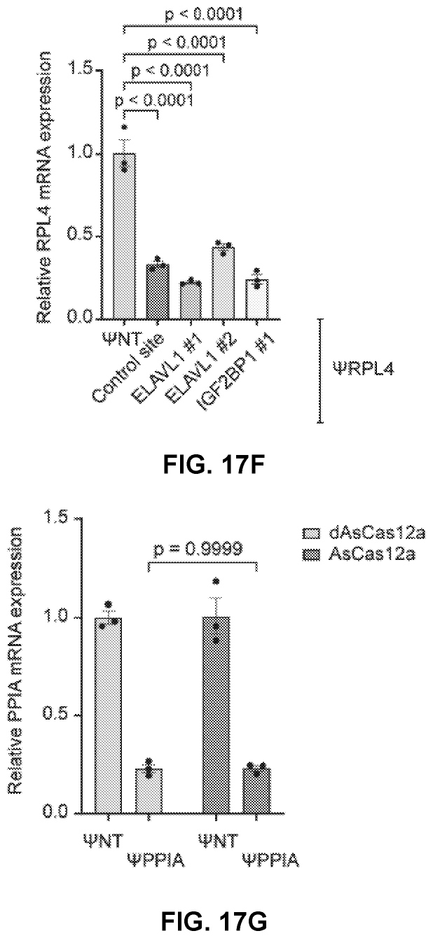

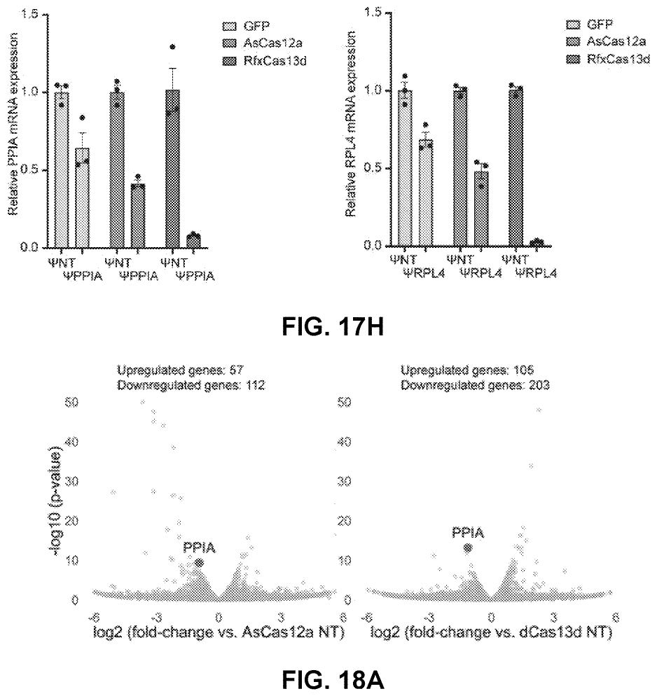

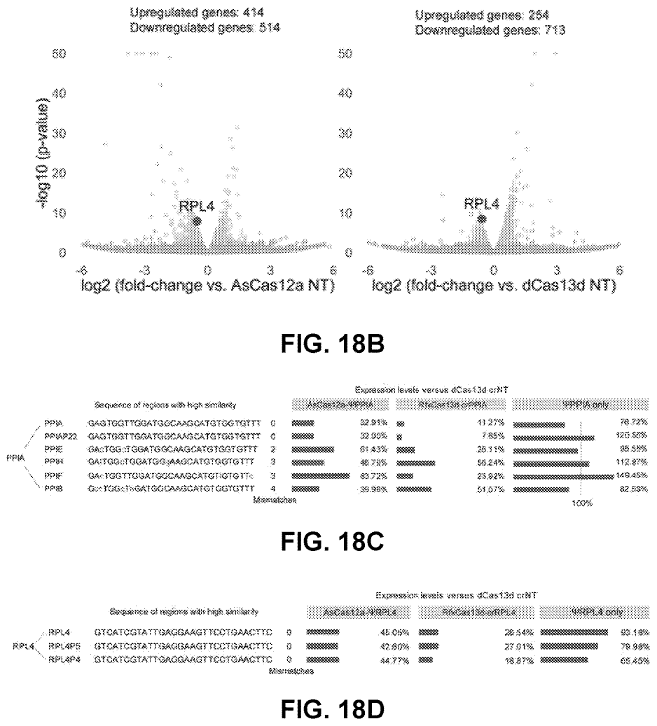

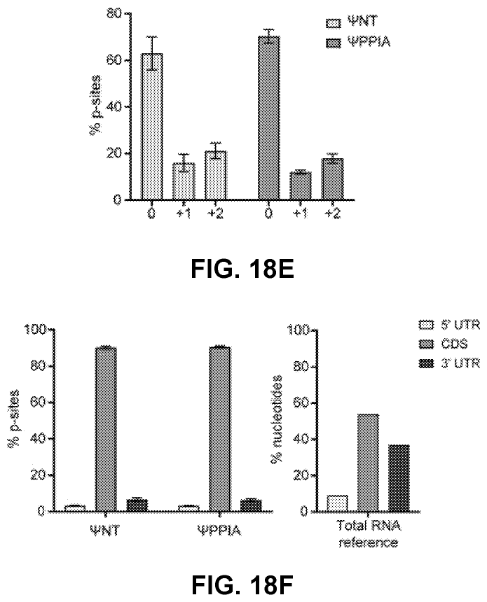

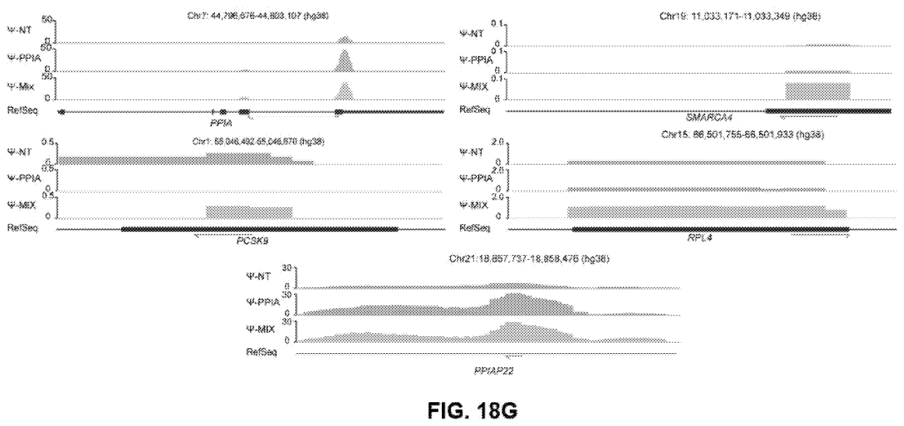

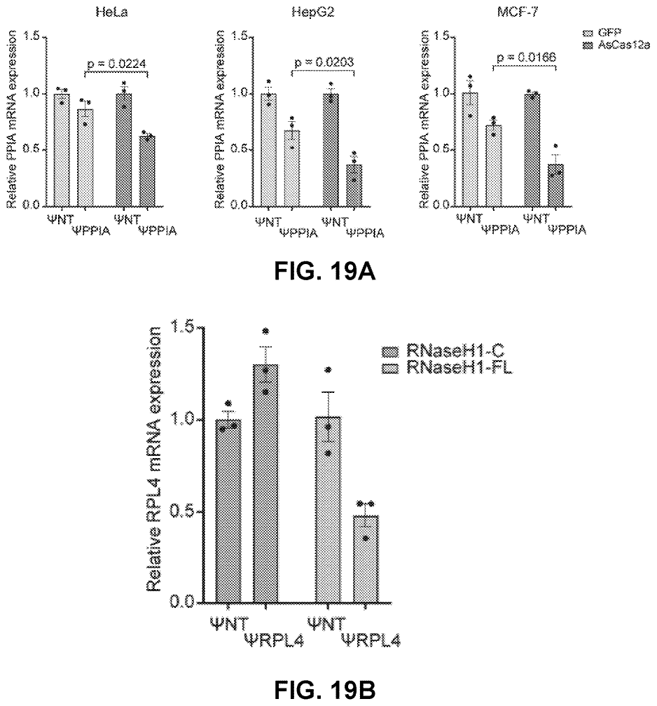

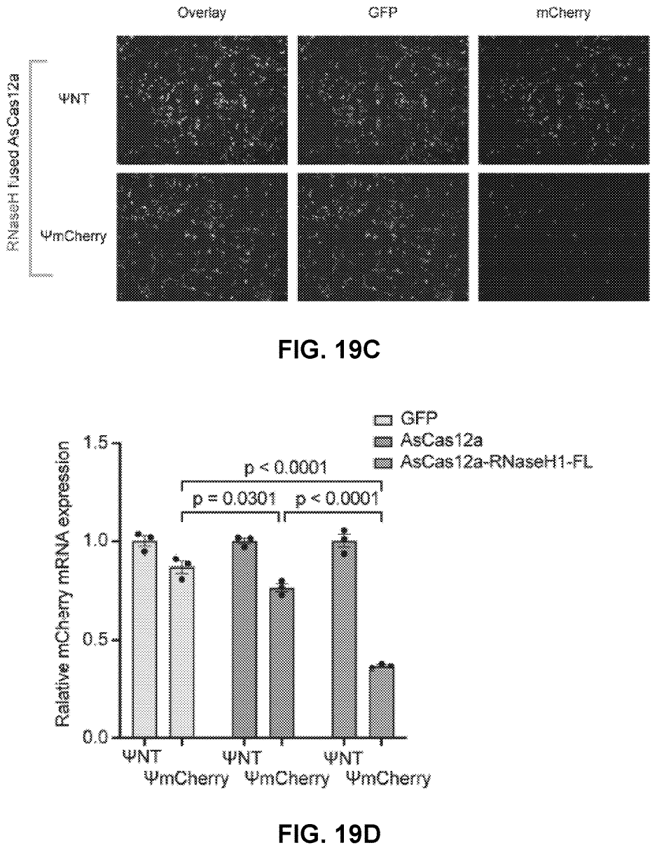

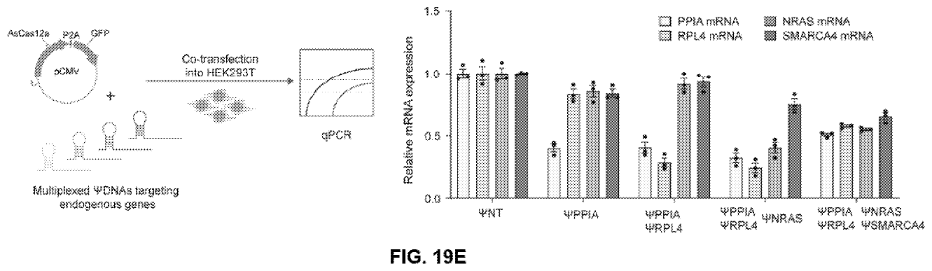

The ability of the ΨDNA-guided CRISPR-Cas12a system to selectively knockdown endogenous RNA transcripts in HEK 293T cells was assessed. To this end, the relative expression levels of the PPIA, RPL4, and PCSK9 mRNAs were measured following transfection with ΨDNAs specific to these transcripts (ΨPPIA, ΨRPL4, and ΨPCSK9) in combination with either GFP (control) or AsCas12a (FIGS. 8A and 17A). In the control cells transfected with GFP, partial knockdown of the target transcripts was observed, potentially due to low-level ASO knockdown activity exerted by ΨDNAs alone. However, co-transfection of AsCas12a with the ΨDNAs led to a significant enhancement in transcript depletion. Overall, the RNA knockdown resulted in a 50% to 70% reduction in mRNA levels compared to the non-targeting (NT) control. Moreover, similar knockdown effects were reproducibly observed in multiple cancer cell lines, including HeLa, HepG2, and MCF-7 (FIG. 18A). These results demonstrate that while ΨDNA alone can induce partial knockdown, co-expression with AsCas12a substantially increases the efficiency and specificity of RNA transcript depletion, confirming the dependence of ΨDNA-mediated knockdown on the presence of AsCas12a. Importantly, comparable knockdown efficiencies were observed when ΨDNA was co-expressed with either wild-type AsCas12a or a catalytically inactive mutant (dAsCas12a), further supporting that ΨDNA-mediated RNA knockdown does not arise from direct enzymatic RNA cleavage by AsCas12a (FIG. 17G).

To validate that AsCas12a is specifically recruiting and binding target RNA transcripts in the presence of ΨDNAs, RNA immunoprecipitation (RIP) was conducted using an HA-tagged version of AsCas12a. Cells delivered with either HA-GFP (control) or HA-AsCas12a and ΨPPIA were subjected to RIP, followed by qPCR analysis of the immunoprecipitated RNA to quantify the binding (FIG. 8B). It was observed that PPIA transcripts were significantly enriched in samples expressing HA-AsCas12a compared to those expressing the control HA-GFP, demonstrating that AsCas12a binds directly to its targeted PPIA transcript when guided by ΨDNA. These results confirm that ΨDNA is facilitating direct interaction between AsCas12a and its endogenous RNA targets.

To assess the flexibility of ΨDNA, multiple target sites in PPIA, RPL4, and PCSK9 were designed based on SHAPE scores from the RASP v2.0 database and found no significant correlation between RNA knockdown efficiency and local RNA secondary structure. Using CLIP-seq data from the POSTAR3 database, 6 target sites overlapping RNA-binding protein (RBP) binding regions in PPIA and RPL4 transcripts were further designed. RBPs influenced ΨDNA-mediated RNA knockdown at PPIA sites but had little effect at RPL4 sites, indicating that RBP effects on ΨDNA targeting are target-dependent (FIGS. 17B-17F). ΨDNA sequences for correlating RNA secondary structure with knockdown efficiency are presented in Table 7. ΨDNA sequences for assessing the impact of RNA-binding proteins on knockdown efficiency are presented in Table 8. ΨDNAs of systematic pseudoknot structure testing are presented in Table 9. Primers to amplify pseudoknot ΨDNA are presented in Table 10.

ΨDNA Sequences for Correlating RNA Secondary Structure with Knockdown Efficiency

| Name | Sequence |
|---|---|
| ΨDNA PPIA #1 (SEQ | A*A*A*CACCACATGCTTGCCATCCAACCACTCTAGATGTGAATCATCTTT*A*A*T |
| ID NO. 276) | |
| ΨDNA PPIA #2 (SEQ | C*A*G*TGCCATTATGGCGTGTGAAGTCACCACTAGATGTGAATCATCTTT*A*A*T |
| ID NO. 277) | |
| ΨDNA PPIA #3 (SEQ | G*T*C*TTGGCAGTGCAGATGAAAAACTGGGAATAGATGTGAATCATCTTT*A*A*T |
| ID NO. 278) | |
| ΨDNA PPIA #4 (SEQ | G*T*A*TGCTTTAGGATGAAGTTCTCATCTTCATAGATGTGAATCATCTTT*A*A*T |
| ID NO. 279) | |
| ΨDNA PPIA #5 (SEQ | C*T*G*GTGGTTAAGATAAAACACAAGTCAAACTAGATGTGAATCATCTTT*A*A*T |
| ID NO. 280) | |
| ΨDNA PPIA #6 (SEQ | T*T*C*ACTTTGCCAAACACCACATGCTTGCCATAGATGTGAATCATCTTT*A*A*T |
| ID NO. 281) | |
| ΨDNA RPL4 #1 | G*A*A*GTTCAGGAACTTCCTCAATACGATGACTAGATGTGAATCATCTTT*A*A*T |
| (SEQ ID NO. 282) | |
| ΨDNA RPL4 #2 | T*C*T*TTGGATCTCTGGGCTTTTCAAGATTCTTAGATGTGAATCATCTTT*A*A*T |
| (SEQ ID NO. 283) | |
| ΨDNA RPL4 #3 | T*A*T*CGTTTTTGGGTTGTGTTCACTCTACGATAGATGTGAATCATCTTT*A*A*T |
| (SEQ ID NO. 284) | |
| ΨDNA RPL4 #4 | T*T*T*TTGATATCATTCCAGGCTTTAAGTTTCTAGATGTGAATCATCTTT*A*A*T |
| (SEQ ID NO. 284) | |
| ΨDNA RPL4#5 (SEQ | A*C*C*AGTGCTGGTAGGGCTGAGGCAGCCAGGTAGATGTGAATCATCTTT*A*A*T |
| ID NO. 286) | |
| ΨDNA RPL4 #6 | A*A*G*GAGCAAAACAGCTTCCTTGGTCTTCTTTAGATGTGAATCATCTTT*A*A*T |
| (SEQ ID NO. 287) | |

*signifies phosphorothioate bond

TABLE 8
ΨDNA Sequences for Assessing the Impact of RNA-Binding Proteins on Knockdown Efficiency

| Name | Sequence |
|---|---|
| ΨDNA PPIA | T*A*C*ATTACAGACAGTGAGCCACCATGCCCATAGATGTGAATCAT |
| ELAVL1_binding_#1 (SEQ ID NO. 288) | CTTT*A*A*T |
| ΨDNA PPIA | T*G*G*GACTGGAAAGTAAAAAAAATCTAAGTATAGATGTGAATCAT |
| ELAVL1_binding_#2 (SEQ ID NO. 289) | CTTT*A*A*T |
| ΨDNA PPIA | A*T*G*CCAGGACCCGTATGCTTTAGGATGAAGTAGATGTGAATCAT |
| IGF2BP1_binding_#1 (SEQ ID NO. 290) | CTTT*A*A*T |
| ΨDNA RPL4 | T*G*G*GTTCTTCTTTAGGACTCTGCGATGGATTAGATGTGAATCAT |
| ELAVL1_binding_#1 (SEQ ID NO. 291) | CTTT*A*A*T |
| ΨDNA RPL4 | T*G*A*GGCAGCCAGGGCAGAACAGATGGCGTATAGATGTGAATCA |
| ELAVL1_binding_#2 (SEQ ID NO. 292) | TCTTT*A*A*T |
| ΨDNA RPL4 | C*T*T*TTCGGAGTACACCGATATCAGTGGGCGTAGATGTGAATCAT |
| IGF2BP1 binding_#1 (SEQ ID NO. 293) | CTTT*A*A*T |

*signifies phosphorothioate bond

TABLE 9
ΨDNAs of Systematic Pseudoknot Structure Testing

| pseudoknot ΨDNA | Sequence | index |
|---|---|---|
| 1 (SEQ ID NO. 294) | ACAGCCCATCGACTGGTGTTTAAAAGTGAATCATCTTTAATCTGTC | AAAA |
| | TCTTATACAC | |
| 2 (SEQ ID NO. 295) | ACAGCCCATCGACTGGTGTTTAAACGTGAATCATCTTTAATCTGTC | AAAC |
| | TCTTATACAC | |
| 3 (SEQ ID NO. 296) | ACAGCCCATCGACTGGTGTTTAAAGGTGAATCATCTTTAATCTGTC | AAAG |
| | TCTTATACAC | |
| 4 (SEQ ID NO. 297) | ACAGCCCATCGACTGGTGTTTAAATGTGAATCATCTTTAATCTGTC | AAAT |
| | TCTTATACAC | |
| 5 (SEQ ID NO. 298) | ACAGCCCATCGACTGGTGTTTAACAGTGAATCATCTTTAATCTGTC | AACA |
| | TCTTATACAC | |
| 6 (SEQ ID NO. 299) | ACAGCCCATCGACTGGTGTTTAACCGTGAATCATCTTTAATCTGTC | AACC |
| | TCTTATACAC | |
| 7 (SEQ ID NO. 300) | ACAGCCCATCGACTGGTGTTTAACGGTGAATCATCTTTAATCTGTC | AACG |
| | TCTTATACAC | |
| 8 (SEQ ID NO. 301) | ACAGCCCATCGACTGGTGTTTAACTGTGAATCATCTTTAATCTGTC | AACT |
| | TCTTATACAC | |
| 9 (SEQ ID NO. 302) | ACAGCCCATCGACTGGTGTTTAAGAGTGAATCATCTTTAATCTGTC | AAGA |
| | TCTTATACAC | |
| 10 (SEQ ID NO. 303) | ACAGCCCATCGACTGGTGTTTAAGCGTGAATCATCTTTAATCTGTC | AAGC |
| | TCTTATACAC | |
| 11 (SEQ ID NO. 304) | ACAGCCCATCGACTGGTGTTTAAGGGTGAATCATCTTTAATCTGTC | AAGG |
| | TCTTATACAC | |
| 12 (SEQ ID NO. 305) | ACAGCCCATCGACTGGTGTTTAAGTGTGAATCATCTTTAATCTGTC | AAGT |
| | TCTTATACAC | |
| 13 (SEQ ID NO. 306) | ACAGCCCATCGACTGGTGTTTAATAGTGAATCATCTTTAATCTGTC | AATA |
| | TCTTATACAC | |
| 14 (SEQ ID NO. 307) | ACAGCCCATCGACTGGTGTTTAATCGTGAATCATCTTTAATCTGTC | AATC |
| | TCTTATACAC | |
| 15 (SEQ ID NO. 308) | ACAGCCCATCGACTGGTGTTTAATGGTGAATCATCTTTAATCTGTC | AATG |
| | TCTTATACAC | |
| 16 (SEQ ID NO. 309) | ACAGCCCATCGACTGGTGTTTAATTGTGAATCATCTTTAATCTGTC | AATT |
| | TCTTATACAC | |
| 17 (SEQ ID NO. 310) | ACAGCCCATCGACTGGTGTTTACAAGTGAATCATCTTTAATCTGTC | ACAA |
| | TCTTATACAC | |
| 18 (SEQ ID NO. 311) | ACAGCCCATCGACTGGTGTTTACACGTGAATCATCTTTAATCTGTC | ACAC |
| | TCTTATACAC | |
| 19 (SEQ ID NO. 312) | ACAGCCCATCGACTGGTGTTTACAGGTGAATCATCTTTAATCTGTC | ACAG |
| | TCTTATACAC | |
| 20 (SEQ ID NO. 313) | ACAGCCCATCGACTGGTGTTTACATGTGAATCATCTTTAATCTGTC | ACAT |
| | TCTTATACAC | |
| 21 (SEQ ID NO. 314) | ACAGCCCATCGACTGGTGTTTACCAGTGAATCATCTTTAATCTGTC | ACCA |
| | TCTTATACAC | |
| 22 (SEQ ID NO. 315) | ACAGCCCATCGACTGGTGTTTACCCGTGAATCATCTTTAATCTGTC | ACCC |
| | TCTTATACAC | |
| 23 (SEQ ID NO. 316) | ACAGCCCATCGACTGGTGTTTACCGGTGAATCATCTTTAATCTGTC | ACCG |
| | TCTTATACAC | |
| 24 (SEQ ID NO. 317) | ACAGCCCATCGACTGGTGTTTACCTGTGAATCATCTTTAATCTGTC | ACCT |
| | TCTTATACAC | |
| 25 (SEQ ID NO. 318) | ACAGCCCATCGACTGGTGTTTACGAGTGAATCATCTTTAATCTGTC | ACGA |
| | TCTTATACAC | |
| 26 (SEQ ID NO. 319) | ACAGCCCATCGACTGGTGTTTACGCGTGAATCATCTTTAATCTGTC | ACGC |
| | TCTTATACAC | |
| 27 (SEQ ID NO. 320) | ACAGCCCATCGACTGGTGTTTACGGGTGAATCATCTTTAATCTGTC | ACGG |
| | TCTTATACAC | |
| 28 (SEQ ID NO. 321) | ACAGCCCATCGACTGGTGTTTACGTGTGAATCATCTTTAATCTGTC | ACGT |
| | TCTTATACAC | |
| 29 (SEQ ID NO. 322) | ACAGCCCATCGACTGGTGTTTACTAGTGAATCATCTTTAATCTGTC | ACTA |
| | TCTTATACAC | |
| 30 (SEQ ID NO. 323) | ACAGCCCATCGACTGGTGTTTACTCGTGAATCATCTTTAATCTGTC | ACTC |
| | TCTTATACAC | |
| 31 (SEQ ID NO. 324) | ACAGCCCATCGACTGGTGTTTACTGGTGAATCATCTTTAATCTGTC | ACTG |
| | TCTTATACAC | |
| 32 (SEQ ID NO. 325) | ACAGCCCATCGACTGGTGTTTACTTGTGAATCATCTTTAATCTGTC | ACTT |
| | TCTTATACAC | |
| 33 (SEQ ID NO. 326) | ACAGCCCATCGACTGGTGTTTAGAAGTGAATCATCTTTAATCTGTC | AGAA |
| | TCTTATACAC | |
| 34 (SEQ ID NO. 327) | ACAGCCCATCGACTGGTGTTTAGACGTGAATCATCTTTAATCTGTC | AGAC |
| | TCTTATACAC | |
| 35 (SEQ ID NO. 328) | ACAGCCCATCGACTGGTGTTTAGAGGTGAATCATCTTTAATCTGTC | AGAG |
| | TCTTATACAC | |
| 36 (SEQ ID NO. 329) | ACAGCCCATCGACTGGTGTTTAGATGTGAATCATCTTTAATCTGTC | AGAT |
| | TCTTATACAC | |
| 37 (SEQ ID NO. 330) | ACAGCCCATCGACTGGTGTTTAGCAGTGAATCATCTTTAATCTGTC | AGCA |
| | TCTTATACAC | |
| 38 (SEQ ID NO. 331) | ACAGCCCATCGACTGGTGTTTAGCCGTGAATCATCTTTAATCTGTC | AGCC |
| | TCTTATACAC | |
| 39 (SEQ ID NO. 332) | ACAGCCCATCGACTGGTGTTTAGCGGTGAATCATCTTTAATCTGTC | AGCG |
| | TCTTATACAC | |
| 40 (SEQ ID NO. 333) | ACAGCCCATCGACTGGTGTTTAGCTGTGAATCATCTTTAATCTGTC | AGCT |
| | TCTTATACAC | |
| 41 (SEQ ID NO. 334) | ACAGCCCATCGACTGGTGTTTAGGAGTGAATCATCTTTAATCTGTC | AGGA |
| | TCTTATACAC | |
| 42 (SEQ ID NO. 335) | ACAGCCCATCGACTGGTGTTTAGGCGTGAATCATCTTTAATCTGTC | AGGC |
| | TCTTATACAC | |
| 43 (SEQ ID NO. 336) | ACAGCCCATCGACTGGTGTTTAGGGGTGAATCATCTTTAATCTGTC | AGGG |
| | TCTTATACAC | |
| 44 (SEQ ID NO. 337) | ACAGCCCATCGACTGGTGTTTAGGTGTGAATCATCTTTAATCTGTC | AGGT |
| | TCTTATACAC | |
| 45 (SEQ ID NO. 338) | ACAGCCCATCGACTGGTGTTTAGTAGTGAATCATCTTTAATCTGTC | AGTA |
| | TCTTATACAC | |
| 46 (SEQ ID NO. 339) | ACAGCCCATCGACTGGTGTTTAGTCGTGAATCATCTTTAATCTGTC | AGTC |
| | TCTTATACAC | |
| 47 (SEQ ID NO. 340) | ACAGCCCATCGACTGGTGTTTAGTGGTGAATCATCTTTAATCTGTC | AGTG |
| | TCTTATACAC | |
| 48 (SEQ ID NO. 341) | ACAGCCCATCGACTGGTGTTTAGTTGTGAATCATCTTTAATCTGTC | AGTT |
| | TCTTATACAC | |
| 49 (SEQ ID NO. 342) | ACAGCCCATCGACTGGTGTTTATAAGTGAATCATCTTTAATCTGTC | ATAA |
| | TCTTATACAC | |
| 50 (SEQ ID NO. 343) | ACAGCCCATCGACTGGTGTTTATACGTGAATCATCTTTAATCTGTC | ATAC |
| | TCTTATACAC | |
| 51 (SEQ ID NO. 344) | ACAGCCCATCGACTGGTGTTTATAGGTGAATCATCTTTAATCTGTC | ATAG |
| | TCTTATACAC | |
| 52 (SEQ ID NO. 345) | ACAGCCCATCGACTGGTGTTTATATGTGAATCATCTTTAATCTGTC | ATAT |
| | TCTTATACAC | |
| 53 (SEQ ID NO. 346) | ACAGCCCATCGACTGGTGTTTATCAGTGAATCATCTTTAATCTGTC | ATCA |
| | TCTTATACAC | |
| 54 (SEQ ID NO. 347) | ACAGCCCATCGACTGGTGTTTATCCGTGAATCATCTTTAATCTGTC | ATCC |
| | TCTTATACAC | |
| 55 (SEQ ID NO. 348) | ACAGCCCATCGACTGGTGTTTATCGGTGAATCATCTTTAATCTGTC | ATCG |
| | TCTTATACAC | |
| 56 (SEQ ID NO. 349) | ACAGCCCATCGACTGGTGTTTATCTGTGAATCATCTTTAATCTGTC | ATCT |
| | TCTTATACAC | |
| 57 (SEQ ID NO. 350) | ACAGCCCATCGACTGGTGTTTATGAGTGAATCATCTTTAATCTGTC | ATGA |
| | TCTTATACAC | |
| 58 (SEQ ID NO. 351) | ACAGCCCATCGACTGGTGTTTATGCGTGAATCATCTTTAATCTGTC | ATGC |
| | TCTTATACAC | |
| 59 (SEQ ID NO. 352) | ACAGCCCATCGACTGGTGTTTATGGGTGAATCATCTTTAATCTGTC | ATGG |
| | TCTTATACAC | |
| 60 (SEQ ID NO. 353) | ACAGCCCATCGACTGGTGTTTATGTGTGAATCATCTTTAATCTGTC | ATGT |
| | TCTTATACAC | |
| 61 (SEQ ID NO. 354) | ACAGCCCATCGACTGGTGTTTATTAGTGAATCATCTTTAATCTGTC | ATTA |
| | TCTTATACAC | |
| 62 (SEQ ID NO. 355) | ACAGCCCATCGACTGGTGTTTATTCGTGAATCATCTTTAATCTGTC | ATTC |
| | TCTTATACAC | |
| 63 (SEQ ID NO. 356) | ACAGCCCATCGACTGGTGTTTATTGGTGAATCATCTTTAATCTGTC | ATTG |
| | TCTTATACAC | |
| 64 (SEQ ID NO. 357) | ACAGCCCATCGACTGGTGTTTATTTGTGAATCATCTTTAATCTGTCT | ATTT |
| | CTTATACAC | |
| 65 (SEQ ID NO. 358) | ACAGCCCATCGACTGGTGTTTCAAAGTGAATCATCTTTAATCTGTC | CAAA |
| | TCTTATACAC | |
| 66 (SEQ ID NO. 359) | ACAGCCCATCGACTGGTGTTTCAACGTGAATCATCTTTAATCTGTC | CAAC |
| | TCTTATACAC | |
| 67 (SEQ ID NO. 360) | ACAGCCCATCGACTGGTGTTTCAAGGTGAATCATCTTTAATCTGTC | CAAG |
| | TCTTATACAC | |
| 68 (SEQ ID NO. 361) | ACAGCCCATCGACTGGTGTTTCAATGTGAATCATCTTTAATCTGTC | CAAT |
| | TCTTATACAC | |
| 69 (SEQ ID NO. 362) | ACAGCCCATCGACTGGTGTTTCACAGTGAATCATCTTTAATCTGTC | CACA |
| | TCTTATACAC | |
| 70 (SEQ ID NO. 363) | ACAGCCCATCGACTGGTGTTTCACCGTGAATCATCTTTAATCTGTC | CACC |
| | TCTTATACAC | |
| 71 (SEQ ID NO. 364) | ACAGCCCATCGACTGGTGTTTCACGGTGAATCATCTTTAATCTGTC | CACG |
| | TCTTATACAC | |
| 72 (SEQ ID NO. 365) | ACAGCCCATCGACTGGTGTTTCACTGTGAATCATCTTTAATCTGTC | CACT |
| | TCTTATACAC | |
| 73 (SEQ ID NO. 366) | ACAGCCCATCGACTGGTGTTTCAGAGTGAATCATCTTTAATCTGTC | CAGA |
| | TCTTATACAC | |
| 74 (SEQ ID NO. 367) | ACAGCCCATCGACTGGTGTTTCAGCGTGAATCATCTTTAATCTGTC | CAGC |
| | TCTTATACAC | |
| 75 (SEQ ID NO. 368) | ACAGCCCATCGACTGGTGTTTCAGGGTGAATCATCTTTAATCTGTC | CAGG |
| | TCTTATACAC | |
| 76 (SEQ ID NO. 369) | ACAGCCCATCGACTGGTGTTTCAGTGTGAATCATCTTTAATCTGTC | CAGT |
| | TCTTATACAC | |
| 77 (SEQ ID NO. 370) | ACAGCCCATCGACTGGTGTTTCATAGTGAATCATCTTTAATCTGTC | CATA |
| | TCTTATACAC | |
| 78 (SEQ ID NO. 371) | ACAGCCCATCGACTGGTGTTTCATCGTGAATCATCTTTAATCTGTC | CATC |
| | TCTTATACAC | |
| 79 (SEQ ID NO. 372) | ACAGCCCATCGACTGGTGTTTCATGGTGAATCATCTTTAATCTGTC | CATG |
| | TCTTATACAC | |
| 80 (SEQ ID NO. 373) | ACAGCCCATCGACTGGTGTTTCATTGTGAATCATCTTTAATCTGTC | CATT |
| | TCTTATACAC | |
| 81 (SEQ ID NO. 374) | ACAGCCCATCGACTGGTGTTTCCAAGTGAATCATCTTTAATCTGTC | CCAA |
| | TCTTATACAC | |
| 82 (SEQ ID NO. 375) | ACAGCCCATCGACTGGTGTTTCCACGTGAATCATCTTTAATCTGTC | CCAC |
| | TCTTATACAC | |
| 83 (SEQ ID NO. 376) | ACAGCCCATCGACTGGTGTTTCCAGGTGAATCATCTTTAATCTGTC | CCAG |
| | TCTTATACAC | |
| 84 (SEQ ID NO. 377) | ACAGCCCATCGACTGGTGTTTCCATGTGAATCATCTTTAATCTGTC | CCAT |
| | TCTTATACAC | |
| 85 (SEQ ID NO. 378) | ACAGCCCATCGACTGGTGTTTCCCAGTGAATCATCTTTAATCTGTC | CCCA |
| | TCTTATACAC | |
| 86 (SEQ ID NO. 379) | ACAGCCCATCGACTGGTGTTTCCCCGTGAATCATCTTTAATCTGTC | CCCC |
| | TCTTATACAC | |
| 87 (SEQ ID NO. 380) | ACAGCCCATCGACTGGTGTTTCCCGGTGAATCATCTTTAATCTGTC | CCCG |
| | TCTTATACAC | |
| 88 (SEQ ID NO. 381) | ACAGCCCATCGACTGGTGTTTCCCTGTGAATCATCTTTAATCTGTC | CCCT |
| | TCTTATACAC | |
| 89 (SEQ ID NO. 382) | ACAGCCCATCGACTGGTGTTTCCGAGTGAATCATCTTTAATCTGTC | CCGA |
| | TCTTATACAC | |
| 90 (SEQ ID NO. 383) | ACAGCCCATCGACTGGTGTTTCCGCGTGAATCATCTTTAATCTGTC | CCGC |
| | TCTTATACAC | |
| 91 (SEQ ID NO. 384) | ACAGCCCATCGACTGGTGTTTCCGGGTGAATCATCTTTAATCTGTC | CCGG |
| | TCTTATACAC | |
| 92 (SEQ ID NO. 385) | ACAGCCCATCGACTGGTGTTTCCGTGTGAATCATCTTTAATCTGTC | CCGT |
| | TCTTATACAC | |
| 93 (SEQ ID NO. 386) | ACAGCCCATCGACTGGTGTTTCCTAGTGAATCATCTTTAATCTGTC | CCTA |
| | TCTTATACAC | |
| 94 (SEQ ID NO. 387) | ACAGCCCATCGACTGGTGTTTCCTCGTGAATCATCTTTAATCTGTC | CCTC |
| | TCTTATACAC | |
| 95 (SEQ ID NO. 388) | ACAGCCCATCGACTGGTGTTTCCTGGTGAATCATCTTTAATCTGTC | CCTG |
| | TCTTATACAC | |
| 96 (SEQ ID NO. 389) | ACAGCCCATCGACTGGTGTTTCCTTGTGAATCATCTTTAATCTGTCT | CCTT |
| | CTTATACAC | |
| 97 (SEQ ID NO. 390) | ACAGCCCATCGACTGGTGTTTCGAAGTGAATCATCTTTAATCTGTC | CGAA |
| | TCTTATACAC | |
| 98 (SEQ ID NO. 391) | ACAGCCCATCGACTGGTGTTTCGACGTGAATCATCTTTAATCTGTC | CGAC |
| | TCTTATACAC | |
| 99 (SEQ ID NO. 392) | ACAGCCCATCGACTGGTGTTTCGAGGTGAATCATCTTTAATCTGTC | CGAG |
| | TCTTATACAC | |
| 100 (SEQ ID NO. 393) | ACAGCCCATCGACTGGTGTTTCGATGTGAATCATCTTTAATCTGTC | CGAT |
| | TCTTATACAC | |
| 101 (SEQ ID NO. 394) | ACAGCCCATCGACTGGTGTTTCGCAGTGAATCATCTTTAATCTGTC | CGCA |
| | TCTTATACAC | |
| 102 (SEQ ID NO. 395) | ACAGCCCATCGACTGGTGTTTCGCCGTGAATCATCTTTAATCTGTC | CGCC |
| | TCTTATACAC | |
| 103 (SEQ ID NO. 396) | ACAGCCCATCGACTGGTGTTTCGCGGTGAATCATCTTTAATCTGTC | CGCG |
| | TCTTATACAC | |
| 104 (SEQ ID NO. 397) | ACAGCCCATCGACTGGTGTTTCGCTGTGAATCATCTTTAATCTGTC | CGCT |
| | TCTTATACAC | |
| 105 (SEQ ID NO. 398) | ACAGCCCATCGACTGGTGTTTCGGAGTGAATCATCTTTAATCTGTC | CGGA |
| | TCTTATACAC | |
| 106 (SEQ ID NO. 399) | ACAGCCCATCGACTGGTGTTTCGGCGTGAATCATCTTTAATCTGTC | CGGC |
| | TCTTATACAC | |
| 107 (SEQ ID NO. 400) | ACAGCCCATCGACTGGTGTTTCGGGGTGAATCATCTTTAATCTGTC | CGGG |
| | TCTTATACAC | |
| 108 (SEQ ID NO. 401) | ACAGCCCATCGACTGGTGTTTCGGTGTGAATCATCTTTAATCTGTC | CGGT |
| | TCTTATACAC | |
| 109 (SEQ ID NO. 402) | ACAGCCCATCGACTGGTGTTTCGTAGTGAATCATCTTTAATCTGTC | CGTA |
| | TCTTATACAC | |
| 110 (SEQ ID NO. 403) | ACAGCCCATCGACTGGTGTTTCGTCGTGAATCATCTTTAATCTGTC | CGTC |
| | TCTTATACAC | |
| 111 (SEQ ID NO. 404) | ACAGCCCATCGACTGGTGTTTCGTGGTGAATCATCTTTAATCTGTC | CGTG |
| | TCTTATACAC | |
| 112 (SEQ ID NO. 405) | ACAGCCCATCGACTGGTGTTTCGTTGTGAATCATCTTTAATCTGTC | CGTT |
| | TCTTATACAC | |
| 113 (SEQ ID NO. 406) | ACAGCCCATCGACTGGTGTTTCTAAGTGAATCATCTTTAATCTGTC | CTAA |
| | TCTTATACAC | |
| 114 (SEQ ID NO. 407) | ACAGCCCATCGACTGGTGTTTCTACGTGAATCATCTTTAATCTGTC | CTAC |
| | TCTTATACAC | |
| 115 (SEQ ID NO. 408) | ACAGCCCATCGACTGGTGTTTCTAGGTGAATCATCTTTAATCTGTC | CTAG |
| | TCTTATACAC | |
| 116 (SEQ ID NO. 409) | ACAGCCCATCGACTGGTGTTTCTATGTGAATCATCTTTAATCTGTC | CTAT |
| | TCTTATACAC | |
| 117 (SEQ ID NO. 410) | ACAGCCCATCGACTGGTGTTTCTCAGTGAATCATCTTTAATCTGTC | CTCA |
| | TCTTATACAC | |
| 118 (SEQ ID NO. 411) | ACAGCCCATCGACTGGTGTTTCTCCGTGAATCATCTTTAATCTGTC | CTCC |
| | TCTTATACAC | |
| 119 (SEQ ID NO. 412) | ACAGCCCATCGACTGGTGTTTCTCGGTGAATCATCTTTAATCTGTC | CTCG |
| | TCTTATACAC | |
| 120 (SEQ ID NO. 413) | ACAGCCCATCGACTGGTGTTTCTCTGTGAATCATCTTTAATCTGTCT | CTCT |
| | CTTATACAC | |
| 121 (SEQ ID NO. 414) | ACAGCCCATCGACTGGTGTTTCTGAGTGAATCATCTTTAATCTGTC | CTGA |
| | TCTTATACAC | |
| 122 (SEQ ID NO. 415) | ACAGCCCATCGACTGGTGTTTCTGCGTGAATCATCTTTAATCTGTC | CTGC |
| | TCTTATACAC | |
| 123 (SEQ ID NO. 416) | ACAGCCCATCGACTGGTGTTTCTGGGTGAATCATCTTTAATCTGTC | CTGG |
| | TCTTATACAC | |
| 124 (SEQ ID NO. 417) | ACAGCCCATCGACTGGTGTTTCTGTGTGAATCATCTTTAATCTGTC | CTGT |
| | TCTTATACAC | |
| 125 (SEQ ID NO. 418) | ACAGCCCATCGACTGGTGTTTCTTAGTGAATCATCTTTAATCTGTC | CTTA |
| | TCTTATACAC | |
| 126 (SEQ ID NO. 419) | ACAGCCCATCGACTGGTGTTTCTTCGTGAATCATCTTTAATCTGTCT | CTTC |
| | CTTATACAC | |
| 127 (SEQ ID NO. 420) | ACAGCCCATCGACTGGTGTTTCTTGGTGAATCATCTTTAATCTGTC | CTTG |
| | TCTTATACAC | |
| 128 (SEQ ID NO. 421) | ACAGCCCATCGACTGGTGTTTCTTTGTGAATCATCTTTAATCTGTCT | CTTT |
| | CTTATACAC | |
| 129 (SEQ ID NO. 422) | ACAGCCCATCGACTGGTGTTTGAAAGTGAATCATCTTTAATCTGTC | GAAA |
| | TCTTATACAC | |
| 130 (SEQ ID NO. 423) | ACAGCCCATCGACTGGTGTTTGAACGTGAATCATCTTTAATCTGTC | GAAC |
| | TCTTATACAC | |
| 131 (SEQ ID NO. 424) | ACAGCCCATCGACTGGTGTTTGAAGGTGAATCATCTTTAATCTGTC | GAAG |
| | TCTTATACAC | |
| 132 (SEQ ID NO. 425) | ACAGCCCATCGACTGGTGTTTGAATGTGAATCATCTTTAATCTGTC | GAAT |
| | TCTTATACAC | |
| 133 (SEQ ID NO. 426) | ACAGCCCATCGACTGGTGTTTGACAGTGAATCATCTTTAATCTGTC | GACA |
| | TCTTATACAC | |
| 134 (SEQ ID NO. 427) | ACAGCCCATCGACTGGTGTTTGACCGTGAATCATCTTTAATCTGTC | GACC |
| | TCTTATACAC | |
| 135 (SEQ ID NO. 428) | ACAGCCCATCGACTGGTGTTTGACGGTGAATCATCTTTAATCTGTC | GACG |
| | TCTTATACAC | |
| 136 (SEQ ID NO. 429) | ACAGCCCATCGACTGGTGTTTGACTGTGAATCATCTTTAATCTGTC | GACT |
| | TCTTATACAC | |
| 137 (SEQ ID NO. 430) | ACAGCCCATCGACTGGTGTTTGAGAGTGAATCATCTTTAATCTGTC | GAGA |
| | TCTTATACAC | |
| 138 (SEQ ID NO. 431) | ACAGCCCATCGACTGGTGTTTGAGCGTGAATCATCTTTAATCTGTC | GAGC |
| | TCTTATACAC | |
| 139 (SEQ ID NO. 432) | ACAGCCCATCGACTGGTGTTTGAGGGTGAATCATCTTTAATCTGTC | GAGG |
| | TCTTATACAC | |
| 140 (SEQ ID NO. 433) | ACAGCCCATCGACTGGTGTTTGAGTGTGAATCATCTTTAATCTGTC | GAGT |
| | TCTTATACAC | |
| 141 (SEQ ID NO. 434) | ACAGCCCATCGACTGGTGTTTGATAGTGAATCATCTTTAATCTGTC | GATA |
| | TCTTATACAC | |
| 142 (SEQ ID NO. 435) | ACAGCCCATCGACTGGTGTTTGATCGTGAATCATCTTTAATCTGTC | GATC |
| | TCTTATACAC | |
| 143 (SEQ ID NO. 436) | ACAGCCCATCGACTGGTGTTTGATGGTGAATCATCTTTAATCTGTC | GATG |
| | TCTTATACAC | |
| 144 (SEQ ID NO. 437) | ACAGCCCATCGACTGGTGTTTGATTGTGAATCATCTTTAATCTGTC | GATT |
| | TCTTATACAC | |
| 145 (SEQ ID NO. 438) | ACAGCCCATCGACTGGTGTTTGCAAGTGAATCATCTTTAATCTGTC | GCAA |
| | TCTTATACAC | |
| 146 (SEQ ID NO. 439) | ACAGCCCATCGACTGGTGTTTGCACGTGAATCATCTTTAATCTGTC | GCAC |
| | TCTTATACAC | |
| 147 (SEQ ID NO. 440) | ACAGCCCATCGACTGGTGTTTGCAGGTGAATCATCTTTAATCTGTC | GCAG |
| | TCTTATACAC | |
| 148 (SEQ ID NO. 441) | ACAGCCCATCGACTGGTGTTTGCATGTGAATCATCTTTAATCTGTC | GCAT |
| | TCTTATACAC | |
| 149 (SEQ ID NO. 442) | ACAGCCCATCGACTGGTGTTTGCCAGTGAATCATCTTTAATCTGTC | GCCA |
| | TCTTATACAC | |
| 150 (SEQ ID NO. 443) | ACAGCCCATCGACTGGTGTTTGCCCGTGAATCATCTTTAATCTGTC | GCCC |
| | TCTTATACAC | |
| 151 (SEQ ID NO. 444) | ACAGCCCATCGACTGGTGTTTGCCGGTGAATCATCTTTAATCTGTC | GCCG |
| | TCTTATACAC | |
| 152 (SEQ ID NO. 445) | ACAGCCCATCGACTGGTGTTTGCCTGTGAATCATCTTTAATCTGTC | GCCT |
| | TCTTATACAC | |
| 153 (SEQ ID NO. 446) | ACAGCCCATCGACTGGTGTTTGCGAGTGAATCATCTTTAATCTGTC | GCGA |
| | TCTTATACAC | |
| 154 (SEQ ID NO. 447) | ACAGCCCATCGACTGGTGTTTGCGCGTGAATCATCTTTAATCTGTC | GCGC |
| | TCTTATACAC | |
| 155 (SEQ ID NO. 448) | ACAGCCCATCGACTGGTGTTTGCGGGTGAATCATCTTTAATCTGTC | GCGG |
| | TCTTATACAC | |
| 156 (SEQ ID NO. 449) | ACAGCCCATCGACTGGTGTTTGCGTGTGAATCATCTTTAATCTGTC | GCGT |
| | TCTTATACAC | |
| 157 (SEQ ID NO. 450) | ACAGCCCATCGACTGGTGTTTGCTAGTGAATCATCTTTAATCTGTC | GCTA |
| | TCTTATACAC | |
| 158 (SEQ ID NO. 451) | ACAGCCCATCGACTGGTGTTTGCTCGTGAATCATCTTTAATCTGTC | GCTC |
| | TCTTATACAC | |
| 159 (SEQ ID NO. 452) | ACAGCCCATCGACTGGTGTTTGCTGGTGAATCATCTTTAATCTGTC | GCTG |
| | TCTTATACAC | |
| 160 (SEQ ID NO. 453) | ACAGCCCATCGACTGGTGTTTGCTTGTGAATCATCTTTAATCTGTC | GCTT |
| | TCTTATACAC | |
| 161 (SEQ ID NO. 454) | ACAGCCCATCGACTGGTGTTTGGAAGTGAATCATCTTTAATCTGTC | GGAA |
| | TCTTATACAC | |
| 162 (SEQ ID NO. 455) | ACAGCCCATCGACTGGTGTTTGGACGTGAATCATCTTTAATCTGTC | GGAC |
| | TCTTATACAC | |
| 163 (SEQ ID NO. 456) | ACAGCCCATCGACTGGTGTTTGGAGGTGAATCATCTTTAATCTGTC | GGAG |
| | TCTTATACAC | |
| 164 (SEQ ID NO. 457) | ACAGCCCATCGACTGGTGTTTGGATGTGAATCATCTTTAATCTGTC | GGAT |
| | TCTTATACAC | |
| 165 (SEQ ID NO. 458) | ACAGCCCATCGACTGGTGTTTGGCAGTGAATCATCTTTAATCTGTC | GGCA |
| | TCTTATACAC | |
| 166 (SEQ ID NO. 459) | ACAGCCCATCGACTGGTGTTTGGCCGTGAATCATCTTTAATCTGTC | GGCC |
| | TCTTATACAC | |
| 167 (SEQ ID NO. 460) | ACAGCCCATCGACTGGTGTTTGGCGGTGAATCATCTTTAATCTGTC | GGCG |
| | TCTTATACAC | |
| 168 (SEQ ID NO. 461) | ACAGCCCATCGACTGGTGTTTGGCTGTGAATCATCTTTAATCTGTC | GGCT |
| | TCTTATACAC | |
| 169 (SEQ ID NO. 462) | ACAGCCCATCGACTGGTGTTTGGGAGTGAATCATCTTTAATCTGTC | GGGA |
| | TCTTATACAC | |
| 170 (SEQ ID NO. 463) | ACAGCCCATCGACTGGTGTTTGGGCGTGAATCATCTTTAATCTGTC | GGGC |
| | TCTTATACAC | |
| 171 (SEQ ID NO. 464) | ACAGCCCATCGACTGGTGTTTGGGGGTGAATCATCTTTAATCTGTC | GGGG |
| | TCTTATACAC | |
| 172 (SEQ ID NO. 465) | ACAGCCCATCGACTGGTGTTTGGGTGTGAATCATCTTTAATCTGTC | GGGT |
| | TCTTATACAC | |
| 173 (SEQ ID NO. 466) | ACAGCCCATCGACTGGTGTTTGGTAGTGAATCATCTTTAATCTGTC | GGTA |
| | TCTTATACAC | |
| 174 (SEQ ID NO. 467) | ACAGCCCATCGACTGGTGTTTGGTCGTGAATCATCTTTAATCTGTC | GGTC |
| | TCTTATACAC | |
| 175 (SEQ ID NO. 468) | ACAGCCCATCGACTGGTGTTTGGTGGTGAATCATCTTTAATCTGTC | GGTG |
| | TCTTATACAC | |
| 176 (SEQ ID NO. 469) | ACAGCCCATCGACTGGTGTTTGGTTGTGAATCATCTTTAATCTGTC | GGTT |
| | TCTTATACAC | |
| 177 (SEQ ID NO. 470) | ACAGCCCATCGACTGGTGTTTGTAAGTGAATCATCTTTAATCTGTC | GTAA |
| | TCTTATACAC | |
| 178 (SEQ ID NO. 471) | ACAGCCCATCGACTGGTGTTTGTACGTGAATCATCTTTAATCTGTC | GTAC |
| | TCTTATACAC | |
| 179 (SEQ ID NO. 472) | ACAGCCCATCGACTGGTGTTTGTAGGTGAATCATCTTTAATCTGTC | GTAG |
| | TCTTATACAC | |
| 180 (SEQ ID NO. 473) | ACAGCCCATCGACTGGTGTTTGTATGTGAATCATCTTTAATCTGTC | GTAT |
| | TCTTATACAC | |
| 181 (SEQ ID NO. 474) | ACAGCCCATCGACTGGTGTTTGTCAGTGAATCATCTTTAATCTGTC | GTCA |
| | TCTTATACAC | |
| 182 (SEQ ID NO. 475) | ACAGCCCATCGACTGGTGTTTGTCCGTGAATCATCTTTAATCTGTC | GTCC |
| | TCTTATACAC | |
| 183 (SEQ ID NO. 476) | ACAGCCCATCGACTGGTGTTTGTCGGTGAATCATCTTTAATCTGTC | GTCG |
| | TCTTATACAC | |
| 184 (SEQ ID NO. 477) | ACAGCCCATCGACTGGTGTTTGTCTGTGAATCATCTTTAATCTGTC | GTCT |
| | TCTTATACAC | |
| 185 (SEQ ID NO. 478) | ACAGCCCATCGACTGGTGTTTGTGAGTGAATCATCTTTAATCTGTC | GTGA |
| | TCTTATACAC | |
| 186 (SEQ ID NO. 479) | ACAGCCCATCGACTGGTGTTTGTGCGTGAATCATCTTTAATCTGTC | GTGC |
| | TCTTATACAC | |
| 187 (SEQ ID NO. 480) | ACAGCCCATCGACTGGTGTTTGTGGGTGAATCATCTTTAATCTGTC | GTGG |
| | TCTTATACAC | |
| 188 (SEQ ID NO. 481) | ACAGCCCATCGACTGGTGTTTGTGTGTGAATCATCTTTAATCTGTC | GTGT |
| | TCTTATACAC | |
| 189 (SEQ ID NO. 482) | ACAGCCCATCGACTGGTGTTTGTTAGTGAATCATCTTTAATCTGTC | GTTA |
| | TCTTATACAC | |
| 190 (SEQ ID NO. 483) | ACAGCCCATCGACTGGTGTTTGTTCGTGAATCATCTTTAATCTGTC | GTTC |
| | TCTTATACAC | |
| 191 (SEQ ID NO. 484) | ACAGCCCATCGACTGGTGTTTGTTGGTGAATCATCTTTAATCTGTC | GTTG |
| | TCTTATACAC | |
| 192 (SEQ ID NO. 485) | ACAGCCCATCGACTGGTGTTTGTTTGTGAATCATCTTTAATCTGTCT | GTTT |
| | CTTATACAC | |
| 193 (SEQ ID NO. 486) | ACAGCCCATCGACTGGTGTTTTAAAGTGAATCATCTTTAATCTGTC | TAAA |
| | TCTTATACAC | |
| 194 (SEQ ID NO. 487) | ACAGCCCATCGACTGGTGTTTTAACGTGAATCATCTTTAATCTGTC | TAAC |
| | TCTTATACAC | |
| 195 (SEQ ID NO. 488) | ACAGCCCATCGACTGGTGTTTTAAGGTGAATCATCTTTAATCTGTC | TAAG |
| | TCTTATACAC | |
| 196 (SEQ ID NO. 489) | ACAGCCCATCGACTGGTGTTTTAATGTGAATCATCTTTAATCTGTC | TAAT |
| | TCTTATACAC | |
| 197 (SEQ ID NO. 490) | ACAGCCCATCGACTGGTGTTTTACAGTGAATCATCTTTAATCTGTC | TACA |
| | TCTTATACAC | |
| 198 (SEQ ID NO. 491) | ACAGCCCATCGACTGGTGTTTTACCGTGAATCATCTTTAATCTGTC | TACC |
| | TCTTATACAC | |
| 199 (SEQ ID NO. 492) | ACAGCCCATCGACTGGTGTTTTACGGTGAATCATCTTTAATCTGTC | TACG |
| | TCTTATACAC | |
| 200 (SEQ ID NO. 493) | ACAGCCCATCGACTGGTGTTTTACTGTGAATCATCTTTAATCTGTC | TACT |
| | TCTTATACAC | |
| 201 (SEQ ID NO. 494) | ACAGCCCATCGACTGGTGTTTTAGAGTGAATCATCTTTAATCTGTC | TAGA |
| | TCTTATACAC | |
| 202 (SEQ ID NO. 495) | ACAGCCCATCGACTGGTGTTTTAGCGTGAATCATCTTTAATCTGTC | TAGC |
| | TCTTATACAC | |
| 203 (SEQ ID NO. 496) | ACAGCCCATCGACTGGTGTTTTAGGGTGAATCATCTTTAATCTGTC | TAGG |
| | TCTTATACAC | |
| 204 (SEQ ID NO. 497) | ACAGCCCATCGACTGGTGTTTTAGTGTGAATCATCTTTAATCTGTC | TAGT |
| | TCTTATACAC | |
| 205 (SEQ ID NO. 498) | ACAGCCCATCGACTGGTGTTTTATAGTGAATCATCTTTAATCTGTC | TATA |
| | TCTTATACAC | |
| 206 (SEQ ID NO. 499) | ACAGCCCATCGACTGGTGTTTTATCGTGAATCATCTTTAATCTGTC | TATC |
| | TCTTATACAC | |
| 207 (SEQ ID NO. 500) | ACAGCCCATCGACTGGTGTTTTATGGTGAATCATCTTTAATCTGTC | TATG |
| | TCTTATACAC | |
| 208 (SEQ ID NO. 501) | ACAGCCCATCGACTGGTGTTTTATTGTGAATCATCTTTAATCTGTCT | TATT |
| | CTTATACAC | |
| 209 (SEQ ID NO. 502) | ACAGCCCATCGACTGGTGTTTTCAAGTGAATCATCTTTAATCTGTC | TCAA |
| | TCTTATACAC | |
| 210 (SEQ ID NO. 503) | ACAGCCCATCGACTGGTGTTTTCACGTGAATCATCTTTAATCTGTC | TCAC |
| | TCTTATACAC | |
| 211 (SEQ ID NO. 504) | ACAGCCCATCGACTGGTGTTTTCAGGTGAATCATCTTTAATCTGTC | TCAG |
| | TCTTATACAC | |
| 212 (SEQ ID NO. 505) | ACAGCCCATCGACTGGTGTTTTCATGTGAATCATCTTTAATCTGTC | TCAT |
| | TCTTATACAC | |
| 213 (SEQ ID NO. 506) | ACAGCCCATCGACTGGTGTTTTCCAGTGAATCATCTTTAATCTGTC | TCCA |
| | TCTTATACAC | |
| 214 (SEQ ID NO. 507) | ACAGCCCATCGACTGGTGTTTTCCCGTGAATCATCTTTAATCTGTC | TCCC |
| | TCTTATACAC | |
| 215 (SEQ ID NO. 508) | ACAGCCCATCGACTGGTGTTTTCCGGTGAATCATCTTTAATCTGTC | TCCG |
| | TCTTATACAC | |
| 216 (SEQ ID NO. 509) | ACAGCCCATCGACTGGTGTTTTCCTGTGAATCATCTTTAATCTGTCT | TCCT |
| | CTTATACAC | |
| 217 (SEQ ID NO. 510) | ACAGCCCATCGACTGGTGTTTTCGAGTGAATCATCTTTAATCTGTC | TCGA |
| | TCTTATACAC | |
| 218 (SEQ ID NO. 511) | ACAGCCCATCGACTGGTGTTTTCGCGTGAATCATCTTTAATCTGTC | TCGC |
| | TCTTATACAC | |
| 219 (SEQ ID NO. 512) | ACAGCCCATCGACTGGTGTTTTCGGGTGAATCATCTTTAATCTGTC | TCGG |
| | TCTTATACAC | |
| 220 (SEQ ID NO. 513) | ACAGCCCATCGACTGGTGTTTTCGTGTGAATCATCTTTAATCTGTC | TCGT |
| | TCTTATACAC | |
| 221 (SEQ ID NO. 514) | ACAGCCCATCGACTGGTGTTTTCTAGTGAATCATCTTTAATCTGTC | TCTA |
| | TCTTATACAC | |
| 222 (SEQ ID NO. 515) | ACAGCCCATCGACTGGTGTTTTCTCGTGAATCATCTTTAATCTGTCT | TCTC |
| | CTTATACAC | |
| 223 (SEQ ID NO. 516) | ACAGCCCATCGACTGGTGTTTTCTGGTGAATCATCTTTAATCTGTC | TCTG |
| | TCTTATACAC | |
| 224 (SEQ ID NO. 517) | ACAGCCCATCGACTGGTGTTTTCTTGTGAATCATCTTTAATCTGTCT | TCTT |
| | CTTATACAC | |
| 225 (SEQ ID NO. 518) | ACAGCCCATCGACTGGTGTTTTGAAGTGAATCATCTTTAATCTGTC | TGAA |
| | TCTTATACAC | |
| 226 (SEQ ID NO. 519) | ACAGCCCATCGACTGGTGTTTTGACGTGAATCATCTTTAATCTGTC | TGAC |
| | TCTTATACAC | |
| 227 (SEQ ID NO. 520) | ACAGCCCATCGACTGGTGTTTTGAGGTGAATCATCTTTAATCTGTC | TGAG |
| | TCTTATACAC | |
| 228 (SEQ ID NO. 521) | ACAGCCCATCGACTGGTGTTTTGATGTGAATCATCTTTAATCTGTC | TGAT |
| | TCTTATACAC | |
| 229 (SEQ ID NO. 522) | ACAGCCCATCGACTGGTGTTTTGCAGTGAATCATCTTTAATCTGTC | TGCA |
| | TCTTATACAC | |
| 230 (SEQ ID NO. 523) | ACAGCCCATCGACTGGTGTTTTGCCGTGAATCATCTTTAATCTGTC | TGCC |
| | TCTTATACAC | |
| 231 (SEQ ID NO. 524) | ACAGCCCATCGACTGGTGTTTTGCGGTGAATCATCTTTAATCTGTC | TGCG |
| | TCTTATACAC | |
| 232 (SEQ ID NO. 525) | ACAGCCCATCGACTGGTGTTTTGCTGTGAATCATCTTTAATCTGTC | TGCT |
| | TCTTATACAC | |
| 233 (SEQ ID NO. 526) | ACAGCCCATCGACTGGTGTTTTGGAGTGAATCATCTTTAATCTGTC | TGGA |
| | TCTTATACAC | |
| 234 (SEQ ID NO. 527) | ACAGCCCATCGACTGGTGTTTTGGCGTGAATCATCTTTAATCTGTC | TGGC |
| | TCTTATACAC | |
| 235 (SEQ ID NO. 528) | ACAGCCCATCGACTGGTGTTTTGGGGTGAATCATCTTTAATCTGTC | TGGG |
| | TCTTATACAC | |
| 236 (SEQ ID NO. 529) | ACAGCCCATCGACTGGTGTTTTGGTGTGAATCATCTTTAATCTGTC | TGGT |
| | TCTTATACAC | |
| 237 (SEQ ID NO. 530) | ACAGCCCATCGACTGGTGTTTTGTAGTGAATCATCTTTAATCTGTC | TGTA |
| | TCTTATACAC | |
| 238 (SEQ ID NO. 531) | ACAGCCCATCGACTGGTGTTTTGTCGTGAATCATCTTTAATCTGTC | TGTC |
| | TCTTATACAC | |
| 239 (SEQ ID NO. 532) | ACAGCCCATCGACTGGTGTTTTGTGGTGAATCATCTTTAATCTGTC | TGTG |
| | TCTTATACAC | |
| 240 (SEQ ID NO. 533) | ACAGCCCATCGACTGGTGTTTTGTTGTGAATCATCTTTAATCTGTCT | TGTT |
| | CTTATACAC | |
| 241 (SEQ ID NO. 534) | ACAGCCCATCGACTGGTGTTTTTAAGTGAATCATCTTTAATCTGTC | TTAA |
| | TCTTATACAC | |
| 242 (SEQ ID NO. 535) | ACAGCCCATCGACTGGTGTTTTTACGTGAATCATCTTTAATCTGTC | TTAC |
| | TCTTATACAC | |
| 243 (SEQ ID NO. 536) | ACAGCCCATCGACTGGTGTTTTTAGGTGAATCATCTTTAATCTGTC | TTAG |
| | TCTTATACAC | |
| 244 (SEQ ID NO. 537) | ACAGCCCATCGACTGGTGTTTTTATGTGAATCATCTTTAATCTGTCT | TTAT |
| | CTTATACAC | |
| 245 (SEQ ID NO. 538) | ACAGCCCATCGACTGGTGTTTTTCAGTGAATCATCTTTAATCTGTC | TTCA |
| | TCTTATACAC | |
| 246 (SEQ ID NO. 539) | ACAGCCCATCGACTGGTGTTTTTCCGTGAATCATCTTTAATCTGTCT | TTCC |
| | CTTATACAC | |
| 247 (SEQ ID NO. 540) | ACAGCCCATCGACTGGTGTTTTTCGGTGAATCATCTTTAATCTGTC | TTCG |
| | TCTTATACAC | |
| 248 (SEQ ID NO. 541) | ACAGCCCATCGACTGGTGTTTTTCTGTGAATCATCTTTAATCTGTCT | TTCT |
| | CTTATACAC | |
| 249 (SEQ ID NO. 542) | ACAGCCCATCGACTGGTGTTTTTGAGTGAATCATCTTTAATCTGTC | TTGA |
| | TCTTATACAC | |
| 250 (SEQ ID NO. 543) | ACAGCCCATCGACTGGTGTTTTTGCGTGAATCATCTTTAATCTGTC | TTGC |
| | TCTTATACAC | |
| 251 (SEQ ID NO. 544) | ACAGCCCATCGACTGGTGTTTTTGGGTGAATCATCTTTAATCTGTC | TTGG |
| | TCTTATACAC | |
| 252 (SEQ ID NO. 545) | ACAGCCCATCGACTGGTGTTTTTGTGTGAATCATCTTTAATCTGTCT | TTGT |
| | CTTATACAC | |
| 253 (SEQ ID NO. 546) | ACAGCCCATCGACTGGTGTTTTTTAGTGAATCATCTTTAATCTGTCT | TTTA |
| | CTTATACAC | |
| 254 (SEQ ID NO. 547) | ACAGCCCATCGACTGGTGTTTTTTCGTGAATCATCTTTAATCTGTCT | TTTC |
| | CTTATACAC | |
| 255 (SEQ ID NO. 548) | ACAGCCCATCGACTGGTGTTTTTTGGTGAATCATCTTTAATCTGTCT | TTTG |
| | CTTATACAC | |
| 256 (SEQ ID NO. 549) | ACAGCCCATCGACTGGTGTTTTTTTGTGAATCATCTTTAATCTGTCT | TTTT |
| | CTTATACAC | |

TABLE 10
Primers to Amplify Pseudoknot ΨDNA

| Name | Sequence |
|---|---|
| psi Loop FOR | TCGTCGGCAGCGTCAGATGTGTATAAGAGAC |
| (SEQ ID NO. 550) | AGATTAAAGATG |
| psi Loop REV | GTCTCGTGGGCTCGGAGATGTGTATAAGAGA |
| (SEQ ID NO. 551) | CAGACAGCCCATCGAC |

To further evaluate the cellular feasibility of the AsCas12a-ΨDNA complex, the off-target effects of this therapeutic strategy were systematically assessed. It was further hypothesized that, owing to the absence of RNA trans-cleavage activity in AsCas12a, modulation of the target transcript by this system is likely to impose fewer perturbations on other cellular pathways. mRNA-Seq was performed to quantify the off-target effects of the AsCas12a-ΨDNA complex and ΨDNA alone, and compared them to RfxCas13d (FIGS. 8C-8F and 18A-18D) for two genes, PPIA and RPL4. It was observed that, compared to ΨDNA alone, the AsCas12a-ΨDNA complex not only achieved greater RNA knockdown efficiency but also exhibited 2- to 7-fold lower off-target effects. This suggests that AsCas12a plays a significant role in guiding target binding and enhancing specificity in the presence of the DNA guide. Furthermore, while AsCas12a-ΨDNA exhibits lower knockdown efficiency compared with RfxCas13d (FIG. 17H), AsCas12a showed substantially lower off-target effects than RfxCas13d for both genes, 17.7-fold less for PPIA and 6.3-fold less for RPL4. These results clearly demonstrate that the absence of RNA trans-cleavage significantly reduces off-target effects and that the AsCas12a-ΨDNA complex is evidently less toxic to the transcriptome compared to the widely used RfxCas13d.

To increase RNA knockdown efficiency, it was determined that having AsCas12a already present in the cell at the time of ΨDNA transfection is more effective, as the ΨDNAs can immediately complex with the protein and avoid degradation in the cytoplasm. To enable this, a lentiviral cell line was generated that expresses AsCas12a and GFP on the same transcript. In this cell line, the transfection of DNA guides targeting five different transcripts was tested: PPIA, RPL4, PCSK9, NRAS, and SMARCA4 (FIGS. 8G-8H). Substantial improvements were observed in knockdown efficiency, with PPIA and RPL4 expression reduced by 95% and 80%, respectively. Similarly, PCSK9 and NRAS showed robust knockdown, with approximately 90% reduction in gene expression. In contrast, SMARCA4 exhibited a more moderate knockdown at around 60%. Overall, AsCas12a transduction significantly enhanced the efficiency of the ΨDNA complex.

To determine whether ΨDNA-mediated RNA knockdown is associated with translational repression, Ribo-Seq paired with RNA-Seq was performed on cells co-transfected with AsCas12a and ΨPPIA or a non-targeting ΨDNA control (ΨNT) (FIGS. 81 and 17E-17F). The analysis revealed the target PPIA had an increase ribosomal occupancy and a total of 17 transcripts exhibited significant changes in translational efficiency, including the intended PPIA and its homologous transcripts PPIAL4C and PPIAL4G. These results indicate that ΨDNA-mediated RNA knockdown is accompanied by highly selective translational effects and is associated with minimal off-target perturbation. Taken together, these results provide evidence that ΨDNA-AsCas12a induces ribosomal stalling consistent with a no-go decay-like mechanism, while maintaining specificity at the translational level.

Additionally, CLIP-seq was performed to obtain a more in-depth and granular view of enzyme binding in a cellular context. Following sequencing and data analysis, normalized CLIP-seq peaks were plotted to compare binding profiles between non-targeting and targeting ΨDNAs in the presence of AsCas12a (FIGS. 8J-8K). As shown, binding of the enzyme at the PPIA locus targeted by ΨDNA is highly enriched relative to the non-targeting control, demonstrating strong target specificity in cells. Enrichment was also observed at the PPIAP22 pseudogene, which shares an identical target sequence with PPIA, further indicating that binding is driven by sequence complementarity. Additionally, similar results were observed with ΨMix transfection (targeting PPIA, RPL4, SMARCA4 and PCSK9) (FIG. 17G) showing multiple peaks for the different targeted transcripts.

### Potential Application of AsCas12a and ΨDNA System

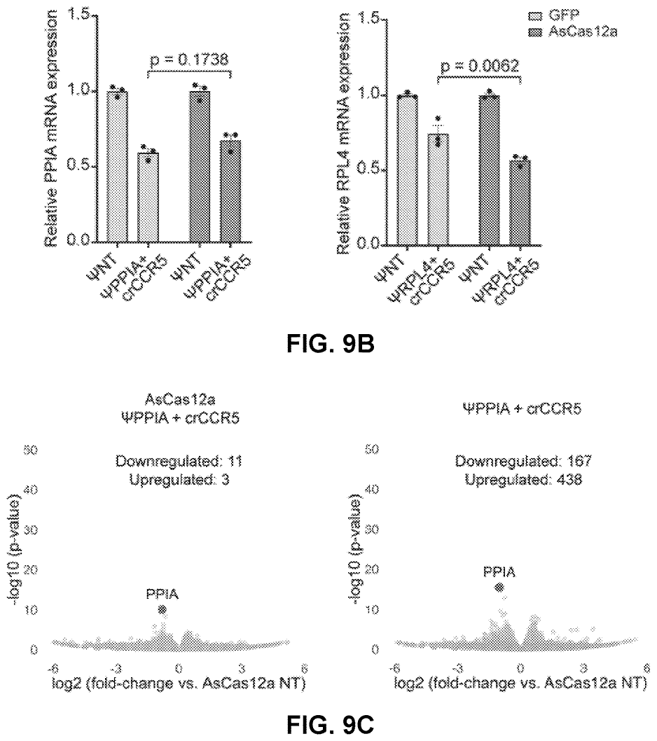

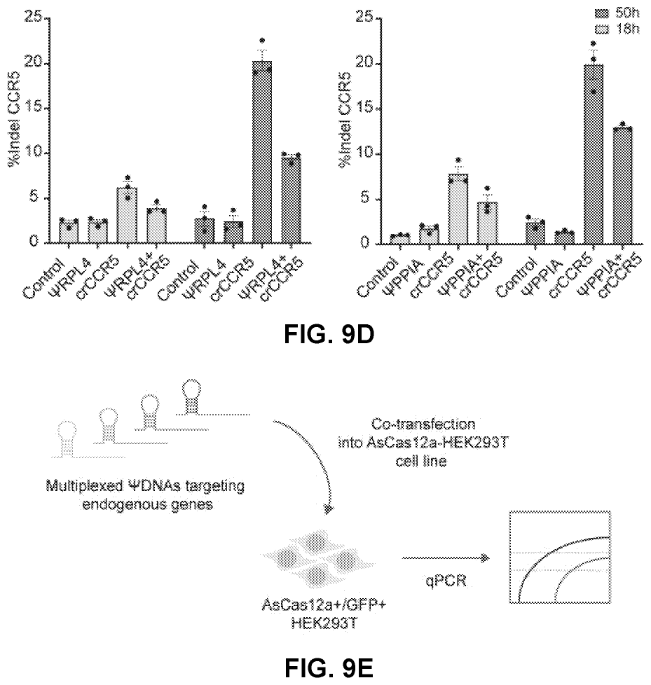

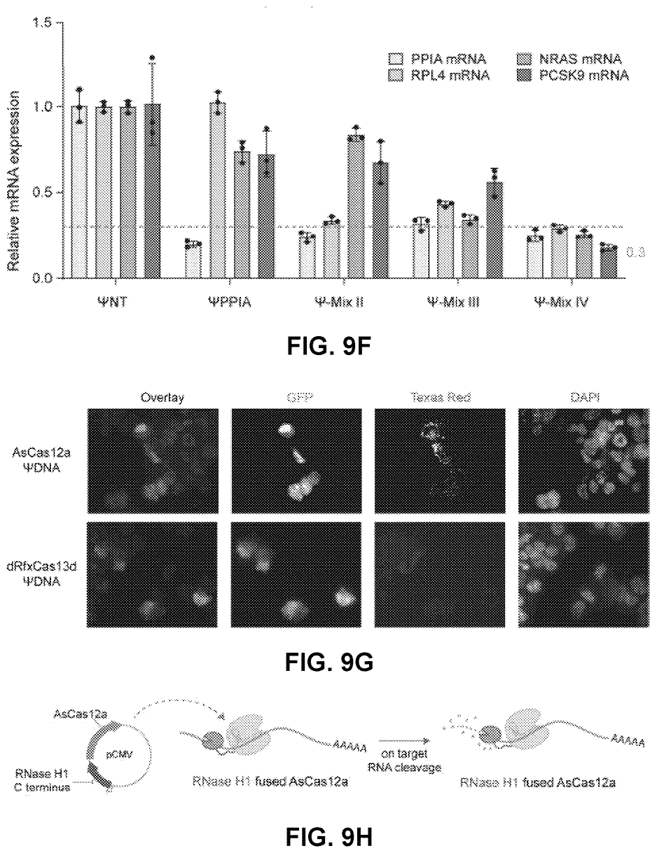

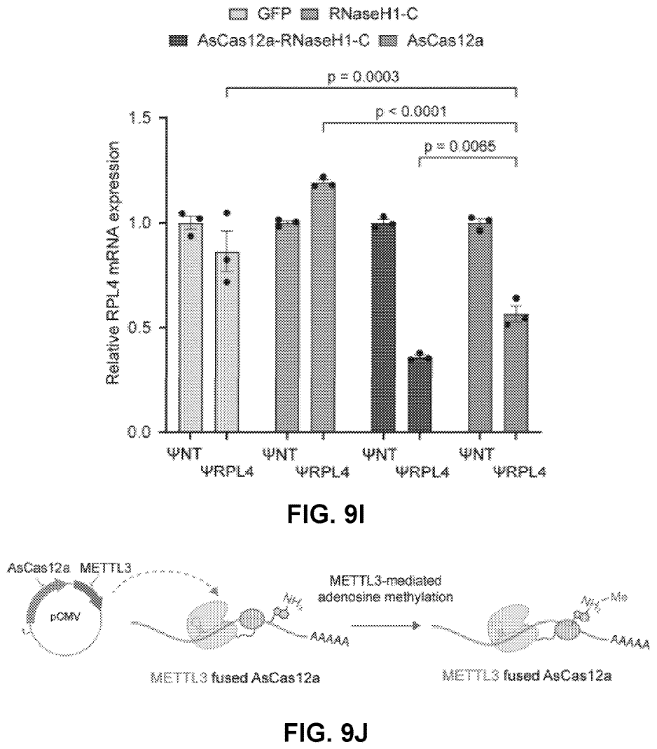

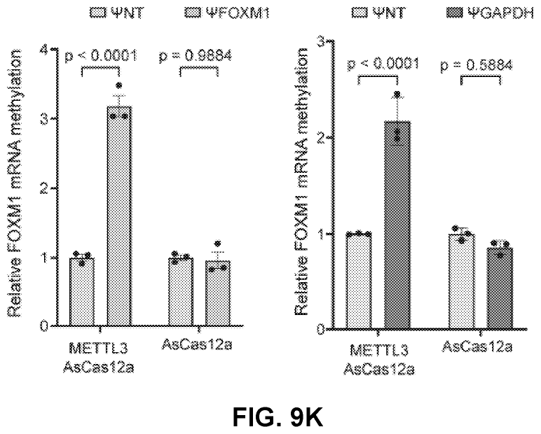

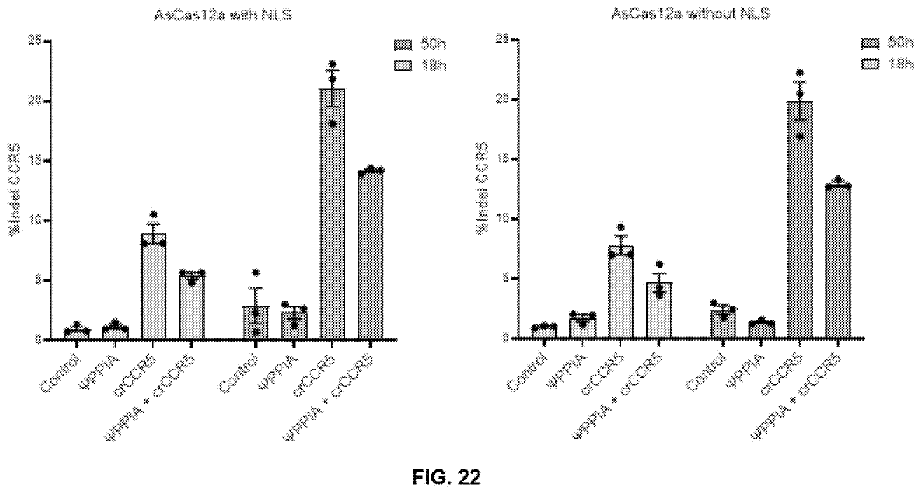

A ΨDNA-based platform for simultaneous DNA editing and RNA targeting was next established using a single CRISPR effector, extending the application of ΨDNA beyond the conventional functional scope of Cas12 or Cas13 based systems. By co-delivering ΨDNA and crRNA, this strategy enables concurrent RNA knockdown and DNA indel formation (FIG. 9A). To achieve this, AsCas12a lacking a nuclear localization signal (NLS) was delivered, together with either ΨPPIA or ΨRPL4, and a crRNA targeting the CCR5 locus. After 18 hours, RNA levels were reduced for ΨPPIA and ΨRPL4 relative to the ΨNT control (FIG. 9B). Although statistical significance between the ΨRPL4-GFP and ΨRPL4-AsCas12a conditions was observed, no significant difference was observed for the ΨPPIA datasets. To determine whether AsCas12a influenced RNA knockdown specificity, mRNA-seq was performed on these samples to evaluate off-target effects and assess whether the presence of the protein improves RNA targeting specificity (FIGS. 9B-9C). The data show that samples containing AsCas12a exhibited a total of 14 off-target transcripts, compared to 605 off-targets in the ΨPPIA-only condition, corresponding to an approximately 43-fold reduction. In parallel, amplicon sequencing confirmed efficient genome editing on CCR5 locus at 18 and 50 hours after transfection, with indel frequencies of approximately 10-15% (FIGS. 9D and 22). Together, these data demonstrate that the AsCas12a-ΨDNA system enables dual DNA-RNA targeting using a single effector, providing a versatile and previously unreported approach for coordinated regulation of genomic DNA and endogenous RNA.

ΨDNA sequences for endogenous gene silencing and qPCR primers and crRNA for DNA indel formation are presented in Table 11.

ΨDNA Sequences for Endogenous Gene Silencing and qPCR
Primers and crRNA for DNA Indel Formation

| Name | Sequence |
|---|---|
| ΨDNA NT derived from crRNA | T*C*A*CCAGAAGCGTACCATACTCACGAACAGTAGATGTGAATCATCTTT*A* |
| (SEQ ID NO. 254) | A*T |
| ΨDNA PPIA derived from crRNA | A*A*A*CACCACATGCTTGCCATCCAACCACTCTAGATGTGAATCATCTTT*A*A |
| (SEQ ID NO. 255) | *T |
| ΨDNA RPL4 derived from crRNA | G*A*A*GTTCAGGAACTTCCTCAATACGATGACTAGATGTGAATCATCTTT*A*A |
| (SEQ ID NO. 256) | *T |
| ΨDNA NRAS derived from | C*T*G*GTCTTGGCTGAGGTTTCTAGATGTGAATCATCTTT*A*A*T |
| crRNA (SEQ ID NO. 257) | |
| ΨDNA SMAR CA4 derived from | C*G*A*TGCGGTGGGCTCGGTCCTAGATGTGAATCATCTTT*A*A*T |
| crRNA (SEQ ID NO. 258) | |
| ΨDNA PCSK9 #1 (SEQ ID NO. | A*A*G*CCAGGAAGAAGGCCATGGAAGACATGCTAGATGTGAATCATCTTT*A* |
| 259) | A*T |
| ΨDNA PCSK9 #2 (SEQ ID NO. | A*T*G*GGGCAACTTCAAGGCCAGCTCCAGCAGTAGATGTGAATCATCTTT*A |
| 260) | *A*T |
| ΨDNA PCSK9 #3 (SEQ ID NO. | C*C*A*GGTTCCACGGGATGCTCTGGGCAAAGATAGATGTGAATCATCTTT*A* |
| 261) | A*T |
| ΨDNA PCSK9 #4 (SEQ ID NO. | G*G*A*AGACATGCAGGATCTTGGTGAGGTATCTAGATGTGAATCATCTTT*A* |
| 262) | A*T |
| ΨDNA PCSK9 #5 (SEQ ID NO. | C*T*C*TGACTGCGAGAGGTGGGTCTCCTCCTTTAGATGTGAATCATCTTT*A* |
| 263) | A*T |
| qPCR PPIA | Hs99999904_m1 |
| qPCR RPL4 | Hs00973287_g1 |
| qPCR PCSK9 | Hs00545399_m1 |
| qPCR NRAS | Hs00180035_m1 |
| qPCR SMAR CA4 | Hs00231324_m1 |
| crRNA CCR5 (SEQ ID NO. 264) | /AITR1/rUrArArUrUrUrCrUrArCrUrCrUrUrGrUrArGrArUrGrCrArGrGrGrCrUrCrCr |
| | GrArUrGrUrArUrArArUrArArUrU/AITR2/ |

*signifies phosphorothioate bond

To evaluate the capability of ΨDNA-guided CRISPR-Cas12a for multiplex RNA transcript knockdown, combinations of ΨDNAs targeting multiple endogenous transcripts were next co-delivered, including PPIA, RPL4, NRAS, and PCSK9. Different ΨDNA combinations in the AsCas12a-transduced cell line were transfected, including ΨPPIA (targeting PPIA only), Ψ-Mix II (targeting PPIA and RPL4), Ψ-Mix III (targeting PPIA, RPL4, and NRAS), and Ψ-Mix IV (targeting PPIA, RPL4, NRAS, and PCSK9) (FIGS. 9E-9F). It was observed that individual ΨDNAs as well as multiplexed combinations (Y′-Mix II, III, IV) achieved efficient transcript depletion across all target RNAs. Specifically, Ψ-Mix IV resulted in robust knockdown of +70% for PPIA, RPL4, NRAS, and PCSK9 simultaneously, highlighting the potential of this system for targeting multiple transcripts in parallel. Multiplexed RNA knockdown was also achieved with AsCas12a transfection, resulting in approximately 50% reduction in expression across four simultaneous target genes (FIG. 18E).

Collectively, these findings demonstrate that the ΨDNA-guided CRISPR-Cas12a system enables precise and efficient depletion of endogenous RNA transcripts, both individually and in a multiplexed manner. The system offers considerable versatility for RNA-targeting applications and holds strong potential for therapeutic interventions aimed at reducing disease-relevant RNA transcripts. Its low off-target effects and high knockdown efficiency make AsCas12a a particularly promising platform for therapeutic use, especially in contexts where preserving transcriptome integrity is essential.

It was further investigated whether RNA knockdown mediated by the AsCas12a-ΨDNA system involves additional cooperative pathways beyond a single mechanism. To test whether RNase H1 is spatially associated with the ΨDNA-AsCas12a complex during RNA targeting, a proximity ligation assay (PLA) was performed. GFP-AsCas12a-ΨPPIA and GFP-dRfxCas13d-ΨPPIA constructs were analyzed using antibodies against the HA tag and endogenous RNase H1. PLA signals, detected as red fluorescence, indicate close spatial proximity between the protein and RNase H1. Notably, AsCas12a-expressing cells exhibited a strong PLA signal, whereas dRfxCas13d showed minimal signal (FIG. 9G). This finding suggests that AsCas12a-ΨDNA-mediated RNA degradation may also involve RNase H1 as well, beyond NGD mechanism previously characterized.

Accordingly, it was explored whether directly fusing RNase H1 to AsCas12a could further enhance RNA knockdown efficiency (FIG. 9H). To compare the effects of RNase H1-fused AsCas12a and AsCas12a alone, a pCMV expressing mCherry plasmid and ΨDNA2 previously characterized were co-transfected (FIG. 7B). Fluorescence microscopy showed that, similar to AsCas12a, RNase H1-fused AsCas12a significantly reduced mCherry signal when targeted by ΨDNA2 (FIG. 18C). To assess changes at the RNA level, the total RNA was isolated from the experimental groups and RT-qPCR was performed. The results indicated that ΨDNA2 induced a significant reduction in mCherry RNA levels when RNase H1-fused AsCas12a was expressed, compared to the non-targeting (NT) control (FIG. 18D). RNA knockdown was still observed with wild-type AsCas12a and GFP, but to a lesser extent. To minimize potential effects arising from RNase H1 overexpression, using ΨDNA targeting the endogenous RPL4 transcript, it was observed that co-transfection of the RNase H1 C-terminal domain (RNase H1-C) did not induce measurable RPL4 knockdown, in contrast to full-length RNase H1 (RNase H1-FL) (FIG. 18B). In light of the observations above, the RNase H1 C-terminal domain was fused to AsCas12a to enhance RNA knockdown of an abundant endogenous transcript such as RPL4 (FIG. 9I). The fused construct lowers the RNA levels ~15% more than normal AsCas12a-ΨDNA showing significant statistical improvement. Additionally, the data demonstrate that the RNase H-AsCas12a fusion construct effectively reduces RPL4 RNA levels in a manner that is dependent on guidance by the AsCas12a enzyme since only the C-terminus was added.

In addition, N6-methyladenosine (m6A) is the most abundant internal RNA modification, known to regulate RNA secondary structure, alternative splicing, and stability. To evaluate site-specific epitranscriptomic editing, the m6A methyltransferase METTL3 was fused to AsCas12a and targeted it to RNA using ΨDNA (FIG. 9J). METTL3-fused AsCas12a or AsCas12a was co-transfected alone with ΨDNAs targeting two sites: A690 in GAPDH and A3488/A3504 in FOXM1.

MeRIP-qPCR analysis revealed that, upon targeting by Ψ-GAPDH or Ψ-FOXM1, METTL3-fused AsCas12a induced a significant increase in m6A modification at the target sites compared to the NT group, whereas wild-type AsCas12a alone had no detectable effect (FIG. 9K). These results demonstrate that AsCas12a, when fused to functional effector proteins like METTL3, can be guided by ΨDNA to achieve site-specific manipulation of RNA. ΨDNA, qPCR primers, and probe sequence for MeRIP-qPCR are presented in Table 12.

TABLE 12
ΨDNA, qPCR Primers and Probe Sequence for MeRIP-qPCR

| Name | Sequence |
|---|---|
| ΨDNA GAPDH A690 derived from crRNA | A*G*C*CCCGCGGCCATCACGCCACAGTTTCCCTAGATGTGAAT |
| (SEQ ID NO. 265) | CATCTTT*A*A*T |
| ΨDNA Foxm1 A3488 & A3504 derived from | G*T*A*TGATTGGGGACATTATCAGAGAAACATTAGATGTGAATCA |
| crRNA (SEQ ID NO. 266) | TCTTT*A*A*T |
| GAPDH A690 target forward primer derived | CATCACTGCCACCCAGAAGA |
| from crRNA (SEQ ID NO. 267) | |
| GAPDH A690 target reverse primer derived | CAGTAGAGGCAGGGATGATGTT |
| from crRNA (SEQ ID NO. 268) | |
| GAPDH A690 target probe derived from | /56-FAM/CCCTCCGGG/ZEN/AAACTGTGGCGT/3IABKFQ/ |
| crRNA (SEQ ID NO. 269) | |
| FOXM1 A3488, A3504 target forward primer | TGCCCAGATGTGCGCTATTA |
| derived from crRNA (SEQ ID NO. 270) | |
| FOXM1 A3488, A3504 target reverse primer | CTTCTCAAGCCTCCACCTGA |
| derived from crRNA (SEQ ID NO. 271) | |
| FOXM1 A3488, A3504 target probe derived | /56-FAM/TCGTCAATG/ZEN/CCAGTCTCCCTGGT/3IABKFQ/ |
| from crRNA (SEQ ID NO. 272) | |
| GAPDH control forward primer derived from | TCAAGGCTGAGAACGGGAAG |
| crRNA (SEQ ID NO. 273) | |
| GAPDH control reverse primer derived from | GGACTCCACGACGTACTCAG |
| crRNA (SEQ ID NO. 274) | |
| GAPDH control probe derived from crRNA | /5Cy55/TCCAGGAGCGAGATCCCTCC/3IAbRQSp |
| (SEQ ID NO. 275) | |

*signifies phosphorothioate bond

### Example 2: Discussion

Overall, this study introduces a novel methodology for RNA targeting that expands the versatility and functional scope of CRISPR-Cas systems. Consistent with the conformational rearrangements observed when Cas proteins bind crRNA followed by target DNA recognition, moves the RuvC domain to the final position to activate trans-cleavage activity. The formation of a ternary complex of AsCas12a, ΨDNA, and the target RNA may likewise induce structural changes and activate collateral activity. This mechanism parallels how crRNA-dsDNA engagement triggers non-specific ssDNA cleavage. Moreover, the trans-cleavage activity of both enzymes, AsCas12a and Cas12i1, is greatly increased when the DNA guide has a 3′ handle that is situated in the same position as the 5′ scaffold of the crRNA. This evidence suggests the 3′ handle in ΨDNA further stabilizes the Cas protein complex and it is translated into a higher fluorescent signal. Additionally, no cis-cleavage or trans-cleavage of RNA was observed, as Cas12 enzymes do not possess a HEPN domain capable of RNA degradation like Cas13. Instead, in most Cas12 enzymes, the RuvC domain is primarily associated with DNA cleavage, except for Cas12g and Cas12a2.

Although ΨDNAs cannot be genetically encoded or expressed from plasmids, current methods for RNA synthesis include solid-phase synthesis, in vitro transcription (IVT), and other enzymatic processes. Solid-phase synthesis of RNA is more expensive and time-consuming than that of DNA, requiring costly reagents, specialized chemistry, and extensive steps35. IVT involves an initial DNA synthesis step, followed by enzymatic processes to produce RNA, adding to the overall cost and complexity. Additionally, RNA has several magnitude shorter shelf-life than DNA, necessitating low temperatures or specialized storage conditions to maintain stability. CRISPR-Cas nucleic acid detection platforms, such as SHERLOCK and DETECTR, predominantly rely on crRNA, making these technologies more expensive and less accessible. This efficiency in time and cost is particularly advantageous in high-throughput applications to allow faster iteration of guides and optimize experimental conditions. Thus, the use of ΨDNA represents a practical and economical alternative for large-scale studies involving RNA detection and targeting.

Most importantly, herein is introduced a new platform for RNA targeting based on a novel mechanism using Cas12 enzymes. While CRISPR-Cas12 systems have been extensively engineered for DNA genome editing in cells, herein is successfully demonstrated a unique RNA-targeting construct that significantly expands the system's applicability to RNA gene regulation. This study shows that AsCas12a can complex with ΨDNA to target mRNA within cells, leading to reduced mRNA levels and decreased protein production. Notably, because this DNA-guided system lacks RNA cis- or trans-cleavage activity, it was found that the ΨDNA-AsCas12a complex exhibits significantly lower off-target effects than RfxCas13d, a widely used RNA-targeting enzyme. The reduced off-target toxicity of DNA-guided AsCas12a presents a novel strategy for transient gene silencing and holds promise for RNA editing within cells, similar to approaches using catalytically inactive Cas13 variants.

Furthermore, it has been demonstrated herein that the ΨDNA-AsCas12a platform enables simultaneous DNA and RNA targeting using a single CRISPR effector. This represents the first integrated CRISPR system capable of combining sequence-specific RNA binding and knockdown with programmable DNA editing. The dual RNA-DNA targeting capability enables multi-layer gene regulation by combining immediate transcript suppression with permanent genomic disruption. This strategy may accelerate functional genomics studies by distinguishing early transcript-level effects from long-term genomic consequences while reducing compensatory cellular responses. Additionally, integrating both functionalities simplifies delivery compared with approaches requiring separate RNA interference and genome editing components, expanding opportunities in multiplexed gene regulation, synthetic biology, and therapeutic development.

In therapeutic contexts, this approach could simultaneously silence pathogenic RNAs while editing the underlying DNA source. For example, while CCR5 knockout is a well-established strategy to prevent HIV entry, viral tropism switching to CXCR4 remains a limitation. Because CXCR4 is essential for hematopoietic stem cell homing, permanent disruption is lethal. In this scenario, the present system could leverage crRNA-mediated CCR5 disruption while ΨDNA transiently suppresses CXCR4 mRNA, potentially limiting viral escape without eliminating an essential gene. Although further engineering and delivery optimization will be required to evaluate such applications in disease models, these results highlight the potential of coordinated RNA and DNA targeting by a single nuclease.

Additionally, multiplexed gene silencing has been demonstrated across four different targets, highlighting the platform's potential for high-throughput applications akin to previous Cas13-based screens. In the present multiplex knockdown system, the Ψ-IV group, designed to simultaneously target four distinct transcripts, achieved robust and efficient knockdown, with efficiencies ranging from 70.7% to 81.9%. These findings underscore the potential of the ΨDNA-AsCas12a platform as a scalable and cost-effective approach for multiplexed CRISPR screening. Additionally, the ability to coordinately modulate multiple gene expressions offers a promising strategy for advancing RNA-targeted therapeutics.

As for the mechanism of ΨDNA-AsCas12a-mediated RNA knockdown, these observations indicate that targeting by the ΨDNA-AsCas12a complex is associated with changes in translational output of the target RNA, and that the complex exhibits spatial proximity to endogenous RNase H1. These findings are consistent with the possibility that multiple regulatory processes contribute to RNA reduction. Lastly, it was demonstrated that AsCas12a fusion proteins, specifically with RNase H and METTL3, can mediate precise and programmable regulation of target RNAs when guided by ΨDNA. The inherent modularity of AsCas12a enables its fusion with a variety of functional protein domains, allowing tailored applications in RNA biology. These include, but are not limited to, locus-specific RNA labeling, targeted base editing, and other epitranscriptomic modifications in cells, thereby broadening the scope of RNA-targeted interventions at the post-transcriptional level.

### Example 3: Materials and Methods

### Plasmid Construction

Plasmids encoding Lb, As, Er and other Cas12a variants and the BrCas12b enzyme were built following the protocol outlined in earlier publications. Plasmid containing E. coli codon-optimized Cas12i1 and Cas12i2 gene was obtained from Addgene, a gift from ArborBiotechnologies (Plasmid #120882 & #120883).

For mammalian cell transfection, NLS was removed from the AsCas12a-P2A-GFP plasmid (Addgene #160140) through a KLD reaction. mCherry plasmid was constructed by inserting the mCherry gene into a pCMV vector with no CMV enhancer sequence. RfxCas13d, PspCas13b (mutated from Addgene #155367) and DisCas7-11 (Addgene #172507) are cloned into the same backbone.

AsCas12a-P2A-GFP transfer plasmid for lentiviral cell line was constructed by cloning the previously described AsCas12a-P2A-GFP (no NLS) construct into lentiCas9-Blast (Addgene #52962) backbone that contains blasticidin resistance. The cloning was made with NEBuilder® HiFi DNA Assembly Master Mix (NEB #E2621L).

RNaseH1 sequence was amplified from ppyCAG-RNaseH1-WT (Addgene #111906) and cloned to the N-terminus of AsCas12a. METTL3 and NLS was amplified from pCMV-dCas13-M3nls (Addgene #155366) and cloned to the C-terminus of AsCas12a.

### Protein Expression and Purification

Rosetta bacterial colonies harboring the plasmid for protein production were cultured at 37° C. overnight on agar. Select colonies were transferred from the agar to 10 mL LB medium (Fisher Scientific, Catalog #BP9723-500) for 12-hour incubation. The culture was then expanded to 1.5 L TB medium (MP Bio #113046042), growing until an optical density (OD) of 0.6-0.8 was reached. The culture was cooled for 45-60 minutes before adding Isopropyl β-D-1-thiogalactopyranoside (IPTG) to a concentration of 0.5 mM, and growth continued for 14-18 hours at 16° C.

Cell harvesting involved centrifugation at 10,000×g for 5 minutes. The pellet was reconstituted in Lysis Buffer (500 mM NaCl, 50 mM Tris-HCl, pH 7.5, 20 mM Imidazole, 0.5 mM TCEP, 1 mM PMSF, 0.25 mg/mL Lysozyme, DNase I). This suspension underwent sonication and further centrifugation at 39,800×g for 30 minutes. The resulting lysate was passed through a 0.22 μm syringe filter (Cytiva, Catalog #9913-2504) and introduced into a 5 mL Histrap FF column (Cytiva, Catalog #17525501, with Ni²⁺ replaced by Co²⁺) linked to a BioLogic DuoFlow™ FPLC system (Bio-rad).

Proteins were eluted using Buffer B (500 mM NaCl, 50 mM Tris-HCl, pH 7.5, 250 mM Imidazole, 0.5 mM TCEP). For all proteins except Cas12i1 and Cas12i2, the eluted fractions were merged and dialyzed in a 10 kDa-14 kDa MWCO bag with TEV protease (sourced from David Waugh, Addgene #8827) and prepared internally. The bag was immersed in Dialysis Buffer (500 mM NaCl, 50 mM HEPES, pH 7, 5 mM MgCl₂, 2 mM DTT) and stirred gently at 4° C. overnight.

The protein blend was concentrated to approximately 10 mL using a 30 kDa MWCO Vivaspin® 20 concentrator. This concentrate was then balanced with 10 ml of Buffer C (150 mM NaCl, 50 mM HEPES, pH 7, 0.5 mM TCEP). It was processed through a 1 mL Hitrap Heparin HP column (prepped with Buffer C) using the BioLogic DuoFlow™ FPLC system (Bio-rad). A gradient flow alternating between Buffer C and Elution Buffer D (2000 mM NaCl, 50 mM HEPES, pH 7, 0.5 mM TCEP) facilitated protein elution. Further size-exclusion chromatography was employed as necessary. The protein passed through a HiLoad® 16/600 Superdex® column (Cytiva, Catalog #28989335), and the highest purity fractions were pooled, concentrated with a 30 kDa MWCO Vivaspin® 20, and flash-frozen in liquid nitrogen for storage at −80° C.

### Oligonucleotide Preparation

All single-stranded DNA and RNA oligos including guide RNA, guide DNA, target activators, primers, and fluorescent reporters were obtained from Integrated DNA Technologies (IDT) and diluted in 1×TE Buffer (10 mM Tris, 0.1 mM EDTA, pH 7.5). For generating long RNA target mimics of HIV, Zika, Dengue and HCV RNA, dsDNA gene fragments containing a T7 promoter region were ordered from Twist Biosciences and in vitro transcribed using HiScribe® T7 High Yield RNA Synthesis Kit (NEB #E2040S) to generate the long RNA fragments. Sequences used for long RNA detection are provided in Table 13.

TABLE 13
DNA and RNA Sequences Used for Long RNA Detection with Synthetic HIV

| Name | Sequences |
|---|---|
| T7-HIV Geneblock 1-DNA | TAATACGACTCACTATAGAAAAGATGGATAATCCTGGGATTAAATAAAA |
| fragment with T7 promoter | TAGTAAGAATGTATAGCCCTACCAGCATTCTGGACATAAGACAAGGAC |
| used to produce a fragment of | CAAAGGAACCCTTTAGAGACTATGTAGACCGGTTCTATAAAACTCTAA |
| genomic HIV RNA through in | GAGCCGAGCAAGCTTCACAGGAGGTAAAAAATTGGATGACAGAAAC |
| vitro transcription with T7 RNA | CTTGTTGGTCCAAAATGCGAACCCAGATTGTAAGACTATTTTAAAAGC |
| Pol (SEQ ID NO. 47) | ATTGGGACCAGCGGCTACACTAGAAGAAATGATGACAGCATGTCAG |
| | GGAGTAGGAGGACCCGGCCATAAGGCAAGAGTTTTGGCTGAAGCA |
| | ATGAGCCAAGTAACAAATTCAGCTACCATAATGATGCAGAGAGGCAA |
| | TTTTAGGAACCAAAGAAAGATTGTTAAGTGTTTCAATTGTGGCAAAGA |
| | AGGGCACACAGCCAGAAATTGCAGGGCCCCTAGGAAAAAGGGCTG |
| | TTGGAAATGTGGAAAGGAAGGACACCAAATGAAAGATTGTACTGAGA |
| | GACAGGCTAATTTTTTAGGGAAGATCTGGCCTTCCTACAAGGGAAGG |
| | CCAGGGAATTTTCTTCAGAGCAGACCAGAGCCAACAGCCCCACAGA |
| | AGAGAGCTTCAGGTCTGGGGTAGAGACAACAACTCCCCCTCAGAAG |
| | CAGGAGCCGATAGACAAGGAACTGTATCCTTTAACTTCCCTCAGGTC |
| | ACTCTTTGGCAACGACCCCTCGTCACAATAAAGATAGGGGGGCAAC |
| | TAAAGGAAGCTCTATTAGATACAGGAGCAGATGATACAGTATTAGAAG |
| | AAATGAGTTTGCCAGGAAGATGGAAACCAAAAATGATAGGGGGAATT |
| | GGAGGTTTTATCAAAGTAAGACAGTATGATCAGATACTCATAGAAATC |
| | TGTGGACATAAAGCTATAGGTACAGTATTAGTAGGACCTACACCTGTC |
| | AACATAATTGGAAGAAATCTGTTGACTCAGATTGGTTGCACTTTAAAT |
| | TTTCCCATTAGCCCTATTGAGACTGTACCAGTAAAATTAAAGCCAGGA |
| | ATGGATGGCCCAAAAGTTAAACAATGGCCATTGACAGAAGAAAAAAT |
| | AAAAGCATTAGTAGAAATTTGTACAGAGATGGAAAAGGAAGGGAAAA |
| | TTTCAAAAATTGGGCCTGAAAATCCATACAATACTCCAGTATTTGCCA |
| | TAAAGAAAAAAGACAGTACTAAATGGAGAAAATTAGTAGATTTCAGAG |
| | AACTTAATAAGAGAACTCAAGACTTCTGGGAAGTTCAATTAGGAATAC |
| | CACATCCCGCAGGGTTAAAAAAGAAAAAATCAGTAACAGTACTGGAT |
| | GTGGGTGATGCATATTTTTCAGTTCCCTTAGATGAAGACTTCAGGAA |
| | GTATACTGCATTTACCATACCTAGTATAAACAATGAGACACCAGGGAT |
| | TAGATATCAGTACAATGTGCTTCCACAGGGATGGAAAGGATCACCAG |
| | CAATATTCCAAAGTAGCATGACAAAAATCTTAGAGCCTTTTAGAAAAC |
| | AAAATCCAGACATAGTTATCTATCAATACATGGATGATTTGTATGTAGG |
| | ATCTGACTTAGAAATAGGGCAGCATAGAACAAAAATAGAGGAGCTGA |
| | GACAACATCTGTTGAGGTGGGGACTTACCACACCAGACAAAAAACA |
| | TCAGAAAGAACCTCCATTCCTTTGGATGGGTTATGAACTCCATCCTG |
| | ATAAATGGACAGTACAGCCTATAGTGCTGCCAGAAAAAGACAGCTGG |
| | ACTGTCAATGACATACAGAA |
| T7-HIV Geneblock 2-DNA | TAATACGACTCACTATAGGGGCGGGAATCAAGCAGGAATTTGGAATT |
| fragment with T7 promoter | CCCTACAATCCCCAAAGTCAAGGAGTAGTAGAATCTATGAATAAAGAA |
| used to produce a fragment of | TTAAAGAAAATTATAGGACAGGTAAGAGATCAGGCTGAACATCTTAAG |
| genomic HIV RNA through in | ACAGCAGTACAAATGGCAGTATTCATCCACAATTTTAAAAGAAAAGGG |
| vitro transcription with T7 RNA | GGGATTGGGGGGTACAGTGCAGGGGAAAGAATAGTAGACATAATAG |
| Pol (SEQ ID NO. 48) | CAACAGACATACAAACTAAAGAATTACAAAAACAAATTACAAAAATTCA |
| | AAATTTTCGGGTTTATTACAGGGACAGCAGAAATCCACTTTGGAAAG |
| | GACCAGCAAAGCTCCTCTGGAAAGGTGAAGGGGCAGTAGTAATACA |
| | AGATAATAGTGACATAAAAGTAGTGCCAAGAAGAAAAGCAAAGATCAT |
| | TAGGGATTATGGAAAACAGATGGCAGGTGATGATTGTGTGGCAAGTA |
| | GACAGGATGAGGATTAGAACATGGAAAAGTTTAGTAAAACACCATAT |
| | GTATGTTTCAGGGAAAGCTAGGGGATGGTTTTATAGACATCACTATGA |
| | AAGCCCTCATCCAAGAATAAGTTCAGAAGTACACATCCCACTAGGGG |
| | ATGCTAGATTGGTAATAACAACATATTGGGGTCTGCATACAGGAGAAA |
| | GAGACTGGCATTTGGGTCAGGGAGTCTCCATAGAATGGAGGAAAAA |
| | GAGATATAGCACACAAGTAGACCCTGAACTAGCAGACCAACTAATTC |
| | ATCTGTATTACTTTGACTGTTTTTCAGACTCTGCTATAAGAAAGGCCT |
| | TATTAGGACACATAGTTAGCCCTAGGTGTGAATATCAAGCAGGACATA |
| | ACAAGGTAGGATCTCTACAATACTTGGCACTAGCAGCATTAATAACAC |
| | CAAAAAAGATAAAGCCACCTTTGCCTAGTGTTACGAAACTGACAGAG |
| | GATAGATGGAACAAGCCCCAGAAGACCAAGGGCCACAGAGGGAGC |
| | CACACAATGAATGGACACTAGAGCTTTTAGAGGAGCTTAAGAATGAA |
| | GCTGTTAGACATTTTCCTAGGATTTGGCTCCATGGCTTAGGGCAACA |
| | TATCTATGAAACTTATGGGGATACTTGGGCAGGAGTGGAAGCCATAAT |
| | AAGAATTCTGCAACAACTGCTGTTTATCCATTTTCAGAATTGGGTGTC |
| | GACATAGCAGAATAGGCGTTACTCGACAGAGGAGAGCAAGAAATGG |
| | AGCCAGTAGATCCTAGACTAGAGCCCTGGAAGCATCCAGGAAGTCA |
| | GCCTAAAACTGCTTGTACCAATTGCTATTGTAAAAAGTGTTGCTTTCA |
| | TTGCCAAGTTTGTTTCATAACAAAAGCCTTAGGCATCTCCTATGGCAG |
| | GAAGAAGCGGAGACAGCGACGAAGAGCTCATCAGAACAGTCAGAC |
| | TCATCAAGCTTCTCTATCAAAGCAGTAAGTAGTACATGTAATGCAACC |
| | TATACCAATAGTAGCAATAGTAGCATTAGTAGTAGCAATAATAATAGCA |
| | ATAGTTGTGTGGTCCATAGTAATCATAGAATATAGGAAAATATTAAGAC |
| | AAAGAAAAATAGACAGGTTAATTGATAGACTAATAGAAAGAGCAGAAG |
| | ACAGTGGCAATGAGAGTGAAGGAGAAATATCAGCACTTGTGGAGAT |
| | GGGGGTGGAGATGGGGCACCATGCTCCTTGGGATGTTGATGATCTG |
| | TAGTGCTACAGAAAAATTGTGG |
| T7-HIV Geneblock 3-DNA | TAATACGACTCACTATAGAGACCCAACAACAATACAAGAAAAAGAATC |
| fragment with T7 promoter | CGTATCCAGAGAGGACCAGGGAGAGCATTTGTTACAATAGGAAAAAT |
| used to produce a fragment of | AGGAAATATGAGACAAGCACATTGTAACATTAGTAGAGCAAAATGGAA |
| genomic HIV RNA through in | TAACACTTTAAAACAGATAGCTAGCAAATTAAGAGAACAATTTGGAAA |
| vitro transcription with T7 RNA | TAATAAAACAATAATCTTTAAGCAATCCTCAGGAGGGGACCCAGAAAT |
| Pol (SEQ ID NO, 49) | TGTAACGCACAGTTTTAATTGTGGAGGGGAATTTTTCTACTGTAATTC |
| | AACACAACTGTTTAATAGTACTTGGTTTAATAGTACTTGGAGTACTGA |
| | AGGGTCAAATAACACTGAAGGAAGTGACACAATCACCCTCCCATGCA |
| | GAATAAAACAAATTATAAACATGTGGCAGAAAGTAGGAAAAGCAATGT |
| | ATGCCCCTCCCATCAGTGGACAAATTAGATGTTCATCAAATATTACAG |
| | GGCTGCTATTAACAAGAGATGGTGGTAATAGCAACAATGAGTCCGAG |
| | ATCTTCAGACCTGGAGGAGGAGATATGAGGGACAATTGGAGAAGTG |
| | AATTATATAAATATAAAGTAGTAAAAATTGAACCATTAGGAGTAGCACC |
| | CACCAAGGCAAAGAGAAGAGTGGTGCAGAGAGAAAAAAGAGCAGT |
| | GGGAATAGGAGCTTTGTTCCTTGGGTTCTTGGGAGCAGCAGGAAGC |
| | ACTATGGGCGCAGCCTCAATGACGCTGACGGTACAGGCCAGACAAT |
| | TATTGTCTGGTATAGTGCAGCAGCAGAACAATTTGCTGAGGGCTATT |
| | GAGGCGCAACAGCATCTGTTGCAACTCACAGTCTGGGGCATCAAGC |
| | AGCTCCAGGCAAGAATCCTGGCTGTGGAAAGATACCTAAAGGATCA |
| | ACAGCTCCTGGGGATTTGGGGTTGCTCTGGAAAACTCATTTGCACC |
| | ACTGCTGTGCCTTGGAATGCTAGTTGGAGTAATAAATCTCTGGAACA |
| | GATTTGGAATCACACGACCTGGATGGAGTGGGACAGAGAAATTAAC |
| | AATTACACAAGCTTAATACACTCCTTAATTGAAGAATCGCAAAACCAG |
| | CAAGAAAAGAATGAACAAGAATTATTGGAATTAGATAAATGGGCAAGT |
| | TTGTGGAATTGGTTTAACATAACAAATTGGCTGTGGTATATAAAATTAT |
| | TCATAATGATAGTAGGAGGCTTGGTAGGTTTAAGAATAGTTTTTGCTG |
| | TACTTTCTATAGTGAATAGAGTTAGGCAGGGATATTCACCATTATCGTT |
| | TCAGACCCACCTCCCAACCCCGAGGGGACCCGACAGGCCCGAAGG |
| | AATAGAAGAAGAAGGTGGAGAGAGAGACAGAGACAGATCCATTCGA |
| | TTAGTGAACGGATCCTTGGCACTTATCTGGGACGATCTGCGGAGCC |
| | TGTGCCTCTTCAGCTACCACCGCTTGAGAGACTTACTCTTGATTGTA |
| | ACGAGGATTGTGGAACTTCTGGGACGCAGGGGGTGGGAAGCCCTC |
| | AAATATTGGTGGAATCTCCTACAGTATTGGAGTCAGGAACTAAAGAAT |
| | AGTGCTGTTAGCTTGCTCAATGCCACAGCCATAGCAGTAGCTGAGG |
| | GGACAGATAGGGTTATAGAAGTAGTACAAGGAGCTTGTAGAGCTATT |
| | CGCCACATACCTAGAAGAATAAGACAGGGC |
| HIV ΨDNA Cas12i1_1 (SEQ | CTG GTC TAA CCA GAG AGA CCA ATT TTT GTG CCC ATC GTT GGC |
| ID NO. 50) | AC |
| HIV ΨDNA Cas12i1_2 (SEQ | TTG AAG CAC TCA AGG CAA GCA ATT TTT GTG CCC ATC GTT GGC |
| ID NO. 51) | AC |
| HIV ΨDNA Cas12i1_3 (SEQ | TGG CGT ACT CAC CAG TCG CCAATT TTT GTG CCC ATC GTT GGC |
| ID NO. 52) | AC |
| HIV ΨDNA Cas12i1_4 (SEQ | TCC CAG TAT TTG TCT ACA GCA ATT TTT GTG CCC ATC GTT GGC |
| ID NO. 53) | AC |
| HIV ΨDNA Cas12i1_5 (SEQ | CTG ACC TGA TTG CTG TGT CCA ATT TTT GTG CCC ATC GTT GGC |
| ID NO. 54) | AC |
| HIV ΨDNA Cas12i1_6 (SEQ | GCT TCC TCA TTG ATG GTC TCA ATT TTT GTG CCC ATC GTT GGC |
| ID NO. 55) | AC |
| HIV ΨDNA Cas12i1_7 (SEQ | GTC CAG AAT GCT GGT AGG GCA ATT TTT GTG CCC ATC GTT GGC |
| ID NO. 56) | AC |
| HIV ΨDNA Cas12i1_8 (SEQ | GGT CCT CCT ACT CCC TGA CAA ATT TTT GTG CCC ATC GTT GGC |
| ID NO. 57) | AC |
| HIV ΨDNA Cas12i1_9 (SEQ | ATT AGC CTG TCT CTC AGT ACA ATT TTT GTG CCC ATC GTT GGC |
| ID NO. 58) | AC |
| HIV ΨDNA Cas12i1_10 (SEQ | TTG CCA AAG AGT GAC CTG AGA ATT TTT GTG CCC ATC GTT GGC |
| ID NO. 59) | AC |
| HIV ΨDNA Cas12i1_11 (SEQ | TTG ACA GGT GTA GGT CCT ACA ATT TTT GTG CCC ATC GTT GGC |
| ID NO. 60) | AC |
| HIV ΨDNA Cas12i1_12 (SEQ | TCT TCT GTC AAT GGC CAT TGA ATT TTT GTG CCC ATC GTT GGC |
| ID NO. 61) | AC |
| HIV ΨDNA Cas12i1_13 (SEQ | CCC AGA AGT CTT GAG TTC TCA ATT TTT GTG CCC ATC GTT GGC |
| ID NO. 62) | AC |
| HIV ΨDNA Cas12i1_14 (SEQ | TGG AAT ATT GCT GGT GAT CCA ATT TTT GTG CCC ATC GTT GGC |
| ID NO. 63) | AC |
| HIV ΨDNA Cas12i1_15 (SEQ | TGG CAG CAC TAT AGG CTG TAA ATT TTT GTG CCC ATC GTT GGC |
| ID NO. 64) | AC |
| HIV ΨDNA Cas12i1_16 (SEQ | TGC CAG TTC TAG CTC TGC TTA ATT TTT GTG CCC ATC GTT GGC |
| ID NO. 65) | AC |
| HIV ΨDNA Cas12i1_17 (SEQ | GTT TCC TTT TGT ATG GGC AGA ATT TTT GTG CCC ATC GTT GGC |
| ID NO. 66) | AC |
| HIV ΨDNA Cas12i1_18 (SEQ | TTA GTC TCC CTG TTA GCT GCA ATT TTT GTG CCC ATC GTT GGC |
| ID NO. 67) | AC |
| HIV ΨDNA Cas12i1_19 (SEQ | GTA CCC ATG CCA GAT AGA CCA ATT TTT GTG CCC ATC GTT GGC |
| ID NO. 68) | AC |
| HIV ΨDNA Cas12i1_20 (SEQ | ACA GTC TAC TTG TCC ATG CAA ATT TTT GTG CCC ATC GTT GGC |
| ID NO. 69) | AC |
| HIV ΨDNA Cas12i1_21 (SEQ | TGC TGC CAT TGT CAG TAT GTA ATT TTT GTG CCC ATC GTT GGC |
| ID NO. 70) | AC |
| HIV ΨDNA Cas12i1_22 (SEQ | TTC TTT CCC CTG CAC TGT ACA ATT TTT GTG CCC ATC GTT GGC |
| ID NO. 71) | AC |
| HIV ΨDNA Cas12i1_23 (SEQ | CAA TCA TCA CCT GCC ATC TGA ATT TTT GTG CCC ATC GTT GGC |
| ID NO. 72) | AC |
| HIV ΨDNA Cas12i1_24 (SEQ | TGG GAT GTG TAC TTC TGA ACA ATT TTT GTG CCC ATC GTT GGC |
| ID NO. 73) | AC |
| HIV ΨDNA Cas12i1_25 (SEQ | AGT ATT GTA GAG ATC CTA CCA ATT TTT GTG CCC ATC GTT GGC |
| ID NO. 74) | AC |
| HIV ΨDNA Cas12i1_26 (SEQ | CAG CTT CAT TCT TAA GCT CCA ATT TTT GTG CCC ATC GTT GGC |
| ID NO. 75) | AC |
| HIV ΨDNA Cas12i1_27 (SEQ | TTC CTG GAT GCT TCC AGG GCA ATT TTT GTG CCC ATC GTT GGC |
| ID NO. 76) | AC |
| HIV ΨDNA Cas12i1_28 (SEQ | ATG GAC CAC ACA ACT ATT GCA ATT TTT GTG CCC ATC GTT GGC |
| ID NO. 77) | AC |
| HIV ΨDNA Cas12i1_29 (SEQ | CTG TAG CAC TAC AGA TCA TCA ATT TTT GTG CCC ATC GTT GGC |
| ID NO. 78) | AC |
| HIV ΨDNA Cas12i1_30 (SEQ | GCT TTA GGC TTT GAT CCC ATA ATT TTT GTG CCC ATC GTT GGC |
| ID NO. 79) | AC |
| HIV ΨDNA Cas12i1_31 (SEQ | TGA CTG AGG TGT TAC AAC TTA ATT TTT GTG CCC ATC GTT GGC |
| ID NO. 80) | AC |
| HIV ΨDNA Cas12i1_32 (SEQ | TGA GTT GAT ACT ACT GGC CTAATT TTT GTG CCC ATC GTT GGC |
| ID NO. 81) | AC |
| HIV ΨDNA Cas12i1_33 (SEQ | TGT TAC AAT GTG CTT GTC TCA ATT TTT GTG CCC ATC GTT GGC |
| ID NO. 82) | AC |
| HIV ΨDNA Cas12i1_34 (SEQ | GTG ATT GTG TCA CTT CCT TCA ATT TTT GTG CCC ATC GTT GGC |
| ID NO. 83) | AC |
| HIV ΨDNA Cas12i1_35 (SEQ | TAT CTC CTC CTC CAG GTC TGA ATT TTT GTG CCC ATC GTT GGC |
| ID NO. 84) | AC |
| HIV ΨDNA Cas12i1_36 (SEQ | TCA GCA AAT TGT TCT GCT GCA ATT TTT GTG CCC ATC GTT GGC |
| ID NO. 85) | AC |
| HIV ΨDNA Cas12i1_37 (SEQ | CAT CCA GGT CGT GTG ATT CCA ATT TTT GTG CCC ATC GTT GGC |
| ID NO. 86) | AC |
| HIV ΨDNA Cas12i1_38 (SEQ | ATC CCT GCC TAA CTC TAT TCA ATT TTT GTG CCC ATC GTT GGC AC |
| ID NO. 87) | |
| HIV ΨDNA Cas12i1_39 (SEQ | AGT AAG TCT CTC AAG CGG TGA ATT TTT GTG CCC ATC GTT GGC |
| ID NO. 88) | AC |
| HIV ΨDNA Cas12i1_40 (SEQ | TAT TCT TCT AGG TAT GTG GCA ATT TTT GTG CCC ATC GTT GGC AC |
| ID NO. 89) | |
| HIV ΨDNA Cas12i1_41 (SEQ | CTC TTG TGC TTC TAG CCA GGA ATT TTT GTG CCC ATC GTT GGC |
| ID NO. 90) | AC |
| HIV ΨDNA Cas12i1_42 (SEQ | CAA CTG GTA CTA GCT TGT AGA ATT TTT GTG CCC ATC GTT GGC |
| ID NO. 91) | AC |
| HIV ΨDNA Cas12i1_43 (SEQ | AAG TCC CTT GTA GCA AGC TCA ATT TTT GTG CCC ATC GTT GGC |
| ID NO. 92) | AC |
| HIV ΨDNA Cas12i1_44 (SEQ | TAT ATG CAG GAT CTG AGG GCA ATT TTT GTG CCC ATC GTT GGC |
| ID NO. 93) | AC |
| HIV ΨDNA Cas12i1_45 (SEQ | GAA GCA CTC AAG GCA AGC TTA ATT TTT GTG CCC ATC GTT GGC |
| ID NO. 94) | AC |
| HIV ΨDNA Cas12i1_46 (SEQ | ATC CCG AAT CCT GCA AAG CTA ATT TTT GTG CCC ATC GTT GGC |
| ID NO. 95) | AC |
| HIV ΨDNA Cas12i1_47 (SEQ | TAT ATC CAC TGG CTA CAT GAA ATT TTT GTG CCC ATC GTT GGC AC |
| ID NO. 96) | |
| HIV ΨDNA Cas12i1_48 (SEQ | GTA CTG TCC ATT TAT CAG GAA ATT TTT GTG CCC ATC GTT GGC |
| ID NO. 97) | AC |
| HIV ΨDNAAsCas12a_1 (SEQ | CTG GTC TAA CCA GAG AGA CCT AGA TGT GAA TCA TCT TTAAT |
| ID NO. 98) | |
| HIV ΨDNAAsCas12a_2 (SEQ | TTGAAG CAC TCAAGG CAA GCT AGA TGT GAA TCA TCT TTAAT |
| ID NO. 99) | |
| HIV ΨDNAAsCas12a_3 (SEQ | TGG CGT ACT CAC CAG TCG CCT AGA TGT GAA TCA TCT TTA AT |
| ID NO. 100) | |
| HIV ΨDNAAsCas12a_4 (SEQ | TCC CAG TAT TTG TCT ACA GCT AGA TGT GAA TCA TCT TTAAT |
| ID NO. 101) | |
| HIV ΨDNAAsCas12a_5 (SEQ | CTG ACC TGA TTG CTG TGT CCT AGA TGT GAA TCA TCT TTA AT |
| ID NO. 102) | |
| HIV ΨDNAAsCas12a_6 (SEQ | GCT TCC TCA TTG ATG GTC TCT AGA TGT GAA TCA TCT TTAAT |
| ID NO. 103) | |
| HIV ΨDNAAsCas12a_7 (SEQ | GTC CAG AAT GCT GGT AGG GCT AGA TGT GAA TCA TCT TTA AT |
| ID NO. 104) | |
| HIV ΨDNAAsCas12a_8 (SEQ | GGT CCT CCT ACT CCC TGA CAT AGA TGT GAA TCA TCT TTAAT |
| ID NO. 105) | |
| HIV ΨDNAAsCas12a_9 (SEQ | ATT AGC CTG TCT CTC AGT ACT AGA TGT GAA TCA TCT TTA AT |
| ID NO. 106) | |
| HIV ΨDNA AsCas 12a_10 | TTG CCA AAG AGT GAC CTG AGT AGA TGT GAA TCA TCT TTA AT |
| (SEQ ID NO. 107) | |
| HIV ΨDNA AsCas12a_11 | TTG ACA GGT GTA GGT CCT ACT AGA TGT GAA TCA TCT TTAAT |
| (SEQ ID NO. 108) | |
| HIV ΨDNA AsCas12a_12 | TCT TCT GTC AAT GGC CAT TGT AGA TGT GAA TCA TCT TTAAT |
| (SEQ ID NO. 109) | |
| HIV ΨDNA AsCas12a_13 | CCC AGAAGT CTT GAG TTC TCT AGA TGT GAA TCA TCT TTAAT |
| (SEQ ID NO. 110) | |
| HIV ΨDNA AsCas12a_14 | TGGAAT ATT GCT GGT GAT CCT AGA TGT GAA TCA TCT TTA AT |
| (SEQ ID NO. 111) | |
| HIV ΨDNA AsCas12a_15 | TGG CAG CAC TAT AGG CTG TAT AGA TGT GAA TCA TCT TTAAT |
| (SEQ ID NO. 112) | |
| HIV ΨDNA AsCas12a_16 | TGC CAG TTC TAG CTC TGC TTT AGA TGT GAA TCA TCT TTAAT |
| (SEQ ID NO. 113) | |
| HIV ΨDNA AsCas12a_17 | GTT TCC TTT TGT ATG GGC AGT AGA TGT GAA TCA TCT TTA AT |
| (SEQ ID NO. 114) | |
| HIV ΨDNA AsCas12a_18 | TTA GTC TCC CTG TTA GCT GCT AGA TGT GAA TCA TCT TTAAT |
| (SEQ ID NO. 115) | |
| HIV ΨDNA AsCas12a_19 | GTA CCC ATG CCA GAT AGA CCT AGA TGT GAA TCA TCT TTAAT |
| (SEQ ID NO. 116) | |
| HIV ΨDNA AsCas12a_20 | ACA GTC TAC TTG TCC ATG CAT AGA TGT GAA TCA TCT TTAAT |
| (SEQ ID NO. 117) | |
| HIV ΨDNA AsCas12a_21 | TGC TGC CAT TGT CAG TAT GTT AGA TGT GAA TCA TCT TTAAT |
| (SEQ ID NO. 118) | |
| HIV ΨDNA AsCas12a_22 | TTC TTT CCC CTG CAC TGT ACT AGA TGT GAA TCA TCT TTAAT |
| (SEQ ID NO. 119) | |
| HIV ΨDNA AsCas12a_23 | CAA TCA TCA CCT GCC ATC TGT AGA TGT GAA TCA TCT TTAAT |
| (SEQ ID NO. 120) | |
| HIV ΨDNA AsCas12a_24 | TGG GAT GTG TAC TTC TGAACT AGA TGT GAA TCA TCT TTAAT |
| (SEQ ID NO. 121) | |
| HIV ΨDNA AsCas12a_25 | AGT ATT GTA GAG ATC CTA CCT AGA TGT GAA TCA TCT TTAAT |
| (SEQ ID NO. 122) | |
| HIV ΨDNA AsCas12a_26 | CAG CTT CAT TCT TAA GCT CCT AGA TGT GAA TCA TCT TTA AT |
| (SEQ ID NO. 123) | |
| HIV ΨDNA AsCas12a_27 | TTC CTG GAT GCT TCC AGG GCT AGA TGT GAA TCA TCT TTAAT |
| (SEQ ID NO. 124) | |
| HIV ΨDNA AsCas12a_28 | ATG GAC CAC ACAACT ATT GCT AGA TGT GAA TCA TCT TTAAT |
| (SEQ ID NO. 125) | |
| HIV ΨDNA AsCas12a_29 | CTG TAG CAC TAC AGA TCA TCT AGA TGT GAA TCA TCT TTA AT |
| (SEQ ID NO. 126) | |
| HIV ΨDNA AsCas12a_30 | GCT TTA GGC TTT GAT CCC ATT AGA TGT GAA TCA TCT TTAAT |
| (SEQ ID NO. 127) | |
| HIV ΨDNA AsCas12a_31 | TGA CTG AGG TGT TAC AAC TTT AGA TGT GAA TCA TCT TTA AT |
| (SEQ ID NO. 128) | |
| HIV ΨDNA AsCas12a_32 | TGA GTT GAT ACT ACT GGC CTT AGA TGT GAA TCA TCT TTAAT |
| (SEQ ID NO. 129) | |
| HIV ΨDNA AsCas12a_33 | TGT TAC AAT GTG CTT GTC TCT AGA TGT GAA TCA TCT TTAAT |
| (SEQ ID NO. 130) | |
| HIV ΨDNA AsCas12a_34 | GTG ATT GTG TCA CTT CCT TCT AGA TGT GAA TCA TCT TTAAT |
| (SEQ ID NO. 131) | |
| HIV ΨDNA AsCas12a_35 | TAT CTC CTC CTC CAG GTC TGT AGA TGT GAA TCA TCT TTAAT |
| (SEQ ID NO. 132) | |
| HIV ΨDNA AsCas12a_36 | TCA GCA AAT TGT TCT GCT GCT AGA TGT GAA TCA TCT TTAAT |
| (SEQ ID NO. 133) | |
| HIV ΨDNA AsCas12a_37 | CAT CCA GGT CGT GTG ATT CCT AGA TGT GAA TCA TCT TTA AT |
| (SEQ ID NO. 134) | |
| HIV ΨDNA AsCas12a_38 | ATC CCT GCC TAA CTC TAT TCT AGA TGT GAA TCA TCT TTAAT |
| (SEQ ID NO. 135) | |
| HIV ΨDNA AsCas12a_39 | AGT AAG TCT CTC AAG CGG TGT AGA TGT GAA TCA TCT TTAAT |
| (SEQ ID NO. 136) | |
| HIV ΨDNA AsCas12a_40 | TAT TCT TCT AGG TAT GTG GCT AGA TGT GAA TCA TCT TTA AT |
| (SEQ ID NO. 137) | |
| HIV ΨDNA AsCas12a_41 | CTC TTG TGC TTC TAG CCA GGT AGA TGT GAA TCA TCT TTA AT |
| (SEQ ID NO. 138) | |
| HIV ΨDNA AsCas12a_42 | CAA CTG GTA CTA GCT TGT AGT AGA TGT GAA TCA TCT TTA AT |
| (SEQ ID NO. 139) | |
| HIV ΨDNA AsCas12a_43 | AAG TCC CTT GTA GCAAGC TCT AGA TGT GAA TCA TCT TTAAT |
| (SEQ ID NO. 140) | |
| HIV ΨDNA AsCas12a_44 | TAT ATG CAG GAT CTG AGG GCT AGA TGT GAA TCA TCT TTA AT |
| (SEQ ID NO. 141) | |
| HIV ΨDNA AsCas12a_45 | GAA GCA CTC AAG GCAAGC TTT AGA TGT GAA TCA TCT TTA AT |
| (SEQ ID NO. 142) | |
| HIV ΨDNA AsCas12a_46 | ATC CCGAAT CCT GCAAAG CTT AGA TGT GAA TCA TCT TTAAT |
| (SEQ ID NO. 143) | |
| HIV ΨDNA AsCas12a_47 | TAT ATC CAC TGG CTA CAT GAT AGA TGT GAA TCA TCT TTA AT |
| (SEQ ID NO. 144) | |
| HIV ΨDNA AsCas12a_48 | GTA CTG TCC ATT TAT CAG GAT AGA TGT GAA TCA TCT TTA AT |
| (SEQ ID NO. 145) | |

### CRISPR-Cas Based Fluorescence Detection Assay

All detection assays using fluorescence were performed in black 384-well plates with a low-volume, flat-bottom design. The crRNA-Cas12 complexes were prepared by mixing in NEB 2.1 buffer and nuclease free water, followed by a 10-minute room temperature incubation. These crRNA-Cas12 preparations were then combined with 250-500 nM of a fluorescent quenched (FQ) reporter and an appropriate amount of the target activator to achieve a reaction volume of 40 μL. The 384-well plate was subsequently placed in a BioTek Synergy fluorescence microplate reader, incubated at temperature of 37° C. for 60 minutes. Fluorescence intensity readings for a FAM-labeled reporter were recorded at 483/20 nm and 530/20 nm excitation/emission wavelengths at 2.5-minute intervals. Standard concentrations in these assays, unless otherwise noted, were 50 nM Cas enzyme (AsCas12a/Cas12i), 100 nM crRNA or ΨDNA, and 25 nM target activator, unless otherwise specified.

For Michaelis-Menten analysis, AsCas12a-Y′DNA-target RNA complexes were prepared as described above, and reaction was initiated by diluting AsCas12a complexes to a final concentration of 50 nM AsCas12a: 1.25 nM ΨDNA: 50 nM target RNA (effective complex=1.25 nM) in a solution containing 1×NEB Buffer 2.1 and 0.01, 0.05, 0.1, 0.2, 0.5, 1 or 2 μM of FQ (systhesized by IDT). Different concentrations of FAM reporter without quencher were used to plot the standard curve of concentration and fluorescence. Reactions were incubated in Applied Biosciences QuantStudio™ 5 Real-Time PCR System for up to 45 minutes at 37° C. with fluorescence measurements taken every 13 seconds. The initial velocity (V0) was calculated by fitting to a linear regression. Slopes, substrate concentration, and Michaelis-Menten constants are calculated and plotted by Prism10.

### Electrophoretic Mobility Shift Assay (EMSA)

EMSA gel was performed by mixing 100 nM of Cas12 protein (AsCas12a or Cas12i1), 100 nM of guide (crRNA or ΨDNA) and 100 nM target (ssDNA or ssRNA) in NEB 2.1 buffer, and incubating the reaction at 4° C. for 30 min. Then, 1 μL of 5×TBE Hi-Density Sample Buffer (Invitrogen #LC6678) was added to 9 μL of sample. The samples were loaded in a native PAGE DNA Retardation Gel (Invitrogen #EC6365BOX) and ran at 200V for 30 min. Later, PAGE gel was stained with SYBR Gold (Invitrogen #S11494) and imaged on Amersham Typhoon. Sequences used for the EMSA Assay are provided in Table 14.

TABLE 14
DNA and RNA Sequences used for EMSA Assay

| Name | Sequence |
|---|---|
| ΨDNA AsCas12a (SEQ ID NO. 27) | AGCACCCAGTCCGCCCTGAGTAGATGTGAATCATCTTTAAT |
| ΨDNA Cas12i1 (SEQ ID NO. 28) | AGCACCCAGTCCGCCCTGAGAATTTTTGTGCCCATCGTTGGCAC |
| RNA Target (SEQ ID NO. 29) | rCrUrCrArGrGrGrCrGrGrArCrUrGrGrGrUrGrCrU |
| CrRNA AsCas12a (SEQ ID NO. 30) | rUrArArUrUrUrCrUrArCrUrArArGrUrGrUrArGrArUrCrUrCrArGrGrGrCrGrGr |
| | ArCrUrGrGrGrUrGrCrU |
| crRNA Cas12i1 (SEQ ID NO. 31) | rArArUrUrUrUrUrGrUrGrCrCrCrArUrCrGrUrUrGrGrCrArCrCrUrCrArGrGrGr |
| | CrGrGrArCrUrGrGrGrUrGrCrU |
| ssDNA Target (SEQ ID NO. 32) | AGCACCCAGTCCGCCCTGAG |

### Bio-Layer Interferometry

The biolayer interferometry (BLI) analysis of the binding interaction between Cas enzymes and DNA or RNA based guides was performed using the GatorBio instrument, employing biotin-coated guides and streptavidin probes from the Flex SA Kit (GatorBio #350001). Priming reagent was switched to NEB 2.1 buffer with 0.05% Triton X. DNA and RNA oligos were synthesized with a 3′ biotin modification through Integrated DNA Technologies (IDT).

The BLI measurements were conducted on the GatorBio system, which allows real-time, label-free analysis of biomolecular interactions. The assay setup involved two key phases: association and dissociation. During the association phase, the streptavidin probes loaded with biotinylated guides at 50 nM were transferred to wells containing the Cas enzyme (Cas12i1 or AsCas12a) at seven concentrations (1 nM, 2.5 nM, 5 nM, 10 nM, 25 nM, 50 nM, and 100 nM). The binding of the Cas enzyme to the immobilized guide was monitored in real-time over a 10-minute period. In the subsequent dissociation phase, the sensors were moved to wells containing only the assay buffer to observe the dissociation of the Cas enzyme from the immobilized guide over a 10-minute period.

The BLI data was processed and analyzed using GatorBio software. Binding curves were generated for each concentration of the Cas enzyme, and the association (kₒₙ) and dissociation (k_{off}) rate constants were determined by fitting the data to a 1:1 binding model. The equilibrium dissociation constant (K_{d}) was calculated using a Michaels-Menten regression curve to fit the response curve at different concentrations. Comparative analyses between DNA and RNA guides were conducted to evaluate the specificity and affinity of the Cas enzyme for each type of guide. Sensors without immobilized guides served as negative controls to confirm the specificity of the streptavidin-biotin interaction and the subsequent guide-Cas binding. Sequences used for the BLI assay are provided in Table 15.

TABLE 15
DNA and RNA Sequences used for BLI Assay

| Name | Sequence |
|---|---|
| AsCas12a crGFP w/biotin (SEQ ID | rUrArArUrUrUrCrUrArCrUrArArGrUrGrUrArGrArUrCrUrCrArGrGrGrCrGrGrAr |
| NO. 33) | CrUrGrGrGrUrGrCrU/3Bio/ |
| Cas1211 crGFP w/biotin (SEQ ID | rArArUrUrUrUrUrGrUrGrCrCrCrArUrCrGrUrUrGrGrCrArCrCrUrCrArGrGrGrCr |
| NO. 34) | GrGrArCrUrGrGrGrUrGrCrU/3Bio/ |
| GFP RNA spacer w/biotin (SEQ ID | rCrUrCrArGrGrGrCrGrGrArCrUrGrGrGrUrGrCrU/3Bio/ |
| NO. 35) | |
| GFP cDNA w/biotin (SEQ ID NO. | AGCACCCAGTCCGCCCTGAG/3Bio/ |
| 36) | |
| AsCas12a GFP cDNA 5′ handle | TAGATGTGAATCATCTTTAATAGCACCCAGTCCGCCCTGAG/3Bio/ |
| w/biotin (SEQ ID NO. 37) | |
| AsCas12a GFP | AGCACCCAGTCCGCCCTGAGTAGATGTGAATCATCTTTAAT/3Bio/ |
| DNA w/biotin (SEQ ID NO. 38) | |
| AsCas12a GFP cDNA double | TAGATGTGAATCATCTTTAATAGCACCCAGTCCGCCCTGAGTAGATGTGAA |
| handle w/biotin (SEQ ID NO. 39) | TCATCTTTAAT/3Bio/ |
| Cas1211 GFP cDNA 5′ handle | AATTTTTGTGCCCATCGTTGGCACAGCACCCAGTCCGCCCTGAG/3Bio/ |
| w/biotin (SEQ ID NO. 40) | |
| Cas1211 GFP ΨDNA w/biotin | AGCACCCAGTCCGCCCTGAGAATTTTTGTGCCCATCGTTGGCAC/3Bio/ |
| (SEQ ID NO. 41) | |
| Cas1211 GFP cDNA double handle | AATTTTTGTGCCCATCGTTGGCACAGCACCCAGTCCGCCCTGAGAATTTT |
| w/biotin (SEQ ID NO. 42) | TGTGCCCATCGTTGGCAC/3Bio/ |

### Systematic ΨDNA Evaluation Assay

All ΨDNAs are synthesized by IDT. 50 nM AsCas12a, 100 nM target RNA and 200 nM ΨDNA were incubated at 37° C. for 30 minutes, then all ΨDNA groups all pooled together into a 15 mL tube and incubated at 37° C. for another 30 minutes. After incubation, pooled ΨDNAs were diluted 100 times for amplicon library preparation. Q5 DNA Polymerase was used for PCR. Lacking AsCas12a library are used as control.

The amplicon libraries were sequenced by Illumina NextSeq 500 with a NextSeq 500/550 Mid Output Kit v2.5 (150 Cycles) (Illumina #20024904). All computational work was performed on the University of Florida's HPC HiperGator.

### In Vitro Transcription

In vitro transcription of long HIV and HCV RNA fragments was done using HiScribe® T7 High Yield RNA Synthesis Kit (NEB #E2040S) following manufacturer's protocol and purified using Monarch RNA cleanup kit (NEB #T2030L).

Endogenous mRNA Detection

Total RNA of 1×10⁶ HEK293T cells (ATCC #CRL-3216) was extracted using Monarch Total RNA Miniprep Kit (NEB #T2010S). Then, cDNA was synthesized using PhotoScript II First Strand cDNA Synthesis Kit (NEB #E6560) with Oligo-dT primers. Lastly, 17 endogenous genes were amplified using KOD One PCR Master Mix (Toyobo #KMM-201) and in vitro transcribed as described above. For detection, 2 μL of product were added to a CRISPR-Cas based fluorescence detection assay.

### Patient Sample Collection

The collection and processing of patient samples were approved by the University of Florida Institutional Review Board (IRB202200294). For clinical validation, a total of 20 human serum samples were obtained from patients with Hepatitis C collected under the HCV-TARGET program, which also supplied the UI/mL of each sample reported in FIGS. 9A-9K. Healthy serum samples were obtained from Boca Biolistics.

### Patient Sample Extraction

The extraction of viral RNA from serum samples was carried out using the Quick-DNA/RNA Viral MagBead Kit (Zymo #R2140). Briefly, 10 μL of Proteinase K (20 mg/mL) was added to 200 μL of the serum samples in 1.5 mL centrifuge tubes, followed by incubation at room temperature for 15 minutes. Afterward, DNA/RNA Shield™ (2× concentrate) was mixed with the serum sample containing Proteinase K at a 1:1 ratio. The resulting mixture was then supplemented with 800 μL of Viral DNA/RNA Buffer, followed by the addition of 20 μL of Magbinding Beads™. The mixture was vortexed for 10 minutes, and then the tubes were placed on a magnetic stand to separate the beads. The supernatant was carefully removed, and the beads were sequentially washed with 250 μL of MagBead DNA/RNA Wash 1, 250 μL of MagBead DNA/RNA Wash 2, and two rounds of 250 μL of 100% ethanol. After allowing the beads to air-dry for 10 minutes, the DNA/RNA was eluted with 30-60 μL of DNase/RNase-Free water and subsequently used for downstream analysis.

### Patient Sample Validation

After extraction, samples were amplified by adding 1 μL of extracted viral RNA to a 50 L SuperScript™ IV One-Step RT PCR reaction (Invitrogen #12594025) and performed thermocycling according to manufacturer's instructions. After amplification, 1 μL of product was added to a T7 and AsCas12a reaction master mix for RNA detection. This master mix contained 1×NEB 2.1 buffer, 62.5 nM AsCas12a, 112.5 nM ΨDNA (IDT), 500 nM FQ reporter (IDT), 1 mM rNTPs Mix (NEB #N0466), 2 U/mL of RNase Inhibitor (NEB #M0314), and 12.5 U/mL of NxGen T7 RNA Polymerase (Biosearch #30221-1) in 20 μL reactions. Samples were then loaded into a 384-well plate which was subsequently placed in a BioTek Synergy fluorescence microplate reader, incubated at temperature of 37° C. for 60 minutes. Fluorescence intensity readings for a FAM-labeled reporter were recorded at 483/20 nm and 530/20 nm excitation/emission wavelengths at 2.5-minute intervals.

### Amplicon Sequencing for Patient Samples

Extracted samples were amplified through a SuperScript™ IV One-Step RT PCR reaction as the step above. A second round of PCR was performed using Q5 DNA Polymerase to append Illumina barcodes. Samples were then pooled together, gel extracted and loaded into an Illumina MiSeqDx with a MiSeq Reagent Nano Kit v2 (Illumina #MS-101-1001). Bowtie2, Samtools, and JBrowse were used to align the sequencing output to an HCV reference genome.

### Mammalian Cell Culture

HEK293T (ATCC #CRL-3216), HeLa, MCF7 cells were cultured in DMEM high glucose GlutaMAX™ supplement pyruvate (Gibco #10569010), 10% Fetal Bovine Serum (Gibco #A3160902) and 1× Penicillin-Streptomycin. Cells were incubated at 37° C. and 5% CO₂.

HepG2 cells were cultured in RPMI-1640 (Corning #10-104-CV), 10% Fetal Bovine Serum (Gibco #A3160902) and 1× Penicillin-Streptomycin. Cells were incubated at 37° C. and 5% CO₂.

HeLa, MCF7 and HepG2 cells were obtained from Dr. Mingyi Xie's Lab at the University of Florida.

### Mammalian Cell Transfection

For RNA extraction and qPCR, plasmids were co-transfected with different ΨDNAs into HEK293T cells using TransIT-X2® transfection reagent (Mirus Bio #MIR6000). For all experiments, 5×10⁵ cells per mL were seeded in plates 48 hours before transfection.

For mCherry reporter system, 200 ng of mCherry plasmid, 350 ng of AsCas12a-GFP (Addgene #160140) or GFP only (Addgene #133962) plasmid, 400 ng of ΨDNA and 2 μL of TransIT-X2® were added to 50 μL of Opti-MEM™ Reduced Serum Medium (Gibco, Cat #31985062). Complexes were allowed to form for 25 minutes and posteriorly added dropwise to each well in seeded 48-well plates. For the control reaction with no ΨDNA, to normalize transfection efficiency among all samples 400 ng of a short-randomized DNA were added to the reaction instead of ΨDNA. Transfected cells were harvested 16 hours post-transfection for flow cytometry and gene expression analysis.

For endogenous RNA gene knockdown with AsCas12a transfection in HEK293T cells, 350 ng of AsCas12a-GFP or GFP-only plasmid, 650 ng of total ΨDNA (targeting or non-targeting), and 2 μL of TransIT-X2® were added to 50 μL of Opti-MEM™ Reduced Serum Medium. Complexes were allowed to form for 25 minutes and then added dropwise to each well of pre-seeded 48-well plates containing HEK293T cells. Similarly, for endogenous RNA gene knockdown with AsCas12a transduction, 650 ng of total ΨDNA (targeting or non-targeting), and 2 μL of TransIT-X2® were added to 50 μL of Opti-MEM™ Reduced Serum Medium. Complexes were allowed to form for 25 minutes and then added dropwise to each well of pre-seeded 48-well plates containing AsCas12a+/GFP+ HEK293T cells. In HeLa, MCF7 and HepG2 cells, the transfection amount of ΨDNAs were decreased to 175 ng.

For multiplex gene targeting, different ΨDNAs were equally mixed to a total of 650 ng before being transfected. Cells were harvested 24 hours of post-transfection for gene expression analysis.

For RIP-qPCR and MeRIP-qPCR, 2×10⁶ per well HEK293T cells were plated in 6-well plates 24 hours before transfection. 1.33 μg of AsCas12a-GFP or HA-GFP or AsCas12a-METTL3-GFP, 1.07 μg ΨDNA, and 6 μL of TransIT-X2® were added to 250 μL of Opti-MEM™ Reduced Serum Medium. Transfected cells were harvested 24 hours of post-transfection.

For ΨDNA guided RNase H1 fused AsCas12a, plasmid and ΨDNA transfection amount was performed same as mCherry reporter system. Transfected cells were harvested 24 hours post-transfection for gene expression analysis.

Flow Cytometry for Quantification of mCherry Expression

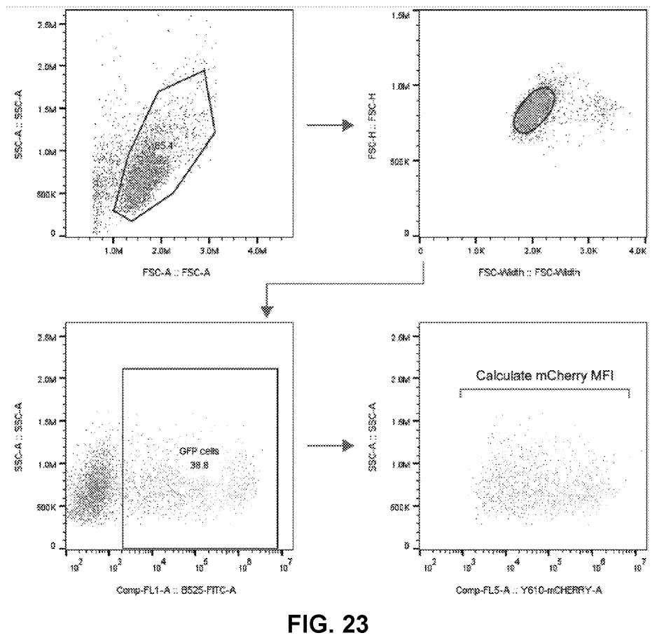

16 hours after cells were transfected as described above, these were trypsinized with 1× Trypsin-EDTA (Gibco #15400054). Then the cells were resuspended in FluoroBrite™ DMEM with 10% FBS. Each sample was passed through a 35 μm cell strainer (FALCON #352235). Cells were then analyzed in a Beckman Coulter CytoFLEX LX flow cytometer. FCS files were then analyzed in FloJo™ v.10.10 to obtain the MFI of mCherry expression in GFP-positive cells. Before analyzing samples compensation for GFP and mCherry was performed using GFP cells, mCherry cells, and GFP & mCherry BrightComp eBeads™ (Invitrogen #A10514 and #A54743). Gating illustrated in FIG. 23.

RT-qPCR for Relative Quantification of mCherry and Endogenous mRNA

Total RNA of samples was extracted using Monarch Total RNA Miniprep Kit (NEB #T2010S) as per manufacturer's instructions. Extracted RNA was later added to the RT-qPCR mix TaqMan™ Fast Virus 1-Step Master Mix (Thermo #4444434). The reaction was performed on an Applied Biosciences QuantStudio™ 5 Real-Time PCR System and multiplexed with FAM probes for mCherry and endogenous genes, and Cy5 probe for GAPDH as the housekeeping gene. mCherry primers and probes were designed using PrimeQuest™ Tool from IDT and GAPDH primers and probes are from Asahi-Ozaki et al. For endogenous genes (PPIA, RPL4, SMARCA4, NRAS, and PCSK9), primers and probes were ordered from Thermo Fisher. Fold-change was calculated relative to non-target ΨDNA using the ddCt method. One-way or two-way ANOVA with multiple comparison correction was performed to assess the statistical significance of transcript changes, using Prism10.

mRNA-Seg for Off-Target Effect Quantification

Similarly to what was described in the mammalian cell transfection section, HEK293T cells were seeded at 5×10⁵ cells per well and were seeded in 6-well plates 48 hours before transfection. Then, 1 μg of AsCas12a-GFP, RfxCas13d-GFP, dCas13d-GFP or GFP-only plasmid, 2 μg of ΨDNA, and 8 μL of TransIT-X2® were added to 250 μL of Opti-MEM™ Reduced Serum Medium. After 18 hours, cells were trypsinized and resuspended in 2% FBS in PBS for GFP cell sorting on a BD FACSymphony™ S6 flow cytometer.

After GFP positive cells were sorted, total RNA was extracted from them using Monarch Total RNA Miniprep Kit (NEB #T2010S) as per manufacturer's instructions. RNA integrity (RIN) score of the samples was measured with a QIAxcel Advance with an QIAxcel RNA QC Kit v2.0 (Qiagen #929104) cartridge to proceed with RNA library preparation for Illumina NGS sequencing,

Libraries were prepared with NEBNext® Ultra™ ∥ Directional RNA Library Prep (NEB #E7765S) in conjunction with NEBNext Poly(A) mRNA Magnetic Isolation Module (NEB #E7490) to isolate only mRNA from the samples. Indexes used were NEBNext® Multiplex Oligos for Illumina® Set 1, 2, and 3 (NEB #E7335S, #E7500S, #E7710S). Final libraries were loaded in a QIAxcel DNA Screening Kit (Qiagen #929004) to check for correct size distribution before sequencing and quantifies using Qubit Flex and qPCR.

Samples were pooled together and loaded into an Illumina NovaSeq X Plus with a NovaSeq X Series 1.5B Reagent Kit (100 Cycle) (Illumina #20104703) or Illumina NextSeq 500 with a NextSeq 500/550 Mid Output Kit v2.5 (150 Cycles) (Illumina #20024904) for 2×50 pair-end reads. Around 30M reads per sample were obtained.

Output pair-end reads from sequencing were aligned with HISAT2 and then converted to sorted bam files with samtools. Then these files were processed using featureCounts to get a count matrix of each gene. Finally, the matrices were used as input for DESeq2 to quantify off-target effects in the transcriptome. Volcano plots were created using R. All computational work was performed on the University of Florida's HPC HiperGator.

### Lentiviral Cell Line Production of AsCas12a-GFP-Expressing HEK293T Cells

One day before transfection, 7×10⁶ cells were seeded in a 10 cm plate in 12 mL of a 50% mixture of DMEM high glucose GlutaMAX™ supplement pyruvate (Gibco #10569010) with 10% Fetal Bovine Serum (Gibco #A3160902), and 50% Opti-MEM™ Reduced Serum Medium (Gibco, #31985062). On the day of transfection, two tubes were prepared, Tube A contained 1.5 mL of Opti-MEM and 41 μL of Lipofectamine 3000 Reagent (Invitrogen, #L3000001). Tube B contained 1.5 mL of Opti-MEM, 35 μL of P3000 reagent, 1.3 μmol of psPAX2 plasmid (Addgene #12260), 0.72 μmol of pMD2.G plasmids (Addgene #12259), and 1.64 μmol of AsCas12a/GFP transfer plasmid. Then Tube A was added to Tube B, pipette mixed and incubated for 20 min. At the moment of transfection, 6 mL of media were withdrawn from the 10 cm plate and ~3 mL of the transfection mixture was added dropwise. 6 hours post transfection, all packaging medium was removed and 12 mL of the mixture of 50% DMEM (w/10% FBS) and 50% Opti-MEM was added. 24 hours post transfection the cell supernatant was harvested and stored at 4° C. and media was replenished with 12 mL of the DMEM/OptiMEM mixture. 52 hours post transfection the supernatant was harvested for a second time and added to the first collection. Cells were discarded afterwards.

After two collections the 24 mL of cell supernatant were centrifuged at 2,000 rpm and then filtered with 45 μm syringe filter (Sigma, #SLHPR33RS). After filtration, the viral supernatant was concentrated on 10× with Lenti-XTM Concentrator buffer (Takara, #631231) as per the manufacturer's instructions.

For virus infection, reverse transduction was performed. In a 6-well plate, varying volumes between 15-150 μL of concentrated viral particles were added to each well along with 5×10⁴ HEK293T cells in DMEM with 10 μg/mL of Polybrene (Sigma, #TR-1003). After 72 hours, media was replaced with DMEM (+10% FBS) with 10 μg/mL of Blasticidin S HCl (Gibco, #A1113903) for antibiotic selection. For all RNA KD experiments, this cell line was passaged a maximum of 3 times before AsCas12a epigenetic downregulation.

### RNA Immunoprecipitation and Quantitative PCR (RIP-qPCR)

RNA immunoprecipitation was performed as previously described. HA-AsCas12a or HA-GFP and ΨPPIA transfected HEK293T cells were fixed with 1% paraformaldehyde (ChemCruz #sc281692) for 15 minutes at room temperature. After fixation, the paraformaldehyde was removed, and 125 mM glycine in PBS was added to quench the crosslinking, followed by a 10-minute incubation. Cells were washed twice with ice-cold PBS, harvested by scraping, and the cell suspension was centrifuged at 1000 g for 5 minutes to pellet the cells. The cell pellets were re-suspended in 2 volumes of lysis buffer (150 mM NaCl, 10 mM Tris-HCl PH 7.6, 2 mM EDTA, 0.5% NP-40, 0.5 mM DTT, 400 U/mL RNase inhibitor (NEB #M0314S)) and mixed thoroughly. The lysates were incubated on ice for 20 minutes. Insoluble material was pelleted by centrifugation at 15,000 g for 15 minutes at 4° C., and the supernatant, containing the cleared lysate, was used for pulldown with Protein G Agarose beads (Sigma #16-201). Set aside 30 μL of the supernatant as input.

To conjugate antibodies to agarose beads, 20 μL of Protein G Agarose beads per sample for immunoprecipitation were washed twice and resuspended in 300 μL of wash buffer (150 mM NaCl, 50 mM Tris-HCl, pH 7.6, 2 mM EDTA, 0.05% NP-40, 0.5 mM DTT, 200 U/mL RNase inhibitor). Then, 2 μL of anti-HA antibody (Cell Signaling Technology #3724) was added. The sample was incubated for 2 hours at 4° C. on a rotator to allow the antibody to conjugate to the beads. After incubation, the beads were pelleted by centrifugation, the supernatant was removed, and 100 μL of sample lysate was added to the beads and rotated overnight at 4° C.

After incubation with sample lysate, beads were pelleted, washed three times with high-salt buffer (300 mM NaCl, 50 mM Tris-HCl PH 7.6, 2 mM EDTA, 0.05% NP-40, 0.5 mM DTT, 200 U/mL RNase inhibitor), and then resuspend with SDS solution (1% SDS, 10 mM EDTA (pH 8.0), 50 mM Tris-HCl, pH 7.4, 200 U/mL RNase inhibitor). Proteins were then digested by addition of Proteinase K (NEB #P8102S) to a final concentration of 1.2 mg/mL and incubated at 55° C. for 4 hours. TRIzol (Invitrogen #15596018) was added for denaturation and RNA purification. Purified RNA was reverse transcribed and amplified by Luna® Universal Probe One-Step RT-qPCR Kit. Fold change was calculated relative to input using the ddCt method.

### Methylated RNA Immunoprecipitation and Quantitative PCR (MeRIP-qPCR)

MeRIP-qPCR is modified on previously described. 5 μg Total RNA was fragmented in solution of 50 mM Tris-HCl, pH 8.0, 50 mM MgCl2 and heated at 95° C. for 8 min. 10% of the sample was saved as input. Remaining fragmented RNA was processed to m6A immunoprecipitation:

30 μL of Protein G beads (Sigma #16-201) were washed twice by IP reaction buffer (150 mM NaCl, 10 mM Tris-HCl, pH 7.5, 0.1% NP-40), resuspended in 500 μL of reaction buffer, and tumbled with 3 μL of anti-m6A antibody (CST #56593S) at 4° C. overnight. Wash twice by reaction buffer, the antibody-bead mixture was resuspended in 500 μL of the reaction buffer containing 4.5 μg of fragmented total RNA and 2 μL of RNase Inhibitor (NEB #M0314S). Incubated at 4 hours at 4° C.

Samples were washed 5 times with reaction buffer. Add 5 μL of proteinase K (Thermo #EO0491) and incubate the sample at 37° C. for 60 min, and 56° C. for 15 min. Input and IP RNA samples were extracted by TRIzol (Invitrogen #15596018).

Purified RNA was reverse transcribed and amplified by TaqMan™ Fast Virus 1-Step Master Mix (Thermo #4444436). Fold change was calculated relative to input using the ddCt method.

### Ribo-Seg for Ribosomal Profiling

Similarly to what was described in the mammalian cell transfection section, HEK293T cells were seeded at 5×10⁵ cells per well and were seeded in 6-well plates 48 hours before transfection. Then, 1 μg of AsCas12a-GFP and 2 μg of ΨDNA (ΨPPIA or ΨNT), and 8 μL of TransIT-X2® were added to 250 μL of Opti-MEM™ Reduced Serum Medium. This per well of 6-well plate. After 15 hours, cells were trypsinized and resuspended in 2% FBS in PBS. Per each condition (ΨPPIA or ΨNT) all six wells in a plate were pooled together as a single biological replicate, two plates per condition were made. Cells were later subjected to GFP cell sorting on a BD FACSymphony™ S6 flow cytometer. Around 5 million positive GFP cells were obtained per sample.

After sorting, 20% of the total GFP+ cells were separated for total RNA extraction using Monarch Total RNA Miniprep Kit (NEB #T2010S) as per the manufacturer's instructions. The extracted RNA was used to perform mRNA-Seq as described above. The remaining 80% of the cells were used for ribosomal profiling. The Ribo-Seq library was constructed using ALL-IN-ONE RiboLace Gel Free (IMMAGINA Biotechnology #GF001-12). The steps were performed as per the manufacturer's instructions, but in short cells were first treated with 10 μg/mL of cycloheximide in DMEM for 5 min at 37° C. and later washed once in cold PBS with 10 μg/mL of cycloheximide. Then the cells lysed with the provided lysis buffer and later digested with the provided nuclease. Then ribosomes were pulled down using proprietary magnetic beads bound to a puromycin conjugate to bind to active ribosomes. RNA was then extracted and then library was prepared by phosphorylation, adaptor ligation, circularization, reverse transcription and two rounds of PCR all using the provided enzymes on the kit. QC was performed on the final libraries through a 2100 Agilent Bioanalyzer, qPCR, and Qubit.

Samples were pooled together and loaded into an Illumina NovaSeq X Plus with a NovaSeq X Series 1.5B Reagent Kit (100 Cycle) (Illumina #20104703) for 1×10⁰ single-end reads. Around 300M reads per sample were obtained.

To analyze the sequencing data demultiplexing and QC was performed using bcl-convert and fastp. Then cutadapt used to discard shorter reads (--minimum-length 29) and trim the linker sequenced used to prepare the library. Then UMI-tools was used to extract the UMIs. Bowtie2 was later used to filter out rRNA and tRNA from the trimmed reads. The final reads were aligned to the hg38 genome using STAR.

BAM files output by STAR were later fed into RiboWaltz for quality control of the ribosomal profiling data. P-site offset was determined to be 12 nucleotides from the 5′ end for all samples and regions of the p-sites and trinucleotide periodicity were of high quality (FIGS. 18E-18F).

Lastly the mRNA-Seq and Ribo-Seq data were fed into featureCounts to obtain gene counts. The count matrix was later fed into RiboDiff to detect changes in translation efficiency. Translation efficiency data for each gene was then plotted on a volcano plot using R Studio.

### CLIP-seq

CLIP-seq was performed as previously described with minor modifications. Briefly, 4-thiouridine (4SU) was added to a final concentration of 200 UM to HEK293T cells (15 million cells per sample) stably expressing AsCas12a, followed by incubation at 37° C. Cells were UV-crosslinked twice at 365 nm with an energy of 150 mJ/cm² using a UV crosslinker.

Following crosslinking, cells were washed, lysed, and partially digested with RNase T1 (Thermo Fisher #EN0541) to a final concentration of 0.2 U/μL. Clarified lysates were subjected to immunoprecipitation using Dynabeads™ Protein G (Invitrogen #10-003-D) pre-conjugated with a monoclonal anti-AsCas12a antibody (Cell Signaling Technology #E1U7C). Ten micrograms of antibody were used per 100 μL bead slurry for each 1 mg of input lysate.

After immunoprecipitation, beads were washed three times with IP wash buffer and subjected to a second RNase T1 digestion at a final concentration of 10 U/μL for 10 min at 22° C. Beads were subsequently washed three times with high-salt buffer and then resuspended in 300 μL G-50 buffer (20 mM Tris-HCl, pH 7.5, 300 mM sodium acetate, 2 mM EDTA, 0.25% SDS, RNaseOUT) and treated with 2 μL proteinase K (Invitrogen #25530049) at 37° C. for 15 min with shaking at 1,200 rpm.

RNA was extracted using phenol/chloroform/isoamyl alcohol, and enriched RNA fragments were used for library construction with the NEBNext Ultra II Directional RNA Library Prep Kit for Illumina (NEB #E7760S). Purified cDNA libraries were sequenced at the University of Florida Next-Generation Sequencing Core Facility on an Illumina NovaSeq S4 2×150 platform, generating approximately 50 million reads per sample.

Dual RNA-DNA Editing with AsCas12a

5×10⁵ cells per mL were seeded in plates 48 hours before transfection as previously stated. Then 350 ng of AsCas12a-GFP or GFP-only plasmid and 650 ng of ΨNT or 325 ng ΨPPIA/RPL4 with 325 ng of crCCR5 were transfected with 2 μL of TransIT-X2® and 50 μL of Opti-MEM™ Reduced Serum Medium. Complexes were allowed to form for 25 minutes and then added dropwise to each well of pre-seeded 48-well plates containing HEK293T cells. After 18 hours, cells were trypsinized and washed with PBS and then split on three equal fractions. One fraction was used for RNA extraction using the method stated previously, the other fraction was used for DNA extraction with 30 μL of QuickExtract™ DNA Extraction Solution (Biosearch #QE09050), lastly the third fraction was reseeded and posteriorly DNA extracted after 50 hours. The extracted RNA was subjected to qPCR and mRNA-Seq as described above, and DNA was subjected to amplicon sequencing as described as well. DNA sequencing data was interpreted and quantified using CRISPResso2.

### Proximity Ligation Assay (PLA)

5×10⁵ cells per mL were seeded in Nunc™ Lab-Tek™ Chamber Slide System (Thermo Scientific #177445PK) 48 hours before transfection as previously stated. Then 350 ng of AsCas12a-GFP or dRfxCas13d-GFP plasmid and 650 ng ΨPPIA with 2 μL of TransIT-X2® and 50 μL of Opti-MEM™ Reduced Serum Medium. After 12 hours of transfection, cells were washed twice with PBS for 5 min, and then fixed with 4% PFA in PBS for 10 min. at RT (Thermo Scientific #28906) and then washed twice again for 5 min. each. Cells were then permeabilized with 0.5% Triton X-100 (Sigma #X100-100ML) for 10 min. at RT. Cells were then washed again twice in PBS for 5 min. Then the PLA assay was performed using Duolink® In Situ Red Starter Kit Mouse/Rabbit (Sigma #DUO92101-1KT) as per manufacturer's instructions. The antibodies used for protein labeling are HA-Tag Antibody (F-7) (Santa Cruz #sc-7392) for AsCas12a and dRfxCas13d, and Anti-RNase H1/RNH1 antibody (abcam #ab229078) and samples were incubated at 4° C. overnight.

Remaining steps were performed exactly as detailed on the instructions. Microscopy images were taken with a Leica DFC 7000 T.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Abudayyeh, O. O. et al. A cytosine deaminase for programmable single-base RNA editing. Science 365, 382-386 (2019).
2. Abudayyeh, O. O. et al. RNA targeting with CRISPR-Cas13. Nature 550, 280-284 (2017).
3. Apostolopoulos, A. et al. dCas13-mediated translational repression for accurate gene silencing in mammalian cells. Nature Communications 15, 2205 (2024).
4. Asahi-Ozaki, Y., et al. Quantitative analysis of Kaposi Sarcoma-Associated Herpesvirus (KSHV) in KSHV-associated diseases. The Journal of infectious diseases 193, 773-782 (2006).
5. Chen, J. et al. Trans-nuclease activity of Cas9 activated by DNA or RNA target binding. Nature Biotechnology, 1-11 (2024).
6. Chen, J. S. et al. CRISPR-Cas12a target binding unleashes indiscriminate single-stranded DNase activity. Science 360, 436-439 (2018).
7. Cong, L. et al. Multiplex genome engineering using CRISPR/Cas systems. Science 339, 819-823 (2013).
8. Cox, D. B. et al. RNA editing with CRISPR-Cas13. Science 358, 1019-1027 (2017).
9. Deltcheva, E. et al. CRISPR RNA maturation by trans-encoded small RNA and host factor RNase III. Nature 471, 602-607 (2011).
10. Doma, M. K. et al. Endonucleolytic cleavage of eukaryotic mRNAs with stalls in translation elongation. Nature 440, 561-564 (2006).
11. Donohoue, P. D. et al. Conformational control of Cas9 by CRISPR hybrid RNA-DNA guides mitigates off-target activity in T cells. Molecular Cell 81, 3637-3649. e3635 (2021).
12. Filippova, J., et al. Guide RNA modification as a way to improve CRISPR/Cas9-based genome-editing systems. Biochimie 167, 49-60 (2019).
13. Fu, Y., et al. Improving CRISPR-Cas nuclease specificity using truncated guide RNAs. Nature biotechnology 32, 279-284 (2014).
14. Gasiunas, G., et al. Cas9-crRNA ribonucleoprotein complex mediates specific DNA cleavage for adaptive immunity in bacteria. Proceedings of the National Academy of Sciences 109, E2579-E2586 (2012).
15. Gootenberg, J. S. et al. Multiplexed and portable nucleic acid detection platform with Cas13, Cas12a, and Csm6. Science 360, 439-444 (2018).
16. Gootenberg, J. S. et al. Nucleic acid detection with CRISPR-Cas13a/C2c2. Science 356, 438-442 (2017).
17. Hafner, M. et al. PAR-CliP-a method to identify transcriptome-wide the binding sites of RNA binding proteins. Journal of visualized experiments: JoVE, 2034 (2010).
18. He, P. C. et al. m6A RNA methylation: from mechanisms to therapeutic potential. The EMBO journal 40, e105977 (2021).
19. Jiang, F. et al. CRISPR-Cas9 structures and mechanisms. Annual review of biophysics 46, 505-529 (2017).
20. Jinek, M. et al. A programmable dual-RNA-guided DNA endonuclease in adaptive bacterial immunity. science 337, 816-821 (2012).
21. Kartje, Z. J., et al. Chimeric guides probe and enhance Cas9 biochemical activity. Biochemistry 57, 3027-3031 (2018).
22. Kim, H. et al. Enhancement of target specificity of CRISPR-Cas12a by using a chimeric DNA-RNA guide. Nucleic acids research 48, 8601-8616 (2020).
23. Konermann, S. et al. Transcriptome engineering with RNA-targeting type VI-D CRISPR effectors. Cell 173, 665-676. e614 (2018).
24. Li, B. et al. Engineering CRISPR-Cpf1 crRNAs and mRNAs to maximize genome editing efficiency. Nature biomedical engineering 1, 0066 (2017).
25. Liu, L. et al. C2c1-sgRNA complex structure reveals RNA-guided DNA cleavage mechanism. Molecular cell 65, 310-322 (2017).
26. Mir, A. et al. Heavily and fully modified RNAs guide efficient SpyCas9-mediated genome editing. Nature Communications 9, 2641 (2018).
27. Moon, J. et al. Asymmetric CRISPR enabling cascade signal amplification for nucleic acid detection by competitive crRNA. Nature Communications 14, 7504 (2023).
28. Mu, K., et al. RASP v2. 0: an updated atlas for RNA structure probing data. Nucleic Acids Research 53, D211-D219 (2025).
29. Nguyen, L. T. et al. A thermostable Cas12b from Brevibacillus leverages one-pot discrimination of SARS-COV-2 variants of concern. EBioMedicine 77 (2022).
30. Nguyen, L. T. et al. Harnessing noncanonical crRNAs to improve functionality of Cas12a orthologs. Cell reports 43 (2024).
31. Nguyen, L. T., et al. Clinical validation of engineered CRISPR/Cas12a for rapid SARS-COV-2 detection. Communications Medicine 2, 7 (2022).
32. Nguyen, L. T., et al. Enhancement of trans-cleavage activity of Cas12a with engineered crRNA enables amplified nucleic acid detection. Nature communications 11, 4906 (2020).
33. Qiao, J. et al. A split crRNA with CRISPR-Cas12a enables highly sensitive, selective, and multiplexed detection of RNA and DNA. bioRxiv, 2024.2001. 2004.574169 (2024).
34. Ramachandran, A. et al. CRISPR enzyme kinetics for molecular diagnostics. Analytical chemistry 93, 7456-7464 (2021).
35. Rananaware, S. R. et al. PAM-free diagnostics with diverse type V CRISPR-Cas systems. medRxiv, 2024.2005. 2002.24306194 (2024).
36. Rananaware, S. R. et al. Programmable RNA detection with CRISPR-Cas12a. Nature communications 14, 5409 (2023).
37. Rauch, S. et al. Programmable RNA-guided RNA effector proteins built from human parts. Cell 178, 122-134. e112 (2019).
38. Rozners, E. Chemical modifications of CRISPR RNAs to improve gene-editing activity and specificity. Journal of the American Chemical Society 144, 12584-12594 (2022).
39. Ryczek, M., et al. Overview of methods for large-scale RNA synthesis. Applied Sciences 12, 1543 (2022).
40. Shebanova, R. et al. Efficient target cleavage by Type V Cas12a effectors programmed with split CRISPR RNA. Nucleic acids research 50, 1162-1173 (2022).
41. Stella, S., et al. Structure of the Cpf1 endonuclease R-loop complex after target DNA cleavage. Nature 546, 559-563 (2017).
42. Swarts, D. C., et al. Structural basis for guide RNA processing and seed-dependent DNA targeting by CRISPR-Cas12a. Molecular cell 66, 221-233. e224 (2017).
43. Teng, F. et al. CDetection: CRISPR-Cas12b-based DNA detection with sub-attomolar sensitivity and single-base specificity. Genome biology 20, 1-7 (2019).
44. Tieu, V. et al. A versatile CRISPR-Cas13d platform for multiplexed transcriptomic regulation and metabolic engineering in primary human T cells. Cell 187, 1278-1295. e1220 (2024).
45. Tong, H. et al. High-fidelity Cas13 variants for targeted RNA degradation with minimal collateral effects. Nature biotechnology 41, 108-119 (2023).
46. Yang, L.-Z. et al. Dynamic imaging of RNA in living cells by CRISPR-Cas13 systems. Molecular cell 76, 981-997. e987 (2019).
47. Zetsche, B. et al. Cpf1 is a single RNA-guided endonuclease of a class 2 CRISPR-Cas system. cell 163, 759-771 (2015).
48. Zhang, H., et al. Mechanisms for target recognition and cleavage by the Cas12i RNA-guided endonuclease. Nature structural & molecular biology 27, 1069-1076 (2020).
49. Zhao, W. et al. POSTAR3: an updated platform for exploring post-transcriptional regulation coordinated by RNA-binding proteins. Nucleic acids research 50, D287-D294 (2022).

What is claimed is:

1. A nucleic acid detection system comprising:
a CRISPR system comprising a Cas12 protein and one or more guide nucleotides; and
an ssDNA comprising a fluorophore and a quencher;
wherein each guide nucleotide comprises a guide sequence capable of binding a target nucleic acid sequence and forming a complex with the Cas12 protein;
wherein the guide nucleotide comprises a fluorophore and a quencher; and
wherein when the guide nucleotide binds the target nucleic acid sequence and forms a complex with the Cas12 protein, the Cas12 protein performs trans cleavage on the ssDNA, separating the fluorophore from the quencher.

2. The nucleic acid detection system of claim 1, wherein the Cas12 protein comprises a Cas12i protein, a Cas12j protein, a Cas12b protein, a Cas12a protein, or any combination thereof.

3. The nucleic acid detection system of claim 2, wherein the Cas12i protein comprises Cas12i1, Cas12i2, or any combination thereof.

4. The nucleic acid detection system of claim 2, wherein the Cas12a protein comprises AsCas12a, ErCas12a, or any combination thereof.

5. The nucleic acid detection system of claim 1, wherein the guide nucleotide comprises a spacer, wherein the spacer comprises the guide sequence, and wherein the spacer is from about 20 to about 23 nucleotides long.

6. The nucleic acid detection system of claim 1, wherein the guide nucleotide further comprises a scaffold.

7. The nucleic acid detection system of claim 6, wherein the scaffold is located at a 3′ end of the guide sequence and wherein the scaffold is from about 19 to about 25 nucleotides long.

8. The nucleic acid detection system of claim 1, wherein the guide nucleotide comprises DNA and wherein the target nucleic acid sequence comprises RNA.

9. A method for detecting a target nucleic acid, the method comprising:
(a) contacting a sample suspected of containing the target nucleic acid with the nucleic acid detection system of claim 8; and
(b) measuring a fluorescence signal from the nucleic acid detection system;
wherein when the fluorescence signal is present, the sample contains the target nucleic acid sequence.

10. The method of claim 9, wherein the sample comprises a biological sample from a subject, wherein the biological sample is selected from blood, saliva, urine, stool, cerebrospinal fluid (CSF), sputum, tissue biopsy, amniotic fluid, bone marrow, plasma, serum, or mucus.

11. The method of claim 9, wherein the target nucleic acid sequence is associated with a cardiovascular disease, a neurological disorder, cancer, a bacterial disease, or a viral disease, an agricultural pest, or an environmental contaminant.

12. The method of claim 11, wherein the viral disease comprises HIV, hepatitis C virus (HCV), dengue virus, zika virus, or any combination thereof.

13. The method of claim 11, wherein the bacterial disease is caused by Francisella hispaniensis FSC454, Moraxella bovoculi, Helcococcus kunzii, Sneathia amnii SN35, Alicyclobacillus acidoterrestris, Francisella tularensis, or any combination thereof.

14. The method of claim 9, wherein the method is capable of detecting from about 50 pM to about 250 pM of the target nucleic acid in the biological sample, optionally wherein the target sequence does not need to be amplified prior to performing the method.

15. A method for silencing a target nucleic acid, the method comprising contacting a cell, tissue, or living organism containing the target nucleic acid with the nucleic acid detection system of claim 1, optionally wherein silencing is transient.

16. The method of claim 15, wherein the target nucleic acid is an mRNA molecule and wherein performing the method induces ribosome stalling, thereby repressing protein translation.

17. The method of claim 15, wherein the cell is a cancer cell.

18. The method of claim 17, wherein the cancer cell comprises a cervical cancer cell, a liver cancer cell, or a breast cancer cell.

19. A method for site-specific modification of a target RNA, the method comprising contacting the RNA with a CRISPR system comprising a Cas12 protein fused to an N6-methyladenosine methyltransferase and one or more guide nucleotides;
wherein each guide nucleotide comprises a guide sequence capable of binding the target RNA and forming a complex with the Cas12 protein; and
wherein when the guide nucleotide binds the target RNA and forms a complex with the Cas12 protein, the N6-methyladenosine methyltransferase methylates an N6 position of at least one adenine in the target RNA.

20. The method of claim 19, wherein the N6-methyladenosine methyltransferase comprises METTL3 and wherein the Cas12 protein comprises AsCas12a.
